(19) United States
(12) Patent Application Publication
(10) Pub. No.: US 2023/0404441 A1
(43) Pub. Date: Dec. 21, 2023

(54) SYSTEMS, DEVICES, AND METHODS FOR MEAL-RELATED ANALYTE RESPONSE MONITORING

(71) Applicant: ABBOTT DIABETES CARE INC., Alameda, CA (US)

(72) Inventors: Sebastian Kracht, Kronberg im Taunus (DE); Matthew Withersby, Bicester (GB); Nithin Baby, Wiesbaden (DE); Alicia del Carmen Moder-Pinana, Alameda, CA (US); Sofia Giovannella, Rome (IT); Irene Sanchez Turon, Esplugues de Llobregat (ES); Sarit Guha Thakurta, Aalen (DE); Rikke Borgesen, Hellerup (DK)

(21) Appl. No.: 18/139,314

(22) Filed: Apr. 25, 2023

Related U.S. Application Data
(60) Provisional application No. 63/452,263, filed on Mar. 15, 2023.
(60) Provisional application No. 63/335,030, filed on Apr. 26, 2022.

(51) Int. Cl. A61B 5/145; A61B 5/00; G16H 20/60

(52) U.S. Cl. CPC A61B 5/14532; A61B 5/7435; A61B 5/7264; G16H 20/60; A61B 5/7282

(57) ABSTRACT

Systems, devices, and methods for detecting, measuring and classifying meals for an individual based on analyte measurements. These results and related information can be presented to the individual to show the individual which meals are causing the most severe analyte response. These results can be organized and categorized based on preselected criteria or previous meals and results so as to organize and present the results in a format with reference to glucose as the monitored analyte. Various embodiments disclosed herein relate to methods, systems, and software applications intended to engage an individual by providing direct and timely feedback regarding the individual's meal-related glycemic response.

### CROSS-REFERENCE TO RELATED APPLICATIONS

This application claims priority to U.S. Application Ser. No. 63/452,263, filed Mar. 15, 2023, and U.S. Application Ser. No. 63/335,030, filed Apr. 26, 2022, both of which are hereby expressly incorporated by reference in their entireties for all purposes.

### FIELD

The present subject matter broadly relates to systems, devices, and methods for the collection of information about analyte levels of individuals and information about meals that those individuals consume. The present subject matter further relates to processing, analyzing, and/or presenting this information for the purpose of meal-related analyte response monitoring, and providing insights, activities, and recommendations for those individuals.

### BACKGROUND

The increased prevalence of Type 2 diabetes and metabolic syndrome over the past few decades has been attributed to changing diet and activity levels. For example, consumption of more readily available high glycemic index foods can cause rapid post-prandial increase of blood glucose and insulin levels, which has a positive association with weight gain and obesity. These conditions can be further traced to an increased risk of developing these and other diseases.

Most people generally understand the importance of their diet. However, in practice, many people struggle with translating this general awareness to their specific food choices. These problems exist primarily because people cannot directly see the impact of their choices. This can lead to misconceptions around food portion size, misunderstandings about which foods are relatively healthy, and a general lack of awareness regarding the necessary duration and intensity of activity to maintain good health. These problems are further exacerbated by advertisements, habits, peer pressure, food preferences, and recommendations based on generalizations.

To address these issues, an individual's physiological responses can be tracked and better understood by analyte monitoring systems. Because high glucose levels are primarily driven by the consumption of food, the level of post-prandial glucose can relate to the amount of carbohydrates and other meal components consumed by the individual, as well as to the individual's physiological response to meals. However, a challenge for analysis of this influx of data is to represent the data in a meaningful manner that enables efficient action. Data relating to meal selection, and the subsequent impact, should be understood on a clinical basis, as well as a personal basis for the individual, the meal administrator, and/or the medical professional to understand and moderate glucose excursions, such as episodes of hyperglycemia.

Prior attempts to implement software for tracking a user's meal consumption and correlating that to the user's analyte data suffer from numerous deficiencies. For example, some systems require that the individual perform numerous inconvenient and uncomfortable discrete blood glucose measurements (e.g., finger stick blood glucose tests). These solutions can also suffer from an insufficient number of data points to adequately determine a glycemic response to a meal. For example, the individual may perform a discrete blood glucose measurement at a time before or after the time when the user's glycemic response peaks, making it difficult to accurately ascertain the glycemic response, and to meaningfully compare meals based on the glycemic response. A deficiency in data points can also make it difficult to automatically detect the occurrence of a meal event in the user's analyte data. Thus, some prior systems place significant reliance upon manual logging of meals by the user.

Prior art systems that seek to detect meal events based simply on the existence of a rise in glucose levels, such as U.S. Patent Publication No. 2003/0208113, are inadequate because they fail to take into account the user's prior meal history and thus can overestimate the number of meals the user has consumed.

Thus, improved systems, devices, and methods for meal information collection, meal assessment and detection, and correlation to analyte levels are needed.

### SUMMARY

Provided herein are example embodiments of systems, devices, and methods for detecting, measuring, and classifying meals for a human individual in relation to that individual's analyte measurements. These individuals can be those exhibiting or diagnosed with a diabetic condition, those considered as pre-diabetic, those with metabolic syndrome, and even those without diabetes, pre-diabetic, or metabolic syndrome conditions. These individuals can be any person motivated to improve his or her health by adjustment to his or her diet and/or activity practices. Resulting information can be presented to the individual to show which meals or aspects of the meals are causing the most impact on analyte levels.

According to a first aspect of the present disclosure, there is provided a system for monitoring meal-related analyte responses in a user, the system comprising: a reader device, comprising: wireless communication circuitry configured to receive data indicative of an analyte level of the user, one or more processors coupled with a memory, the memory storing a meal monitoring application that, when executed by the one or more processors, causes the one or more processors to output a first challenge graphical user interface (GUI) reflecting a list of one or more challenges relating to the user's analyte response, wherein the one or more challenges comprise one or more active challenges, one or more completed challenges, and one or more unattempted challenges, the first challenge GUI comprising a first challenge card, a second challenge card, and a third challenge card, wherein the first challenge card reflects the one or more active challenges, wherein each of the one or more active challenges reflects a challenge currently in progress by the user on the meal monitoring application, wherein the second challenge card reflects the one or more completed challenges, wherein each of the one or more completed challenges reflects a challenge completed by the user, and, wherein the third challenge card reflects one or more unattempted challenges, wherein each of the one or more unattempted challenges reflects a challenge in which the user has not yet participated in.

A meal monitoring application can store challenges in a database. The stored challenges can be outputted to every user in the same manner or may be personalized based on data analyzed by the meal monitoring application. Challenges may be outputted to a category of users based on shared characteristics, demographics, location, behavior or activities. For example, a pizza challenge may be outputted to users in which the meal monitoring application determines eat pizza (e.g., to users that ate a threshold amount of pizza within a predetermined period of time). Challenges can be generated to the user based on various different criteria or behaviors. For example, challenges can be outputted to a user based on a particular user cohorts or characteristics identified by the meal monitoring application; psychographics; information inputted to the meal monitoring application by the user, such as diary entries; timing of meals consumed by the user; demographics; geographical considerations, such as activities happening in a particular location or region, and; seasonal activities. Challenges may be generated and displayed on the meal monitoring application in a predetermined order or an order based on particular criteria or behaviors analyzed by the meal monitoring application.

The data indicative of an analyte level of the user may include the user's analyte response. The user's analyte response may include a meal-related analyte response. The user's analyte response may include a user's glucose levels.

In some embodiments, the first challenge card of the system comprises one or more selectable first challenge icons, wherein each of the one or more selectable first challenge icons reflects a challenge currently in progress by the user.

In some embodiments, each of the one or more selectable first challenge icons comprises a first indicator, a picture and a textual description relating to the challenge currently in progress, wherein the first indicator is displayed on the picture and is configured to indicate that the challenge is currently in progress. In some embodiments, the first indicator is a green dot.

In some embodiments, the second challenge card comprises one or more selectable second challenge icons, wherein each of the one or more selectable second challenge icons reflects a challenge completed by the user.

In some embodiments, each of the one or more selectable second challenge icons comprises a second indicator, a picture and a textual description relating to the completed challenge, wherein the second indicator is overlayed on the picture and is configured to indicate that the challenge has been completed by the user. In some embodiments, the second indicator is a colored check mark.

In some embodiments, the third challenge card comprises one or more selectable third challenge icons, wherein each of the one or more selectable third challenge icons reflects a challenge not yet tried by the user. In some embodiments, each of the one or more selectable third challenge icons comprises a picture and a textual description relating to the completed challenge.

In some embodiments, each of the first challenge card, the second challenge card, and the third challenge card include a plurality of selectable challenge icons, wherein a first set of the plurality of selectable challenge icons is displayed on the first challenge GUI, wherein the reader device further comprises a touchscreen, and wherein the meal monitoring application, when executed by the one or more processors, further causes the one or more processors to: receive input from the touchscreen corresponding to a swipe gesture or a drag gesture, and in response to the received input, display a second set of the plurality of selectable challenge icons on the first challenge GUI, wherein at least one or more of the plurality of selectable challenge icons of the second set is different than at least one or more of the plurality of selectable challenge icons of the first set.

In some embodiments, the list of one or more challenges includes one or more selectable challenge icons, wherein each of the one or more selectable challenge icons corresponds to one of the one or more challenges relating to the user's analyte response or glucose levels, wherein the meal monitoring application, when executed by the one or more processors, further causes the one or more processors to: in response to a selection of one of the one or more selectable challenge icons, output a second challenge GUI reflecting contextual information related to the one of the one or more challenges corresponding to the selected one of the one or more selectable challenge icons.

The contextual information can be a separate screen explaining the one of the one or more challenges corresponding to the selected one of the one or more selectable challenge icons. The contextual information can provide information related to a respective challenge. For example, if the one of the one or more challenges corresponding to the selected one of the one or more selectable challenge icons relates to eating vegetables and fruits, the contextual information can provide a contextual description of the importance or relevance of consuming vegetables and fruits along with a description of the challenge.

In some embodiments, the second challenge GUI comprises: a challenge profile section comprising the selected one of the one or more selectable challenge icons, a picture, and a challenge title providing a textual description of the one of the one or more challenges corresponding to the selected one of the one or more selectable challenge icons; an attempt indicator configured to indicate when the one of the one or more challenges corresponding to the selected one of the one or more selectable challenge icons was last attempted by the user; and a completion indicator configured to indicate when the one of the one or more challenges corresponding to the selected one of the one or more selectable challenge icons was last successfully completed by the user.

In some embodiments, if the one of the one or more challenges corresponding to the selected one of the one or more selectable challenge icons is an unattempted challenge, then the second challenge GUI further comprises a start button, wherein the meal monitoring application, when executed by the one or more processors, further causes the one or more processors to: in response to a selection of the start button, begin the unattempted challenge on the meal monitoring application.

In some embodiments, the meal monitoring application, when executed by the one or more processors, further causes the one or more processors to: in response to a selection of the start button, begin the unattempted challenge on the meal monitoring application on a following day.

In some embodiments, if the one of the one or more challenges corresponding to the selected one of the one or more selectable challenge icons is an active challenge, then the second challenge GUI further comprises a stop button, wherein the meal monitoring application, when executed by the one or more processors, further causes the one or more processors to: in response to a selection of the stop button, cease continuation of the active challenge.

In some embodiments, if the one of the one or more challenges corresponding to the selected one of the one or more selectable challenge icons is an active challenge, the second challenge GUI further comprises a progress card configured to indicate progress the user has made towards the active challenge, wherein the progress card comprises a unit of measure and a unit of time to indicate the progress. In some embodiments, the unit of measure includes a fractional unit and the unit of time includes a number of days.

In some embodiments, a modal is displayed on the second challenge GUI if the one of the one or more challenges corresponding to the selected one of the one or more selectable challenge icons is an active challenge, wherein the modal is configured to prompt the user to provide progress information related to the active challenge in the meal monitoring application.

In some embodiments, the meal monitoring application is configured to detect whether the user successfully completed the active challenge based on the tracked progress, wherein the meal monitoring application, when executed by the one or more processors, further causes the one or more processors to: in response to the meal monitoring application detecting the user successfully completed the active challenge, output a third challenge GUI, wherein the third challenge GUI comprises a challenge profile section, an attempt indicator, a completion indicator, and a message congratulating the user on successfully completed the active challenge, and in response to the meal monitoring application detecting the user successfully completed the active challenge, identify the active challenge as a completed challenge, wherein the third challenge GUI further comprises a first button which, when selected by the user, is configured to restart the completed challenge, and wherein the third challenge GUI further comprises a second button which, when selected by the user, outputs the first challenge GUI, wherein the user selects a different challenge from the list of one or more challenges reflected by the first challenge GUI.

The meal monitor application can be configured to automatically detect whether progress was successfully made towards a challenge based on meal entries or the analyte level variance value associated with meal entries. For example, the meal monitor application can automatically detect what the user consumed four consecutive “green impact” or low glycemic response meals.

In some embodiments, a modal is displayed on the third challenge GUI in response to the user selecting the first button, and wherein the modal is configured to prompt the user to confirm whether the user would like to restart the completed challenge.

The modal may comprise information related to a completed challenge. The modal may be a more vibrant and visual modal within the application that provides more context for prompting action. The modal may present a graphic and text directed to prompting the user to take a particular action. The modal may include possible answers such that the user can indicate whether they would like to restart a challenge.

In some embodiments, the meal monitoring application is configured to detect whether the user successfully completed the active challenge based on the tracked progress, wherein the meal monitoring application, when executed by the one or more processors, further causes the one or more processors to: in response to the meal monitoring application detecting the user did not successfully complete the active challenge, output a fourth challenge GUI, wherein the fourth challenge GUI comprises a challenge profile section, an attempt indicator, a completion indicator, and a message notifying the user that the active challenge was not successfully completed, and in response to the meal monitoring application detecting the active challenge was not successfully completed, identify the active challenge as a completed challenge, wherein the fourth challenge GUI further comprises a first button which, when selected by the user, is configured to restart the completed challenge, and wherein the fourth challenge GUI further comprises a second button which, when selected by the user, outputs the first challenge GUI, wherein the user selects a different challenge from the list of one or more challenges reflected by the first challenge GUI.

In some embodiments, a modal is displayed on the fourth challenge GUI in response to the user selecting the first button, and wherein the modal is configured to prompt the user to confirm whether the user would like to restart the completed challenge.

In some embodiments, the meal monitoring application comprises a home GUI comprising a challenges card, wherein the challenges card comprises a selectable link, wherein the meal monitoring application, when executed by the one or more processors, further causes the one or more processors to: in response to the user selecting the link, output the first challenge GUI.

In some embodiments, each of the one or more challenges is configured to represent a challenge directed to the user's behavior or activity that can affect the user's analyte levels.

According to a second aspect of the present disclosure, there is provided a system for monitoring meal-related analyte responses in a user, the system comprising: a reader device, comprising: wireless communication circuitry configured to receive data indicative of an analyte level of the user, one or more processors coupled with a memory, the memory storing a meal monitoring application that, when executed by the one or more processors, causes the one or more processors to: receive meal information inputted by the user, wherein the meal information is configured to reflect the user's food choices; output a home GUI, wherein the home GUI comprises: a plurality of selectable sections, the plurality of selection sections comprising a user profile section, a meal entry section, a trends section, a diary section, and a reports section; a meals card configured to display one or more meal listings comprising the inputted meal information related to one or more consumed meals by the user; a trends card comprising a graphical representation reflecting information related to an analyte response associated with the user's food choices; a challenges card reflecting a list of one or more challenges relating the user's analyte response or glucose levels; and a recommendations card reflecting one or more recommendations relating to the user's food choices or analyte response.

The meal monitoring application can store recommendations in a database. Additionally, the meal monitoring application may output a recommendation from a trusted source. The stored recommendations can be outputted to every user in the same manner or may be personalized based on data analyzed by the meal monitoring application. Recommendations may be outputted to a category of users based on shared characteristics, demographics, location, behavior or activities. Recommendations can be generated based on various different criteria or behaviors. For example, recommendations can be outputted to the user based on particular user cohorts or characteristics identified by the meal monitoring application; psychographics; information inputted to the meal monitoring application by the user, such as diary entries; timing of meals consumed by the user; the amount of time the user has used the meal monitoring application or a particular sensor; demographics; geographical considerations, such as activities occurring in a particular location or region, and; seasonal activities. Recommendations may be generated and displayed on the meal monitoring application in a predetermined order based on particular criteria or behaviors analyzed by the meal monitoring application. The meal monitoring application may analyze data and generate relevant recommendations based on the data received. Recommendations may become more personalized as a function of time. For example, after 30 days, a recommendation may be outputted to the user which relates to directions on how to remove a sensor. The generated recommendations may be refined or updated as more data is received and analyzed by the meal monitoring application.

The data indicative of an analyte level of the user may include the user's analyte response. The user's analyte response may include a meal-related analyte response. The user's analyte response may include a user's glucose levels.

In some embodiments, the recommendations card comprises one or more selectable recommendation icons, wherein each of the one or more selectable recommendation icons corresponds to one of the one or more recommendations.

In some embodiments, each of the one or more selectable recommendation icons comprise a picture relating to the corresponding one of the one or more recommendations, and a recommendation title providing a textual description of the corresponding one of the one or more recommendations.

In some embodiments, the recommendations card comprises a plurality of selectable recommendation icons, wherein a first set of the plurality of selectable recommendations icons is displayed on the recommendations card, wherein the reader device further comprises a touchscreen, and wherein the meal monitoring application, when executed by the one or more processors, further causes the one or more processors to: receive input from the touchscreen corresponding to a swipe gesture or a drag gesture, and in response to the received input, display a second set of the plurality of selectable recommendations icons on the recommendations card, wherein at least one or more of the plurality of selectable recommendations icons of the second set is different than at least one or more of the plurality of selectable recommendations icons of the first set.

In some embodiments, the recommendations card comprises one or more selectable recommendation icons, wherein each of the one or more selectable recommendation icons corresponds to one of the one or more recommendations, wherein the meal monitoring application, when executed by the one or more processors, further causes the one or more processors to: in response to a selection of one of the one or more selectable recommendation icons, output a modal on the home GUI, wherein the modal provides contextual information related to the corresponding one of the one or more recommendations, and wherein the modal is configured to direct the user to act in accordance with the corresponding one of the one or more recommendations.

In some embodiments, the meal monitoring application, when executed by the one or more processors, further causes the one or more processors to: detect the user's food choices; analyze the inputted meal information; and based on the analysis, display one or more selectable recommendation icons on the recommendations card, wherein each of the one or more selectable recommendation icons reflects a recommendation related to the user's food choices or analyte response.

In some embodiments, the recommendations card comprises one or more selectable recommendation icons, wherein each of the one or more selectable recommendation icons corresponds to one of the one or more recommendations, wherein the meal monitoring application, when executed by the one or more processors, further causes the one or more processors to: in response to a selection of one of the one or more selectable recommendation icons, remove the selected one of the one or more selectable recommendation icons from the recommendation card, and display a new selectable recommendation icon on the recommendation card in place of the removed recommendation icon.

In some embodiments, the recommendations card comprises one or more selectable recommendation icons, wherein each of the one or more selectable recommendation icons corresponds to one of the one or more recommendations, wherein each of the one or more selectable recommendation icons is configured to be displayed on the recommendation card for a predetermined period of time.

In some embodiments, the meal monitoring application, when executed by the one or more processors, further causes the one or more processors to: detect when the predetermined period of time has been reached, in response to the predetermined period of time being reached, replace the one or more selectable recommendation icons on the recommendation card with a new set of one or more selectable recommendation icons, wherein at least one of the one or more selectable recommendation icons in the new set is different than at least one of the replaced one or more selectable recommendation icons.

In some embodiments, the home GUI is configured to transition between a plurality of views, wherein the plurality of views comprises at least a first view and a second view.

In some embodiments, the home GUI is in the first view, wherein the home GUI is configured to display the user profile section, the meal entry section, the diary section, and the meals card in the first view, wherein the reader device further comprises a touchscreen, wherein the meal monitoring application, when executed by the one or more processors, further causes the one or more processors to: receive input from the touchscreen corresponding to a scroll gesture, a swipe gesture, a pull gesture, or a drag gesture, and wherein, in response to the received input, the home GUI is configured to transition from the first view to the second view, wherein the trends card, the challenges card, and the recommendations card are displayed on the home GUI in the second view.

In some embodiments, the home GUI is configured to transition between a plurality of views, wherein each view of the plurality of views is different, wherein the reader device further comprises a touchscreen, wherein the meal monitoring application, when executed by the one or more processors, further causes the one or more processors to: receive input from the touchscreen corresponding to a scroll gesture, a swipe gesture, a pull gesture, or a drag gesture, and in response to the received input, display one view of the plurality of views of the home GUI.

In some embodiments, the meals card is configured to display one or more meal listings comprising meal information related to one or more of the most recently consumed meals.

In some embodiments, each of the one or more meal listings includes details of a meal consumed by the user, wherein the one or more meals listings are displayed on the meals card in chronological order, wherein a meal listing corresponding to a most recently consumed meal is displayed at a top portion of the meals card.

In some embodiments, each of the one or more meal listings includes details of a meal consumed by the user and the meal's corresponding meal-related analyte response.

In some embodiments, each of the one or more meal listings comprises: a text description of a meal consumed by the user; a portion size indicator comprising information indicating the meal was either smaller, medium, or large compared to a usual meal serving of the user; a datestamp associated with a date the meal was consumed by the user; a time stamp associated with a time the meal was consumed; and a graphical representation of an analyte response associated with the meal.

In some embodiments, the graphical representation comprises a plurality of segments.

In some embodiments, the plurality of segments includes a first segment, and wherein the first segment is indicative of the analyte response comprising a low glycemic response, wherein the plurality of segments includes a second segment, wherein the second segment is indicative of the analyte response comprising a medium glycemic response, and wherein the plurality of segments includes a third segment, wherein the third segment is indicative of the analyte response comprising a high glycemic response.

In some embodiments, the first segment, the second segment, and the third segment are each a different color.

In some embodiments, the graphical representation of the trends card is indicative of the analyte response associated with the user's food choices for a predetermined time period.

In some embodiments, the graphical representation of the trends card comprises a plurality of colored segments comprising a first colored segment, a second colored segment, and a third colored segment.

In some embodiments, the first colored segment comprises a green color indicative of a low glycemic response, wherein the second colored segment comprises a yellow color indicative of a medium glycemic response, and wherein the third colored segment comprises an orange color indicative of a high glycemic response.

In some embodiments, the trends card comprises a summary panel configured to provide an overall assessment of the user's food choices for a predetermined period of time.

In some embodiments, the trends card comprises a summary panel comprising information indicative of the analyte response associated with the user's food choices, wherein the trends card is configured to be dynamic, wherein the meal monitoring application, when executed by the one or more processors, further causes the one or more processors to: detect whether the analyte response associated with the user's food choices has provided new trend information, and in response to the new trend information being detected, populate an updated summary panel on the trends card.

In some embodiments, the trends card is not displayed on the home GUI when data indicative of an analyte level has not been received or associated with the inputted meal information.

In some embodiments, the challenges card on home GUI comprises one or more selectable challenge icons, wherein each of the one or more selectable challenge icons is configured to reflect a challenge relating to the user's analyte response or glucose levels.

In some embodiments, each of the one or more selectable challenge icons comprises a picture associated with the challenged reflected by the selected challenge icon, and a challenge title providing a textual description of the challenge reflected by the selected challenge icon.

In some embodiments, a live indicator is displayed on the picture to indicate the challenge reflected by picture is an active challenge on the meal monitoring application.

In some embodiments, the challenges card comprises a selectable link, wherein the meal monitoring application, when executed by the one or more processors, further causes the one or more processors to: in response to the user selecting the link, output a first challenge GUI comprising information on all challenges provided on the meal monitoring application.

In some embodiments, the challenges card comprises a plurality of selectable challenge icons, wherein a first set of the plurality of selectable challenge icons is displayed on the challenge card, wherein the reader device further comprises a touchscreen, and wherein the meal monitoring application, when executed by the one or more processors, further causes the one or more processors to: receive input from the touchscreen corresponding to a swipe gesture or a drag gesture, and in response to the received input, display a second set of the plurality of selectable challenge icons on the challenges, wherein at least one or more of the plurality of selectable challenge icons of the second set is different than at least one or more of the plurality of selectable challenge icons of the first set.

In some embodiments, the challenges card comprises a plurality of selectable challenge icons, wherein the challenges card is configured to display two or three of the plurality of selectable challenge icons on the home GUI at a same time.

In some embodiments, the recommendations card comprises a plurality of selectable recommendation icons, wherein the recommendations card is configured to display two or three of the plurality of selectable recommendation icons at a same time.

In some embodiments, the home GUI further comprises a navigation bar.

In some embodiments, the home GUI further comprises banner comprising a message relating to scanning a sensor and a meal impact.

According to a third aspect of the present disclosure, there is provided a system for monitoring meal-related analyte responses in a subject, the system comprising: a reader device, comprising: wireless communication circuitry configured to receive data indicative of an analyte level of the subject, one or more processors coupled with a memory, the memory storing a meal monitoring application that, when executed by the one or more processors, causes the one or more processors to: identify a peak analyte level value within a predetermined time period for the received data indicative of the analyte level of the subject, determine an estimated meal start time and an initial analyte level value based on the peak analyte level value, determine an analyte level variance value, prompt the subject to enter meal information, and associate the entered meal information with the analyte level variance value.

In some embodiments, the reader device comprises a smart phone.

In some embodiments, the data indicative of the analyte level of the subject comprises data indicative of a glucose level.

In some embodiments, the system further comprises a trusted computer system, wherein the trusted computer system is a cloud-computing platform comprising one or more servers. In some embodiments, the trusted computer system is configured to transmit the data indicative of the analyte level of the subject to the reader device.

In some embodiments, the system further comprises a sensor control device, wherein the sensor control device comprises an analyte sensor, and wherein at least a portion of the analyte sensor is configured to be positioned under a skin layer of the subject and in contact with a bodily fluid of the subject. In some embodiments, the sensor control device further is configured to transmit the data indicative of the analyte level of the subject to the reader device.

In some embodiments, the wireless communication circuitry of the reader device is configured to receive the data indicative of the analyte level of the subject according to a Bluetooth or a Near Field Communication wireless protocol.

In some embodiments, the peak analyte level value comprises a highest glucose value over a predetermined analyte level threshold. In some embodiments, the predetermined analyte level threshold is 170 mg/dL. In some embodiments, the predetermined analyte level threshold is 180 mg/dL. In some embodiments, the predetermined analyte level threshold is 190 mg/dL.

In some embodiments, the predetermined time period for the received data indicative of the analyte level of the subject comprises a last two hours of analyte data. In some embodiments, the predetermined time period for the received data indicative of the analyte level of the subject comprises a last four hours of analyte data. In some embodiments, the predetermined time period for the received data indicative of the analyte level of the subject comprises a last eight hours of analyte data.

In some embodiments, the estimated meal start time is determined by counting two hours back from a time of the peak analyte level value. In some embodiments, the estimated meal start time is determined by counting three hours back from a time of the peak analyte level value. In some embodiments, the estimated meal start time is determined by counting four hours back from a time of the peak analyte level value.

In some embodiments, the analyte level variance value is determined by subtracting the initial analyte level value from the peak analyte level value.

In some embodiments, the meal monitoring application, when executed by the one or more processors, further causes the one or more processors to store the meal information and associated analyte level variance value in the memory of the reader device.

According to a fourth aspect of the present disclosure, there is provided a system for monitoring meal-related analyte responses in a subject, the system comprising: a reader device, comprising: wireless communication circuitry configured to receive data indicative of an analyte level of the subject, one or more processors coupled with a memory, the memory storing a meal monitoring application that, when executed by the one or more processors, causes the one or more processors to: receive meal information inputted by the subject, receive the data indicative of the analyte level of the subject within a predetermined amount of time after the meal information is inputted by the subject, identify a peak analyte level value for the received data indicative of the analyte level of the subject, determine an initial analyte level value, determine an analyte level variance value, and associate the entered meal information with the analyte level variance value.

In some embodiments, the reader device comprises a smart phone.

In some embodiments, the data indicative of the analyte level of the subject comprises data indicative of a glucose level.

In some embodiments, the system further comprises a trusted computer system, wherein the trusted computer system is a cloud-computing platform comprising one or more servers. In some embodiments, the trusted computer system is configured to transmit the data indicative of the analyte level of the subject to the reader device.

In some embodiments, the system further comprises a sensor control device, wherein the sensor control device comprises an analyte sensor, and wherein at least a portion of the analyte sensor is configured to be positioned under a skin layer of the subject and in contact with a bodily fluid of the subject. In some embodiments, the sensor control device further is configured to transmit the data indicative of the analyte level of the subject to the reader device.

In some embodiments, the wireless communication circuitry of the reader device is configured to receive the data indicative of the analyte level of the subject according to a Bluetooth or a Near Field Communication wireless protocol.

In some embodiments, the peak analyte level value comprises a highest glucose value over a predetermined analyte level threshold. In some embodiments, the predetermined analyte level threshold is 170 mg/dL. In some embodiments, the predetermined analyte level threshold is 180 mg/dL. In some embodiments, the predetermined analyte level threshold is 190 mg/dL.

In some embodiments, the analyte level variance value is determined by subtracting the initial analyte level value from the peak analyte level value.

In some embodiments, the meal monitoring application, when executed by the one or more processors, further causes the one or more processors to store the meal information and associated analyte level variance value in the memory of the reader device.

In some embodiments, the meal monitoring application, when executed by the one or more processors, further causes the one or more processors to display a notification that a meal entry has not been entered after a predetermined reminder time period.

In some embodiments, the predetermined reminder time period is one week. In some embodiments, the predetermined reminder time period is three days. In some embodiments, the predetermined reminder time period is one day.

In some embodiments, the initial analyte level value is determined based on a time of the meal information inputted by the subject.

According to a fifth aspect of the present disclosure, there is provided a system for monitoring meal-related analyte responses in a subject, the system comprising: a reader device, comprising: wireless communication circuitry configured to receive data indicative of an analyte level of the subject, one or more processors coupled with a memory, the memory storing a meal monitoring application that, when executed by the one or more processors, causes the one or more processors to output a diary GUI, the diary GUI comprising a plurality of meal entries, wherein each meal entry of the plurality of meal entries comprises: a date of the each meal entry, a meal name, a graphical representation of an analyte level variance value associated with the each meal entry, and a numerical representation of the analyte level variance value associated with the each meal entry.

In some embodiments, the graphical representation of the analyte level variance value comprises a plurality of segments.

In some embodiments, the plurality of segments includes a first segment, wherein the first segment is indicative of the analyte level variance value in a first analyte level variance range, and wherein the plurality of segments includes a second segment, wherein the second segment is indicative of the analyte level variance value in a second analyte level variance range that is different from the first analyte level variance range.

In some embodiments, the first segment is a different color from the second segment.

In some embodiments, the first segment comprises a different area from the second segment.

In some embodiments, the first analyte level variance range is less than 70 mg/dL.

In some embodiments, the second analyte level variance range is between 70 mg/dL and 120 mg/dL.

In some embodiments, the plurality of segments further includes a third segment indicative of the analyte level variance value in a third analyte level variance range that is different from both the first analyte level variance range and the second analyte level variance range.

In some embodiments, the third segment is a different color from the first segment and the second segment.

In some embodiments, each meal entry of the plurality of meal entries further comprises a time of each meal entry. In some embodiments, each meal entry of the plurality of meal entries further comprises an activity field. In some embodiments, each meal entry of the plurality of meal entries further comprises a notes field.

In some embodiments, the diary GUI further comprises a view setting configured to display the plurality of meal entries by day or by week.

In some embodiments, each meal entry of the plurality of meal entries further comprises a weighted average of the analyte level variance value. In some embodiments, the weighted average of the analyte level variance value is based on a plurality of historical meal entries having a same or similar meal or food to the each meal entry. In some embodiments, the weighted average of the analyte level variance value is determined by a weighted average function comprising a recency factor.

In some embodiments, the recency factor of the weighted average function is configured to decrement the analyte level variance value of a historical meal entry by a predetermined factor for each day between a date of the historical meal entry and a current date.

According to a sixth aspect of the present disclosure, there is provided a system for monitoring meal-related analyte responses in a subject, the system comprising: a reader device, comprising: wireless communication circuitry configured to receive data indicative of an analyte level of the subject, one or more processors coupled with a memory, the memory storing a meal monitoring application that, when executed by the one or more processors, causes the one or more processors to output a trends GUI, the trends GUI comprising a glycemic response view and a meals view, wherein the glycemic response view comprises a graphical representation reflecting a plurality of segments comprising a first segment and a second segment, wherein the first segment is indicative of a first analyte level variance range, and the second segment is indicative of a second analyte level variance range that is different from the first analyte level variance range.

In some embodiments, the first segment is indicative of a first set of meal entries each having an analyte level variance value within the first analyte level variance range.

In some embodiments, the second segment is indicative of a second set of meal entries each having an analyte level variance value within the second analyte level variance range.

In some embodiments, the meals view comprises: a plurality of meal entries, wherein each meal entry of the plurality of meal entries comprises: a date and a time of the each meal entry, a meal name, a graphical representation of an analyte level variance value associated with the each meal entry, and a numerical representation of the analyte level variance value associated with the each meal entry.

According to a seventh aspect of the present disclosure, there is provided a method for monitoring meal-related analyte responses in a user, the method comprising: receiving, through wireless communication circuitry, data indicative of an analyte level of a user; identifying an analyte response of the user based on the data indicative of an analyte level; and outputting, by a processor coupled with a memory storing a meal monitoring application, a first challenge GUI reflecting a list of one or more challenges relating to the user's analyte response, wherein the one or more challenges comprise one or more active challenges, one or more completed challenges, and one or more unattempted challenges, the first challenge GUI comprising a first challenge card, a second challenge card, and a third challenge card; wherein the first challenge card reflects the one or more active challenges, wherein each of the one or more active challenges reflects a challenge currently in progress by the user on the meal monitoring application, wherein the second challenge card reflects the one or more completed challenges, wherein each of the one or more completed challenges reflects a challenge completed by the user, and, wherein the third challenge card reflects one or more unattempted challenges, wherein each of the one or more unattempted challenges reflects a challenge in which the user has not yet participated in.

According to an eight aspect of the present disclosure, there is provided a method for monitoring meal-related analyte responses in a user, the method comprising: receiving, by a processor coupled with a memory storing a meal monitoring application, inputted meal information by the user, wherein the meal information is reflects the user's food choices; and outputting, by the processor, a home GUI comprising: a plurality of selectable sections, wherein the plurality of selection sections comprises a user profile section, a meal entry section, a trends section, a diary section, and a reports section; a meals card configured to display one or more meal listings comprising the inputted meal information related to one or more consumed meals by the user; a trends card comprising a graphical representation reflecting information related to an analyte response associated with the user's food choices; a challenges card reflecting a list of one or more challenges relating the user's analyte response or glucose levels; and a recommendations card reflecting one or more recommendations relating to the user's food choices or analyte response.

According to a ninth aspect of the present disclosure, there is provided a method for monitoring meal-related analyte responses in a user, the method comprising: receiving, through wireless communication circuitry, data indicative of an analyte level of a user; identifying, by a processor coupled with a memory storing a meal monitoring application, a peak analyte level value within a predetermined time period for the received data indicative of the analyte level of the subject; determining, by the processor, an estimated meal start time and an initial analyte level value based on the peak analyte level value; determining, by the processor, an analyte level variance value; prompting, by the processor, the subject to enter meal information; and associating, by the processor, the entered meal information with the analyte level variance value.

According to a tenth aspect of the present disclosure, there is provided a method for monitoring meal-related analyte responses in a user, the method comprising: receiving, through wireless communication circuitry, data indicative of an analyte level of a user; receiving, by a processor coupled with a memory storing a meal monitoring application, meal information inputted by the user; receiving, by the processor, the data indicative of the analyte level of the user within a predetermined amount of time after the meal information is inputted by the user; identifying, by the processor, a peak analyte level value for the received data indicative of the analyte level of the subject; determining, by the processor, an initial analyte level value; determining, by the processor, an analyte level variance value; and associating, by the processor, the entered meal information with the analyte level variance value.

According to an eleventh aspect of the present disclosure, there is provided a method for monitoring meal-related analyte responses in a user, the method comprising: receiving, through wireless communication circuitry, data indicative of an analyte level of a user; outputting, by a processor coupled with a memory storing a meal monitoring application, a diary GUI comprising a plurality of meal entries, wherein each meal entry of the plurality of meal entries comprises: a date of the each meal entry, a meal name, a graphical representation of an analyte level variance value associated with the each meal entry, and a numerical representation of the analyte level variance value associated with the each meal entry.

According to a twelfth aspect of the present disclosure, there is provided a method for monitoring meal-related analyte responses in a user, the method comprising: receiving, through wireless communication circuitry, data indicative of an analyte level of a user; and outputting, by a processor coupled with a memory storing a meal monitoring application, a trends GUI comprising a glycemic response view and a meals view, wherein the glycemic response view comprises a graphical representation reflecting a plurality of segments comprising a first segment and a second segment, wherein the first segment is indicative of a first analyte level variance range, and the second segment is indicative of a second analyte level variance range that is different from the first analyte level variance range.

In many embodiments, the individual's meal-related analyte responses collected by an analyte monitoring system, such as an in vivo analyte monitoring system, can be compared with or linked to meal information to discover common consistencies (or inconsistencies) along with trends therein based on related historical glucose readings and associated algorithms, variables, weights, and comparisons.

Many embodiments disclosed herein are intended to engage the individual by providing direct and timely feedback regarding the individual's meal-related analyte response. In some embodiments, this analyte response can be provided to the individual in an easy-to-understand format to characterize the effects of meal consumption.

The present embodiments can be immediately informative to the individual, thereby encouraging the individual to take actions to better understand how their own diet impacts their body's analyte response. The individual can compare and contrast their current and historical analyte data to see their how their own efforts are related to better diet and meal selection, and how these choices directly affect their health.

Many of the embodiments provided herein are improved GUIs or GUI features for analyte monitoring systems that are highly intuitive, user-friendly, and provide for rapid access to physiological information of a user. More specifically, these embodiments may allow a user to easily navigate through and between different user interfaces that can quickly indicate to the user various physiological conditions and/or actionable responses and correlate analyte data with meals, exercise, stress, or other factors, without requiring the user (or an HCP) to go through the arduous task of examining large volumes of analyte data. Furthermore, in many of the embodiments, some of the GUIs and GUI features allow for users (and/or their caregivers) to better understand and improve their diet, eating habits, and managing other stressors as they see the correlations with these activities and their glucose levels. Likewise, in many embodiments, improved digital interfaces and/or features for meal monitoring systems may improve upon the visualization of the impact of food choices on analyte (glucose) levels. Other improvements and advantages are provided as well. The various configurations of these devices are described in detail by way of the embodiments which are only examples.

The improvements to the GUIs in the various aspects described and claimed herein produce a technical effect at least in that they assist the user of the device to operate the device more accurately, more efficiently, and more safely. It will be appreciated that the information that is provided to the user on the GUIs, the order in which that information is provided, and the clarity with which that information is structured can have a significant effect on the way the user interacts with the system and the way the system operates. The GUIs therefore guide the user in the technical task of operating the system to take the necessary readings and/or obtain information accurately and efficiently.

Aspects of the present disclosure can be provided in conjunction with each other and features of one aspect can be applied to other aspects. Any feature in one aspect of the present disclosure can be applied to other aspects of the present disclosure, in any appropriate combination. For instance, features of the system of the first aspect can be used in combination with features of the method of the seventh or eighth aspect. It should also be appreciated that particular combinations of the various features described and defined in any aspects of the present disclosure can be implemented and/or supplied and/or used independently.

Other systems, devices, methods, features, and advantages of the subject matter described herein will be or will become apparent to one with skill in the art upon examination of the following figures and detailed description. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be depicted schematically rather than literally or precisely.

FIG. 1 is a high level diagram depicting an example embodiment of an analyte monitoring system for analyte (e.g., glucose) measurement, data acquisition and/or processing.

FIG. 2A is a block diagram depicting an example embodiment of a reader device configured as a smartphone.

FIG. 2B is a block diagram depicting an example embodiment of a sensor control device.

FIG. 3A is a flow diagram depicting an example embodiment of a method for meal information gathering and assessment.

FIGS. 3B to 3E are high level diagrams depicting example embodiments of various analyte monitoring systems for use with a meal monitor application.

FIG. 3F is a flow diagram depicting an example embodiment of a method for associating analyte data with meal information.

FIG. 3G is a flow diagram depicting another example embodiment of a method for associating analyte data with meal information.

FIG. 4A-1 is an example embodiment of a home GUI for a meal monitor application.

FIG. 4A-2 is an example embodiment of a home GUI for a meal monitor application.

FIGS. 4A-3 and 4A-4 are example embodiments of a home GUI for a meal monitor application.

FIGS. 4A-5 and 4A-6 are example embodiments of a home GUI for a meal monitor application.

FIG. 4A-7 is an example embodiment of a home screen modal for a meal monitor application.

FIG. 4B is an example embodiment of an “About Us” GUI for a meal monitor application.

FIG. 4C is an example embodiment of a “Contact Us” modal for a meal monitor application.

FIGS. 4D to 4E are example embodiments of a “Frequently Asked Questions” GUI for a meal monitor application.

FIGS. 4F to 4I are example embodiments of connection GUIs for a meal monitor application.

FIGS. 4J to 4N are example embodiments of connection GUIs for a meal monitor application.

FIG. 4O is an example embodiment of a notifications GUI for a meal monitor application.

FIGS. 5A and 5B are example embodiments of a profile GUI for a meal monitor application.

FIGS. 5C to 5I are example embodiments of a profile GUI for a meal monitor application.

FIGS. 6A to 6E are example embodiments of an “Add Food” GUI for a meal monitor application.

FIGS. 6F to 6J are example embodiments of an “Add Food” GUI for a meal monitor application.

FIGS. 7A to 7I are example embodiments of a diary GUI for a meal monitor application.

FIGS. 7J to 7R are example embodiments of a diary GUI for a meal monitor application.

FIGS. 8A to 8D are example embodiments of a trends GUI for a meal monitor application.

FIGS. 8E to 8H are example embodiments of a trends GUI for a meal monitor application.

FIGS. 9A to 9B are example embodiments of a reports GUI for a meal monitor application.

FIGS. 9C to 9E are example embodiments of a reports GUI for a meal monitor application.

FIG. 9F is an example embodiment of a reports GUI for a meal monitor application.

FIG. 10 is a flow diagram depicting an example embodiment of a method for on-boarding a user of a meal monitor application.

FIGS. 11A-1 to 11N-3 are example embodiments of an on-boarding GUI for a meal monitor application.

FIGS. 12A to 12K are example embodiments of challenges GUI for a meal monitor application.

FIGS. 13A to 13C are example embodiments of notifications on GUIs for a meal monitor application.

### DETAILED DESCRIPTION

Provided herein are example embodiments of systems, devices, and methods for monitoring and measuring analyte responses to meals for a human individual. In particular, based on the analyte data collected, meal-related events and their impact on the individual's analyte levels can be further understood by a user, and eventually used to modify future meal selection and dietary habits.

Before describing this subject matter in greater detail, it is worthwhile to describe example embodiments of systems, devices, and methods with which the subject matter can be implemented.

A number of systems have been developed for the automatic monitoring of the analyte(s), like glucose, in bodily fluid such as in the blood stream, in interstitial fluid (“ISF”), dermal fluid of the dermal layer, or in other biological fluid. Some of these systems are configured so that at least a portion of a sensor is positioned below a skin surface of a user, e.g., in a blood vessel or in the subcutaneous tissue of a user, to obtain information about at least one analyte of the body.

As such, these systems can be referred to as “in vivo” monitoring systems. In vivo analyte monitoring systems include “Continuous Analyte Monitoring” systems (or “Continuous Glucose Monitoring” systems) that can transmit data from a sensor control device to a reader device continuously without prompting, e.g., automatically according to a schedule. In vivo analyte monitoring systems also include “Flash Analyte Monitoring” systems (or “Flash Glucose Monitoring” systems or simply “Flash” systems) that can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

The in vivo analyte monitoring systems can be differentiated from “in vitro” systems that contact a biological sample outside of the body (or rather “ex vivo”) and that typically include a meter device that has a port for receiving an analyte test strip carrying bodily fluid of the user, which can be analyzed to determine the user's blood sugar level. While in many of the present embodiments the monitoring is accomplished in vivo, the embodiments disclosed herein can be used with in vivo analyte monitoring systems that incorporate in vitro capability, as well has purely in vitro or ex vivo analyte monitoring systems.

The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “reader device” (or simply a “reader”), “handheld electronics” (or a handheld), a “portable data processing” device or unit, a “data receiver,” a “receiver” device or unit (or simply a receiver), or a “remote” device or unit, to name a few. Other devices such as personal computers have also been utilized with or incorporated into in vivo and in vitro monitoring systems.

### Embodiments of In Vivo Analyte Monitoring Systems

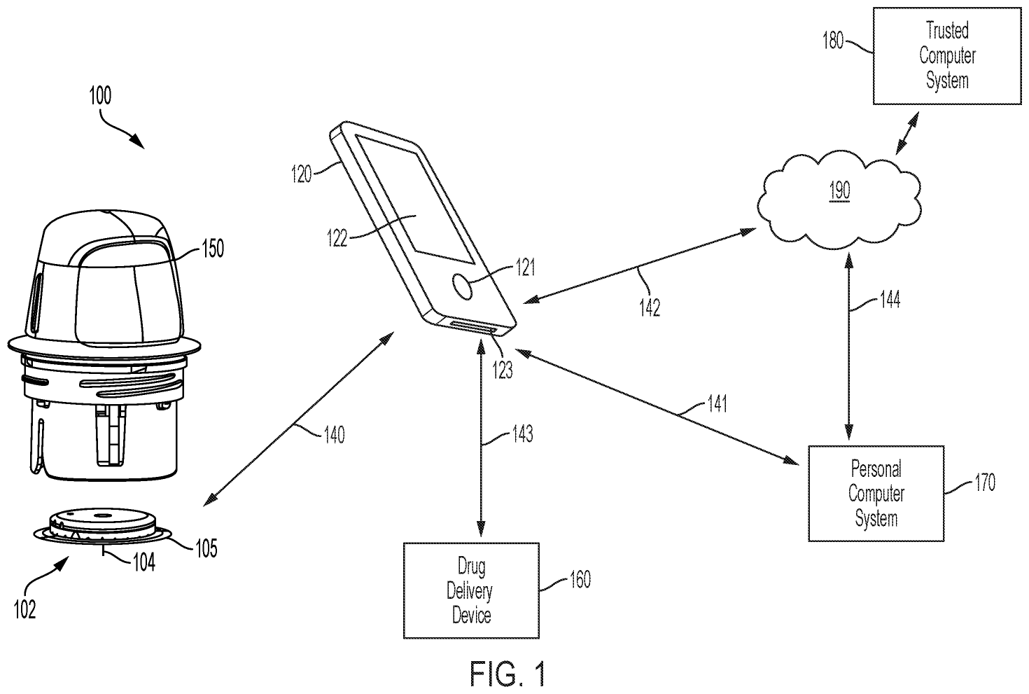

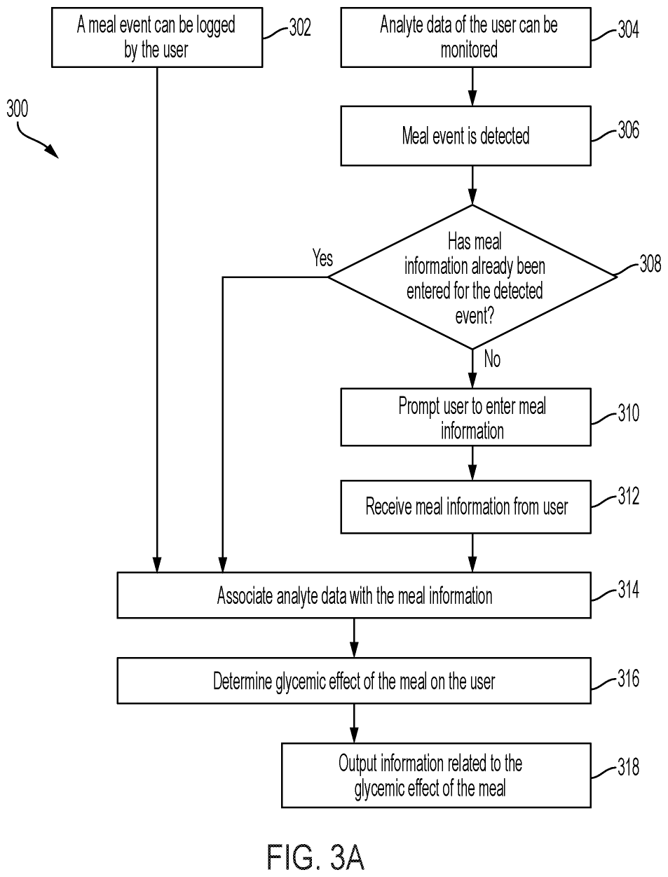

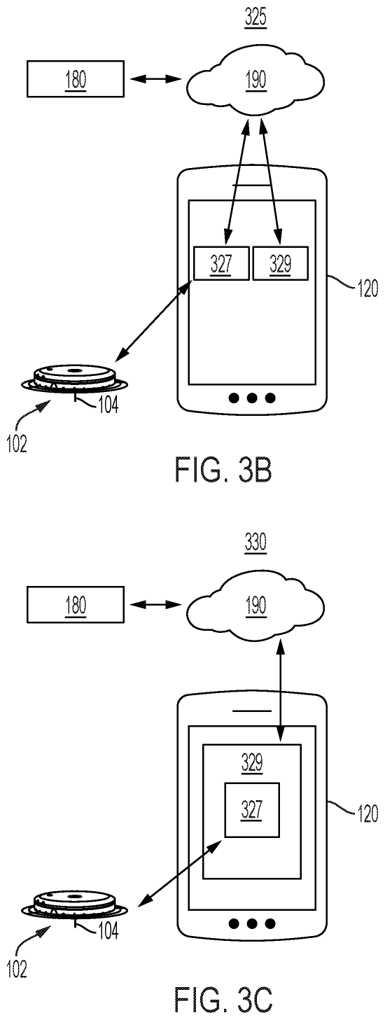

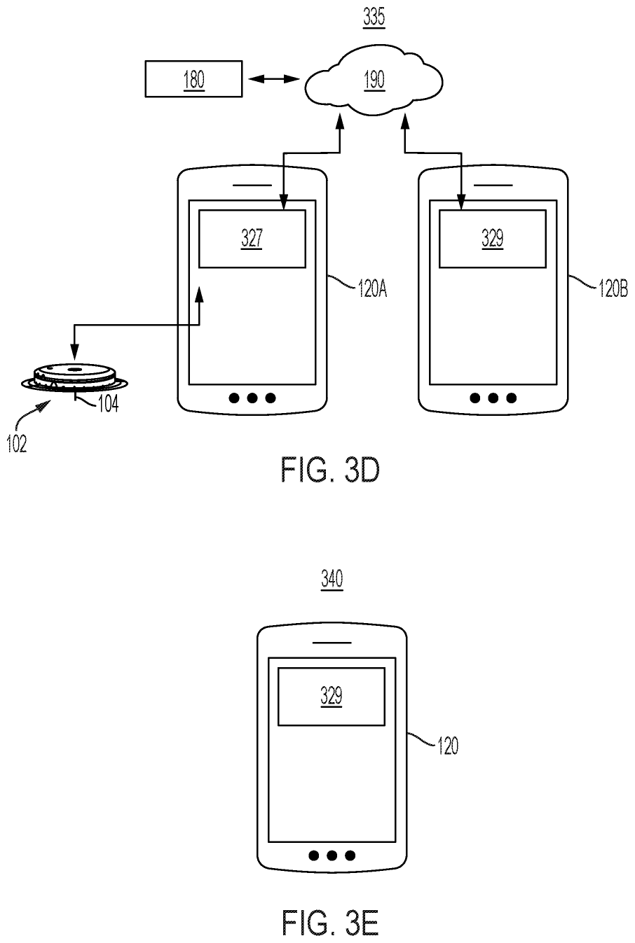

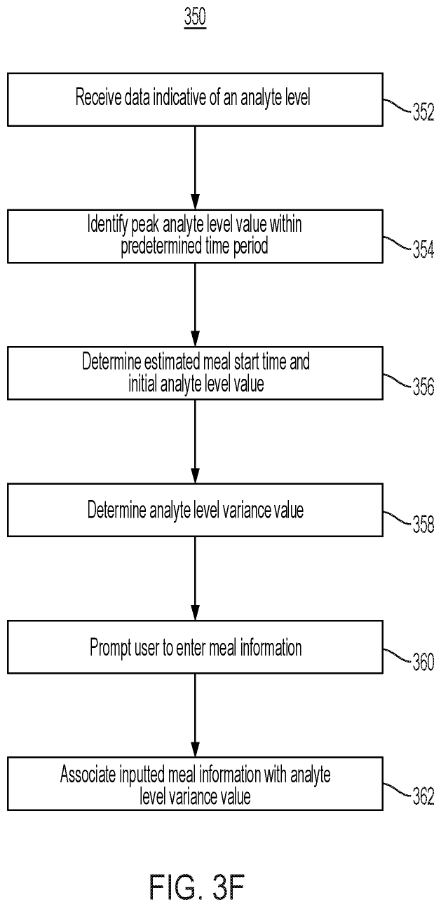

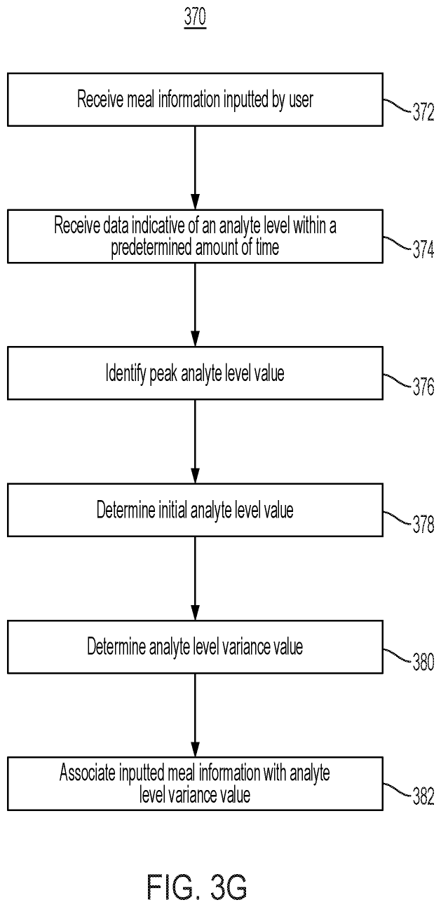

For purpose of illustration, and not limitation, the GUIs and associated software described herein may be used in connection with an exemplary analyte monitoring system as depicted in FIG. 1, as well as exemplary systems described below with respect to FIGS. 3B to 3E. FIG. 1 is an illustrative view depicting an example in vivo analyte monitoring system 100 with which any and/or all of the embodiments described herein can be used. System 100 can have a sensor control device 102 and a reader device 120 that communicate with each other over a local communication path (or link) 140, which can be wired or wireless, and uni-directional or bi-directional. In embodiments where local communication path 140 is wireless, any near field communication (NFC) protocol, RFID protocol, Bluetooth or Bluetooth Low Energy protocol, Wi-Fi protocol, proprietary protocol, or the like can be used, including those communication protocols in existence as of the date of this filing or their later developed variants.

Bluetooth is a well-known standardized short range wireless communication protocol, and Bluetooth Low Energy is a version of the same that requires less power to operate. Bluetooth Low Energy (Bluetooth LE, BTLE, BLE) is also referred to as Bluetooth Smart or Bluetooth Smart Ready. A version of BTLE is described in the Bluetooth Specification, version 4.0, published Jun. 30, 2010, which is explicitly incorporated by reference herein for all purposes. The term “NFC” applies to a number of protocols (or standards) that set forth operating parameters, modulation schemes, coding, transfer speeds, frame format, and command definitions for NFC devices. The following is a non-exhaustive list of examples of these protocols, each of which (along with all of its sub-parts) is incorporated by reference herein in its entirety for all purposes: ECMA-340, ECMA-352, ISO/IEC 14443, ISO/IEC 15693, ISO/IEC 16000-3, ISO/IEC 18092, and ISO/IEC 21481.

Reader device 120 is also capable of wired, wireless, or combined communication, either bi-directional or uni-directional, with either or all of: a drug delivery device 160 over communication path (or link) 143, a local computer system 170 over communication path (or link) 141, and with a network 190 over communication path (or link) 142. The same wireless protocols described for link 140 can likewise be used for all or part of links 141, 142, and 143.

Reader device 120 can communicate with any number of entities through network 190, which can be part of a telecommunications network, such as a Wi-Fi network, a local area network (LAN), a wide area network (WAN), the internet, or other data network for uni-directional or bi-directional communication. A trusted computer system 180 can be accessed through network 190. In an alternative embodiment, communication paths 141 and 142 can be the same path which can include the network 190 and/or additional networks. All communications over paths 140, 141, 142, 143, and 144 can be encrypted, and sensor control device 102, reader device 120, drug delivery device 160, remote computer system 170, and trusted computer system 180 can each be configured to encrypt and decrypt those communications sent and received.

Variants of devices 102 and 120, as well as other components of an in vivo-based analyte monitoring system that are suitable for use with the system, device, and method embodiments set forth herein, are described in U.S. Patent Publication No. 2011/0213225 (the '225 Publication), which is incorporated by reference herein in its entirety for all purposes.

Sensor control device 102 can include a housing 103 containing in vivo analyte monitoring circuitry and a power source (not shown). The in vivo analyte monitoring circuitry can be electrically coupled with an analyte sensor 104 that can extend through an adhesive patch 105 and project away from housing 103. Adhesive patch 105 contains an adhesive layer (not shown) for attachment to a skin surface of the body of the user. Other forms of body attachment to the body may be used, in addition to or instead of adhesive.

Sensor 104 is adapted to be at least partially inserted into the body of the user, where it can make fluid contact with that user's body fluid (e.g., interstitial fluid (ISF), dermal fluid, or blood) and be used, along with the in vivo analyte monitoring circuitry, to measure analyte-related data of the user. Generally, sensor control device 102 and its components can be applied to the body with a mechanical applicator 150 in one or more steps, as described in the incorporated '225 Publication, or in any other desired manner.

After activation, sensor control device 102 can wirelessly communicate the collected analyte data (such as, for example, data corresponding to monitored analyte level and/or monitored temperature data, and/or stored historical analyte related data) to reader device 120 where, in certain embodiments, it can be algorithmically processed into data representative of the analyte level of the user and then displayed to the user and/or otherwise incorporated into a diabetes monitoring regime.

Various embodiments disclosed herein relate to reader device 120, which can have a user interface including one or more of a display 122, keyboard, optional user interface component 121, and the like. Here, display 122 can output information to the user and/or accept an input from the user (e.g., if configured as a touch screen). Reader device 120 can include one or more optional user interface components 121, such as a button, actuator, touch sensitive switch, capacitive switch, pressure sensitive switch, jog wheel or the like. Reader device 120 can also include one or more data communication ports 123 for wired data communication with external devices such as local computer system 170. Reader device 120 may also include an integrated or attachable in vitro meter, including an in vitro test strip port (not shown) to receive an in vitro analyte test strip for performing in vitro blood analyte measurements.

Drug delivery device 160 is capable of injecting or infusing a drug, such as but not limited to insulin, into the body of the individual wearing sensor control device 102. Like reader device 120, the drug delivery device can include processing circuitry, non-transitory memory containing instructions executable by the processing circuitry, wireless or wired communication circuitry, and a user interface including one or more of a display, touchscreen, keyboard, an input button or instrument, and the like. Drug delivery device 160 can include a drug reservoir, a pump, an infusion tube, and an infusion cannula configured for at least partial implantation into the user's body. The pump can deliver insulin from the reservoir, through the tube, and then through the cannula into the user's body. Drug delivery device 160 can include instructions, executable by the processor, to control the pump and the amount of insulin delivered. These instructions can also cause calculation of insulin delivery amounts and durations (e.g., a bolus infusion and/or a basal infusion profile) based on analyte level measurements obtained directly or indirectly from sensor control device 102. Alternatively, calculations of insulin delivery amounts and durations, and the control of the pump, can be performed by reader device 120 directly. The drug delivery device can be configured to communicate directly with reader device 120 in the form of a closed loop or semi-closed loop system. Alternatively, the drug delivery device can include the functionality of reader device 120 described herein, or vice versa, to arrive at one integrated reader and drug delivery device.

Computer system 170 may be a personal or laptop computer, a tablet, or other suitable data processing device. Computer 170 can be either local (e.g., accessible via a direct wired connection such as USB) or remote to reader device 120 and can be (or include) software for data management and analysis and communication with the components in analyte monitoring system 100. Operation and use of computer 170 is further described in the '225 Publication incorporated herein by reference. Analyte monitoring system 100 can also be configured to operate with a data processing module (not shown), also as described in the incorporated '225 Publication.

Trusted computer system 180 can be used to perform authentication of the user, the sensor control device 102, and/or reader device 120; used to store confidential data received from devices 102 and/or 120; used to output confidential data to devices 102 and/or 120; to name only a few functions. Trusted computer system 180 can include one or more computers, servers, networks, databases, and the like. In some embodiments, trusted computer system 180 can comprise a cloud-computing platform comprising one or more servers. Trusted computer system 180 can be within the possession of the manufacturer or distributor of sensor control device 102, either physically or virtually through a secured connection, or can be maintained and operated by a different party (e.g., a third party).

Trusted computer system 180 can be trusted in the sense that system 100 can assume that computer system 180 provides authentic data or information. Trusted computer system 180 can be trusted simply by virtue of it being within the possession or control of the manufacturer, e.g., like a typical web server. Alternatively, trusted computer system 180 can be implemented in a more secure fashion such as by requiring additional password, encryption, firewall, or other internet access security enhancements that further guard against counterfeiter attacks or attacks by computer hackers.

The processing of data and the execution of software within system 100 can be performed by one or more processors of reader device 120, computer system 170, and/or sensor control device 102. For example, raw data measured by sensor 104 can be algorithmically processed into a value that represents the analyte level and that is readily suitable for display to the user, and this can occur in sensor control device 102, reader device 120, or computer system 170. This and any other information derived from the raw data can be displayed in any of the manners described above (with respect to display 122) on any display residing on any of sensor control device 102, reader device 120, or computer system 170. The information may be utilized by the user to determine any necessary corrective actions to ensure the analyte level remains within an acceptable and/or clinically safe range.

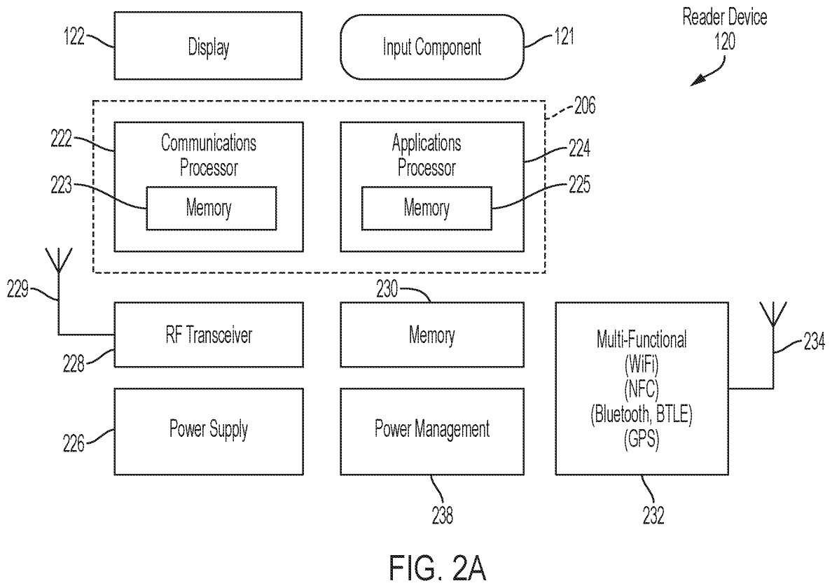

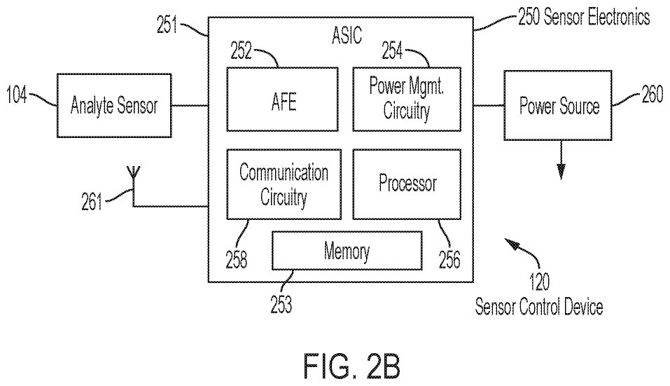

FIGS. 2A and 2B depict example embodiments of reader device 120 and sensor control device 102, respectively. As discussed above, reader device 120 can be a mobile communication device such as, for example, a Wi-Fi or internet enabled smartphone, tablet, or personal digital assistant (PDA). Examples of smartphones can include, but are not limited to, those phones based on a WINDOWS® operating system, ANDROID® operating system, IPHONE® operating system, PALM WEBOS®, BLACKBERRY® operating system, or SYMBIAN® operating system, with network connectivity for data communication over the internet or a local area network (LAN).

Reader device 120 can also be configured as a mobile smart wearable electronics assembly, such as an optical assembly that is worn over or adjacent to the user's eye (e.g., a smart glass or smart glasses). This optical assembly can have a transparent display that displays information about the user's analyte level (as described herein) to the user while at the same time allowing the user to see through the display such that the user's overall vision is minimally obstructed. The optical assembly may be capable of wireless communications similar to a smartphone. Other examples of wearable electronics include devices that are worn around or in the proximity of the user's wrist (e.g., a watch, etc.), neck (e.g., a necklace, etc.), head (e.g., a headband, hat, etc.), chest, or the like.

FIG. 2A is a block diagram of an example embodiment of a reader device 120 according to various embodiments disclosed herein. In this example, the reader device 120 is in the form of a smartphone, upon which the various software, applications, and graphical user interfaces disclosed herein can reside. Here, reader device 120 includes an input component 121, display 122, and processing hardware 206, which can include one or more processors, microprocessors, controllers, and/or microcontrollers, each of which can be a discrete chip or distributed amongst (and a portion of) a number of different chips. Here, processing hardware 206 includes a communications processor 222 having on-board non-transitory memory 223 and an applications processor 224 having on-board non-transitory memory 225. Reader device 120 further includes an RF transceiver 228 coupled with an RF antenna 229, a memory 230, multi-functional circuitry 232 with one or more associated antennas 234, a power supply 226, and power management circuitry 238. FIG. 2A is an abbreviated representation of the internal components of a smartphone, and other hardware and functionality (e.g., codecs, drivers, glue logic, etc.) can of course be included.

Communications processor 222 can interface with RF transceiver 228 and perform analog-to-digital conversions, encoding and decoding, digital signal processing and other functions that facilitate the conversion of voice, video, and data signals into a format (e.g., in-phase and quadrature) suitable for provision to RF transceiver 228, which can then transmit the signals wirelessly. Communications processor 222 can also interface with RF transceiver 228 to perform the reverse functions necessary to receive a wireless transmission and convert it into digital data, voice, and video.

Applications processor 224 can be adapted to execute the operating system and any software applications that reside on reader device 120 (such as any sensor interface application or analyte monitoring application that includes, e.g., SLL 304), process video and graphics, and perform those other functions not related to the processing of communications transmitted and received over RF antenna 229. Any number of applications can be running on reader device 120 at any one time, and will typically include one or more applications that are related to a diabetes monitoring regime, in addition to the other commonly used applications that are unrelated to such a regime, e.g., email, calendar, weather, etc.

Memory 230 can be shared by one or more of the various functional units present within reader device 120, or can be distributed amongst two or more of them (e.g., as separate memories present within different chips). Memory 230 can also be a separate chip of its own. Memory 230 is non-transitory, and can be volatile (e.g., RAM, etc.) and/or non-volatile memory (e.g., ROM, flash memory, F-RAM, etc.).

Multi-functional circuitry 232 can be implemented as one or more chips and/or components, including communication circuitry, that perform other functions such as local wireless communications (e.g., Wi-Fi, Bluetooth, Bluetooth Low Energy) and determining the geographic position of reader device 120 (e.g., global positioning system (GPS) hardware). One or more other antennas 234 are associated with the functional circuitry 232 as needed.

Power supply 226 can include one or more batteries, which can be rechargeable or single-use disposable batteries. Power management circuitry 238 can regulate battery charging and power supply monitoring, boost power, perform DC conversions, and the like. As mentioned, reader device 120 may also include one or more data communication ports such as USB port (or connector) or RS-232 port (or any other wired communication ports) for data communication with a remote computer system 170 (see FIG. 1), or sensor control device 102, to name a few.

FIG. 2B is a block schematic diagram depicting an example embodiment of sensor control device 102 having analyte sensor 104 and sensor electronics 250 (including analyte monitoring circuitry). Although any number of chips can be used, in many embodiments, the majority of the sensor electronics 250 are incorporated on a single semiconductor chip 251 that can be, e.g., a custom application specific integrated circuit (ASIC). Shown within ASIC 251 are several high-level functional units, including an analog front end (AFE) 252, power management circuitry 254, processor 256, and communication circuitry 258 (which can be implemented as a transmitter, receiver, transceiver, passive circuit, or otherwise according to the communication protocol). In the embodiment shown in FIG. 2B, both AFE 252 and processor 256 are used as analyte monitoring circuitry, but in other embodiments either circuit can perform the analyte monitoring function. Processor 256 can include one or more processors, microprocessors, controllers, and/or microcontrollers.

A non-transitory memory 253 is also included within ASIC 251 and can be shared by the various functional units present within ASIC 251, or can be distributed amongst two or more of them. Memory 253 can be volatile and/or non-volatile memory. In this embodiment, ASIC 251 is coupled with power source 260, which can be a coin cell battery, or the like. AFE 252 interfaces with in vivo analyte sensor 104 and receives measurement data therefrom and outputs the data to processor 256 in digital form, which in turn processes the data to arrive at the end-result analyte discrete and trend values, etc. This data can then be provided to communication circuitry 258 for sending, by way of antenna 261, to reader device 120 (not shown) where further processing can be performed by, e.g., the sensor interface application. It should be noted that the functional components of ASIC 251 can also be distributed amongst two or more discrete semiconductor chips. For example, in some embodiments, communication circuitry 258 can be a separate semiconductor chip from ASIC 251, and communication circuitry 258 can be configured to process signals received from in vivo analyte sensor 104, e.g., via ASIC 251, into end-result analyte discrete and trend values.

Performance of the data processing functions within the electronics of the sensor control device 102 provides the flexibility for system 100 to schedule communication from sensor control device 102 to reader device 120, which in turn limits the number of unnecessary communications and can provide further power savings at sensor control device 102.

Information may be communicated from sensor control device 102 to reader device 120 automatically and/or continuously when the analyte information is available, or may not be communicated automatically and/or continuously, but rather stored or logged in a memory of sensor control device 102, e.g., for later output.

Data can be sent from sensor control device 102 to reader device 120 at the initiative of either sensor control device 102 or reader device 120. For example, in many example embodiments, sensor control device 102 can communicate data periodically in an unprompted fashion, such that an eligible reader device 120, if in range and in a listening state, can receive the communicated data (e.g., sensed analyte data). This is at the initiative of sensor control device 102 because reader device 120 does not have to send a request or other transmission that first prompts sensor control device 102 to communicate. Transmissions can be performed, for example, using an active Wi-Fi, Bluetooth, or BTLE connection, and can occur according to a schedule that is programmed within device 102 (e.g., about every 1 minute, about every 5 minutes, about every 10 minutes, or the like). Transmissions can also occur in a random or pseudorandom fashion, such as whenever sensor control device 102 detects a change in the sensed analyte data. Further, transmissions can occur in a repeated fashion regardless of whether each transmission is actually received by a reader device 120.

System 100 can also be configured such that reader device 120 sends a transmission that prompts sensor control device 102 to communicate its data to reader device 120. This is generally referred to as “on-demand” data transfer. An on-demand data transfer can be initiated based on a schedule stored in the memory of reader device 120, or at the behest of the user via a user interface of reader device 120. For example, if the user wants to check his or her analyte level, the user could perform a scan of sensor control device 102 using an NFC, Bluetooth, BTLE, or Wi-Fi connection. Data exchange can be accomplished using broadcasts, session-based transfers, on-demand transfers, or any combination thereof.

Accordingly, once a sensor control device 102 is placed on the body so that at least a portion of sensor 104 is in contact with the bodily fluid and electrically coupled to the electronics within device 102, sensor derived analyte information may be communicated in on-demand or in an autonomous fashion from the sensor control device 102 to a reader device 120. On-demand transfer can occur by first powering on reader device 120 (or it may be continually powered) and executing a software algorithm stored in and accessed from a memory of reader device 120 to generate one or more requests, commands, control signals, or data packets to send to sensor control device 102. The software algorithm executed under, for example, the control of processing hardware 206 of reader device 120 may include routines to detect the position of the sensor control device 102 relative to reader device 120 to initiate the transmission of the generated request command, control signal and/or data packet.

Example Embodiments of Methods for Associating Analyte Data with Meal Information

In many embodiments, the subject matter described herein is implemented by a software application program that is stored in a memory of and executed by a processor-based device, such as any one of the reader devices (e.g., a smart phone), drug delivery devices, or any of the other computing devices described herein. In certain embodiments, the software is implemented as one or more downloadable software applications (“an App”) on a reader device such as a mobile communication device or a smartphone.

The software can provide a mechanism for the user to define consumables (e.g., a type of food, type of drink, or portion thereof), in a fashion that is convenient to the user. These consumables will be referred to generally herein as a meal or meals, and these terms are used broadly to denote all types of food and drink.

This software can perform a number of functions related to the collection of meal information and association of that meal information with analyte information collected by in vivo analyte sensor 104 or by in vitro test strip and meter, or from trusted computer system 180. The software will be generally referred to, hereinafter, as the “meal monitor application,” “meal monitoring application,” or “meal monitoring app.”

The meal monitor application can allow an individual to log information about each meal that the individual consumes (i.e., each “meal event”). The meal monitor application can associate analyte data from the pertinent time period where the user's log entry indicated that a meal was consumed.

The meal monitor application can also monitor the user's analyte data, and identify when the analyte data changes in a manner indicating or suggesting the occurrence of a potential meal event, and seek to associate meal information from the pertinent time period with that potential meal event. The meal monitor application can prompt the individual to enter meal information relating to a potential meal event, and allow the individual to modify a time of the detected meal event. In some embodiments, if a meal event has been detected, and the meal monitor application determines that meal information has already been entered, then the user may not be prompted.

The meal monitor application can also associate a measured analyte response with a meal event and store the results in a non-transitory memory or a database. In particular, the meal monitor application can display each meal with its associated analyte (e.g., glucose or other analyte) response to the user, for example, as a list where each meal is sorted by descending degree of, using glucose as an example, glycemic response magnitude. Furthermore, the analyte response for each meal event can be depicted in an easy-to-understand graphical representation. For example, if the analyte response to a meal is relatively low (i.e., favorable), the name of the meal can be displayed adjacent to a green indicator to convey that the meal elicited an analyte response that fell within a predetermined low analyte response range. Likewise, if the analyte response to a meal is relatively high (i.e., undesirable), the name of the meal can be displayed adjacent to an orange indicator to convey that the meal elicited an analyte response that fell within a predetermined high analyte response range. In some embodiments, a numeric value to indicate the increase in an analyte level can also be displayed adjacent to the graphical representation.

Another aspect of the meal monitor application includes detection of peak analyte values from analyte responses following a meal. Methods for determining this peak analyte metric are described further herein. Another example embodiment of the analyte response magnitude is determining a difference from the peak analyte value after a meal and the analyte value at the start of the detected meal.

Yet another aspect of the meal monitor application includes analyzing analyte responses for the same or similar meals. For example, if the user consumes the same or a similar meal on repeated occasions, then a weighted average can be determined for that meal and displayed. Furthermore, in some embodiments, the weighted average can give more weight to analyte data that was collected more recently.

Example embodiments of the meal monitor application can utilize analyte data analysis software or software implementable processes, for example, as disclosed in any of U.S. Patent Publication Nos. 2013/0085358, 2014/0350369, 2014/0088393, 2018/0128007, 2017/0185748, 2020/0105397, 2021/0030323, 2022/0000399, or in Int'l Publ. No. WO 2015/153482 or PCT/US20/12134, all of which are incorporated herein in their entirety and for all purposes. Example embodiments of this software are collectively referred to herein as the “meal event detector.” The meal event detector can be an algorithm, routine, or other set of instructions (part of or separate from the meal monitor application) that can detect and/or quantify the occurrence of an actual or potential meal event in the individual's monitored analyte data.

Reference is now made to FIG. 3A, which is a flow diagram depicting an example embodiment of a method 300 for meal information collection, association with analyte data, and determination of glycemic impact of that meal. Method 300 includes acts that may be described as performed by an electronic device, such as reader device 120 (e.g., a smart phone), drug delivery device 160, or computer system 170 or 180, or processors thereof. The user may be an individual or diabetic, clinical administrator, medical professional, dietary profession, or other person. By way of example only, method 300 will be described by reference to a diabetic using the meal monitor software as a downloaded app on a reader device 120 configured as a smart phone. For ease of illustration, the monitored analyte in this and other embodiments described below will be glucose, although other analytes can be monitored as well, as is noted herein.

Referring to FIG. 3A, a meal event can be logged by the user at 302. The user can input meal information directly into reader device 120 (via a user interface) at his or her own discretion, before, during, or after consumption of the meal. In some embodiments, the user inputs meal information in response to a reminder generated by the meal monitor application according to a predetermined schedule, which can be set and/or modified by the user.

Analyte data of the user is monitored at 304. This analyte data monitoring step can be performed in a variety of different ways by a variety of different systems, as illustrated by the system diagrams depicted in FIG. 1 and FIGS. 3B to 3E. In one example embodiment, system 325 (FIG. 3B), analyte data of a user can be transmitted from sensor control device 102 (with analyte sensor 104) worn on the user's body to reader device 120, where the analyte data is received by a sensor interface application 327 residing thereon. In turn, sensor interface application 327 can transmit analyte data to trusted computer system 180, via network 190, where the analyte data can be further processed or aggregated with other analyte data. Subsequently, relevant analyte data is then communicated from trusted computer system 180 to meal monitor application 329, which also resides on reader device 120. In one aspect, according to system 325, sensor interface application 327 and meal monitor application 329 can be separate software programs residing on a single reader device 120.

FIG. 3C depicts another example embodiment of a system 330 for monitoring analyte data of a user, except that sensor interface functionality 327 comprises a module within meal monitor application 329. According to one aspect of system 330, sensor interface functionality 327, which can be authorized to communicate directly with sensor control device 102, can receive analyte data from sensor control device 102, which can then be analyzed by meal monitor application 329. Subsequently, meal monitor application 329 can transmit analyte data to trusted computer system 180, via network 190, where further processing of the analyte data can occur. In some embodiments, meal monitor application 329 can also be configured to transmit meal information to trusted computer system 180.

In another embodiment, FIG. 3D depicts a diagram of another example embodiment of a system 335, in which sensor interface application 327 resides on a first reader device 120A, and meal monitor application 329 resides on a second reader device 120B. According to an aspect of system 335, sensor control device 102 can transmit analyte data directly to sensor interface application 327 which, in turn, communicates the analyte data to trusted computer system 180 via network 190. Subsequently, meal monitor application 329, which resides on a separate reader device 120B, can receive analyte data from trusted computer system 180. In some embodiments, meal monitor application 329 can also be configured to transmit meal information to trusted computer system 180.

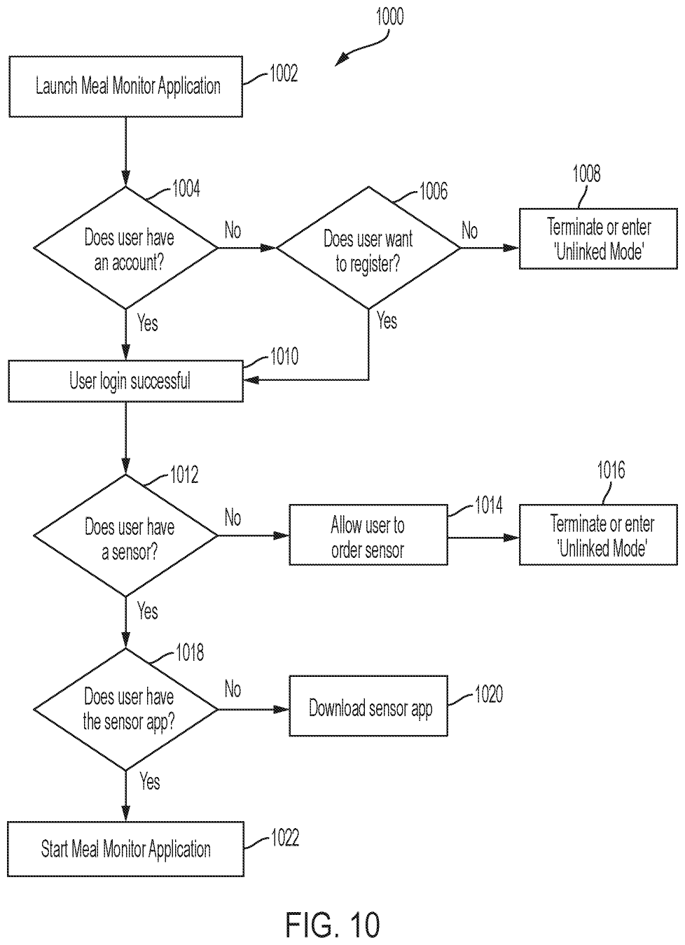

In yet another embodiment, FIG. 3E depicts a diagram of another example embodiment of a system 340, in which analyte data is manually entered or transferred into meal monitor application 329, without direct communication with a sensor control device or trusted computer system. System 340 can be utilized, for example, with meal monitor application 329 in an “unlinked mode,” as further described below with respect to FIG. 10.

In any of the aforementioned systems, information indicative of the time at which each analyte data measurement is collected (e.g., a timestamp) can also be transferred to reader device 120. As already described herein, this data transfer can occur in an on-demand fashion (e.g., the performance of a scan by the user), in a streaming fashion, or other regularly occurring fashion. Analyte data collected by a discrete blood glucose measurement (e.g., such as the reading of a test strip with a meter) can also be entered into reader device 120 manually or automatically.

Referring back to FIG. 3A, reader device 120 can algorithmically process the collected analyte data and determine whether a meal event has occurred at 306. This can occur by use of a meal event detection method, such as described with respect to FIG. 3F, which examines the analyte data for one or more conditions indicative of the occurrence of a meal event. This algorithmic processing can likewise be frequently repeated, e.g., each time new analyte data is received from sensor control device 102, such as in response to a user performed NFC scan of sensor control device 102 with reader device 120 or otherwise. The manual logging of meal information by the user (302) can occur contemporaneously with the monitoring (304) and processing (306) of the analyte data. Those of skill in the art will appreciate that these methods can be implemented in systems where analyte data is autonomously and wirelessly transmitted from the sensor control device to the reader at a predetermined interval.

Referring still to FIG. 3A, each time new data is transmitted to reader device 120, the algorithmic processing can be applied to the new data, which might represent a multiple hour time period (e.g., such as the last 8 hours) to detect meal events. Steps 308-320 can be performed for each meal that is detected, starting with the most recent detected meal and repeating for every other detected meal event.

If a meal event is detected at 306, then, at 308, the meal monitor application assesses whether meal information has already been entered (such as by the user at 302) that corresponds to the detected meal event. This assessment can be performed by examining a period of time before, and optionally to a limited extent after (e.g., to compensate for inaccuracies in time keeping or entry), the times at which the detected event occurred to see if any meal information was entered during that time period. If so, then the meal monitor application can associate that meal information with the detected event at 314. In some embodiments, if multiple meal information entries are found, then the meal monitor application can associate all with the detected event, or can associate the detected event with only the meal information which occurred closest in time.

If no meal information has been entered that can be associated with the detected event, then at 310, the user is prompted to enter the meal information. If no meal event has occurred, the user can decline or ignore the prompt. Otherwise, the meal information can be entered at 312.

Prompting can take the form of an alarm notification, such as a vibration or sound that clues the user into activating the meal monitor application to see the prompt. Alternatively, the prompt may be a notification that appears when the user next views the application.

Regardless of whether the user logs meal information at his or her own discretion (302) or in response (312) to a prompt (310), the meal information can include various levels of detail and can be entered in the same or similar fashion.

Referring back to FIG. 3A, once the information for a meal event has been entered at 312, the meal monitor application will associate the analyte data with that meal information in memory at 314. Specifically, the analyte data occurring around the time of the meal event can be associated with the information for that meal event. The analyte data selected to be associated with the meal event can be chosen based on the time during which the meal event occurred and optionally a period of time after which the meal event occurred to reflect changes in the analyte level due to digestion of the meal.

In some embodiments, analyte data occurring from the time at which the meal began until a period of time after the meal ceased can be associated with the meal event. Also, analyte data occurring from the time at which the meal began until the conclusion of a detected glucose excursion can be associated with the meal event. In some embodiments, analyte data collected during a fixed range of time around the meal event is associated with the meal event, for example, any combination of from one, two, or three hours before the meal event (e.g., as measured by initiation of the meal event, a median time of the meal event, or a conclusion of the meal event) until one, two, three, four, five, six, seven or eight hours after the meal event. In each case, times over which the meal event occurred can be identified based on information entered by the user or can be identified algorithmically through analysis of the analyte data.

The association of analyte data with a meal event can be used in determining a glycemic impact of the meal event at 316. In some embodiments, determination of the glycemic impact of the meal event can be done algorithmically with reference to analyte data contemporaneous with the meal event, and this algorithmic processing can constitute both steps 314 and 316. The determined glycemic impact can be determined quantitatively in terms of maximum (peak) or minimum glucose level, median or mean glucose level, minimum-to-maximum change in glucose level (delta (A) glucose value), percent variability of glucose level, duration of glucose response, rate of change of glucose level, area of the glucose response, and any combination thereof.

The meal event detector outputs information about the glycemic response to the meal that can be used to characterize the magnitude or severity of the response. For example, the meal event detector can output the start time and the peak time for each detected meal event. The meal-start glucose can be the glucose value at the meal event start time and the peak glucose can be the glucose value when a rise episode of the detected meal event peaks. The difference in these glucose values can be determined to provide a delta glucose measure of the glycemic response to the meal.

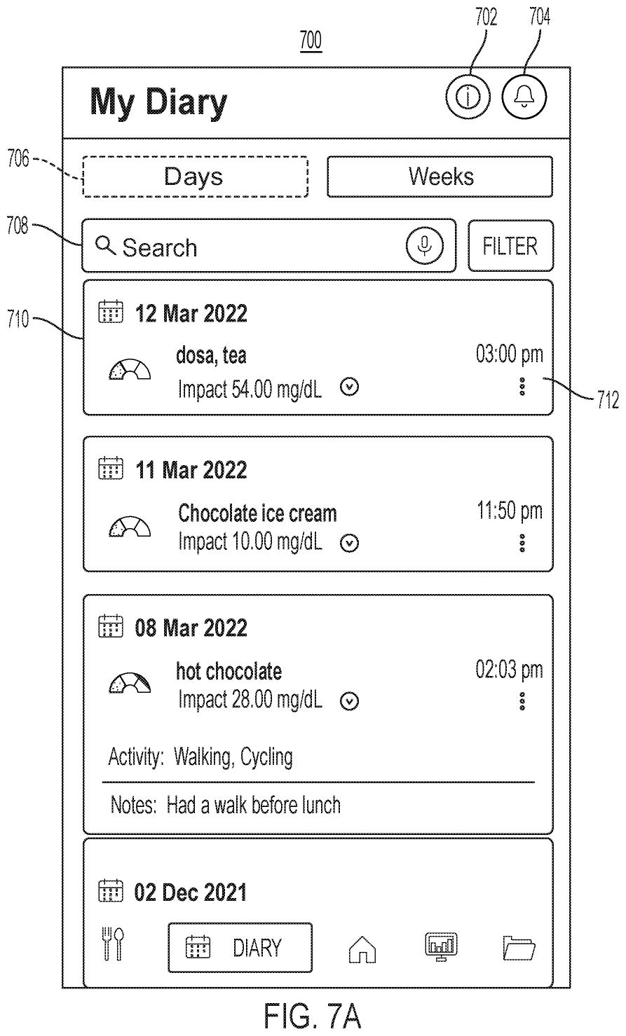

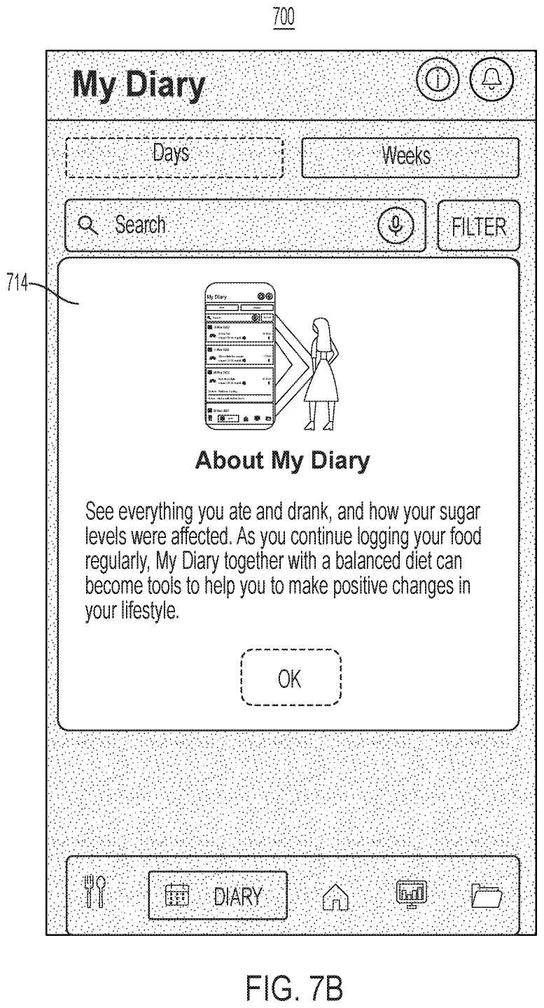

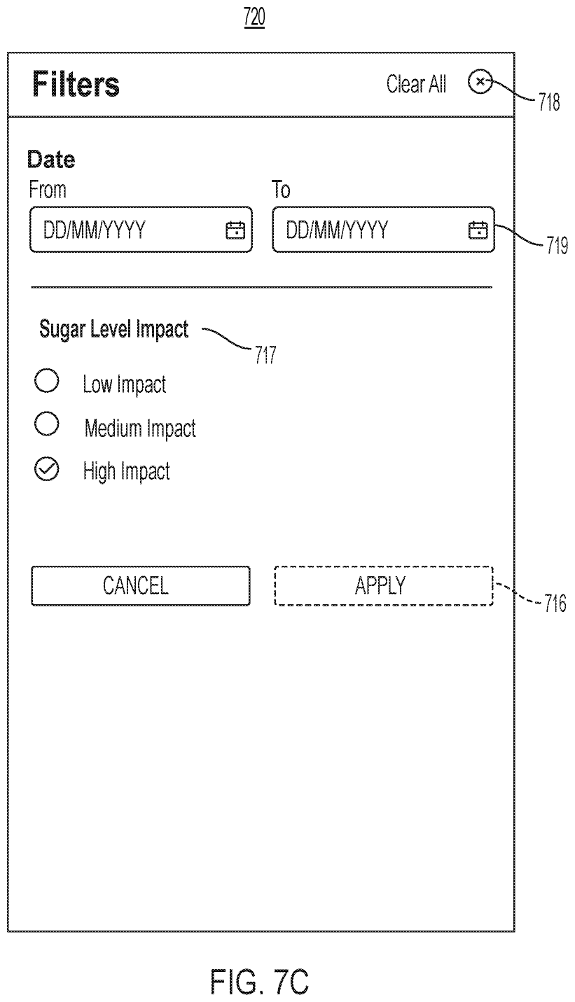

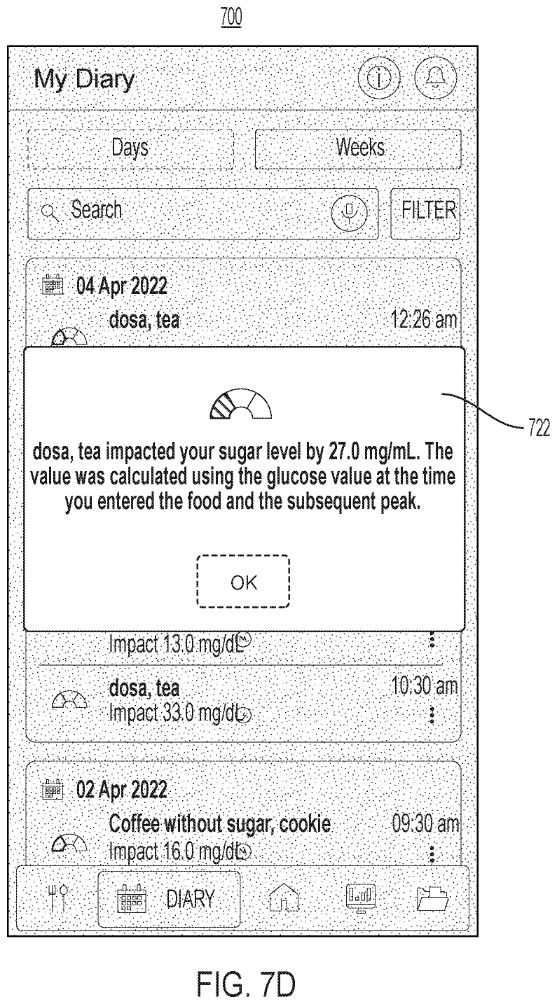

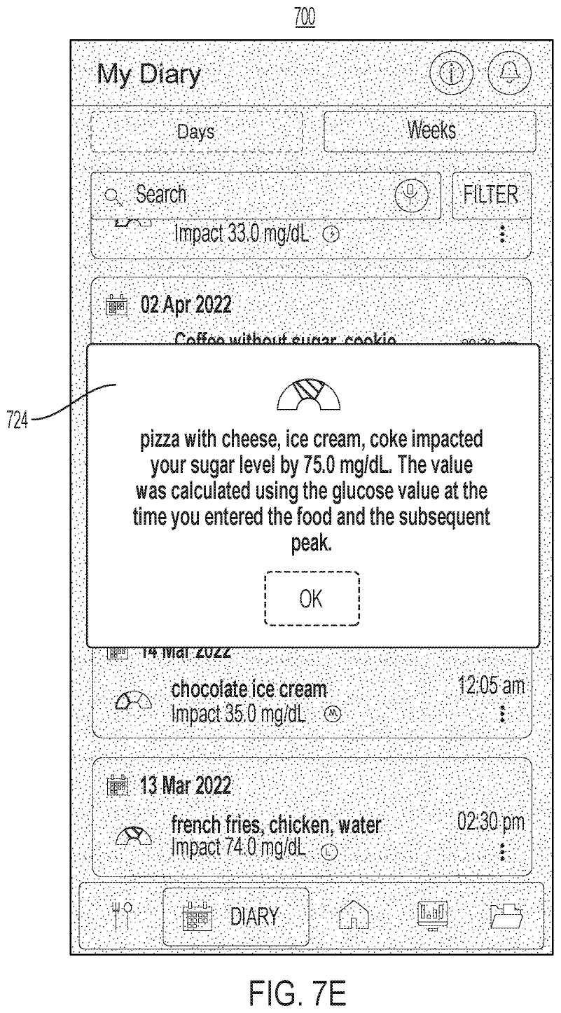

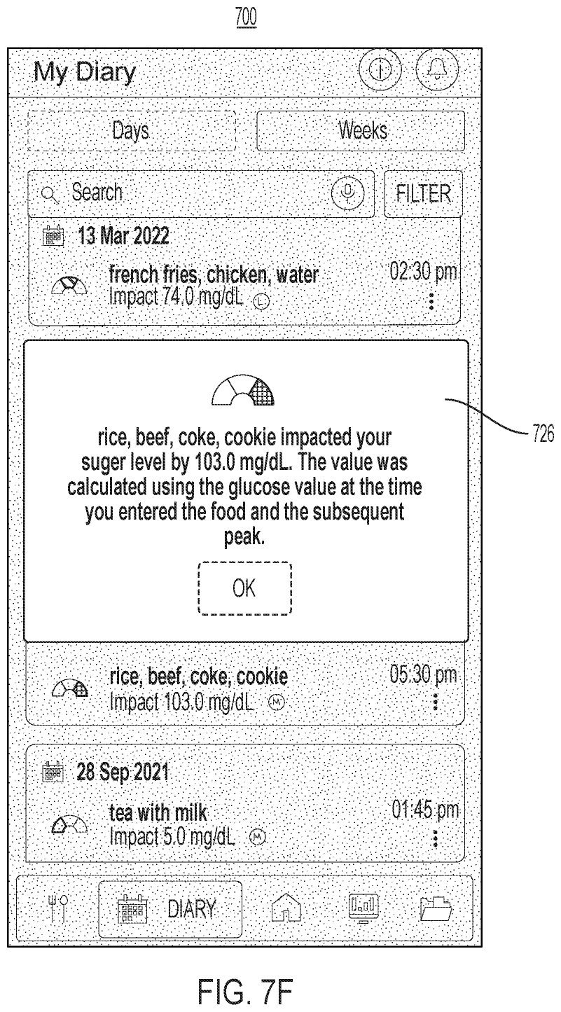

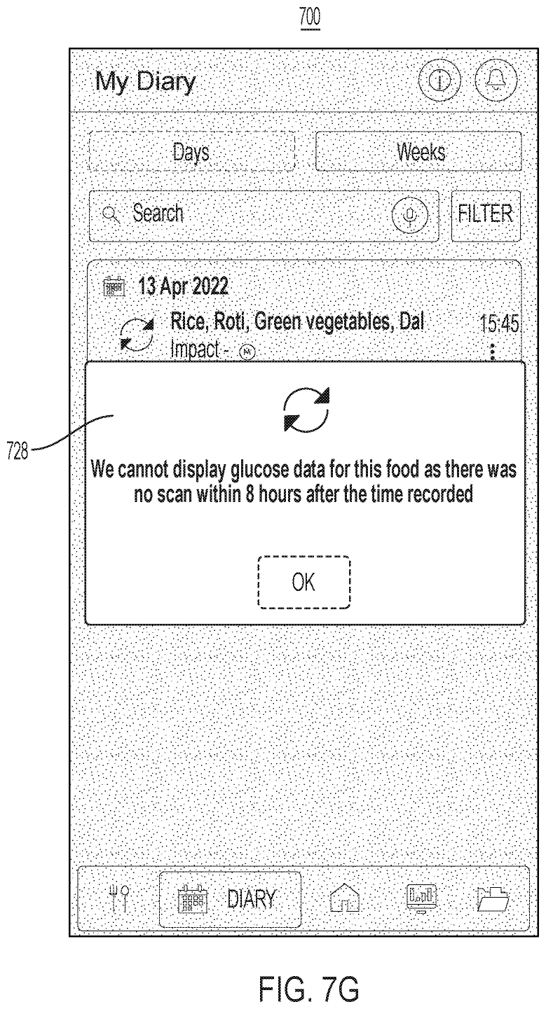

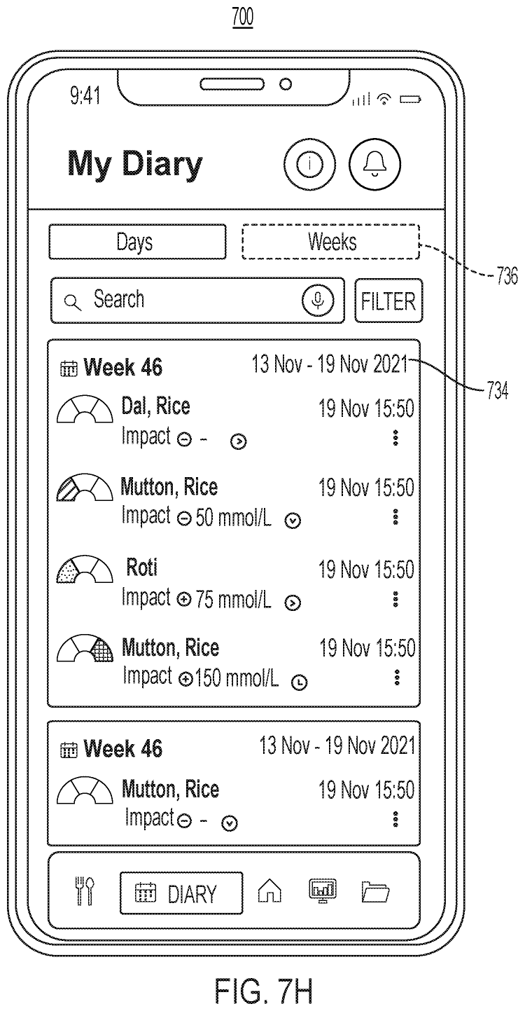

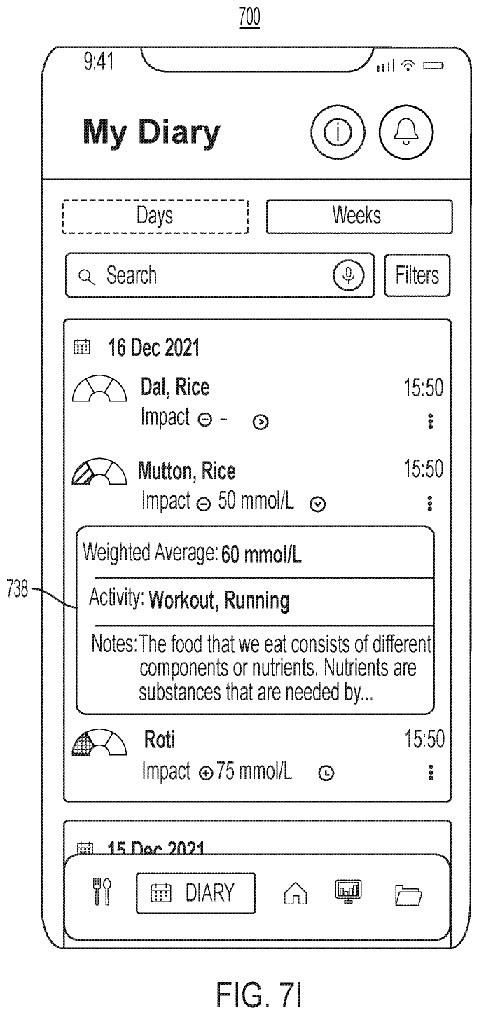

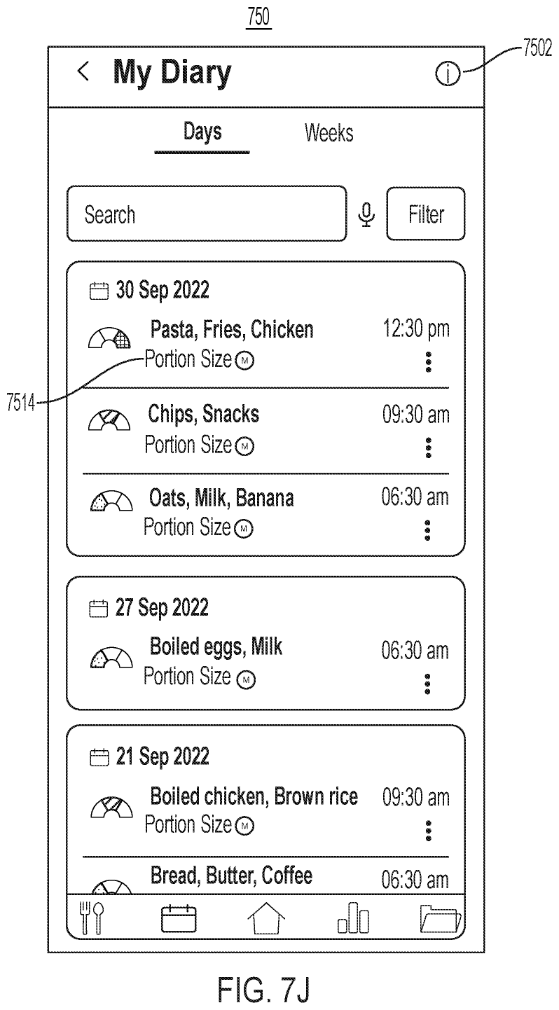

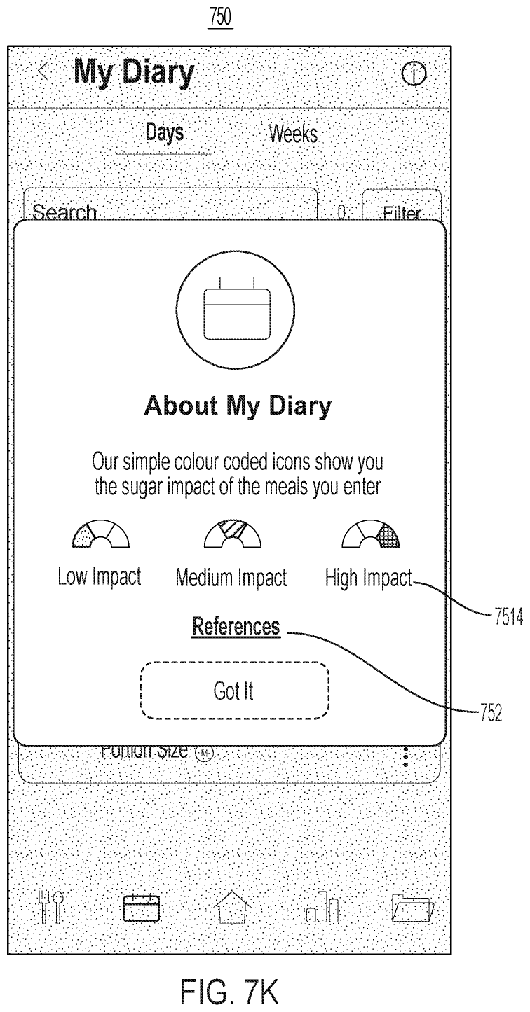

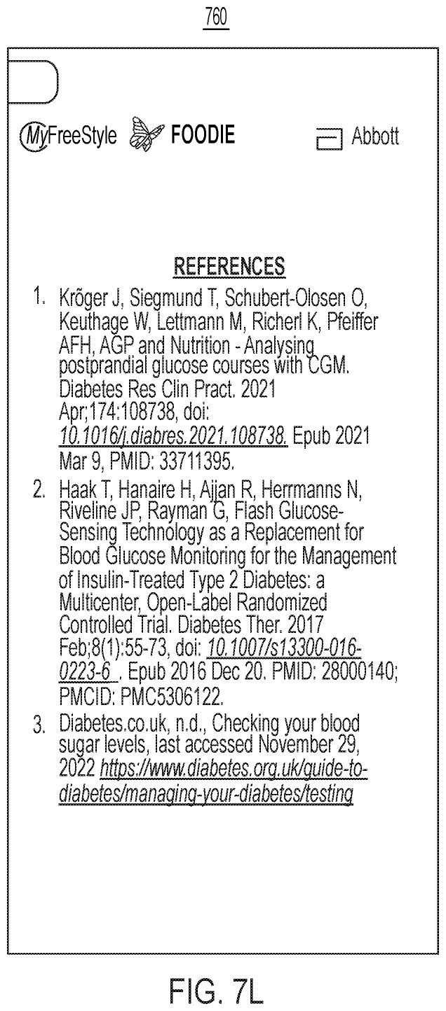

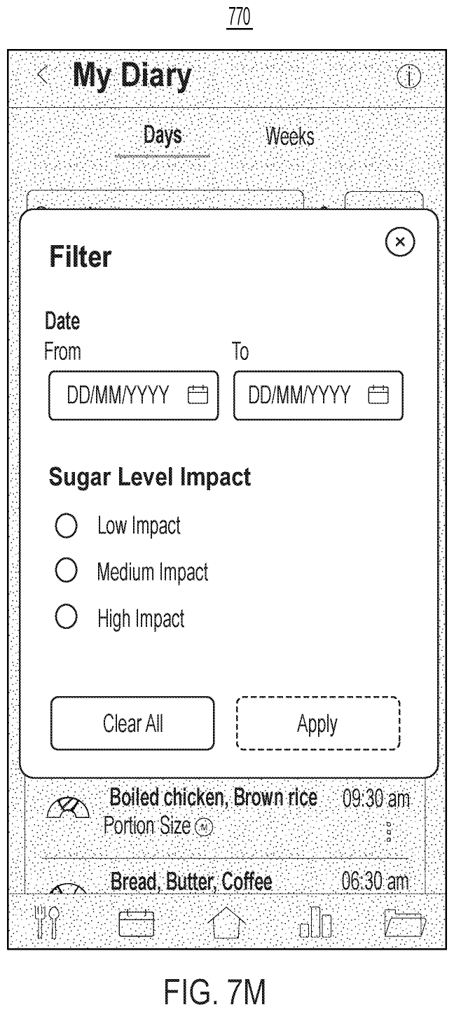

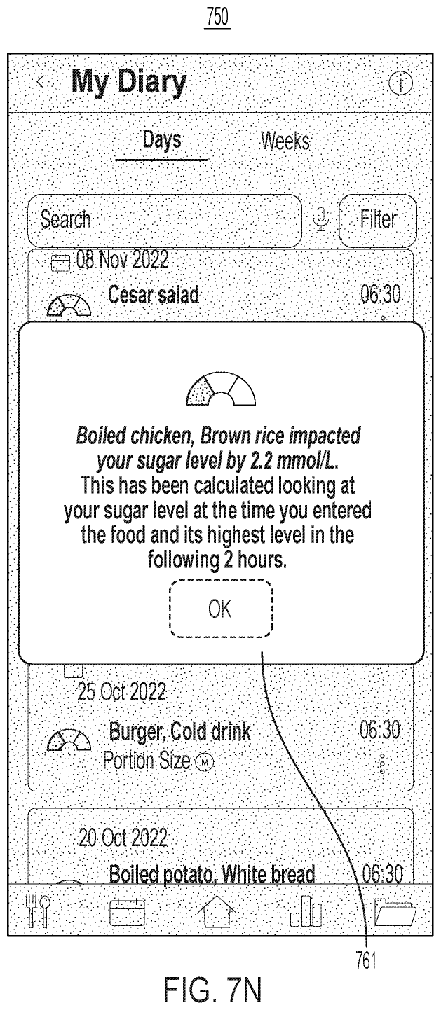

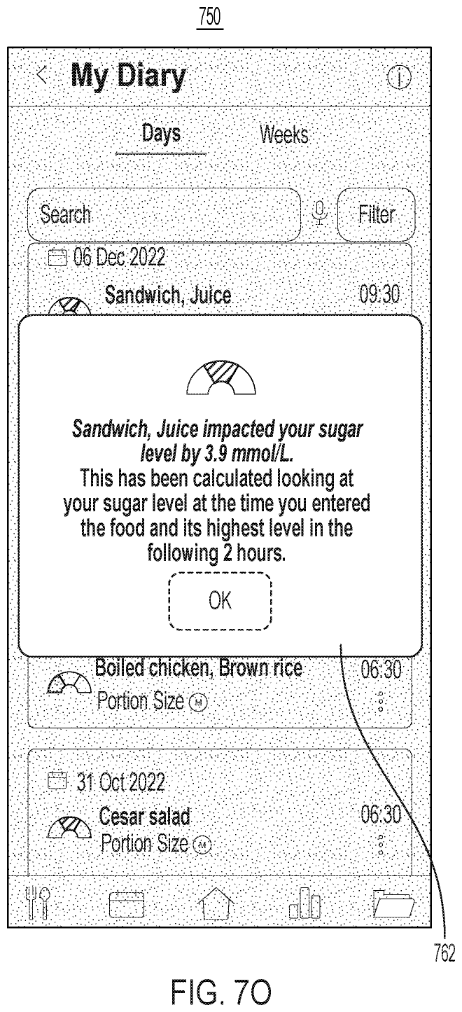

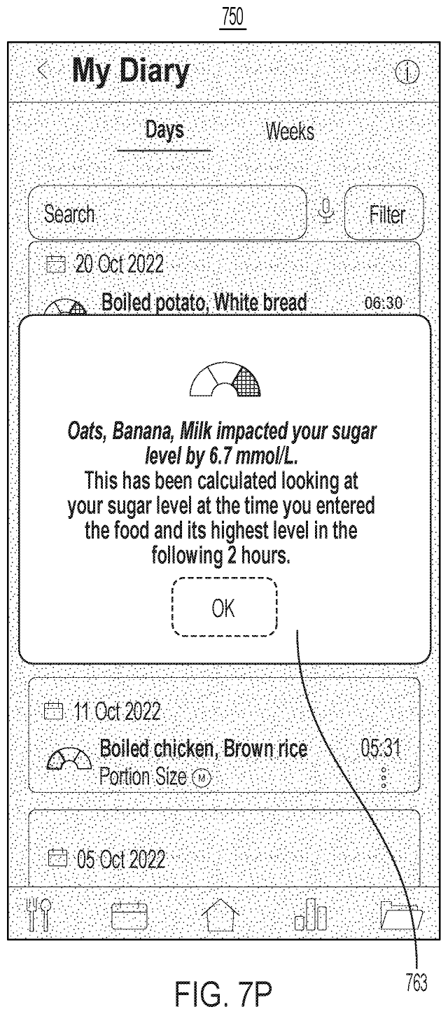

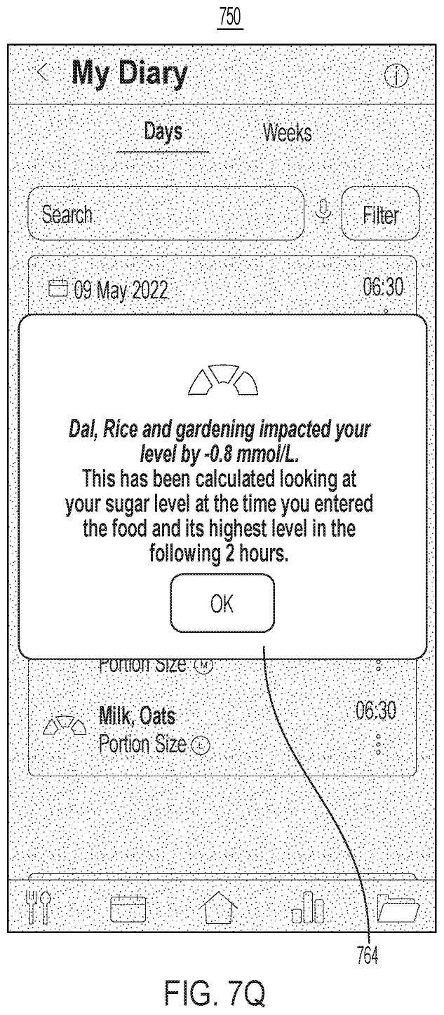

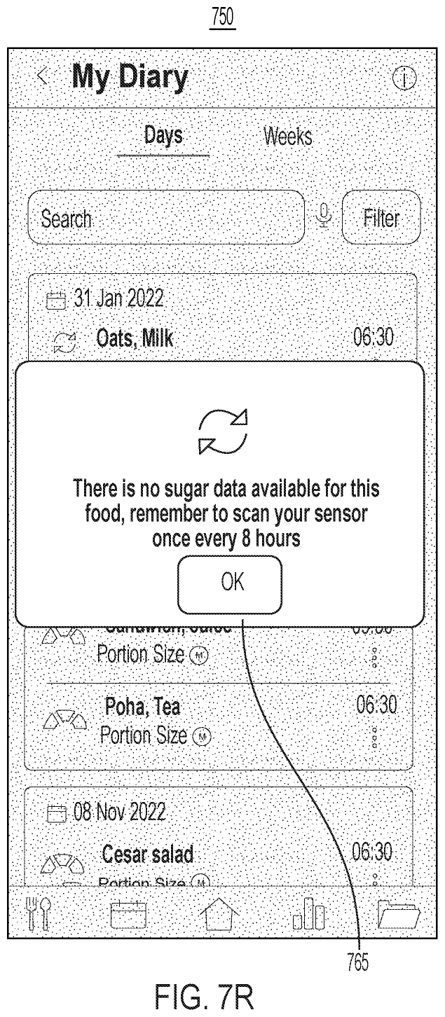

Referring back to FIG. 3A, the determined glycemic response or impact can then be output to the user at 318, such as visually on a display of the smartphone, as will be described below with respect to FIGS. 7A to 7I. The determined glycemic response can be output in quantitative and/or qualitative terms. For example, the determined glycemic response can be output in the same quantitative measure in which it was determined at 316. This can be done in text and/or graphical form. Also, the determined glycemic response can be output qualitatively, for example, described in text as low or minor, moderate or medium, or high or severe, or any synonym thereof. Less or more graduations of magnitude can be used as well. The determined glycemic response can also or alternatively be output as an icon or other imagery (e.g., colored shapes of various degrees of magnitude).

Referring back to FIG. 3A, method 300 can repeat itself such that the meal monitor application continues to receive, monitor and store the analyte data (e.g., step 304 of method 300) and search for detected events 306 continually. The recently entered information about a detected or logged meal can be included in the options presented to the user for entering meal information each time a new event is detected.

As will be described below, the information output by the meal monitor application provides the user with concrete, easy-to-understand, and immediate information regarding the impact of their meals on their analyte levels. This output information can help users learn to avoid or minimize certain meals in their diet that they did not realize were impacting their glucose levels, e.g., that they did not realize were driving their glucose levels so high. This output information can also help users learn to control the portion sizes of their meals by seeing the relative impact of different portion sizes on their glucose levels.

Example embodiments of methods for associating analyte data with meal information will now be described. Turning to FIG. 3F, a flow diagram depicts an example embodiment of a method 350 for associating analyte data with meal information, where the meal information was not previously entered by the user. As an initial matter, those of skill in the art will recognize that method 350, either in its entirety or any one or more of the individual steps, can be combined with, or implemented as part of, method 300 of FIG. 3A. Method 350 begins at 352, where data indicative of a user's analyte level is received. In some embodiments, for example, this can occur when a user scans their sensor control device with their reader device. In other embodiments, this can occur autonomously if the sensor control device is configured to wirelessly stream analyte data to the reader device. At 354, a peak analyte level value within a predetermined time period of the received analyte data is identified. In some embodiments, for example, this can entail identifying the highest glucose value over a predetermined analyte level threshold (e.g., 170 mg/dL, 180 mg/dL, 190 mg/dL, etc.) within the received analyte data. Furthermore, in some embodiments, the predetermined time period can comprise a set number of hours of analyte data (e.g., last two hours of analyte data, last four hours of analyte data, last eight hours of analyte data, last twelve hours of analyte data, etc.) within the received analyte data.

Referring still to FIG. 3F, at 356, the estimated meal start time can be determined, as well as an initial analyte level value associated therewith. In some embodiments, for example, the estimated meal start time can be identified by counting back from the time of the peak analyte level value (e.g., two hours before the peak analyte level value, three hours before the peak analyte level value, four hours before the peak analyte level value, etc.). The initial analyte level value can be determined by referencing the analyte level reading at the estimated meal start time.

Subsequently, at 358, the analyte level variance value is determined by subtracting the initial analyte level value from the peak analyte level value. Then, at 360, method 350 can prompt the user to enter meal information. At 362, the inputted meal information is associated with and stored in memory with the analyte level variance value.

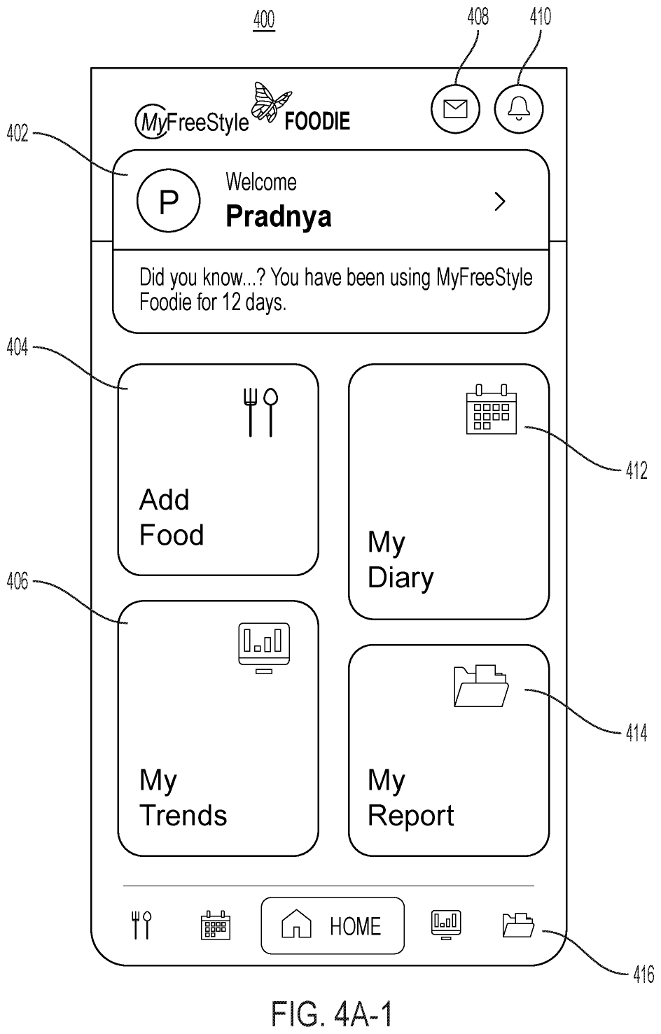

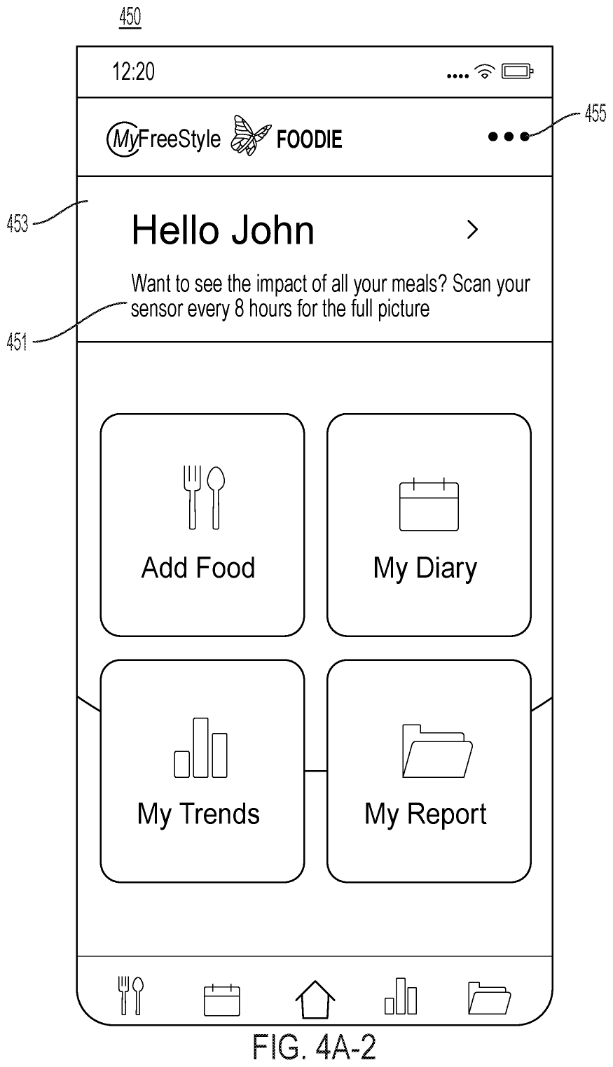

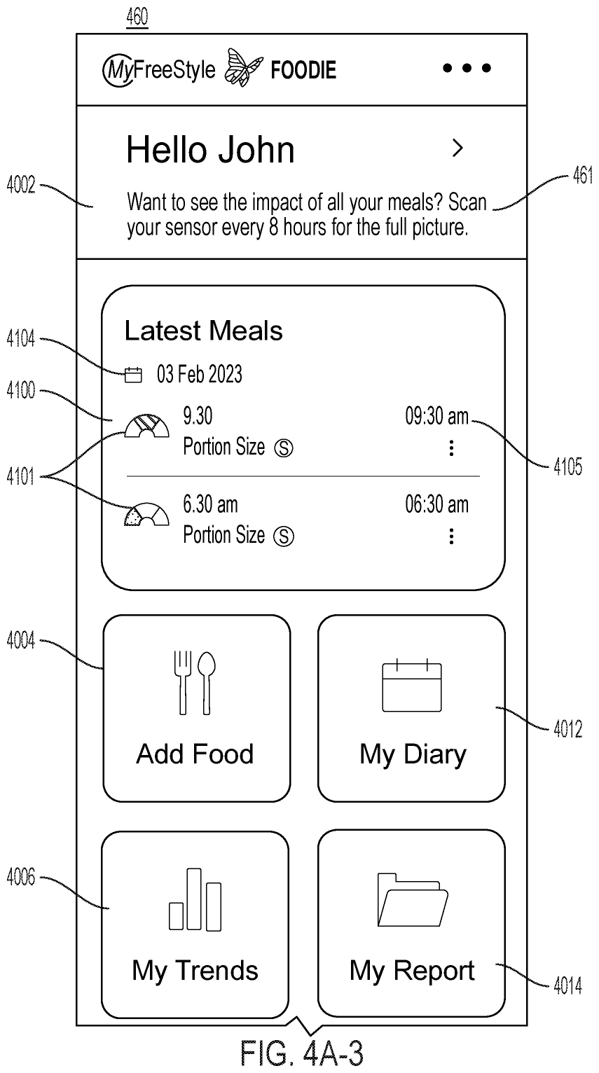

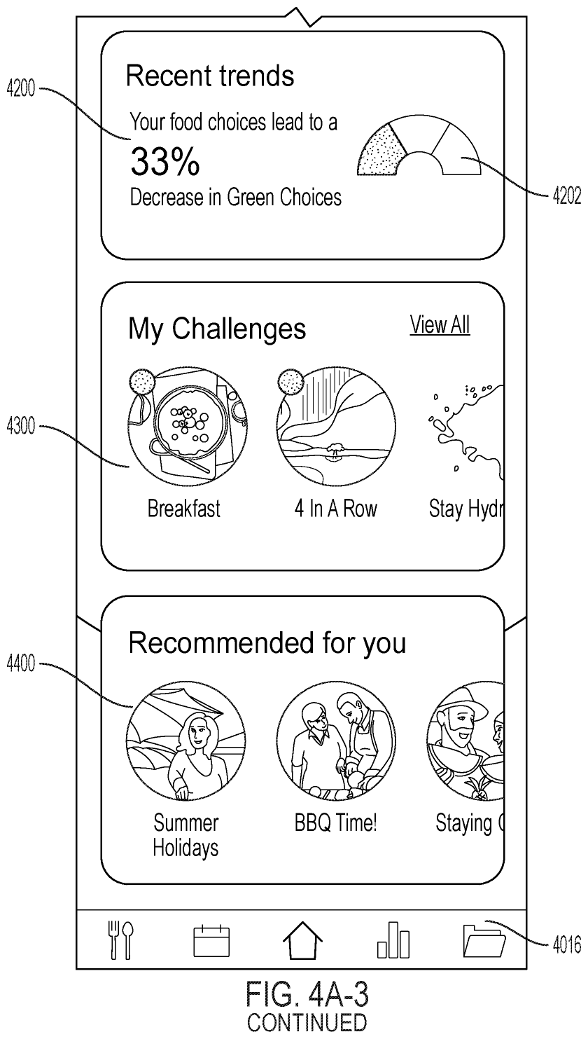

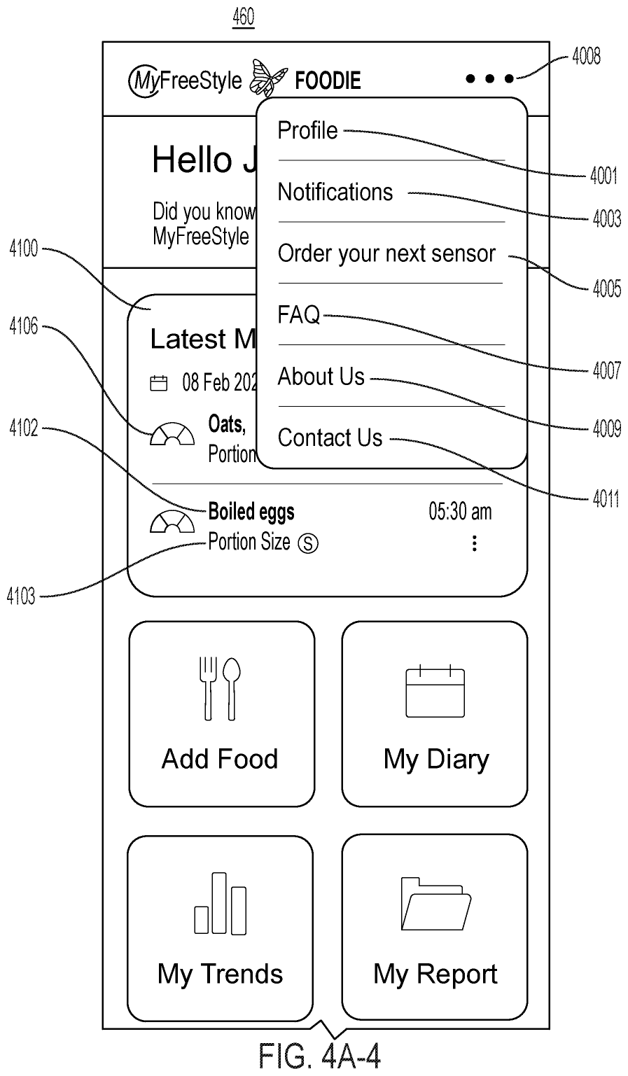

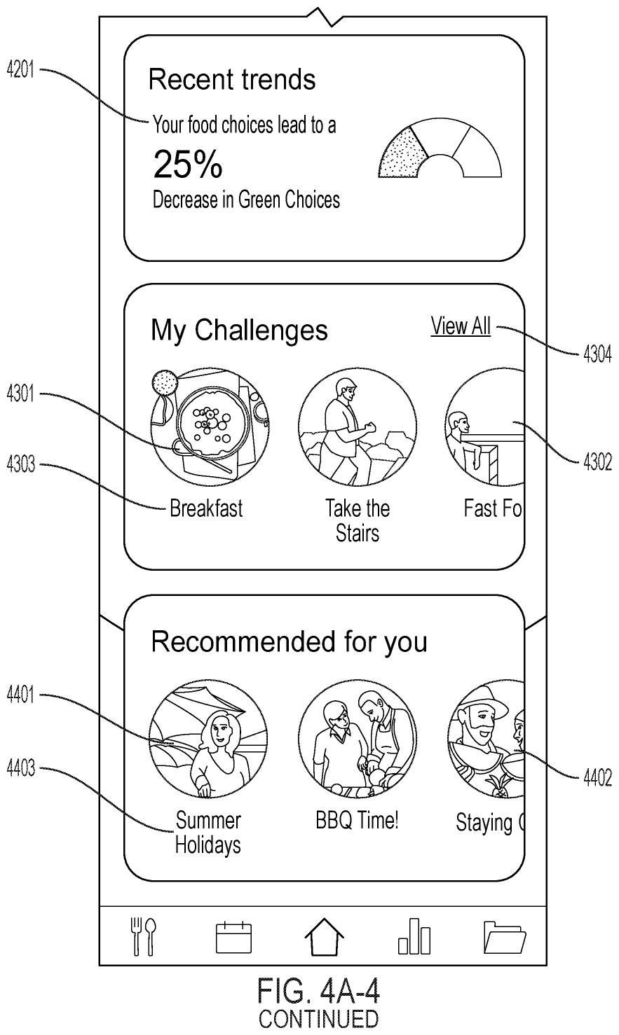

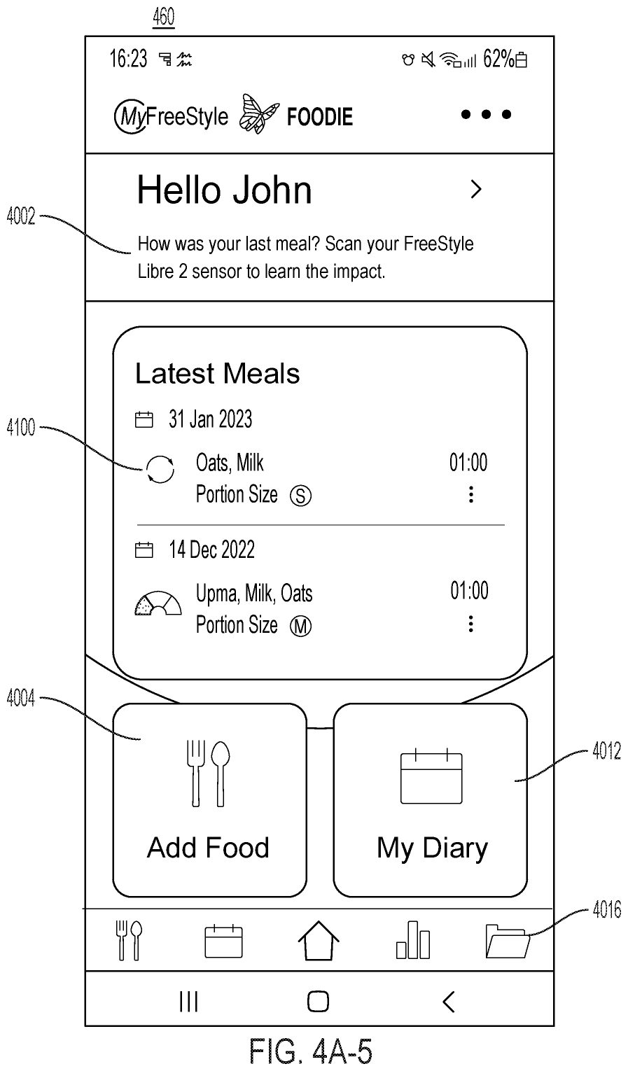

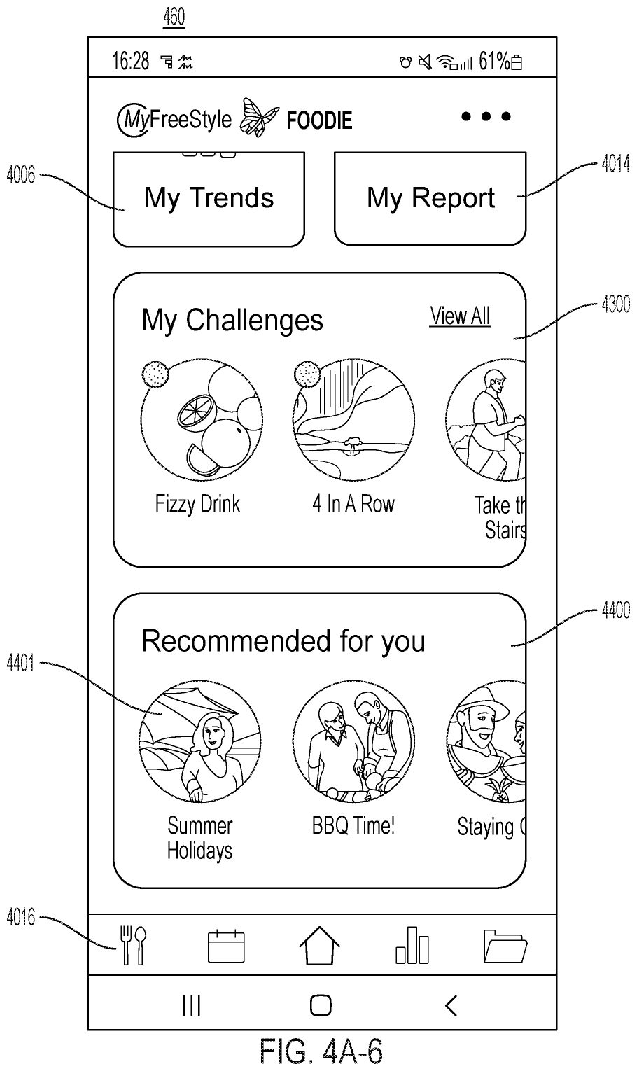

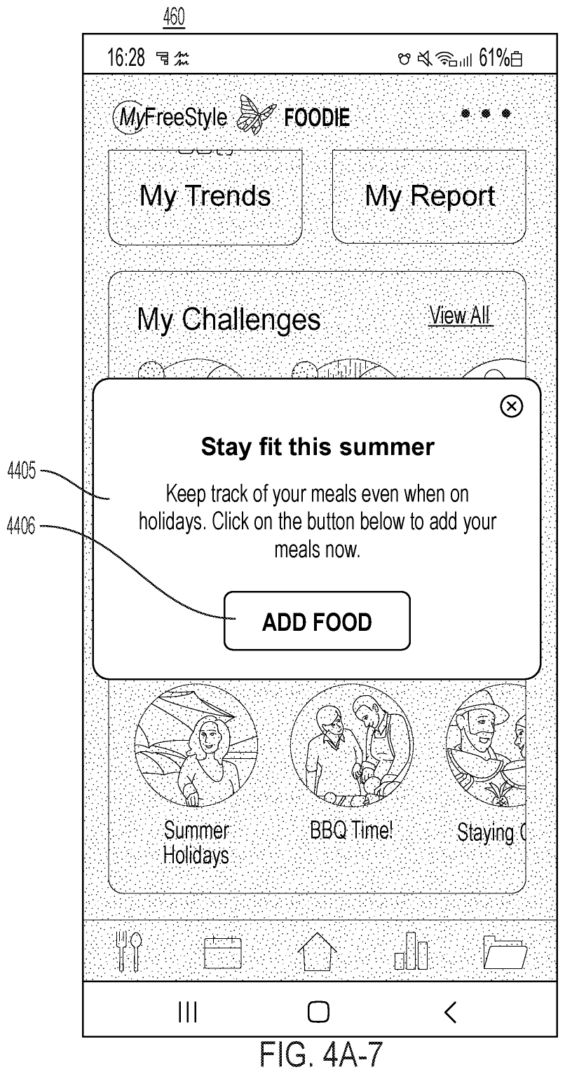

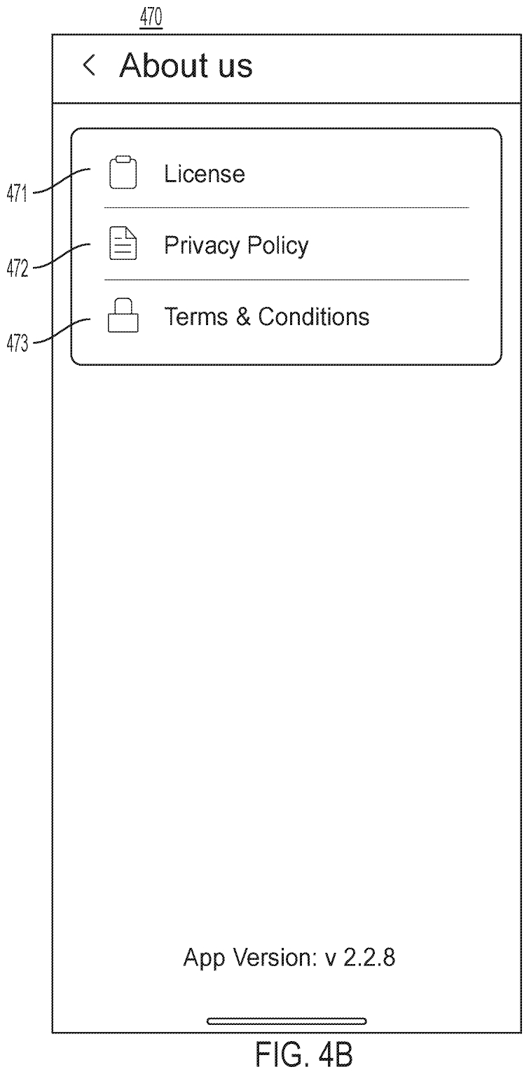

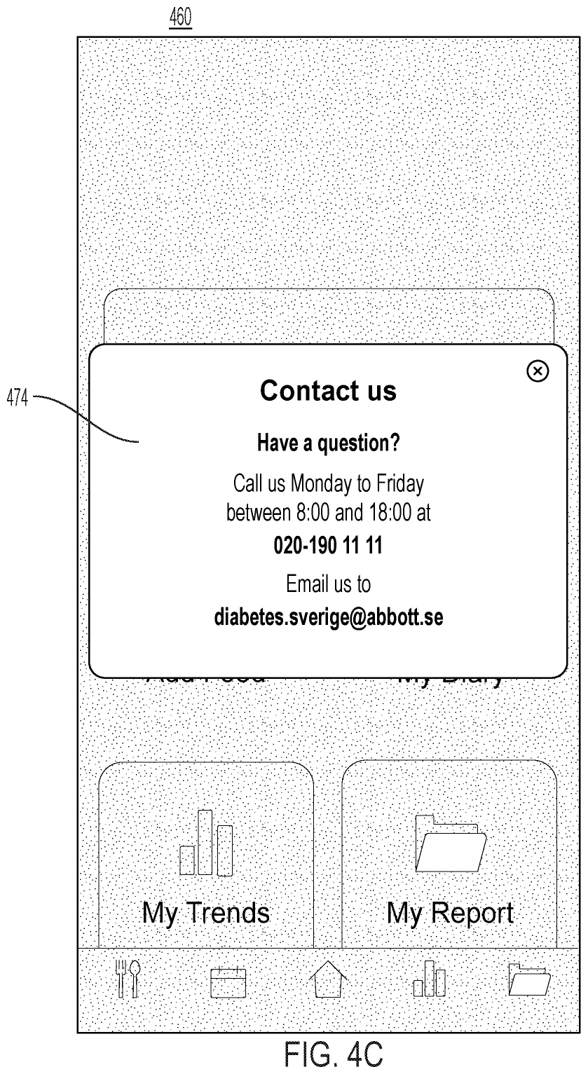

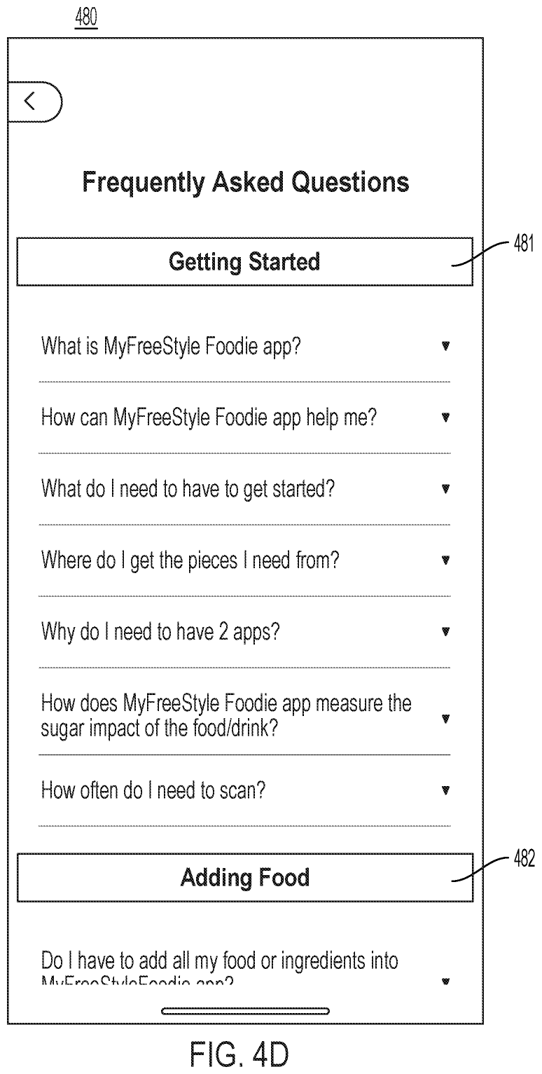

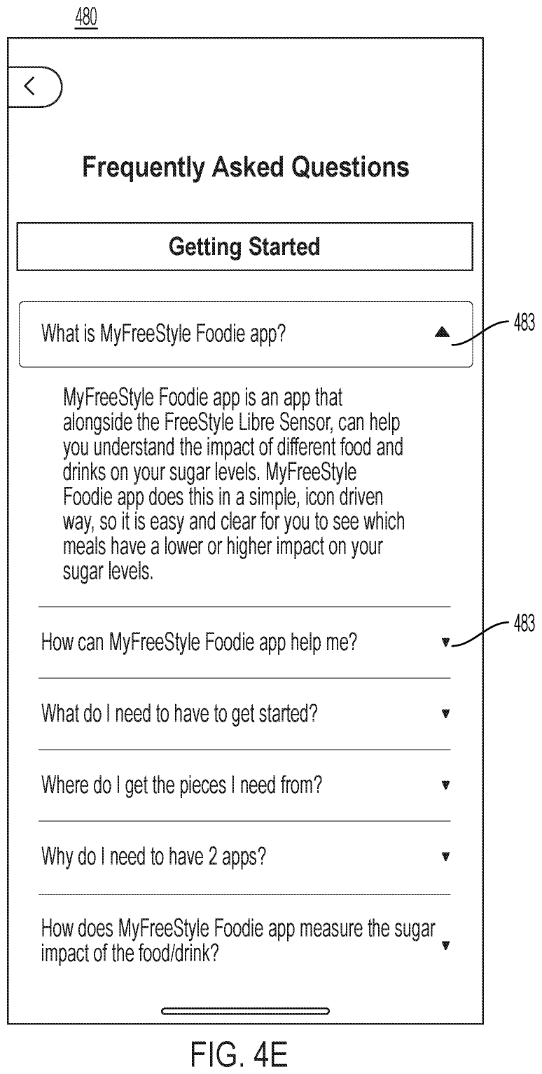

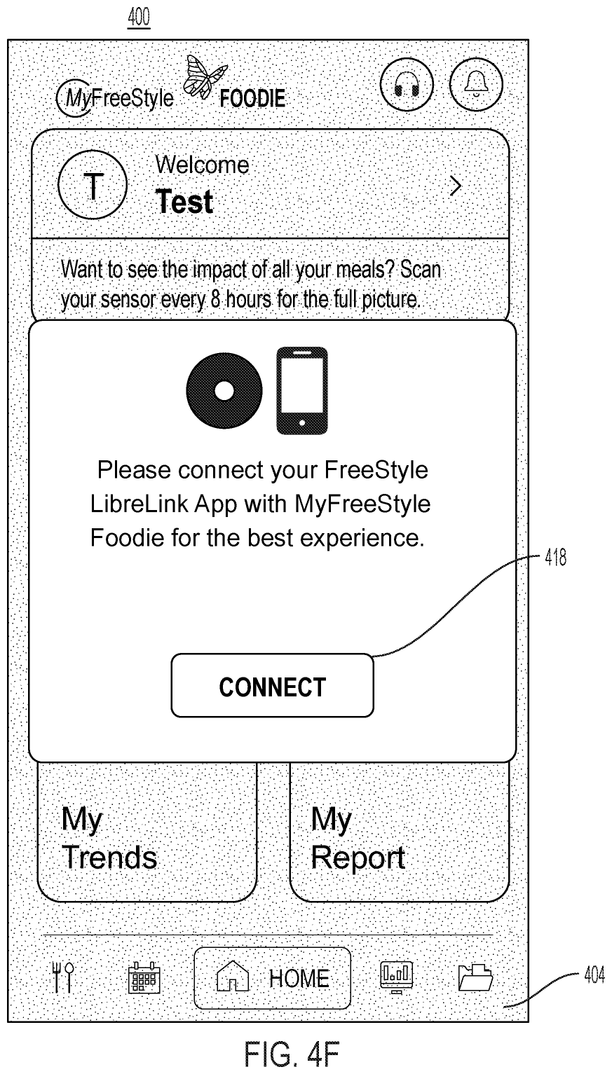

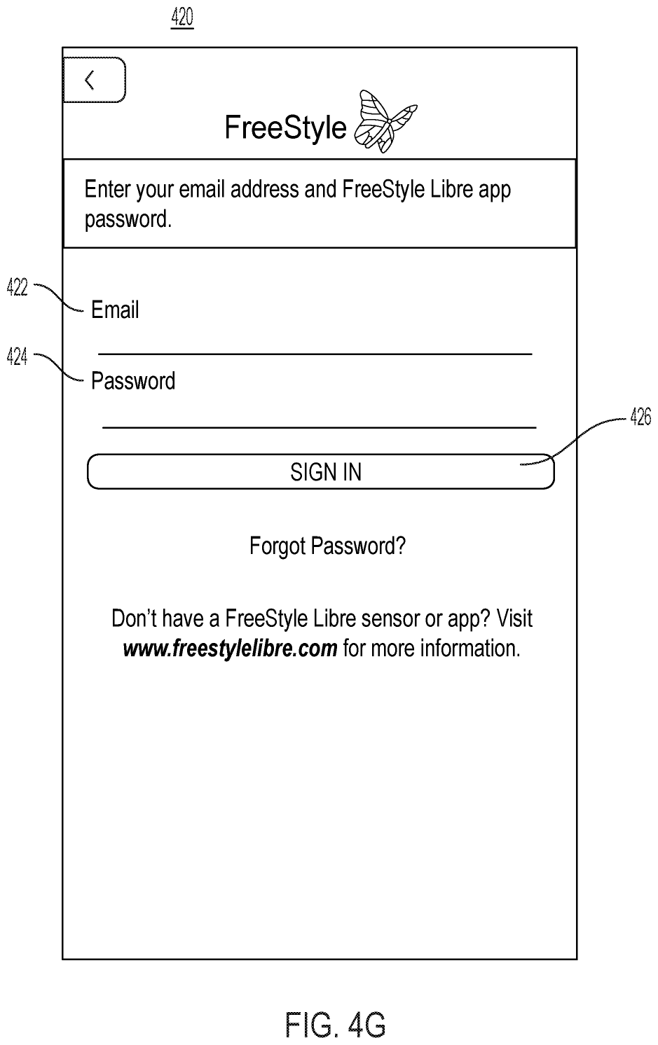

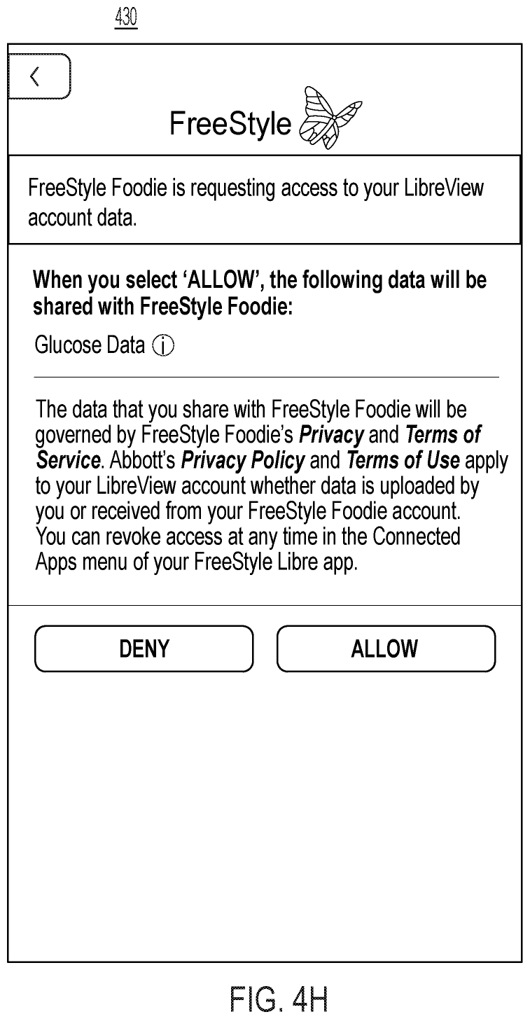

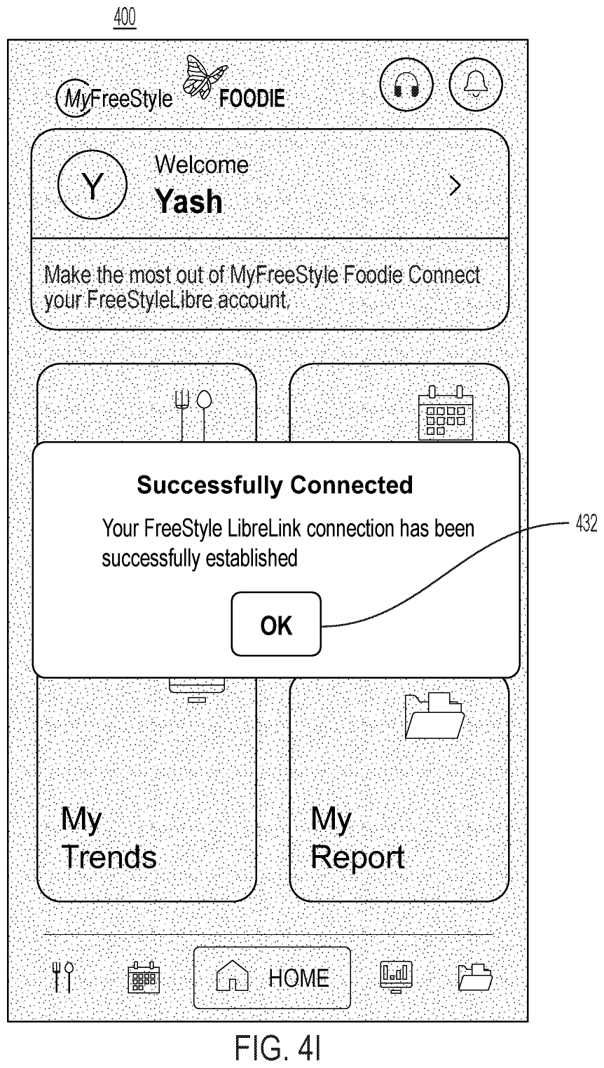

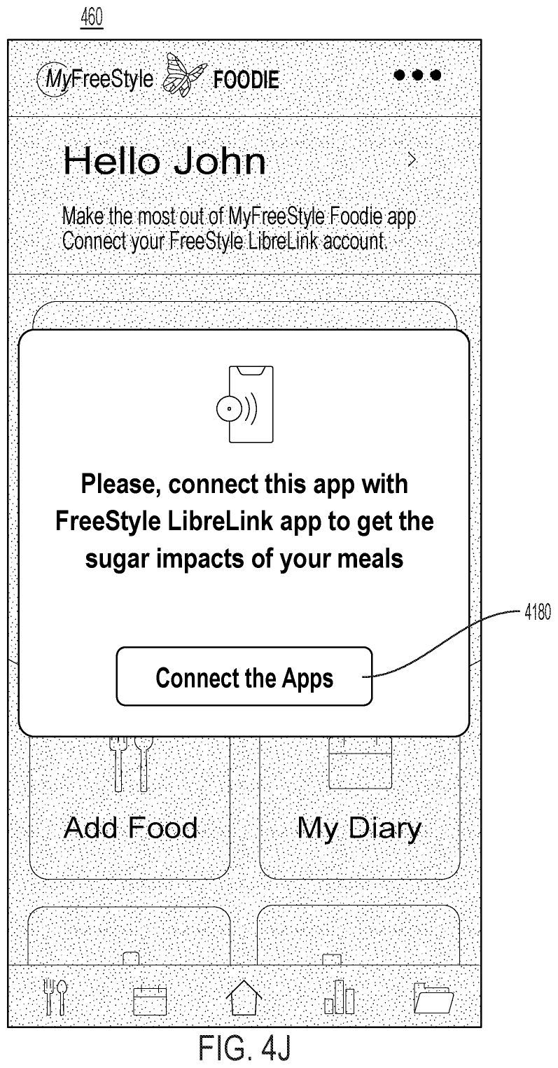

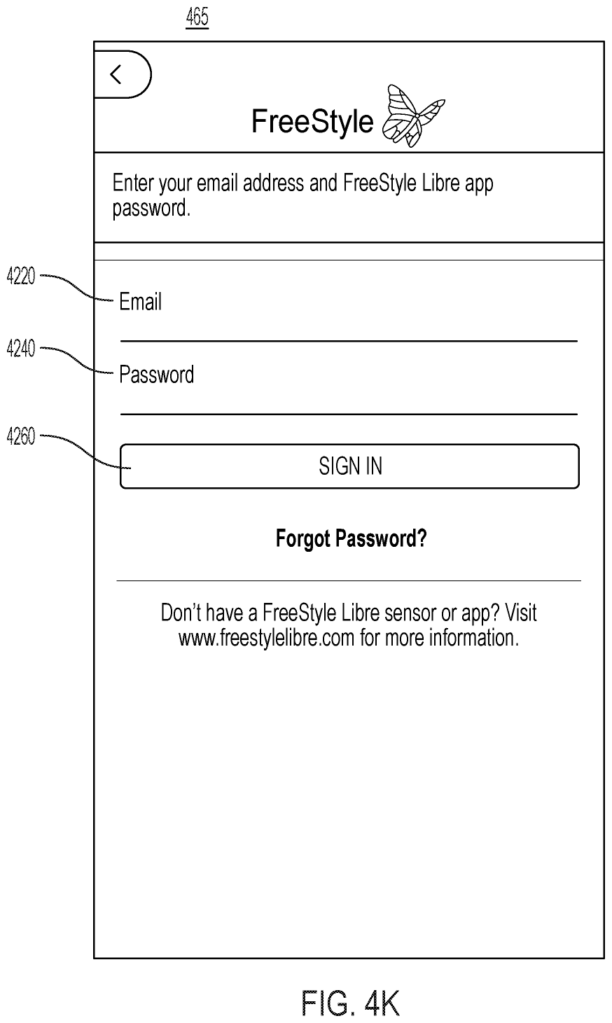

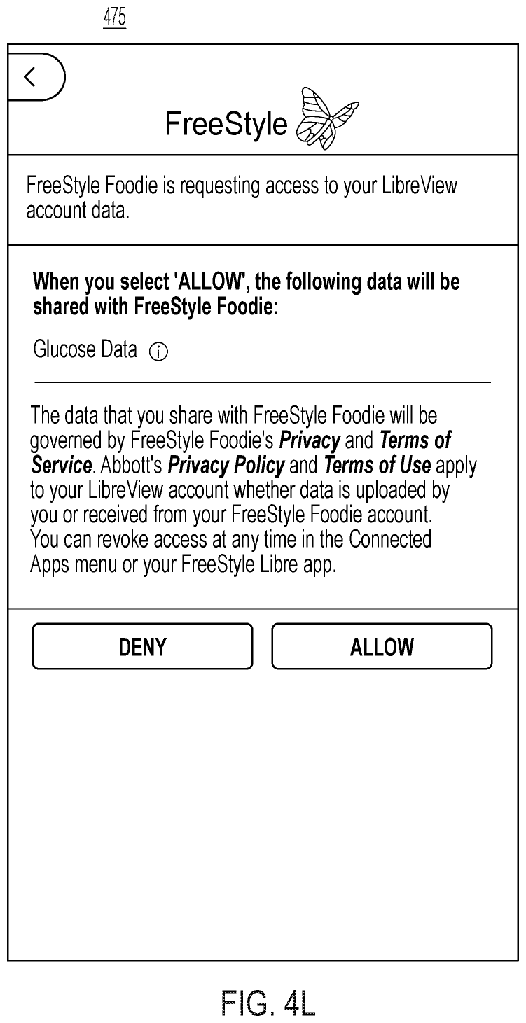

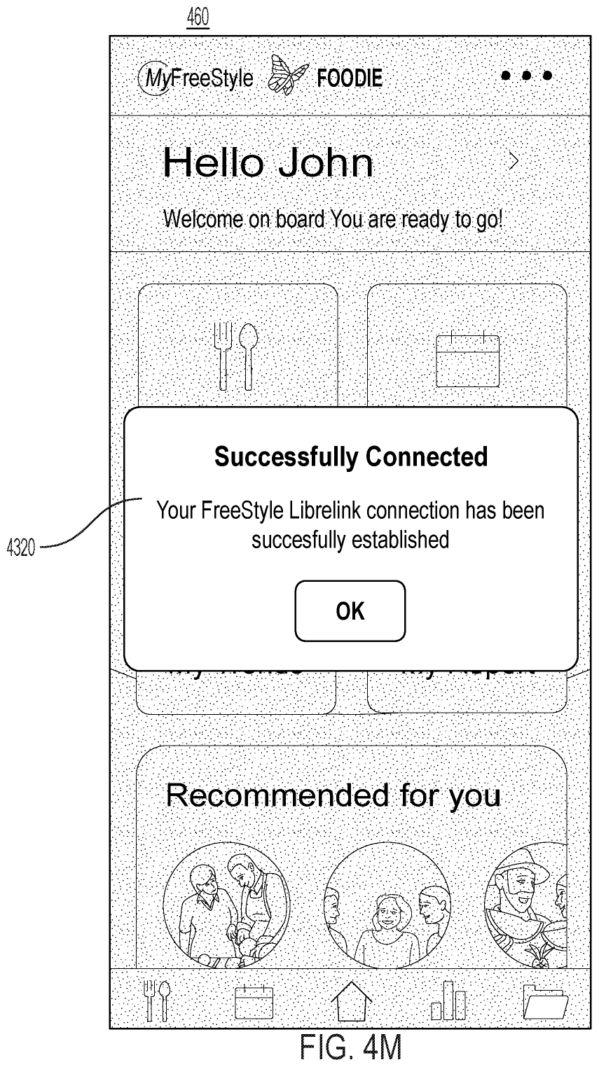

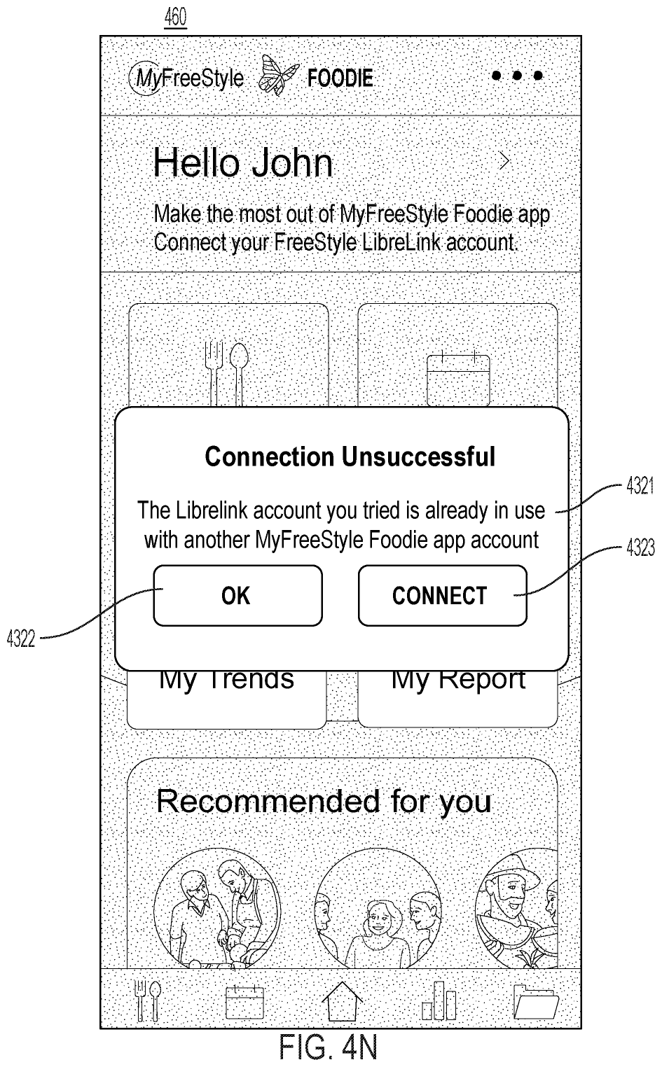

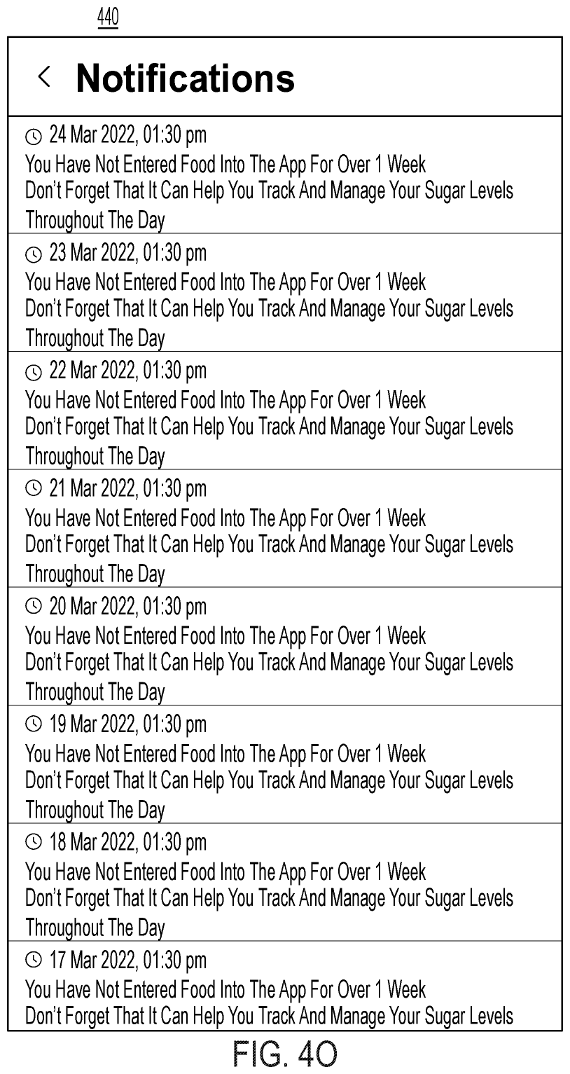

Turning to FIG. 3G, a flow diagram depicts an example embodiment of a method 370 for associating analyte data with meal information, where the meal information has already been entered by the user. As an initial matter, those of skill in the art will recognize that method 370, either in its entirety or any one or more of the individual steps, can be combined with, or implemented as part of, method 300 of FIG. 3A. As seen at the top of FIG. 3G, method 370 begins at 372, where meal information is inputted by the user. In some embodiments, for example, this can be a meal entry proactively entered by the user without any prompting from the meal monitor application. In other embodiments, the user can input meal information in response to a prompt displayed by the meal monitor application. For example, according to some embodiments, the meal monitor application can be configured to display a reminder notification to the user (as shown in Notifications GUI 440 of FIG. 4F) if a meal entry has not been entered after a predetermined reminder time period (e.g., no meal entry in the past week, no meal entry in the past three days, no meal entry in the past day, etc.).

Subsequently, at 374, data indicative of an analyte level of the user is received by the meal monitor application within a predetermined amount of time. In some embodiments, this can entail the user scanning their sensor control device within a predetermined amount of time from the time of the meal entry (e.g., within four hours of the meal entry, within eight hours of the meal entry, within twelve hours of the meal entry, etc.). In other embodiments, this can occur autonomously if the sensor control device is configured to wirelessly stream analyte data to the reader device.

Referring still to FIG. 3G, at 376, a peak analyte value in the analyte data is identified. In some embodiments, this can entail identifying the highest glucose value over a predetermined analyte level threshold (e.g., 170 mg/dL, 180 mg/dL, 190 mg/dL, etc.). The peak analyte value can also be a glucose value over the predetermined analyte level threshold during a time window after the meal entry (e.g., during a two-hour window after meal entry, during a three-hour window after meal entry, during a four-hour window after meal entry). Then, at 378, the initial analyte level value is determined. According to some embodiments, the initial analyte level value can be determined by ascertaining the analyte level value at or near the time of the meal entry.

Subsequently, at 380, an analyte level variance value can be determined, for example, by subtracting the initial analyte level value from the peak analyte level value. Then, at 382, the analyte level variance value can be associated and stored together in memory with the meal entry inputted by the user.

### Example Embodiments of GUIs and Related Features for a Meal Monitor Application

Example embodiments of various GUIs and related software features for a meal monitor application that can perform any of the aforementioned methods 300, 350, and 370, will now be described. Those of skill in the art will understand that these various interfaces can be displayed on any of the embodiments of reader device 120 (e.g., a smart phone), drug delivery device 160, or local computer system 170 described herein.

### Example Embodiments of Home GUIs and Related GUIs

FIGS. 4A-1 to 4A-7 illustrate exemplar embodiments of home GUIs (also herein referred to as “home screen GUIs”) and features related thereto for use with a meal monitor application.

Referring to FIG. 4A-1, an example embodiment of a home GUI 400 for a meal monitor application is depicted. According to one aspect of the embodiments, home GUI 400 can comprise a plurality of selectable sections including, but not limited to, a user profile section 402, a meal entry section 404, a trends section 406, a diary section 412, and a reports section 414. Selection by the user of a section can cause the meal monitor application to display one of the corresponding GUIs described in further detail below. In some embodiments, home GUI 400 can also include a navigation bar 416 (bottom), an e-mail button 408 (top) to request support from the manufacturer, and a notification button 410 (top) configured to relay in-app notifications to the user. Selection of the notification button 410, for example, can cause the meal monitor application to display a log of notifications, as shown in GUI 440 of FIG. 4O.

FIG. 4A-2 depicts an additional example embodiment of a home GUI for use with a meal monitor application. Specifically, FIG. 4A-2 depicts a home GUI 450, which is similar to the home GUI 400 depicted in FIG. 4A-1. According to one aspect of some embodiments, home GUI 450 comprises an informational button 455 (top) configured to display a plurality of selectable links which, when selected by the user, can cause the meal monitor application to display a GUI corresponding to the selected link. Additionally, home GUI 450 further comprises a banner 451 comprising a message reminding the user to scan the sensor every eight hours in order to see the impact of all meals. In some embodiments, the banner 451 is displayed directly adjacent to and distal relative to the greeting and user's name as displayed on the user profile section 453.

FIGS. 4A-3 and 4A-4 illustrate another exemplar embodiment of a home GUI for use with a meal monitor application. FIGS. 4A-3 and 4A-4 depict a home GUI 460, which, similar to home GUIs 400 and 450, can comprise a plurality of selectable sections including, but not limited to, a user profile section 4002, a meal entry section 4004, a trends section 4006, a diary section 4012, and a reports section 4014. In like manner to home GUIs 400 and 450, home GUI 460 can also include a navigation bar 4016 (bottom). According to an aspect of the embodiments, home GUI 460 can further comprise: (1) a meals card 4100; (2) a trends card 4200; (3) a challenges card 4300; and (4) a recommendations card 4400. Further, home GUI 460 can include a banner 461 comprising a message reminding the user to scan the sensor every eight hours in order to see the impact of all meals. In exemplar embodiments, the user profile section 4002 of home GUI 460 displays a user's name, wherein the user's name is displayed directly adjacent to and proximal relative to banner 461.

Further, according to an aspect of the embodiments, home GUI 460 can be configured to be scrollable and dynamic. As such, in some embodiments, the content displayed on home GUI 460 can vary in response to a predetermined input by the user, such as when the user scrolls, drags, pulls the screen, or by some other predetermined gesture. In this manner, the home GUI 460 can comprise a plurality of views, wherein each view is configured to display a portion of home GUI 460 to the user. Specifically, in some embodiments, the home GUI 460 can be configured to transition between a plurality of views, wherein each of the plurality of views is different. More specifically, the home GUI can be configured to transition between the views in response to the predetermined input by the user.

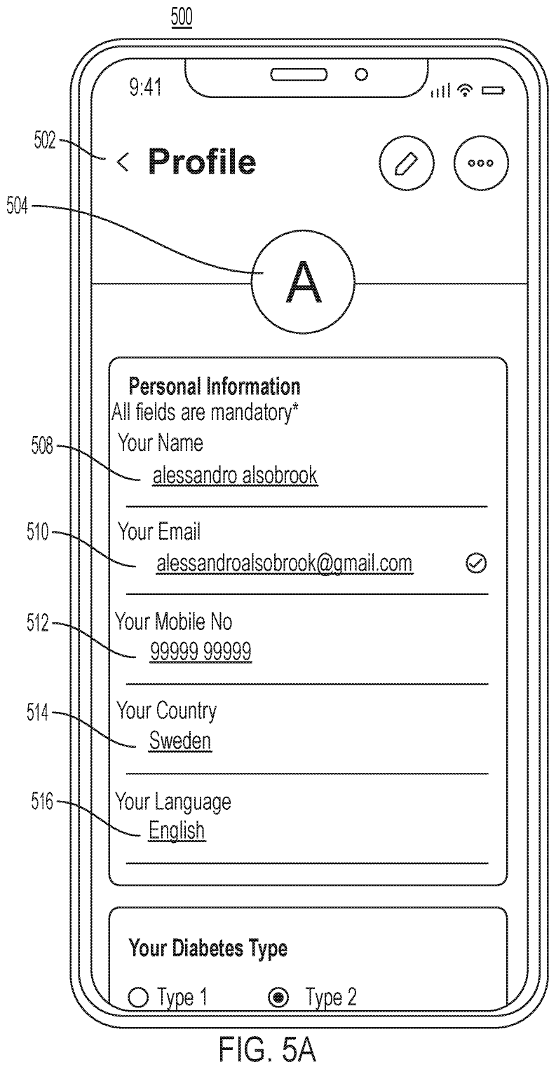

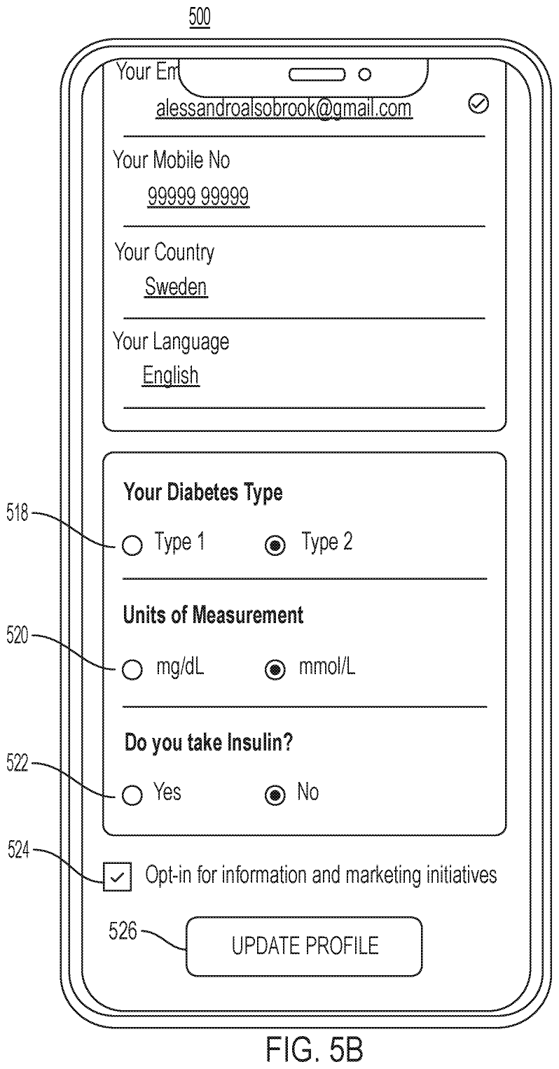

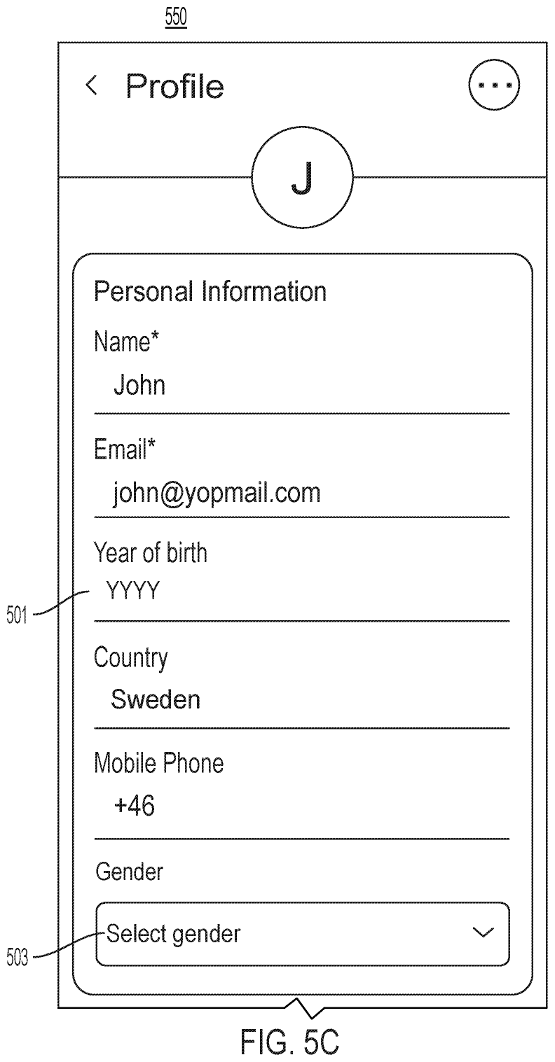

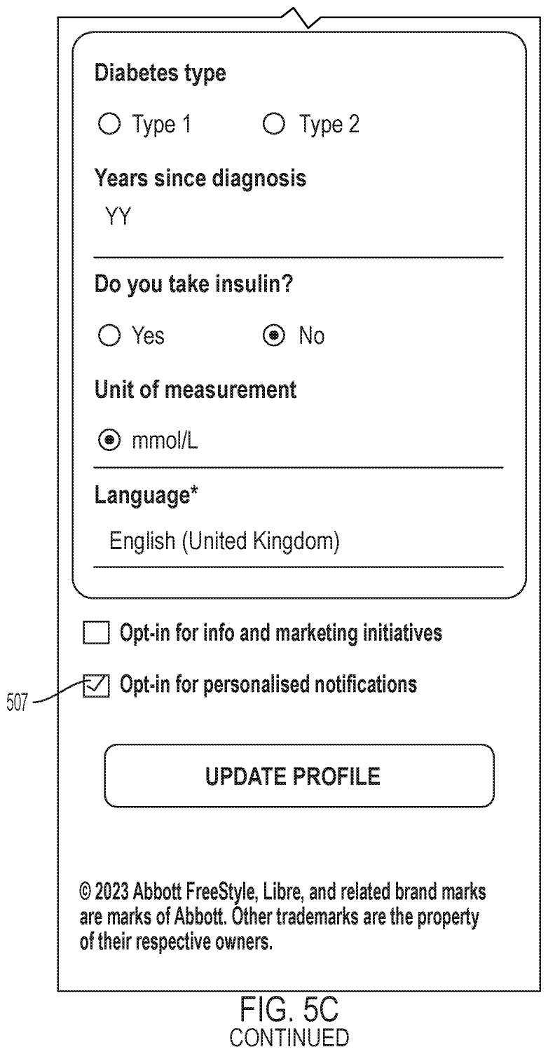

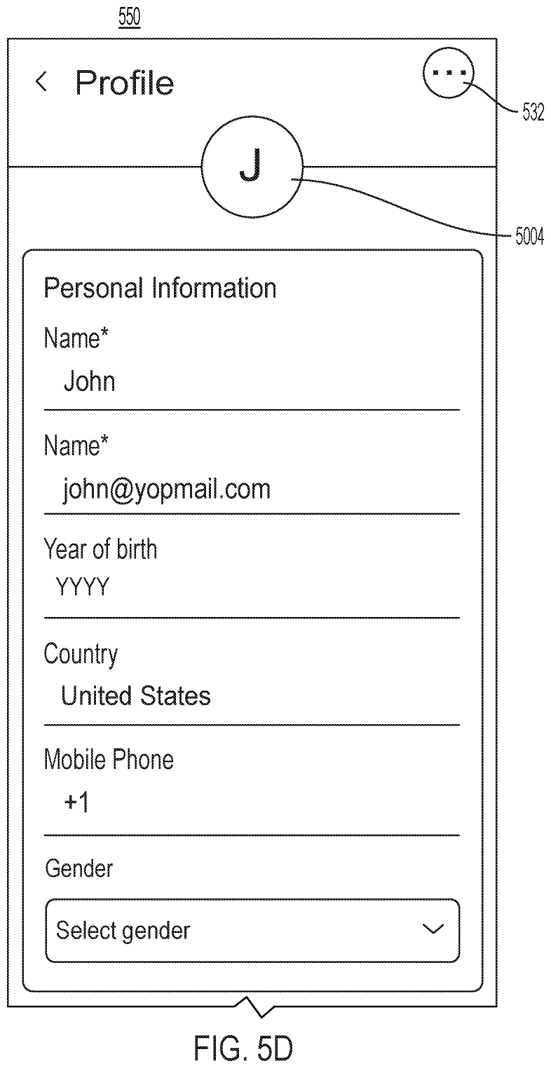

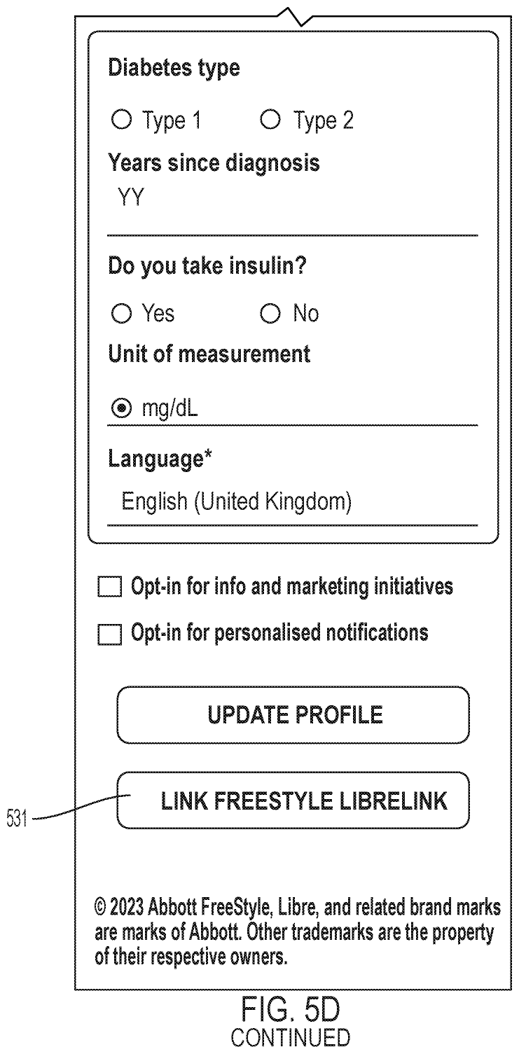

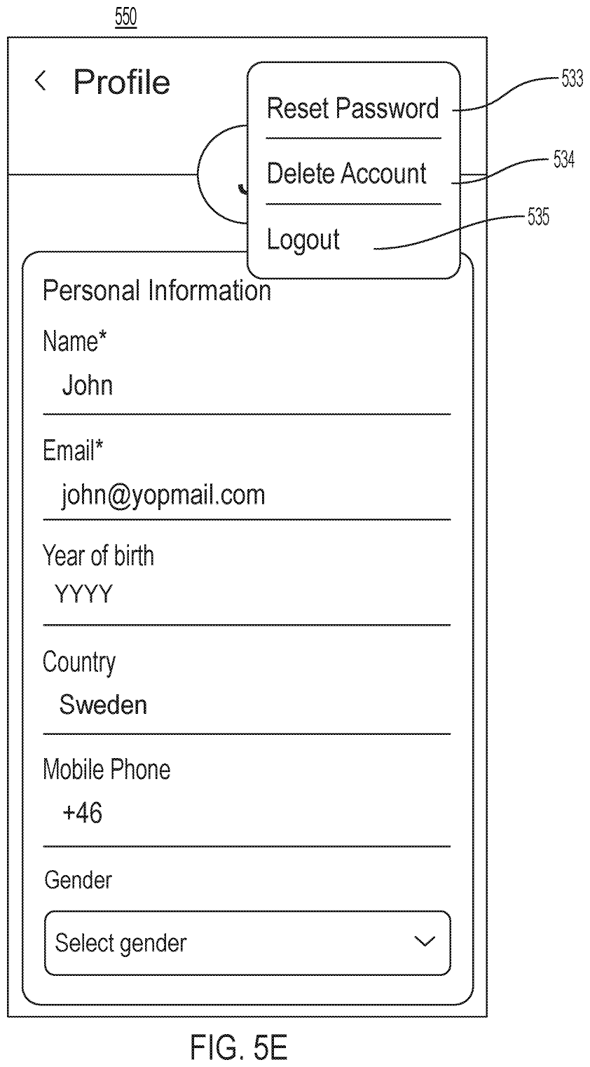

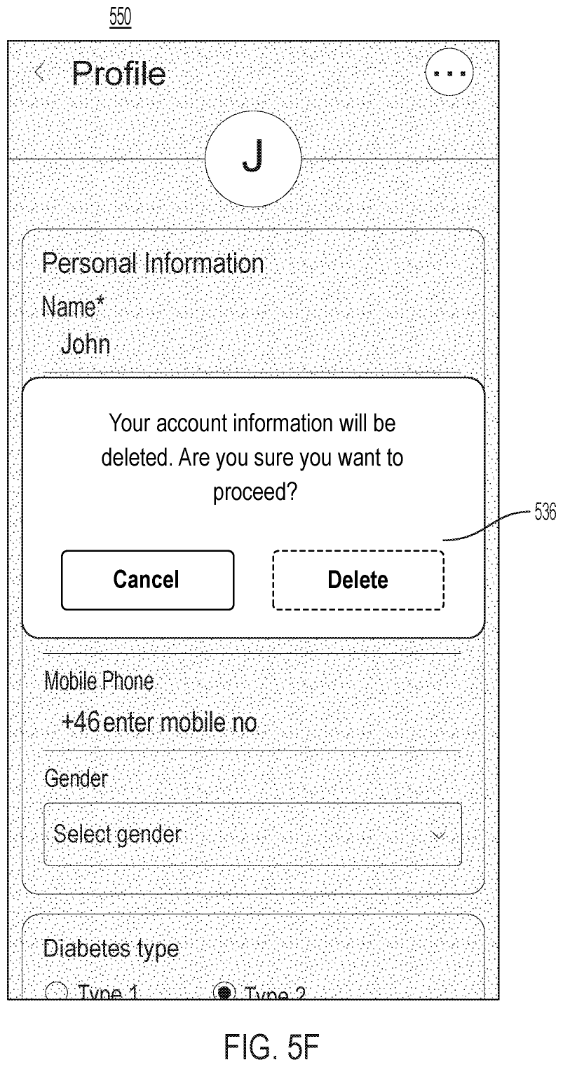

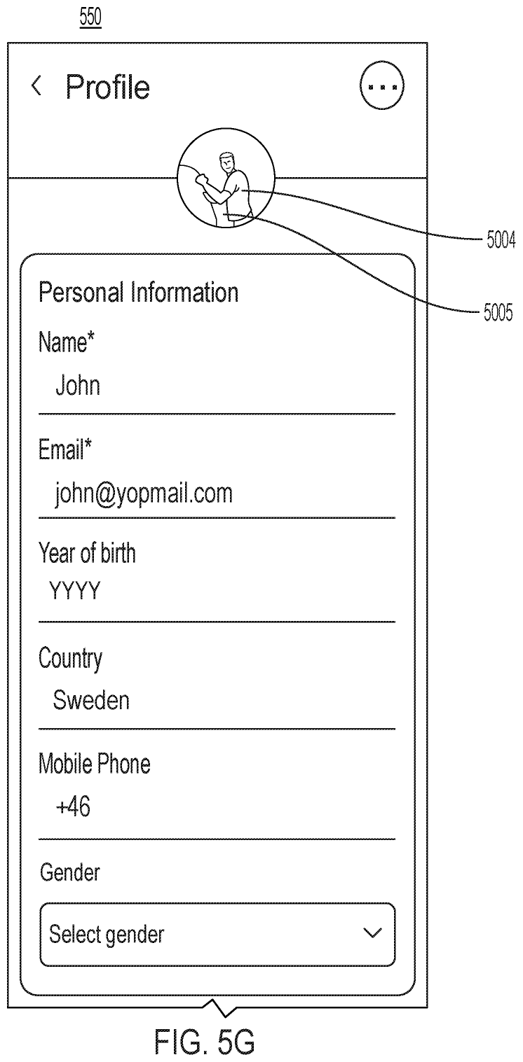

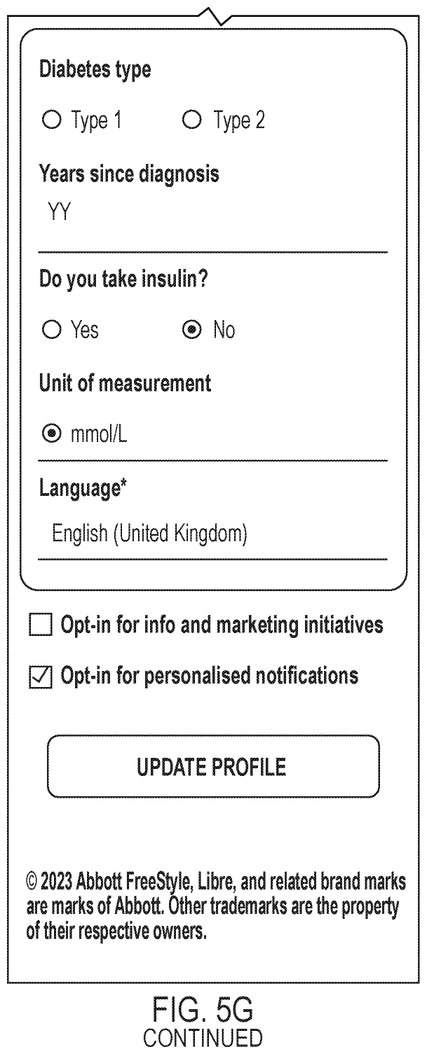

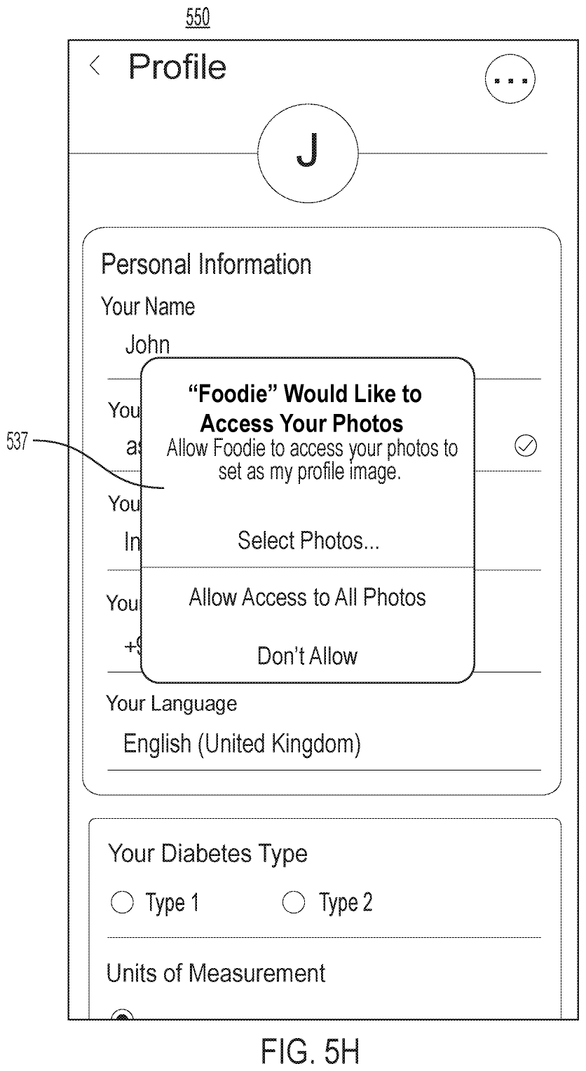

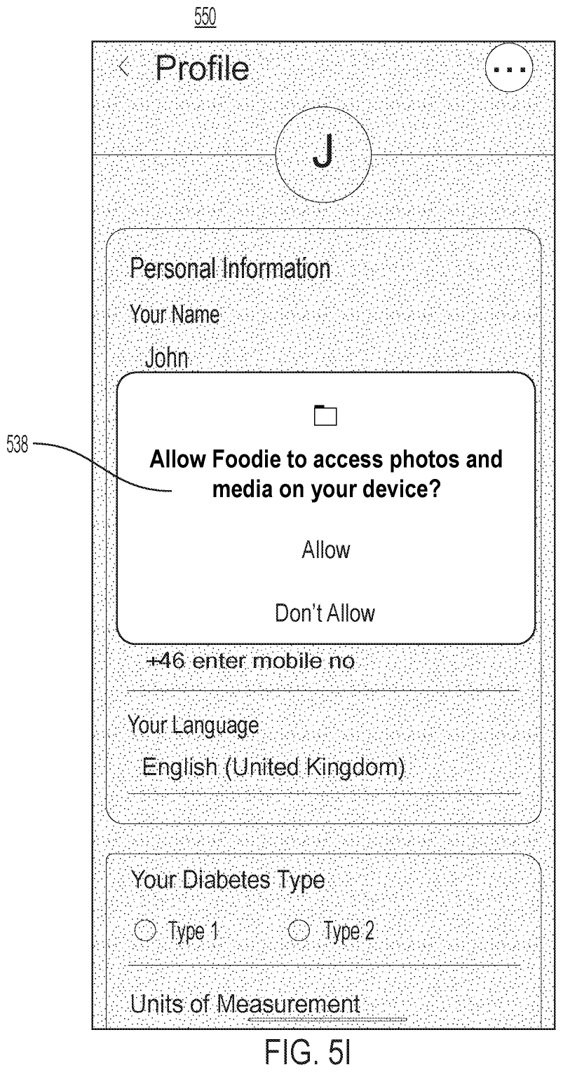

In exemplary embodiments, the home GUI comprises at least a first view and a second view. In some embodiments, and as depicted in FIG. 4A-5, in a first view of the scrollable home GUI 460, the user profile section 4002, the meal entry section 4004, the diary section 4012, and the meals card 4100 can be displayed on the home GUI 460. Further, in a second view of the scrollable home GUI 460, and as illustrated in FIG. 4A-6, the trends card 4200, the challenges card 4300, and the recommendation card 4400 are displayed on home GU 4601. According to an aspect of the embodiments, the navigation bar 4016 is configured to remain fixed on home GUI 460, and is displayed in all views of the scrollable home GUI 460 (as shown in FIGS. 4A-5 and 4A-6).

In some embodiments, and still with reference to FIGS. 4A-3 and 4A-4, the meals card 4100 is configured to display one or more meal listings 4101 comprising meal information related to one or more of the most recently consumed meals. Specifically, each meal listing 4101 provides details of a particular meal consumed by the user along with its corresponding meal-related glycemic response. In some exemplar embodiments, the one or more meal listings 4101 can be ordered such that the meal listing 4101 corresponding to the most recently consumed meal is presented first or at the top of the meals card 100, with subsequent meal listings 4101 being displayed in chronological order. In some embodiments, and as best shown through FIGS. 4A-3 and 4A-4 each meal listing 4101 can include: (1) a text description 4102 of the meal/food (e.g., “boiled eggs,” as illustrated in FIG. 4A-4); (2) a portion size indicator 4103 describing the relative portion size of the meal compared to the user's usual meal serving (e.g., “S” to represent a small meal, “M” to represent a medium meal, “or “L” to represent a large meal); (3) a datestamp 4104 associated with the date the meal was consumed; (4) a timestamp 4105 associated with the time the meal was consumed; and (5) a graphical representation 4106 indicating the user's glycemic response to the corresponding meal.

In many of the embodiments, and as depicted in FIGS. 4A-3 and 4A-4, the graphical representation 4106 can comprise a segmented shape (e.g., arch, circle, bar) with each segment comprising a different color (or no color at all). Each color can correspond with an analyte response range or ranking. For example, as shown on each of the meal listings 4101 depicted in FIGS. 4A-3 and 4A-4, the semi-circle (or “rainbow”) shape can comprise three segments, wherein: (1) the first segment can have a green color to indicate a low glycemic response; (2) the second segment can have a yellow color to indicate a medium glycemic response; or (3) the third segment can have an orange color to indicate a high glycemic response to the meal/food.

According to another aspect of the embodiments, the different colors can each indicate a range in which the analyte level variance value (or “impact”) is determined by methods 300, 350, or 370 (as described with respect to FIGS. 3A, 3F, and 3G). For example, if the analyte level variance value (e.g., the difference between the pre-meal glucose level and post-meal glucose level at a predetermined period of time after the meal) is determined to be within a low analyte level variance range (e.g., less than 60 mg/dL or less than 3.3 mmol/L), then the first segment is green (and the other segments are gray) to indicate that the meal/food elicited a low glycemic response, or had a low impact. As another example, if the analyte level variance value is determined to be within a medium analyte level variance range (e.g., between 60 mg/dL and 100 m/dL, or between 3.3 mmol/L and 5.6 mmol/L), then the second segment is yellow (and the other segments are gray) to indicate that the meal/food elicited a medium glycemic response, or a medium impact. As another example, if the analyte level variance value is determined to be within a high analyte level variance range (e.g., greater than 100 mg/dL or greater than 5.6 mmol/L), then the third segment is orange (and the other segments are gray) to indicate that the meal/food elicited a high glycemic response, or a high impact.

According to another aspect of the embodiments, one or more conditions can cause all segments to be gray, such as if the analyte level variance value is below zero, if the analyte level variance value is above a maximum analyte level variance value (e.g., 170 mg/dL, 180 mg/dL, 190 mg/dL), or if the initial analyte level value is above a maximum initial analyte level value (e.g., 180 mg/dL, 200 mg/dL, 220 mg/dL, 250 mg/dL, etc.). One or more of these conditions may indicate that the determined analyte level variance value is either unreliable, cannot be accurately calculated, or otherwise not representative of an analyte level variance value relating to the consumption of a meal.

Furthermore, although the graphical representation 4106 shown is a segmented semi-circle having three colored segments, those of skill in the art will understand that other geometrical shapes, colors, and numbers of segments can be utilized, and are fully within the scope of the present disclosure. Likewise, as described above, each of the colors can represent a specific range of values. However, those of skill in the art will understand that other ranges can be utilized besides those listed, and that these numbers are not meant to be limiting.

Still referring to FIGS. 4A-3 and 4A-4, and according to another aspect of the embodiments, the trends card 4200 of home GUI 460 can be configured to present recent trend information relating to analyte response associated with the user's food choices. In some exemplar embodiments, the trends card includes a summary panel 4201 which can provide an overall assessment of the user's food choices for a particular time period (e.g., the last day, the last week, or the last month). By way of illustration, the summary panel 4201 of FIG. 4A-3 states that the user's “food choices lead to a 33% decrease in green choices.” Further, the summary panel 4201 of FIG. 4A-4 states that the user's “food choices lead to a 25% decrease in green choices.”

In some embodiments, and as depicted in FIGS. 4A-3 and 4A-4, the trends card 4200 further comprises a graphical representation 4202 indicating the analyte response associated with the user's meal/food choices for the particular time period. In some embodiments, the graphical representation 4202 comprises a segmented shape with each segment comprising a different color (or no color at all), wherein each color can correspond with an analyte response range or ranking. In exemplar embodiments, the graphical representation 4202 can include a semi-circle shape with three segments, wherein: (1) the first segment can have a green color to indicate a low glycemic response; (2) the second segment can have a yellow color to indicate a medium glycemic response; or (3) the third segment can have an orange color to indicate a high glycemic response associated with the user's food choices for a recent or particular time period.

In some embodiments, the trends card 4200 will not be displayed on home GUI 460 if analyte data (e.g., data indicative of an analyte level, such as a glucose level) is not yet received and/or associated with a meal/food entry. According to an aspect of the embodiments, the trends card 4200 is configured to be dynamic. Specifically, the meal monitoring application can detect whether the analyte response associated with the user's food choices includes new trend information. If new trend information is detected, then the trends card 460 is configured to update as to populate an updated summary panel 4201 comprising information related to the new trend information.

In some embodiments, and as depicted in FIGS. 4A-3 and 4A-4, the home GUI 460 comprises a challenges card 4300 configured to display one or more selectable challenge icons 4301. Each challenge icon 4301 represents a different challenge which the user can participate in, wherein each challenge relates to the user's analyte response or glucose levels. The challenges can suggest certain behavior or activities to the user, and provide seasonal and/or behavioral based challenges that can affect the user's analyte levels and in turn help the user manage his or her diabetes. In some exemplar embodiments, a challenge icon 4301 can represent a challenge directed to the user consuming a predetermined number of consecutive meals classified as having a particular analyte response (e.g., “4 In A Row” challenge icon depicted in FIG. 4A-3, wherein the 4 In A Row challenge icon 4301 represents a challenge relating to the user consuming four low impact or low glycemic meals in a row). Further, a challenge icon 4301 can represent a challenge relating to the user consuming a particular type of meal (e.g., the “Breakfast” challenge icon depicted in FIGS. 4A-3 and 4A-4). In yet another example, a challenge icon 4301 can represent a challenge directed to the user consuming a particular number of portions of food (e.g., vegetables or fruits) a day for a predetermined time period. In another example, a challenge icon 4301 can represent a challenge directed to the user lifestyle tendencies (e.g., the user staying hydrated, avoiding carbonated beverages, increasing physical activity, or avoiding fast food for a predetermined time period). Various other challenges for use with the meal monitoring application can be readily recognized by those of skill in the art.

According to an aspect of the embodiments, each challenge icon 4301 can include an image or picture 4302 associated with the particular challenge. Further, the challenge icon 4301 can include a challenge title 4303 which provides a textual description of the challenge represented by the corresponding challenge icon. In some embodiments, the challenges card 4300 can display different challenge icons 4301 in response to a received input by the user (e.g., by a swipe gesture, a drag gesture, or some other predetermined gesture). For example, in some embodiments the challenges card 4300 can display a set of two or three challenge icons 4301. In response to received input, the challenges card 399 can display a new set of challenge icons 4301 on the home GUI 460. In some embodiments, at least one of the challenge icons 4301 in the new set is different than at least one of the challenge icons 4301 in the original set, prior to the received input by the user.

In some embodiments, once the user has selected a challenge icon 4301, a new challenge icon 4301 will be displayed in place of the previously selected challenge icon 4301. In some embodiments, a challenge icon 4301 can be scheduled to remain on the home GUI 460 for a predetermined period of time.

Further, the challenges card 4300 can comprise a selectable “View All” link 4304 which, upon being selected by the user, outputs a challenge GUI 1200 comprising a challenge list, wherein the challenge list displays all live challenges in which the user is participating in, challenges successfully completed by the user, and challenges the user has not yet participated in.

Additionally, in some embodiments, the home GUI 460 displays a recommendations card 4400 configured to display one or more selectable recommendation icons 4401, wherein each recommendation icon 4401 represents a different recommendation presented to the user. The recommendations can relate to the user's food choices and/or analyte response. The recommendation can suggest certain activities to the user, and provide seasonal and/or behavioral based content (e.g., recommendations relating to eating healthy around the holidays, or how to put on or remove a sensor). In some embodiments, the recommendation icon 4401 can link out to credible sources related to a corresponding recommendation. According to an aspect of the embodiments, the meal monitor application can algorithmically process and analyze inputted meal entry data, and detect and/or quantify the user's food choices so as to push recommendations through the recommendation icons 4401 that can be considered more desirable or beneficial suggestions to the user. For example, if the meal monitoring application detects that the user frequently consumes chicken, a recommendation icon 4401 relating to chicken can be displayed to the user. Further, in another example, if the meal monitor application detects that the user has a tendency to consume pizza, a recommendation icon which provides alternatives to pizza can be displayed to the user.

According to another aspect of the embodiments, after the user selects a recommendation icon 4401, a modal 4405 (FIG. 4A-7) relating to the selected recommendation icon 4401 can be displayed on home GUI 460, wherein the modal 4405 can provide contextual information related to a recommendation to direct the user to act in accordance with the recommendation. In some embodiments the contextual information can prompt the user to partake in activities or behavior consistent with the recommendation. For example, if the user selects a “Summer Holidays” recommendation icon 4401, as depicted in FIG. 4A-6, then a modal 4405 providing information related to a summer holidays recommendation will be displayed on the home GUI 460 (see, e.g., FIG. 4A-7, “Keep a track of your meals even when on holidays. Click on the button below to add your meals now.”). In some embodiments, and as shown in FIG. 4A-7, the modal 4405 can include a start button 4406 which, when selected by the user, allows the user to take steps towards complying with the recommendation (e.g., “Add Food” button in FIG. 4A-7 allows the user to add meal or food entries so as to keep track of meals when on holidays, consistent with the summer holidays recommendation).

According to an aspect of the embodiments, and as best shown in FIG. 4A-4, each recommendation icon 4401 can include an image or picture 4402 relating to its particular recommendation. The recommendation icon 4401 can further include a recommendation title 4403 providing a textual description of the corresponding recommendation. Further, in some embodiments, the recommendations card 4400 can display different recommendations icons 4401 in response to a received input by the user (e.g., by a swipe gesture, a drag gesture, or some other second predetermined gesture). For example, in some embodiments the recommendations card 4400 can display a set of two or three recommendation icons 4401. In response to received input, the recommendations card 4400 can display a new set of recommendation icons 4401 on the home GUI 460. In some embodiments, at least one of the recommendation icons 4401 in the new set is different than at least one of the recommendation icons 4401 in the original set, prior to the received input by the user.

In some embodiments, once the user has selected a recommendation icon 4401, a new recommendation icon 4401 will be displayed in place of the previously selected recommendation icon 4401. In some embodiments, a particular recommendation icon 4401 can be scheduled to remain on the home GUI 460 for a predetermined period of time.

In some embodiments, and as best shown in FIG. 4A-4, home GUI 460, can further comprise an informational button 4008 (top) configured to display a plurality of selectable links which, when selected by the user, can cause the meal monitor application to display a GUI corresponding to the selected link. In some embodiments, and as best shown in FIG. 4A-4, selection of the information button 4008 can display: (1) a “Profile” link 4001; (2) a “Notifications” link 4003; (3) an “Order your next sensor” link 4005; (4) a “Frequently asked questions” (“FAQ”) link 4007; (5) an “About us” link 4009; and (6) a “Contact us” link 4011.

For example, FIG. 4B depicts an About Us GUI 470 that is displayed on the meal monitor application after the user selects the About Us link 4009 on home GUI 460. In some embodiments, the About Us GUI 470 includes a plurality of selectable sections, including but not limited to, a license section 471, a privacy policy section 472, and a terms and conditions section 473 which, when selected by the user, can display a corresponding GUI.

Further, FIG. 4C illustrates a Contact Us modal 474 that is displayed on the meal monitor application after the user selects the Contact Us link 4011 on home GUI 460. Specifically, the Contact Us modal 474 can be displayed on the home GUI 460, and can comprise contact information provided for the meal monitor application.

FIGS. 4D and 4E depict an example embodiment of an FAQ GUI 480 that is displayed on the meal monitor application after the user selects the FAQ link 4007 on home GUI 460. According to some embodiments, FAQ GUI 480 comprises a first question section 481 having information related to starting the meal monitor application and a second question section 482 having information related to adding or logging food on the meal monitor application. In some embodiments, the first question section 481 and the second question section 482 each include a list of selectable question links 483 which, upon being selected, are configured to expand to include a list of corresponding answer(s) to the question described in the selected question link 483 (as shown in FIG. 4E). In some embodiments, the first question section 481 and the second question section 482 are configured to expand (FIG. 4E) and minimize (FIG. 4D) in response to a received input by the user (e.g., a tap gesture, or other predetermined gesture). Referring next to FIGS. 4F to 4I, example embodiments of connection GUIs for when a user starts the meal monitor application for the first time are depicted. In some embodiments, after a user successfully starts the meal monitor application (and completes the onboarding process and tutorials), the meal monitor application can prompt the user to provide account information to connect the meal monitor application with a sensor interface application. FIG. 4F, for example, depicts a modal that requests that the user connect the meal monitor application with the sensor interface application. After selecting the connect button 418 (FIG. 4F), meal monitor application can display an account login GUI 420 that includes an e-mail field 422, a password field 424, and a sign-in button 426, as depicted in FIG. 4G. Once the user signs in successfully, a consent GUI 430 can be displayed to the user to obtain the user's consent to share data with the meal monitor application. Once the user consents, they are returned to home GUI 400 with a modal 432 indicating that the meal monitor application was successfully connected with the sensor interface application.

FIGS. 4J-4N depict additional example embodiments of connection GUIs for when a user starts the meal monitoring application for the first time. Specifically, FIG. 4J, depicts a modal that requests that the user connect the meal monitor application with the sensor interface application. After selecting the connect button 4180 (FIG. 4J), meal monitor application can display an account login GUI 465 that includes an e-mail field 4220, a password field 4240, and a sign-in button 4260, as depicted in FIG. 4K. Once the user signs in successfully, a consent GUI 475 (FIG. 4L) can be displayed to the user to obtain the user's consent to share data with the meal monitor application. Once the user consents, they are returned to home GUI 450 or 460 with a modal 4320 (FIG. 4M depicts modal on home GUI 460) indicating that the meal monitor application was successfully connected with the sensor interface application. In some embodiments, if the meal monitoring application is not successfully connected with the sensor interface application, a modal 4321 is displayed on home GUI 450 or 460 indicating as such (e.g., unsuccessful connection due to the account credentials tried are already in use with another meal monitoring application, as shown in FIG. 4N). In some embodiments, and as depicted in FIG. 4N, modal 4321 can comprise an ok button 4322 and a connect button 4323.

### Example Embodiments of User Profile GUIs

Referring next to FIGS. 5A and 5B, an example embodiment of a user profile GUI 500 for a meal monitor application is depicted. User profile GUI 500 is displayed when the user selects the user profile section 402 on home GUI 400. As shown in FIG. 5A, user profile GUI 500 can include a plurality of editable fields configured to receive personal information about the user of the meal monitor application. In some embodiments, user profile GUI 500 can comprise a profile icon 504, a name field 508, an e-mail address field 510, a telephone number field 512, a country field 514, and a language field 516. In addition, as seen in FIG. 5B, user profile GUI 500 can further include a plurality of selectable options to prompt the user for additional profile information or preferences. For example, the plurality of selectable options can include a medical condition field 518, a units of measurement field 520, and a medication field 522. According to another aspect of some embodiments, user profile GUI 500 can also include a consent checkbox 524 to indicate whether the user wishes to opt-in (or opt-out) of receiving additional information and/or marketing solicitations. In many embodiments, user profile GUI includes an update button 526 to save any changes that the user may have made to their profile information. In some embodiments, user profile GUI 500 can also include a “Link Sensor App” button (not shown), if the user has not yet connected the meal monitor application with a sensor app.

FIG. 5C depicts an additional exemplar embodiment of a user profile GUI 550 for a meal monitor application. FIG. 5C depicts user profile GUI 550 which, in some embodiments, can displayed when the user selects the user profile section 4002 on home GUI 460. User profile GUI 550 is similar to user profile GUI 500, except that it further comprises a birthdate field 501, a gender field 503, and a consent checkbox 507 to indicate whether the user wishes to opt-in (or opt-out) of receiving personalized notifications. FIG. 5D depicts user profile GUI 550, further comprising the “Link Sensor App” button 531. FIG. 5E depicts user profile GUI 550, wherein the user has selected an options button 532 (e.g., FIG. 5D). Specifically, when the user selected the options button 532, the user profile GUI 550 is configured to display a plurality of selectable links, including but not limited to a reset password link 533, a delete account link 534, and a logout link 535 (as shown in FIG. 5E). If the user selects the delete account link, meal monitor application can display a modal 536 that requests the user confirm whether they want to proceed with deleting account information (as illustrated in FIG. 5F).

According to another aspect of the embodiments, and as depicted in FIG. 5G, the user can select a profile icon 5004 to upload a photograph/picture 5005 to be associated with the user profile. In some embodiments, the user can select a picture 5005 from a library of photographs. Specifically, and as shown in FIGS. 5H-5I, upon the user selecting the profile icon 5004, a pop-up modal 537 can be displayed on user profile GUI 550, wherein pop-up modal 537 is configured to request access to the user's photos. FIG. 5H depicts pop-up modal 537 presented through a first mobile operating system (e.g., iOS), and FIG. 5I depicts pop-up modal 538 presented through a second mobile operating system (e.g., Android). In some embodiments, when the user does not associate a photograph 5005 with the user profile, a placeholder image will be displayed on the profile icon 5004 instead (as shown, e.g., in FIGS. 5C-5F). In some embodiments, and as shown in FIGS. 5C-5F, the placeholder image is an initial corresponding to the first letter of the user's inputted first name (e.g., “J” for a user having the name “John”).

### Example Embodiments of Meal Entry GUIs

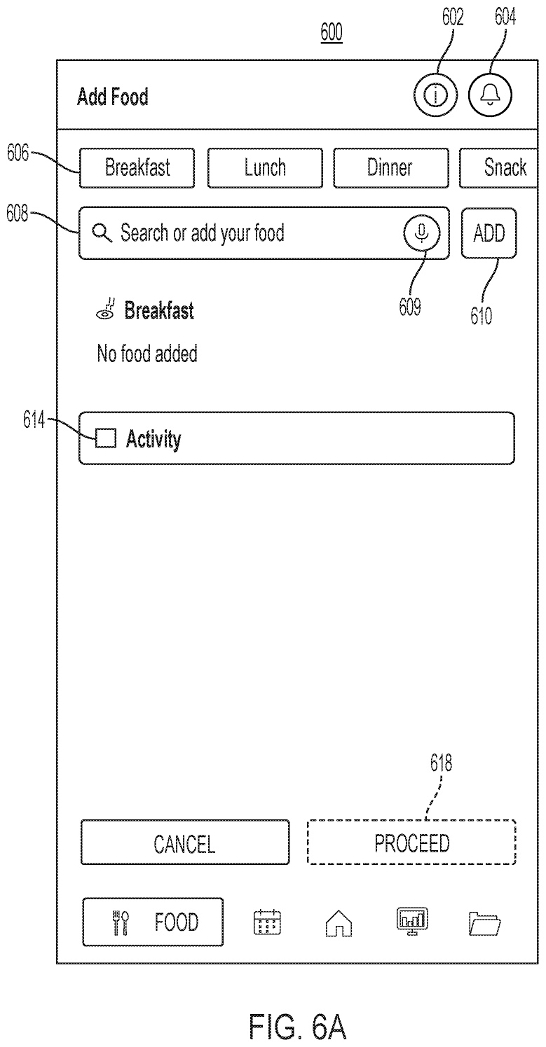

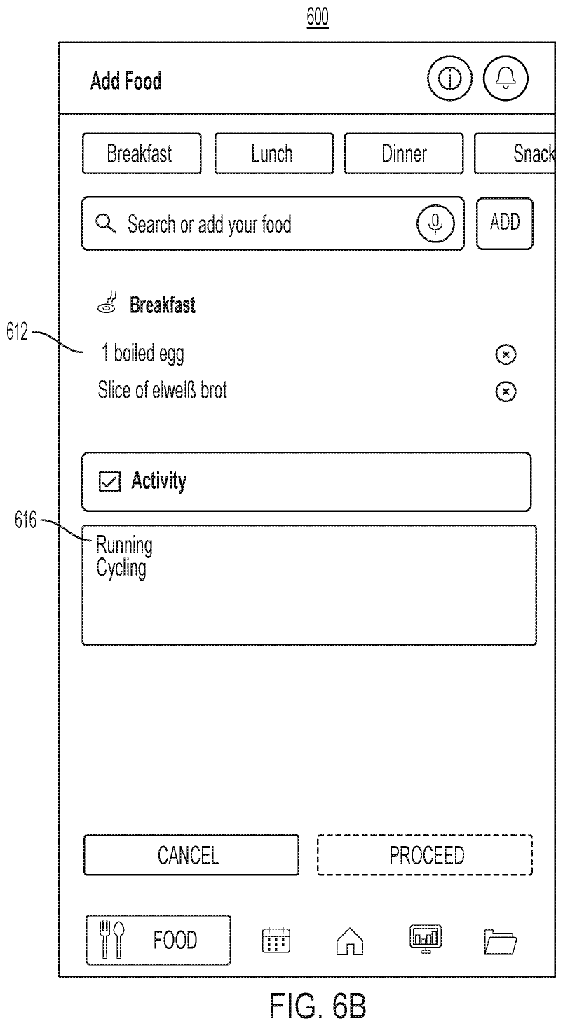

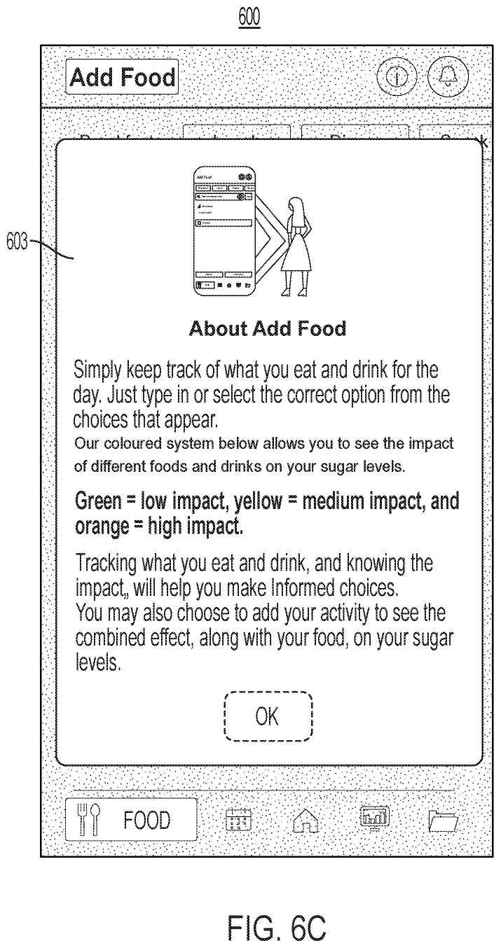

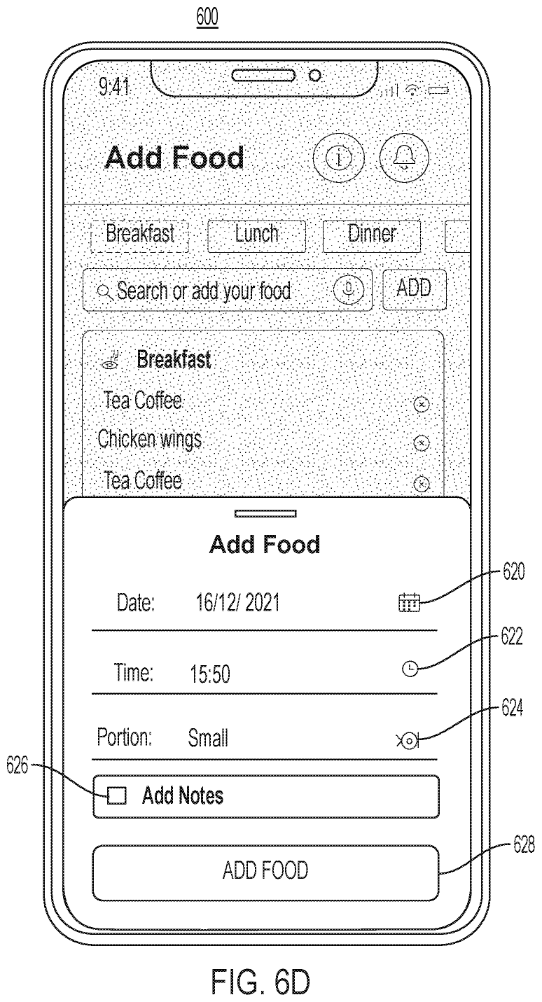

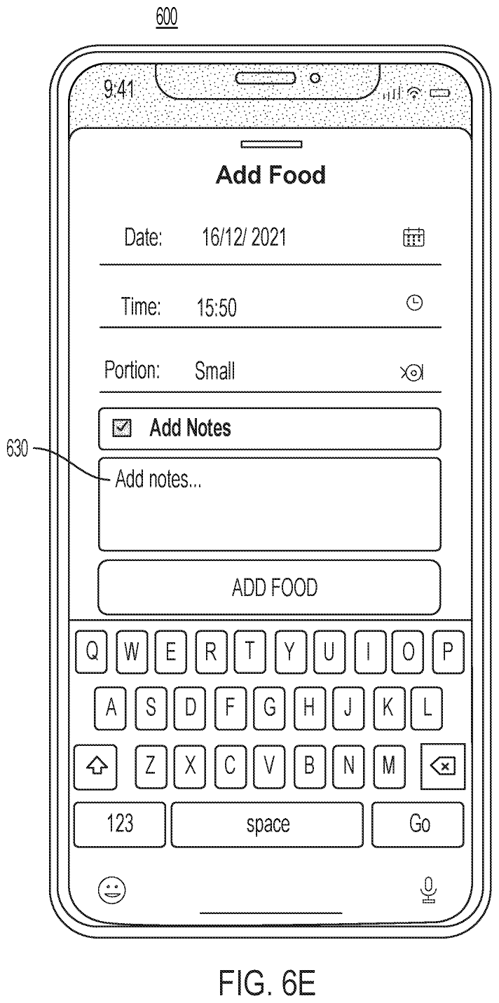

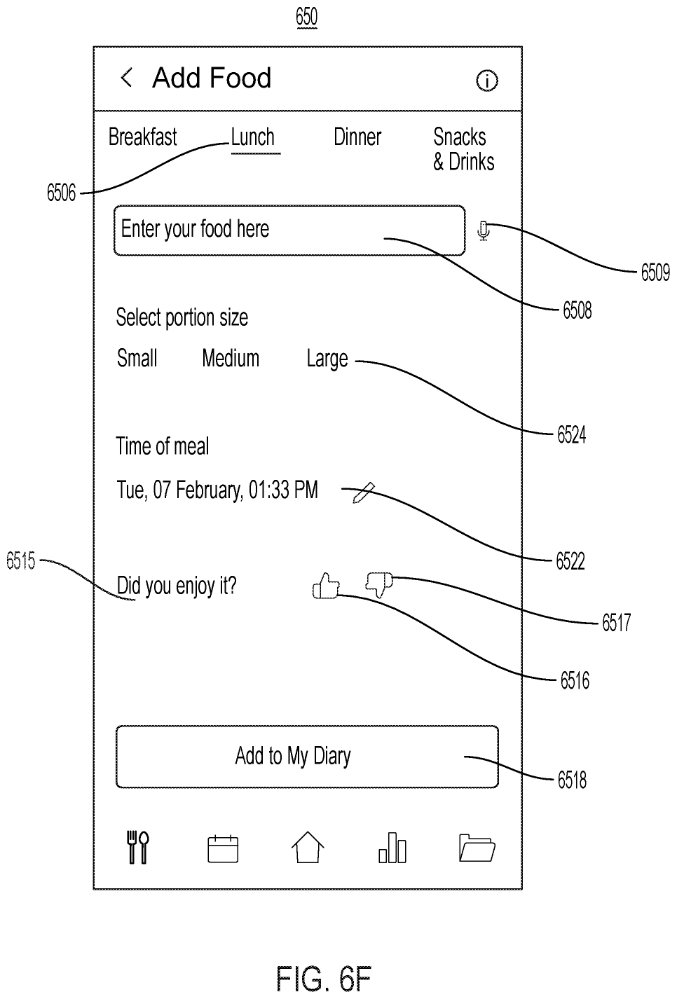

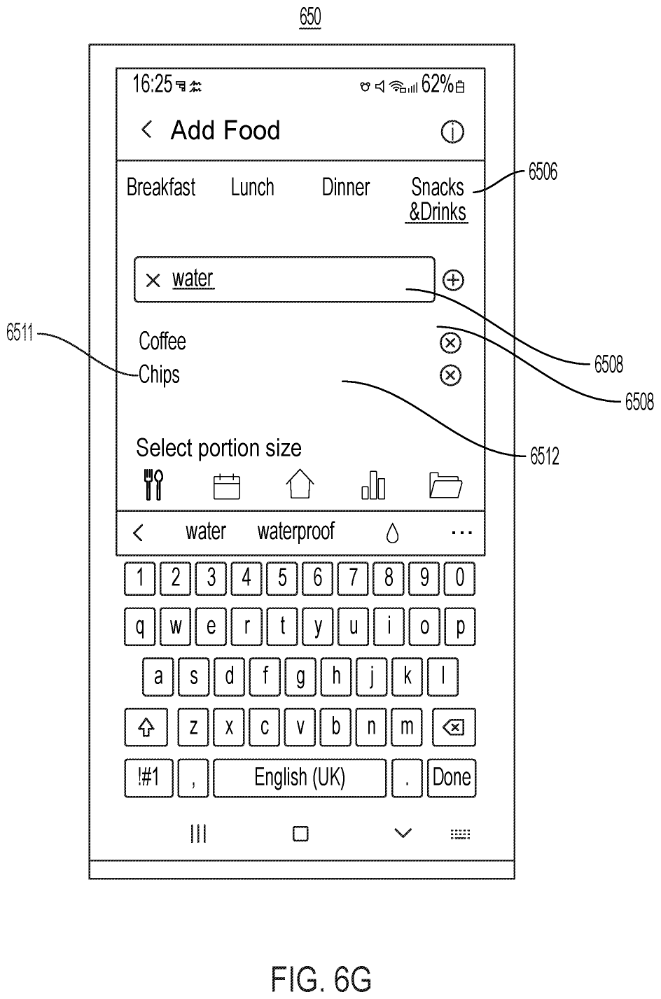

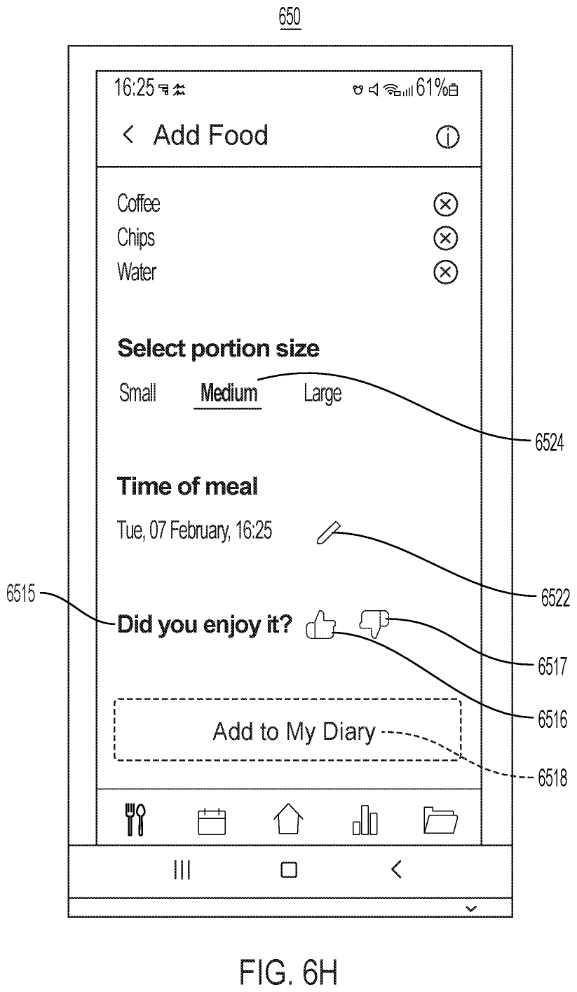

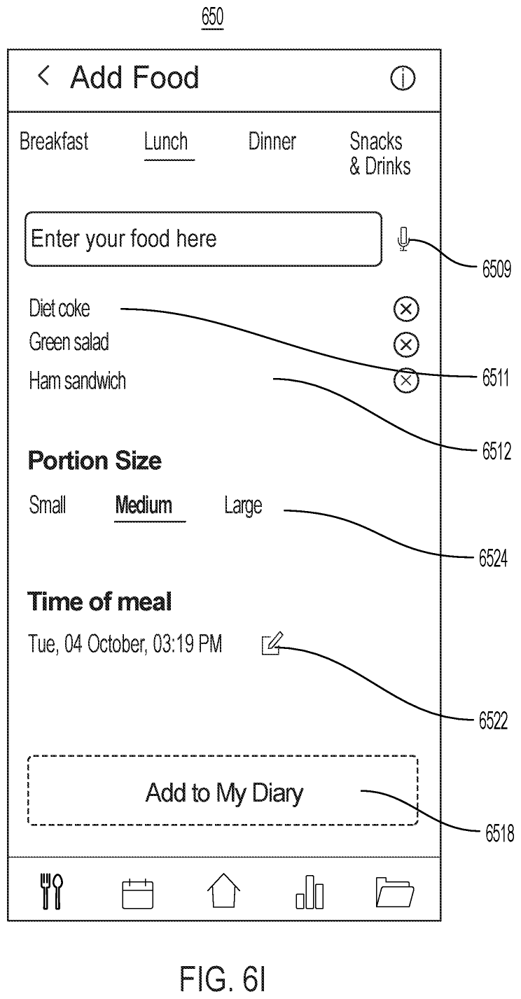

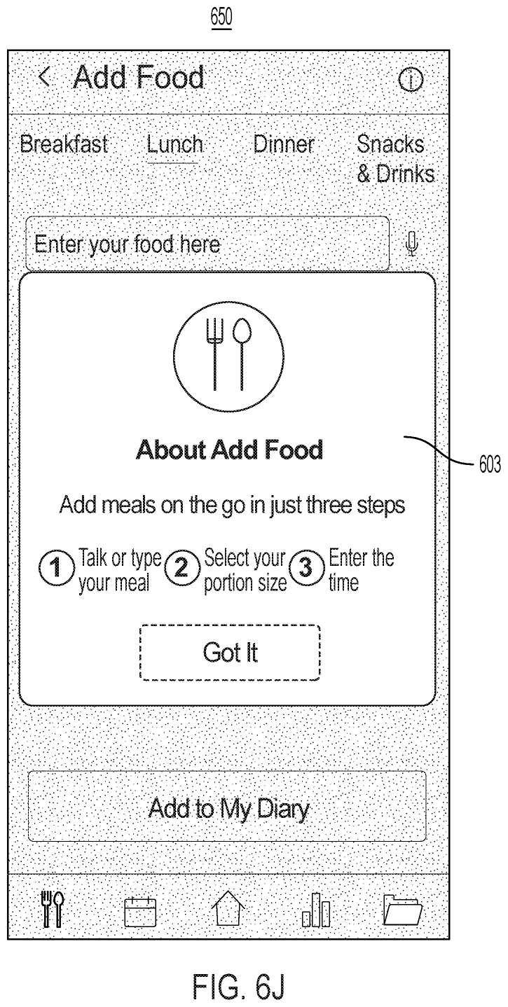

Turning to FIGS. 6A to 6E, an example embodiment of a meal entry GUI 600 for a meal monitor application is depicted. Referring first to FIG. 6A, meal entry GUI 600 is displayed when the user selects the meal entry section 404 on home GUI 400. Alternatively, meal entry GUI 600 can also be displayed if the user selects the corresponding icon in the navigation bar (bottom). Meal entry GUI 600 can also include an information button 602 to provide contextual information 603 (FIG. 6C), and a notification button 604 to relay in-app notifications to the user (FIG. 4O).

One desirable aspect of the embodiments described herein is the ease at which meal information can be entered. This can promote usage of the meal monitor application and allow the user to gain a more intuitive understanding of the glycemic impact of meal consumption, which in turn can improve the user's health. As shown in FIG. 6A, meal entry GUI 600 can include a selectable meal type option 606, by which the user can easily indicate the type of meal for the meal entry (e.g., breakfast, lunch, dinner, snack, dessert, etc.). According to another aspect of the embodiments, meal entry GUI 600 can include a search/add field 608, which allows the user to enter the name of the food for the meal entry. As the user enters the characters of the name of the meal or food into search/add field 608, the meal monitor application can auto-suggest entries based on the user's past meal entries. For example, in some embodiments, the meal monitor application can retrieve information relating to past meal entries with the same (or similar) food name and display it in results section 612, for example, as a list. This list can be ordered such that the most commonly consumed or selected meals are presented first or at the top of the list, with remaining meals presented in order of decreasing frequency of consumption (e.g., the most commonly consumed meal is presented first, the second most commonly consumed meal is presented second, and so forth with the least commonly consumed meal presented at the end). In some embodiments, the list can be ordered based on entries having the lowest impact (e.g., lowest analyte level variance values), so as to suggest more favorable foods to the user. If the desired food name appears in results section 612, user can conveniently select the entry without the need to type the full name of the food or meal, and then press the add button 610.

In some embodiments, meal entry GUI 600 can also include a voice recognition feature. According to one aspect of some embodiments, instead of manually typing the name of the meal or food into search/add field 608, the user can press the voice recognition button 609 and speak the name of the meal or food. In response, the meal monitor application can be configured to display the closest results in result section 612.

Furthermore, although FIG. 6A shows results section 612 in the form of a list, those of skill in the art will appreciate that other interfaces can be implemented, such as by the manual entry of text, by selection of the meal name from a list (e.g., a picklist or drop-down list), by selection of the meal picture from a group of pictures, by selection of recognizable indicia (e.g., a tag or code) of the meal, or any combination thereof.

According to another aspect of the embodiments, meal entry GUI 600 can also include an activity checkbox 614. As seen in FIG. 6B, when activity checkbox 614 is selected, an activity text box 616 is presented on meal entry GUI 600, which allows the user to enter a textual description of any physical activity (e.g., walking, gardening, cycle ride, etc.) that they participated in around the time of the meal event.

According to another aspect of the embodiments, after meal information is entered (and, optionally, activity information), the user can select proceed button 618, which can cause the display of a modal configured to request additional information, as seen in FIG. 6D. The modal can include, for example, a date field 620, a time field 622, and a meal portion size field 624 to capture the details of the meal event. Furthermore, in some embodiments, the modal can include an “Add Notes” checkbox 626 that, when selected by the user, presents a notes text box 630 (FIG. 6E). This allows the user to enter additional notes (e.g., specific information about the meal/food, medication taken by the user around the time of the meal/food) to accompany the meal entry.

In some embodiments, the time field may be auto-populated based on the time that the user selected the proceed button 618. According to one aspect of some embodiments, the auto-populated time field can still be edited by the user. In other embodiments, the time field may include a start time and a stop time, during which the meal was consumed (instead of a single time entry, as shown in FIG. 6D). In other embodiments, the time field can be a generalized or heuristic part of the day (e.g., early morning, late afternoon, etc.), or an approximated time range (e.g., 6-7 am, 5-6 pm, etc.) with any desired level of granularity of the time range (e.g., 10 minute, 15 minute, 30 minutes, 60 minutes, etc.).

FIGS. 6F-6J depict another exemplar embodiment of a meal entry GUI 650 for a meal monitor application. Referring first to FIG. 6F, and in like manner to the meal entry GUI 600 depicted in FIGS. 6A-6E, meal entry GUI 650 can be displayed when the user selects the meal entry section 4004 on home GUI 460. Alternatively, meal entry GUI 650 can also be displayed if the user selects the corresponding icon in the navigation bar (bottom). Meal entry GUI 650 can also include an information button 653 to provide contextual information (FIG. 6J).

As shown in FIG. 6F, meal entry GUI 650 can include a selectable meal type option 6506, by which the user can easily indicate the type of meal for the meal entry (e.g., breakfast, lunch, dinner, snack and drinks, etc.). According to another aspect of the embodiments, meal entry GUI 650 can include a search/add field 6508, which allows the user to enter the name of the food for the meal entry (as shown in FIG. 6G). In like manner to search/add field 6508, as the user enters the characters of the name of the meal or food into search/add field 6508, the meal monitor application can auto-suggest entries based on the user's past meal entries. In some embodiments, once the user enters the name of the meal or food, or selects an entry populated by the auto-suggest functionality, a meal tag 6511 comprising the entered meal or food name is added to a meal library section 6512 (best shown in FIGS. 6G and 6I) The user can associate one or more meal tags 6511 with the meal consumed (e.g., in FIG. 6I, the user has included a “diet coke,” “green salat,” and “ham sandwich” meal tag 6511 for their lunch entry).

In some embodiments, meal entry GUI 650 can also include a voice recognition feature 6509. According to one aspect of some embodiments, instead of manually typing the name of the meal or food into search/add field 6508, the user can press the voice recognition button 6509 (best shown in FIGS. 6F and 6I) and speak the name of the meal or food.

According to another aspect of the embodiments, meal entry GUI 650 can comprise portion size indicator 6524 options that indicate the relative size of the meal consumed, e.g., selectable buttons indicating different sizes of meals such as “Small,” “Medium,” and “Large.” (e.g., the “Medium” portion size indicator 6524 is chosen in FIG. 6H).

Further, meal entry GUI 650 can also comprise a time field 6522 that includes a time and date associated with the meal entry. In some embodiments, the time field 6522 can be automatically associated with the meal entry or be auto-populated based on the time that the user inputs the meal entry. According to one aspect of some embodiments, the auto-populated time field 6522 can still be edited by the user.

According to another aspect of the embodiments, after meal information is entered, the user can select an add button 6518 (as best shown in FIGS. 6H-6I). In some embodiments, and as best shown in FIGS. 6F and 6H, the meal entry GUI 650 can also include a feedback query 6515 requesting whether the user enjoyed a meal (e.g., “Did you enjoy it?”). In some embodiments, the feedback query 6515 can include a first indicator 6516 (e.g., a thumbs up button) which the user can select to indicate the user enjoyed the meal. Further, the feedback query 6515 can include a second indicator 6517 (e.g., a thumbs down button) which the user can select to indicate the user did not enjoy the meal. Those of skill in the art will recognize that the meal entry GUI embodiments described herein can utilize others buttons, icons, or indicators to indicate whether a user enjoyed a meal.

The feedback provided by the user through the feedback query 6515 can be used to recommend food items which elicited a low or medium glycemic response/impact to the user. For example, if the user selected the first indicator in response to the feedback query 6515 requesting whether the user enjoyed a meal, and if the meal elicited a low or medium glycemic impact, then the meal monitor application can subsequently recommend the meal to the user. Further, the feedback provided by the user through the feedback query 6515 can be used to avoid recommending foods which the user did not enjoy, or which had a medium or high glycemic impact. For example, if the user selected the second indicator in response to the feedback query 6515 requesting whether the user enjoyed a meal, then the meal monitor application can avoid subsequently recommending the meal which the user did not enjoy. In this regard, the feedback query 6515 can be utilized to help the user make better food choices by recommending foods to the user which it enjoyed and which elicited a low or medium glycemic impact.

### Example Embodiments of Diary GUIs

Turning to FIGS. 7A to 7I, example embodiments of a diary GUI 700 for a meal monitor application are depicted. In many embodiments, diary GUI 700 is displayed when the user selects the diary section 412 on home GUI 400 (FIG. 4A-1). Alternatively, diary GUI 700 can be displayed when the user selects the corresponding icon in the navigation bar (bottom). Moreover, diary GUI 700 can include an information button 702 (FIG. 7A) to provide contextual information 714 (FIG. 7B), and a notification button 704 (FIG. 7A) to provide the user with in-app notifications (FIG. 4O). In some embodiments, diary GUI 700 can also include a search bar 708 configured to allow the user to search for particular diary entries.

According to one aspect of the embodiments, diary GUI 700 can display the user's previous analyte responses to meals in a manner that is user-friendly and easy to understand. In some embodiments, for example, diary GUI 700 can include a plurality of views by which the analyte responses can be organized and displayed. For example, as shown in FIG. 7A, diary GUI 700 can present analyte responses “by day” when days view option 706 is selected. Similarly, as shown in FIG. 7H, diary GUI 700 can present the analyte responses “by week” when weeks view option 736 is selected.

Referring again to FIG. 7A, an example embodiment of a daily listing 710 of meal-related glycemic responses for diary GUI 700 is depicted. As can be seen in daily listing 710, a plurality of meals and/or foods is presented for the day, “12 Mar. 2022,” including the meal/food entry comprising “dosa, tea.” As seen at the left of each meal/food entry, a graphical representation indicating the user's glycemic response to a corresponding meal/food is displayed. Along with the graphical representation, each meal/food entry can also include a time-of-entry and a numeric value indicating the analyte level variance value (“impact”) associated with the meal/food.

In many of the embodiments, the graphical representation comprises a segmented shape (e.g., arch, circle, bar) with each segment comprising a different color (or no color at all). Each color can correspond with an analyte response range or ranking. For example, as shown in daily listing 710, the semi-circle (or “rainbow”) shape can comprise three segments, wherein: (1) the first segment can have a green color to indicate a low glycemic response; (2) the second segment can have a yellow color to indicate a medium glycemic response; or (3) the third segment can have an orange color to indicate a high glycemic response to the meal/food.

According to another aspect of the embodiments, the different colors can each indicate a range in which the analyte level variance value is determined by methods 300, 350, or 370 (as described with respect to FIGS. 3A, 3F, and 3G). For example, if the analyte level variance value is determined to be within a low analyte level variance range (e.g., less than 60 mg/dL, or less than 3.3 mmol/L), then the first segment is green (and the other segments are gray) to indicate that the meal/food elicited a low glycemic response, or had a low impact. As another example, if the analyte level variance value is determined to be within a medium analyte level variance range (e.g., between 60 mg/dL and 100 m/dL, or between 3.3 mmol/L and 5.6 mmol/L), then the second segment is yellow (and the other segments are gray) to indicate that the meal/food elicited a medium glycemic response, or a medium impact. As another example, if the analyte level variance value is determined to be within a high analyte level variance range (e.g., greater than 100 mg/dL, or greater than 5.6 mmol/L), then the third segment is orange (and the other segments are gray) to indicate that the meal/food elicited a high glycemic response, or a high impact. Examples of these interfaces are shown in modals 722, 724, and 726 (FIGS. 7D, 7E, and 7F).

According to another aspect of the embodiments, one or more conditions can cause all segments to be gray, such as if the analyte level variance value is below zero, if the analyte level variance value is above a maximum analyte level variance value (e.g., 170 mg/dL, 180 mg/dL, 190 mg/dL), or if the initial analyte level value is above a maximum initial analyte level value (e.g., 180 mg/dL, 200 mg/dL, 220 mg/dL, 250 mg/dL, etc.). One or more of these conditions may indicate that the determined analyte level variance value is either unreliable, cannot be accurately calculate, or otherwise not representative of an analyte level variance value relating to the consumption of a meal.

Furthermore, although the graphical representation shown is a segmented semi-circle having three colored segments, those of skill in the art will understand that other geometrical shapes, colors, and numbers of segments can be utilized, and are fully within the scope of the present disclosure. Likewise, as described above, each of the colors can represent a specific range of values. However, those of skill in the art will understand that other ranges can be utilized besides those listed, and that these numbers are not meant to be limiting.

Referring again to FIG. 7A, according to another aspect of the embodiments, when the graphical representation is selected by the user, a pop-up modal can be displayed that provides further information regarding the specific meal/food entry. For example, as shown in FIG. 7D, pop-up modal 722 presents a graphical representation for a “low impact” (green) meal/entry adjacent to a textual description, wherein the textual description provides the analyte level variance value of the meal/entry (e.g., 27 mg/dL), followed by the method by which the analyte level variance value was calculated (e.g., using the glucose value at the time you entered the food and the subsequent peak). Similarly, pop-up modals 724, 726 (FIGS. 7E and 7F) each correlate with “moderate impact” (yellow) and a “high impact” (orange) meal/entries, respectively, along with their corresponding textual descriptions.

In some embodiments, if a meal/food entry has not yet been associated with received analyte data, the selection of the meal/food entry can cause the meal monitor application to display a notification 728 that no analyte data has been received for the entry (as shown in FIG. 7G).

In alternative embodiments, as shown in FIG. 7I, the meal/food entry shown in the daily listing can be configured to expand to display an additional information panel 738. In these embodiments, information panel 738 can include a weighted average metric for the analyte level variance value if the meal has been consumed multiple times in the past.

In some embodiments, the weighted average of the analyte level variance value for a particular food or meal can be based on same (or similar) food entries which were added within a predetermined time period (e.g., in the last sixty days, in the last ninety days, in the last 120 days). In some embodiments, the weighted average can be based on a predetermined number of minimum analyte variance level values and a predetermined number of maximum analyte variance level values within the predetermined time period. Furthermore, in some embodiments, meal/food entries where no analyte variance level value was determined (or less than a minimum threshold) can be discarded as part of the weighted average determination. The weighted average can then be calculated/updated every time a relevant meal/food entry is created and/or updated.

According to some embodiments, the weighted average can also take into consideration the recency of the stored analyte level variance values for a particular meal/food. For example, historical analyte level variance values for a meal/food entry that are more recent than other historical values can be weighed more heavily when determining the weighted average. By way of illustration only, meal/food entries can be decremented by a predetermined factor for each day before the current date when calculating a weighted average.

In other embodiments, a regular average, median, mode, or another measure of central tendency for the analyte level variance values can be utilized instead of a weighted average.

In some embodiments, information panel 738 can also be configured to display activity information associated with the particular meal, such as the information inputted into activity text box 616 (FIG. 6B). As shown in FIG. 7I, for example, “Workout, Running” is displayed in additional information panel 738. In some embodiments, information panel 738 can also display notes, such as the information inputted into notes text box 630 (FIG. 6E).

Referring back to FIG. 7H, an example embodiment of a weekly listing 734 of meal-related glycemic responses for diary GUI 700 is depicted. As can be seen in weekly listing 734, a plurality of meals/foods is presented for a week time period, “13 Nov.-19 Nov. 2021.” As seen at the left portion of each meal/food entry, a graphical representation indicating the user's glycemic response to a corresponding meal/food is displayed. Along with the graphical representation, each meal/food entry can include a date, time-of-entry, and a numeric value indicating the analyte level variance value. Those of skill in the art will appreciate that any of the aforementioned features and processes described with respect to daily listing 710 (FIG. 7A) can also be applied with respect to the weekly listing 734 (FIG. 7H).

Referring back to FIG. 7C, an example embodiment of a filter GUI 720 for diary GUI 700 is depicted. According to some embodiments, filter GUI 720 can include a date filter 719 to limit the display of meal/food entries to a particular date range. In some embodiments, the filter GUI 720 can also include an analyte level variance value filter 717 to limit the display of meal/food entries to a particular ranking of analyte level variance values (e.g., low impact, medium impact, or high impact). Filter GUI 720 can also include an apply button 716 to apply the selected filters to the current set of meal/food entries. Likewise, in some embodiments, the filter feature can also include a clear button 718 to reset or remove any current filters.

With reference to FIGS. 7J-7R, an additional exemplar embodiment of a diary GUI 750 for a meal monitor application is depicted. In many embodiments, diary GUI 750 is displayed when the user selects the diary section 4012 on home GUI 460. Alternatively, diary GUI 750 can be displayed when the user selects the corresponding icon in the navigation bar (bottom). Specifically, FIG. 7J depicts a diary GUI 750 which is similar to diary GUI 700 except that diary GUI 750 does not include a notification button. According to another aspect of the embodiment, unlike diary GUI 700, each meal/food entry in diary GUI 750 includes a portion size indicator 751 describing the relative portion size of the meal (e.g., “S” to represent a small meal, “M” to represent a medium meal, “or “L” to represent a large meal) instead of a numeric value indicating the analyte level variance value associated with the meal/food.

Further, the informational button 7502 on diary GUI 750 can be selected by the user to provide contextual information 7514 (FIG. 7K). In some embodiments, a reference link 752 can be included with the contextual information 7514 which, when selected by the user, outputs a reference GUI 760 (FIG. 7L) comprising references/sources related to the contextual information 7514.

Referring to FIG. 7M, an exemplar embodiment of a filter GUI 770 for diary GUI 750 is provided, which is similar to filter GUI 720 for diary GUI 700.

Additionally, FIGS. 7N-7R depict example embodiments of various pop-up modals 761, 762, 763, 764, and 765, respectively, that provide further information regarding the specific meal/food entry. Specifically, the pop-up modals 761, 762, 763, 764, and 765, in FIGS. 7N-7R can be displayed on the meal monitor application when the user selects a graphical representation on the diary GUI 750. For example, as shown in FIG. 7N, pop-up modal 761 presents a graphical representation for a “low impact” (green) meal/entry adjacent to a textual description, wherein the textual description provides the analyte level variance value of the meal/entry (e.g., 2.2 mmol/L), followed by the method by which the analyte level variance value was calculated (e.g., using the sugar level at the time you entered the food and its highest level in the following two hours). Similarly, pop-up modals 762, 763 (FIGS. 7O and 7P) each correlate with “moderate impact” (yellow) and a “high impact” (orange) meal/entries, respectively, along with their corresponding textual descriptions.

According to another aspect of the embodiments, and as shown in FIG. 7Q, pop-up modal 764 presents a graphical representation for a “no impact” (all gray) meal/entry along with a corresponding textual description, wherein the textual description provides the analyte level variance value of the meal/entry is below zero (e.g., −0.8 mmol/L). In some embodiments, if a meal/food entry has not yet been associated with received analyte data, the selection of the meal/food entry can cause the meal monitor application to display a notification 765 that no sugar data is available for the entry and a reminder to the user to scan the sensor once every eight hours (as shown in FIG. 7R).

### Example Embodiments of Trends GUIs

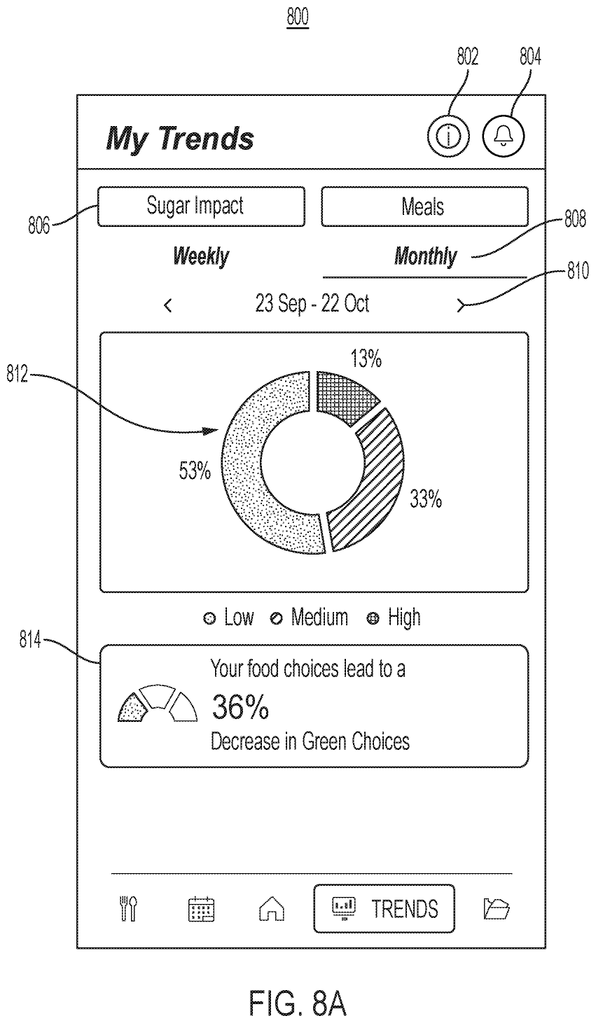

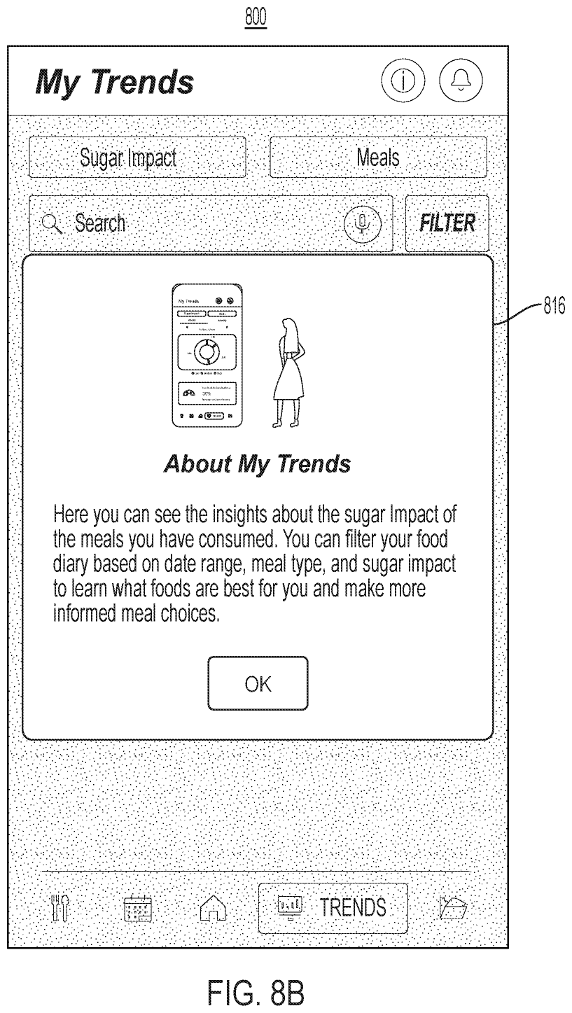

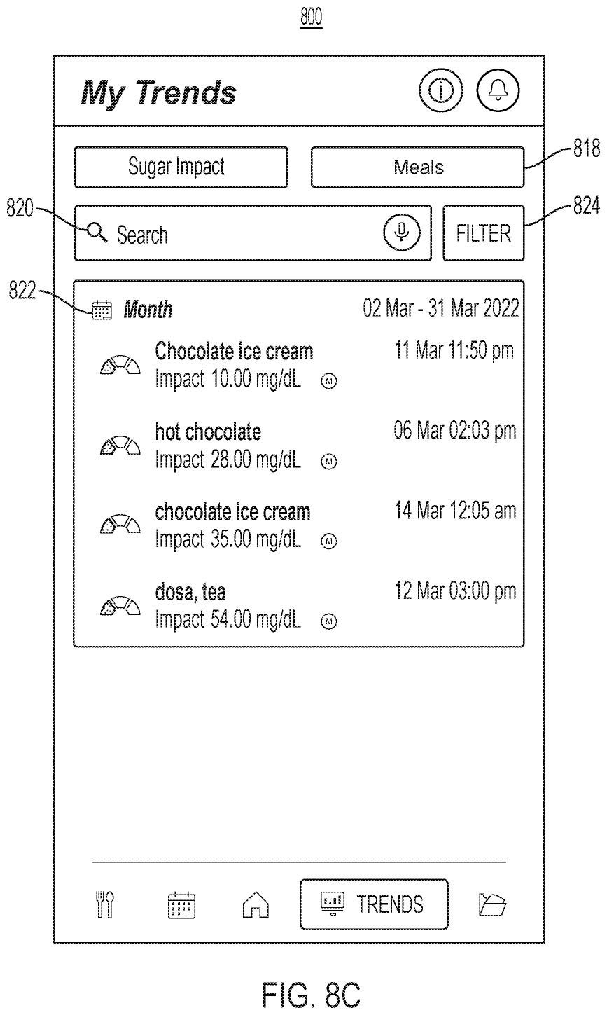

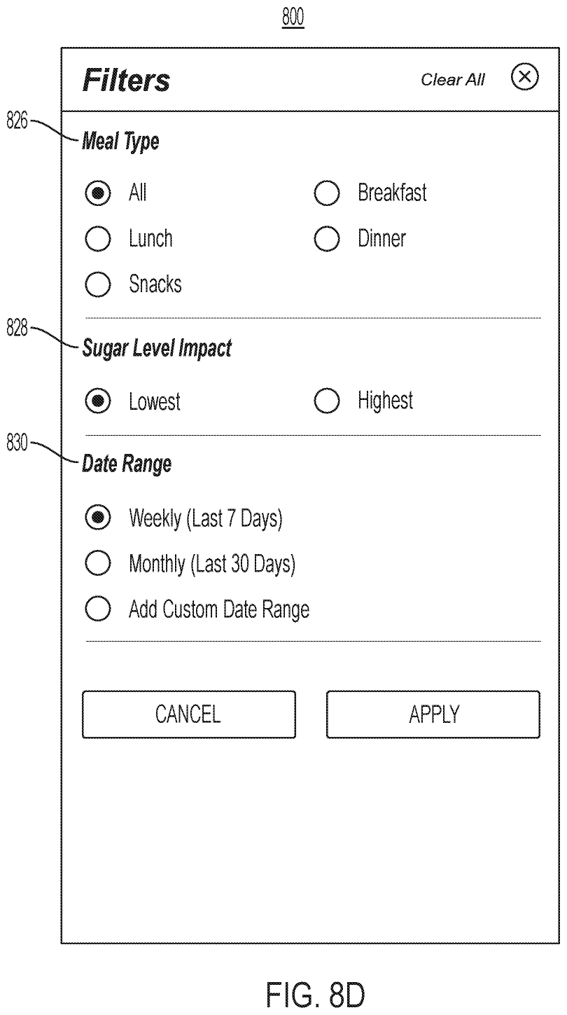

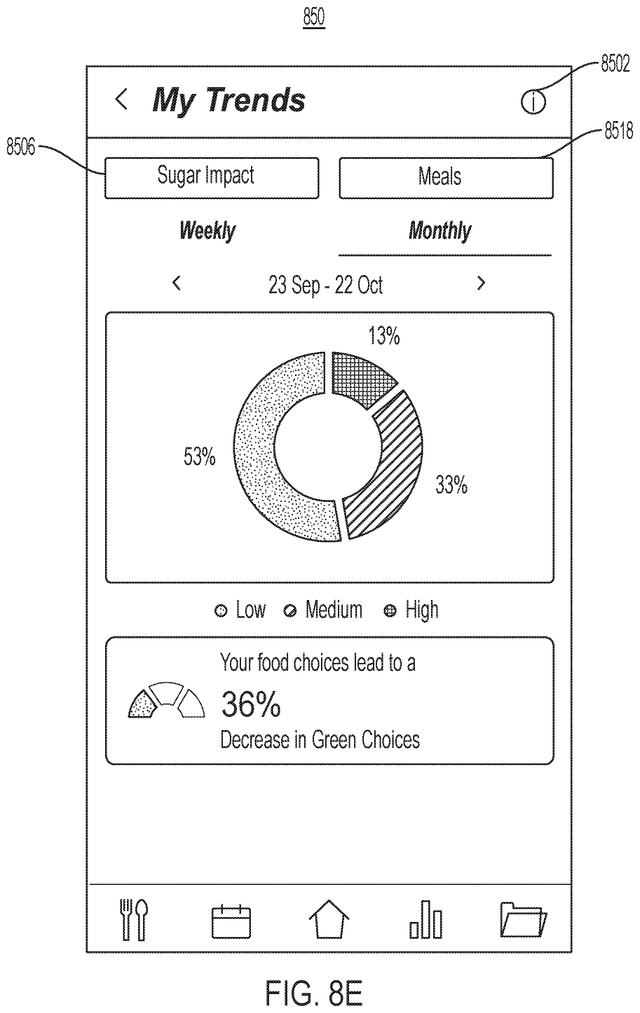

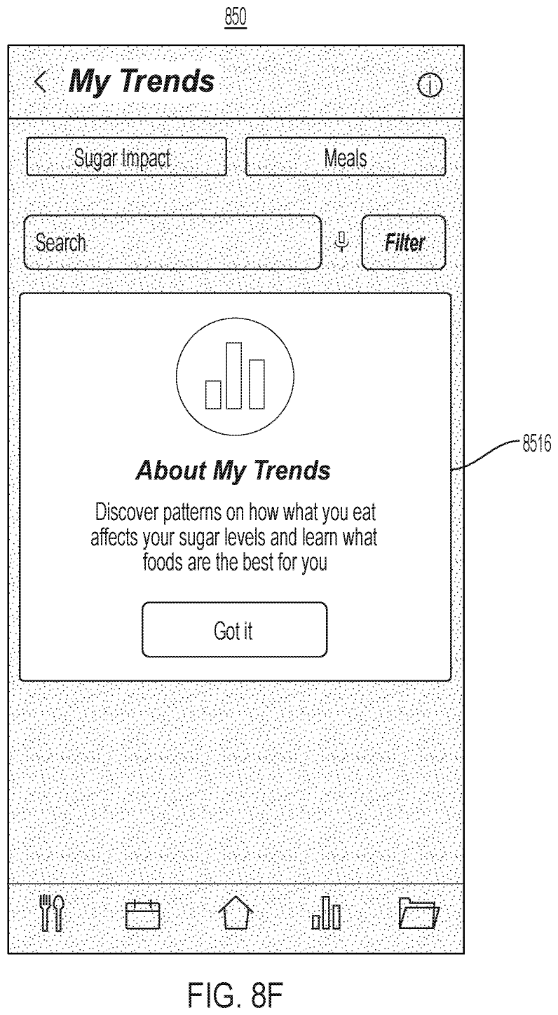

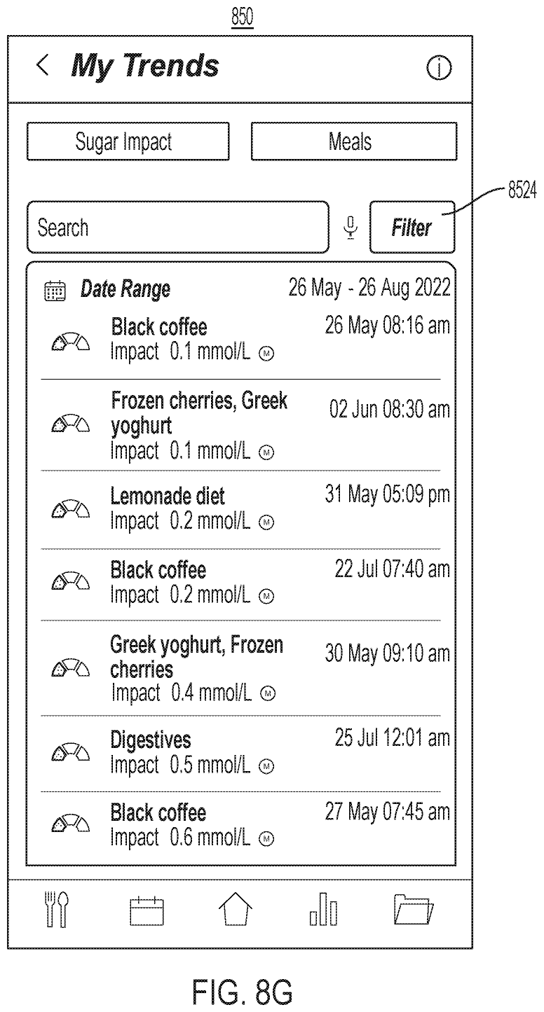

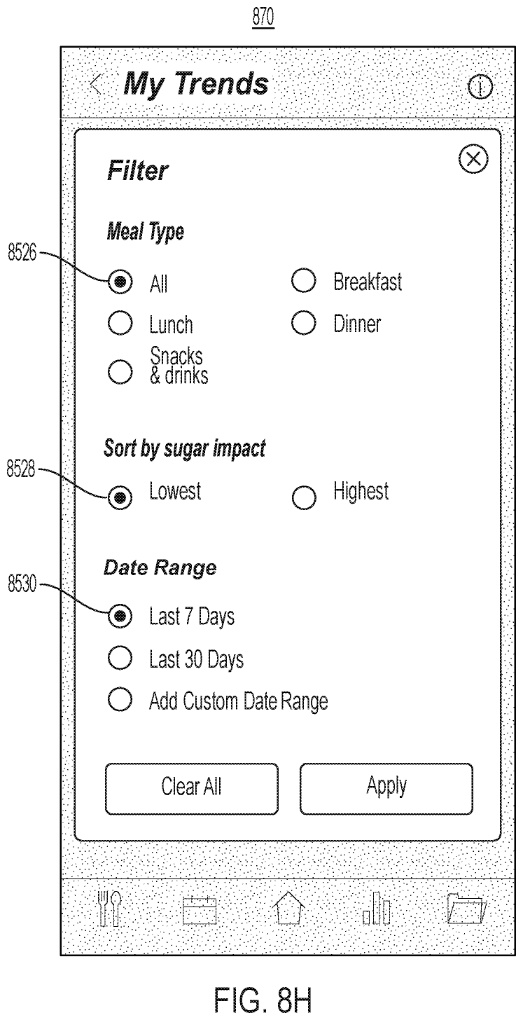

Turning to FIGS. 8A to 8D, example embodiments of a trends GUI and features related thereto for a meal monitor application are depicted. In many embodiments, a trends GUI 800 can be displayed when the user selects the trends section 406 on home GUI 400 (FIG. 4A-1). Alternatively, trends GUI 800 can also be displayed by the selection of the corresponding icon in the navigation bar (bottom). Moreover, trends GUI 800 can include an information button 802 (FIG. 8A) to provide contextual information 816 (FIG. 8B), and a notification button 804 (FIG. 8A) to provide the user with in-app notifications (FIG. 4O).

According to another aspect of the embodiments, trends GUI 800 can display the meal-related trends in a manner that is user-friendly and easy to understand. In some embodiments, for example, trends GUI 800 can include a plurality of views by which the trends can be displayed. For example, as shown in FIG. 8A, trends GUI 800 can present trend information relating to analyte response when the corresponding “sugar impact” option 806 is selected. Similarly, as shown in FIG. 8C, trends GUI 800 can present trend information relating to meals/foods when the “meals” option 818 is selected.

Referring again to FIG. 8A, with respect to the “sugar impact” trends view, trend information can further be presented by week or by month, as indicated by a time period option 808. Additionally, in some embodiments, trends GUI 800 can also include a date changing setting 810 that allows the user to select different time periods. According to one aspect of the embodiments, the trend information presented can automatically change/update if the user selects a different time period.

In many embodiments, trends GUI 800 further comprises an easy-to-comprehend graphical representation 812 of the “sugar impact” of the user's meal/food choices to her analyte levels for the selected time period. By way of illustration, graphical representation 812 of FIG. 8A shows, for example, that 53% of the user's meal/food entries were classified as having a “low impact” (e.g., shown as a green segment) for the monthlong time period between September 23 and October 22. Similarly, graphical representation 812 of FIG. 8A shows that 33% of the user's meal/food entries were classified as having a “medium impact” (e.g., shown as a yellow segment) for the same period. Likewise, about 13% of the user's meal/food entries were classified as having a “high impact” (e.g., shown as an orange segment) for the same period. As can be seen in FIG. 8A, each segment can have a corresponding color with a range of analyte level variance values (as described earlier with, e.g., respect to FIGS. 4A-3 and 4A-4), as well as a corresponding area that reflects the magnitude of the analyte level variance value as a percentage of the total graphical representation. Although graphical representation 812 is shown as a “pie chart” in FIG. 8A, those of skill in the art will appreciate that other types of graphs (e.g., bar charts, line graphs, etc.), color schemes (e.g., red, yellow, green, etc.), and numbers of segments (e.g., 3, 4, 5, 6, etc.) can be utilized, and are fully within the scope of the present disclosure.

According to another aspect of some embodiments, trends GUI 800 can also include a summary panel 814, which can provide an overall assessment of the user's food choices. By way of illustration, summary panel 814 of FIG. 8A states that the user's “food choices lead to a 36% decrease in green choices.”

Turning to FIG. 8C, trends GUI 800 is shown with the meals view option 818 selected. According to one aspect of the embodiments, the meals view can include a meal/food listing 822 displaying the name of each meal/food, a graphical and a numeric representation of the analyte level variance value, and a corresponding date and time. In some embodiments, a search bar 820 can also be provided so that the user can identify specific meals/foods within the selected time period. In some embodiments, a voice recognition feature is provided to allow the user to speak the name of the meal or food, instead of manually typing it into search bar 820. In addition, according to some embodiments, a filter button 824 can be provided to allow the user to filter the trends information in a variety of helpful views. For example, in some embodiments, selecting filter button 824 can cause display of a filter settings GUI 840 that can include a meal type filter 826, a sugar level impact filter 828, and/or a date range filter 830.

FIGS. 8E-8H depict an additional example embodiment of a trends GUI and features related thereto for use with a meal monitoring application. In many embodiments, a trends GUI 850 can be displayed when the user selects the trends section 4006 on home GUI 460 (FIG. 4A). Alternatively, trends GUI 850 can also be displayed by the selection of the corresponding icon in the navigation bar (bottom). The trends GUI 850 depicted in FIG. 8E-8H is similar to the trends GUI 800 depicted in FIG. 8A-8D, except that trends GUI 850 does not include a notification button. FIG. 8E depicts trends GUI 800 presenting trend information relating to analyte response when the corresponding “sugar impact” option 8506 is selected. FIG. 8F illustrates the contextual information 8516 displayed when the user selects an informational button 8502 on trends GUI 850. Further, FIG. 8G shows trends GUI 850 presenting trend information relating to meals/foods when the “meals” option 8518 is selected. FIG. 8H illustrates a filter settings GUI 870 that is displayed after the user has selected a filter button 8524 (FIG. 8G). As shown in FIG. 8H, the filter settings GUI 870 can include a meal type filter 8526, a sugar impact filter 8528, and/or a date range filter 8530.

### Example Embodiments of Reports GUIs

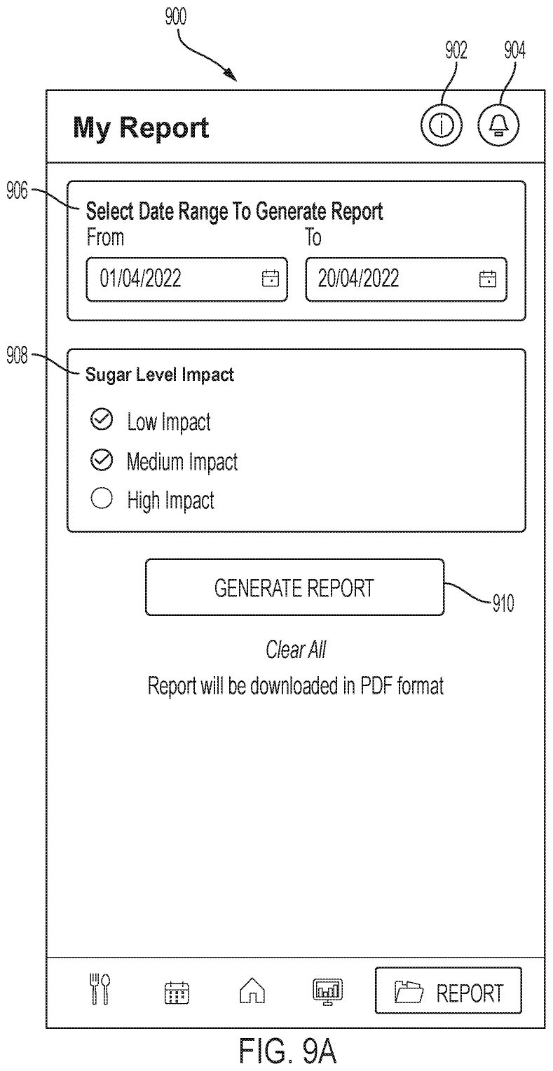

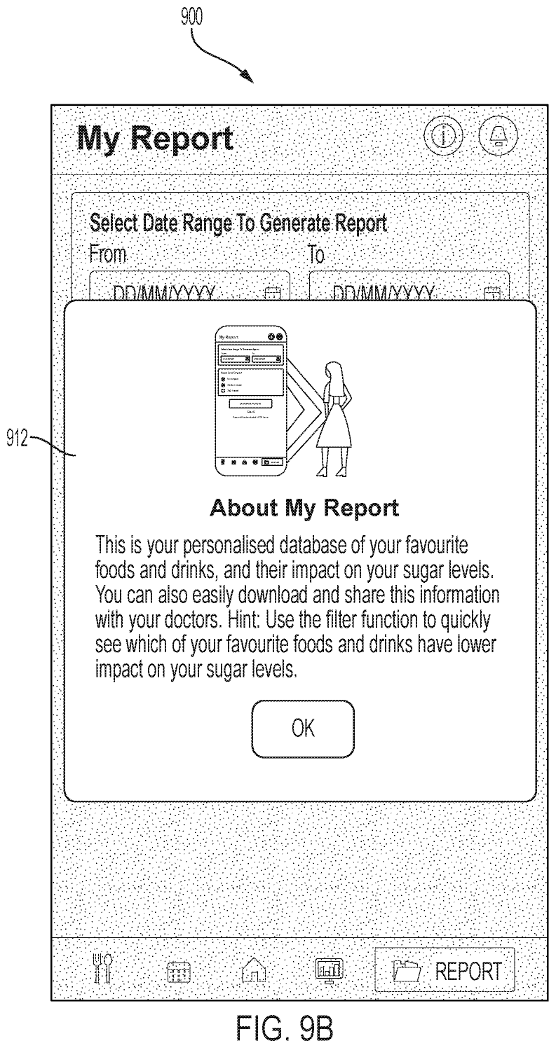

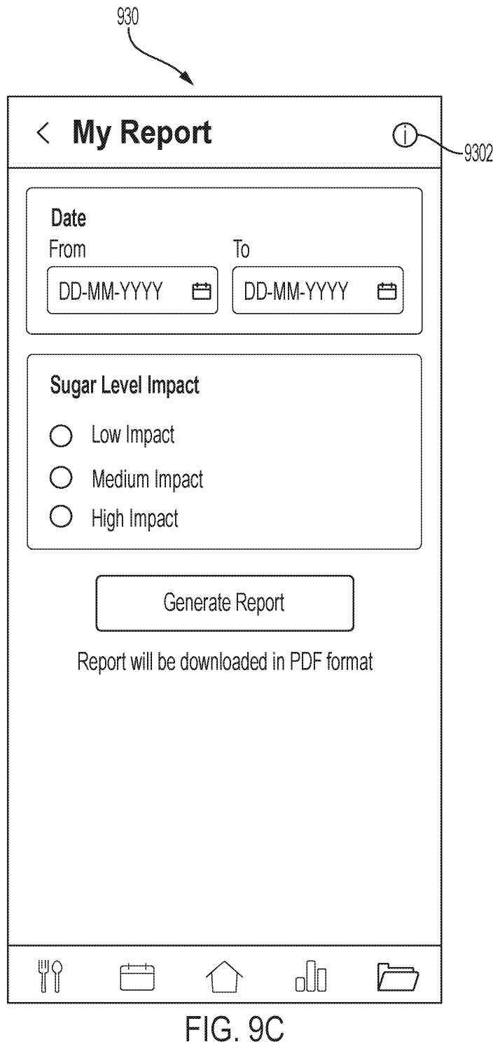

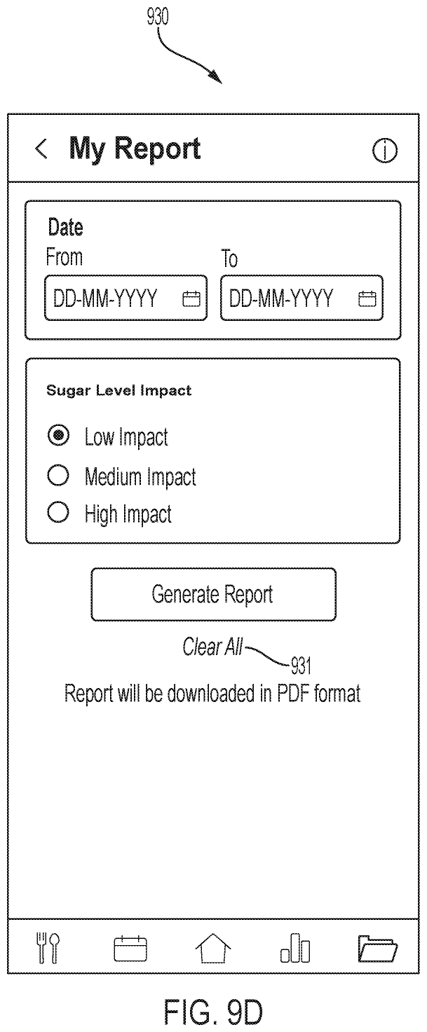

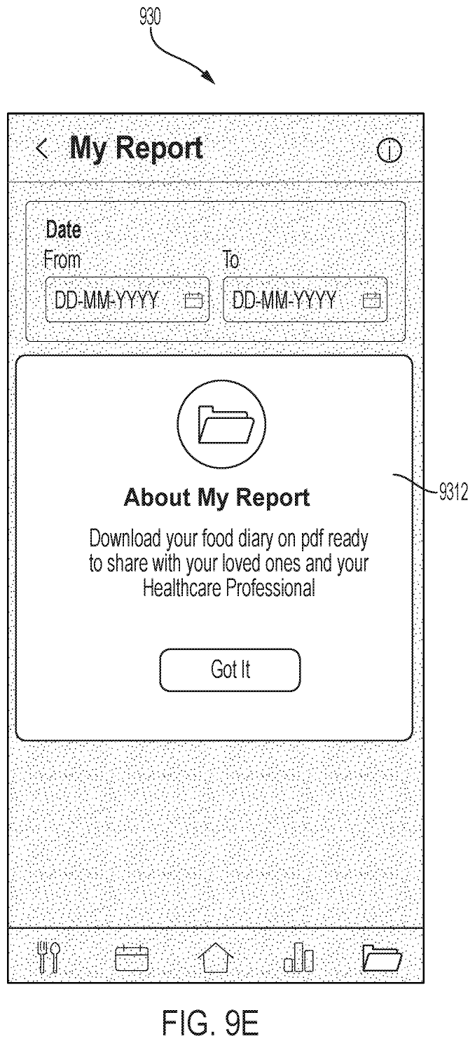

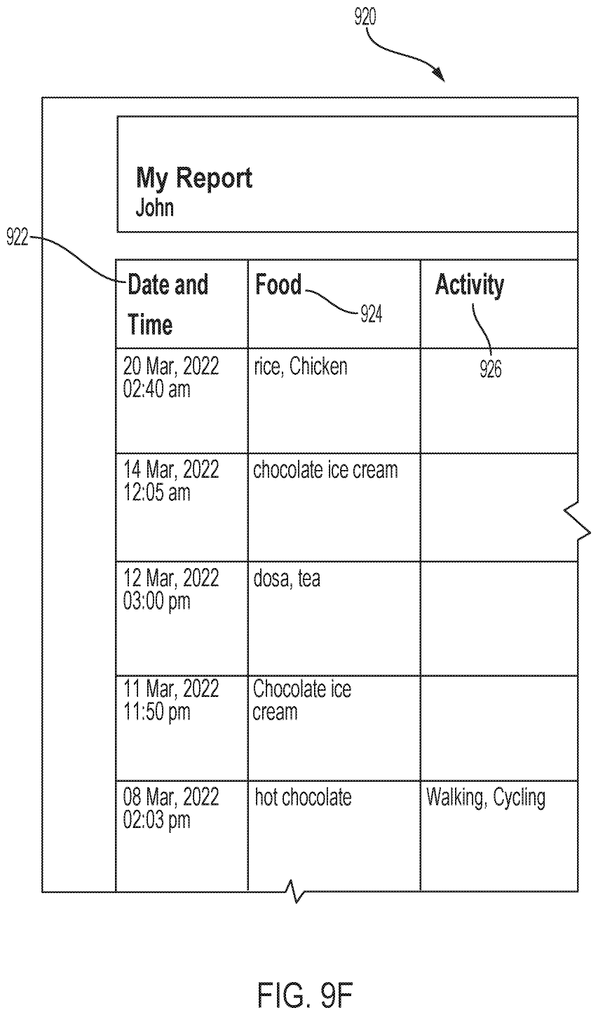

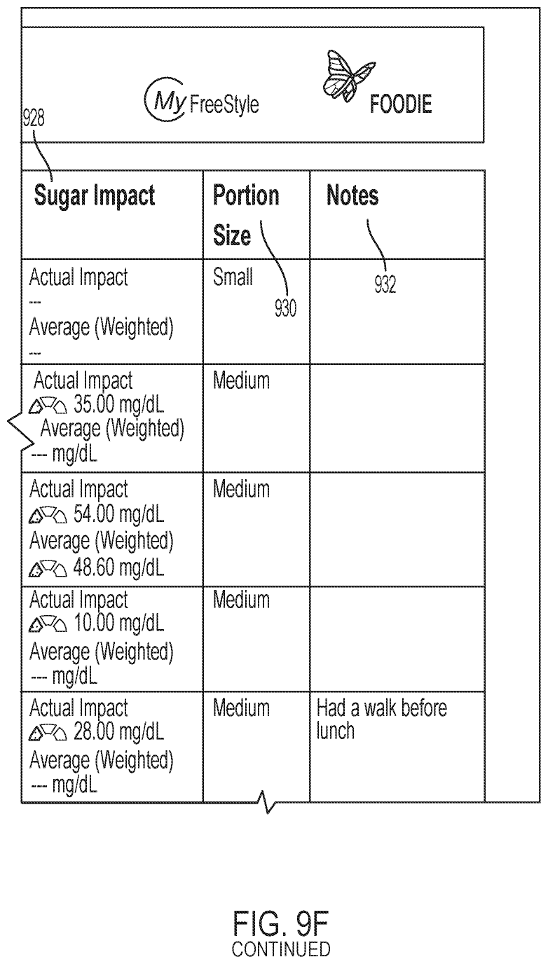

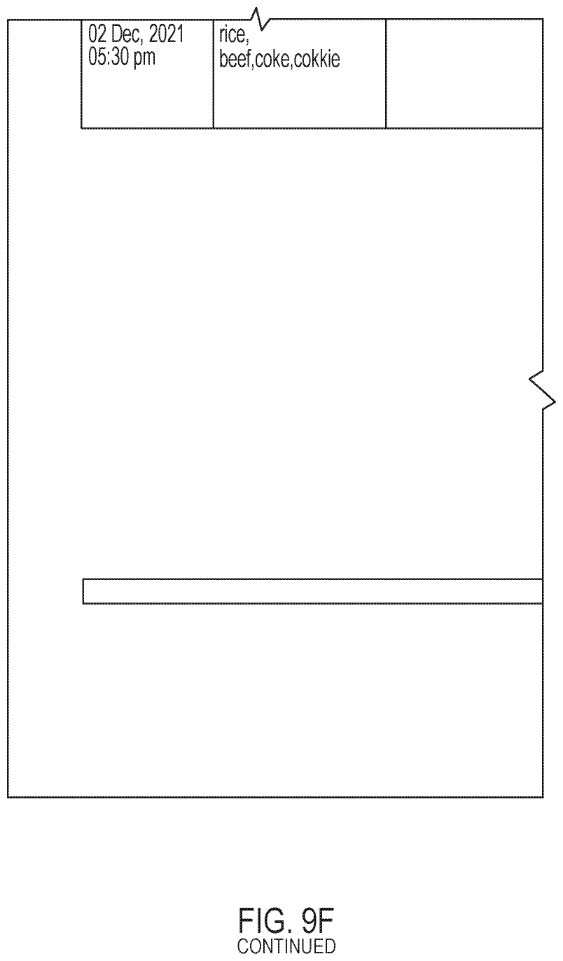

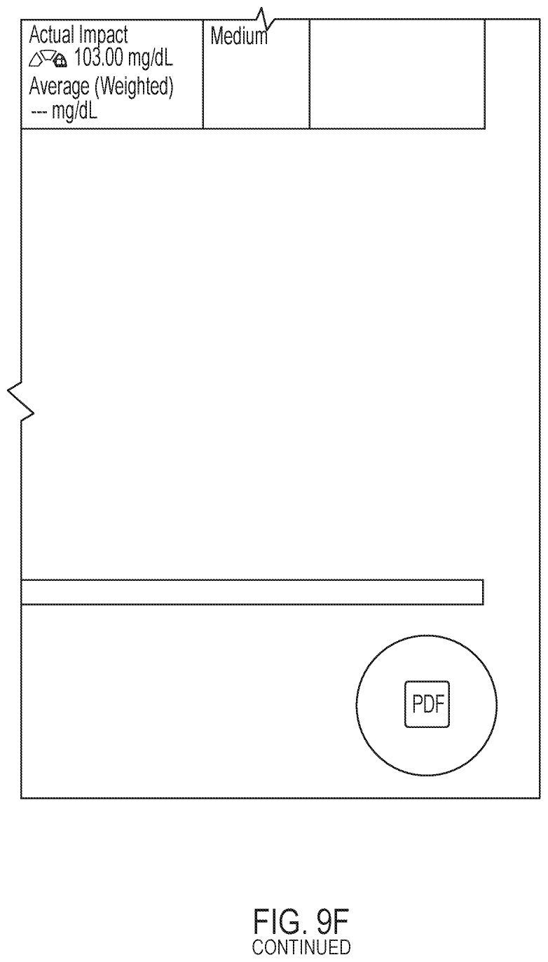

Turning to FIGS. 9A to 9F, example embodiments of a reports GUI 900 for a meal monitor application are depicted. Referring to FIGS. 9A and 9B, in many embodiments, a reports GUI 900 can be displayed when the user selects the reports section 414 on home GUI 400 (FIG. 4A-1). Alternatively, reports GUI 900 can be displayed by the selection of the corresponding icon in the navigation bar (bottom). Moreover, reports GUI 900 can include an information button 902 (FIG. 9A) to provide contextual information 912 (FIG. 9B), and a notification button 904 (FIG. 9A) to provide the user with in-app notifications (FIG. 4O).

According to one aspect of the embodiments, reports GUI 900 can include a date range field 906 to allow the user to select a date range for the report, and a sugar level impact option 908 to allow the user to report on the meal/food entries having certain analyte level variance value rankings (e.g., “low impact,” “medium impact,” and “high impact”). Reports GUI 900 also includes a generate report button 910 that can be selected once the user has chosen the desired settings.

FIGS. 9C-9E depict an additional example embodiment of a reports GUI 930 for use with a meal monitoring application. In many embodiments, reports GUI 930 can be displayed when the user selects the reports section 4014 on home GUI 460. Alternatively, reports GUI 930 can be displayed by the selection of the corresponding icon in the navigation bar (bottom). Specifically, reports GUI 930 is similar to reports GUI 900 except that reports GUI 930 does not include a notification button. Further, reports GUI 930, and as depicted in FIG. 9C, comprises a “clear all” button 931, which the user can select to clear all entries inputted into the reports GUI 930. FIG. 9E depicts contextual information 9312 that is displayed upon the user clicking information button 9302 on reports GUI 930 information button 9302 on reports GUI 930.

Turning to FIG. 9F, an example embodiment of a report 920 generated by the meal monitor application is displayed. According to some embodiments, report 920 can be generated as a PDF file, so that the user can easily print the report or attach it to an e-mail to send to a health care provider. In other embodiments, report 920 can be generated as a Word .DOC file, an Excel spreadsheet, or a comma-separated file. In some embodiments, report 920 can be an in-app report, a WebView, or any other format desired. As can be seen in FIG. 9F, report 920 can include a date and time field 922, a food field 924, an activity field 926, a sugar impact field 928, a portion size field 930, and a notes field 932. Those of skill in the art will understand that these fields are meant to be illustrative only, and that other fields, measurements, values, text fields, and/or options described herein can be displayed in report 920.

### Example Embodiments of Onboarding Methods and Related GUIs

An example embodiment of a method for onboarding a user with respect to a meal monitor application, and the various GUIs and features relating thereto, will now be described. Turning first to FIG. 10, a method 1000 is provided for onboarding a user to any of the meal monitor application embodiments of the present disclosure. As an initial matter, those of skill in the art will understand that the onboarding methods and related GUIs can be software installed in non-transitory memory of any of the embodiments of reader device 120 (e.g., a smart phone), drug delivery device 160, or local computer system 170 described herein.

Referring to FIG. 10, at 1002, the meal monitor application is launched. Before allowing the user to go any further, however, it would be beneficial to ensure that the appropriate requirements are met to fully utilize the meal monitor application. At 1004, the user receives a prompt asking if they have a user account. In some embodiments, the user account can be an account to establish authentication with the trusted computer system, which can then provide the user's analyte data to the meal monitor application. If the user does not have an account, then at 1006, the meal monitor application can prompt the user register for an account. In some embodiments, if the user declines to register for an account, the meal monitor application can terminate. In other embodiments, if the user declines to register for an account, the meal monitor application can enter into an “unlinked” mode, in which the user can use the meal monitor application as a disconnected meal diary for the manual entry of meal-related information.

If the user registers for an account, or already has an account, then, at 1010, the user logs into the account successfully. Subsequently, at 1012, the meal monitor application inquires as to whether the user is wearing an active sensor control device. In some embodiments, if the user does not have a sensor control device then, at 1014, meal monitor application can display an interface through which the user can order a sensor. At 1016, the meal monitor application can terminate or enter into an “unlinked” mode.

If the user is wearing an active sensor control device, then, at 1018, the meal monitor application inquires as to whether the user has installed a sensor app. If the user has not installed the sensor app, then, at 1020, the meal monitor application can prompt the user to download the sensor app. In some embodiments, for example, the meal monitor can automatically open the “app store” to the page of the sensor application.

According to many of the embodiments, once the requirements are determined to be satisfied, then, at 1022, the meal monitor application starts, and the home GUI of FIG. 4A can be displayed.

FIGS. 11A-1 to 11N-3 depict example embodiments of onboarding GUIs for a meal monitor application, any of which can be utilized with the embodiments described herein. In some embodiments, when the meal monitor application is first launched (as described with respect to step 1002 in FIG. 10), GUIs 1100, 1105 and 1110 (FIGS. 11A-1 to 11A-3) can provide the user with a brief introduction to the meal monitor application.

FIGS. 111B-1 to 11B-6 depict additional exemplar embodiments of onboarding GUIs for use with a meal monitoring application. Specifically, FIGS. 11B-1 to 11B-6 depict onboarding GUIs 1101, 1102, 1103, 1104, 1105, and 1106, respectively, which can provide the user with a brief introduction to the meal monitor application after it is first launched.

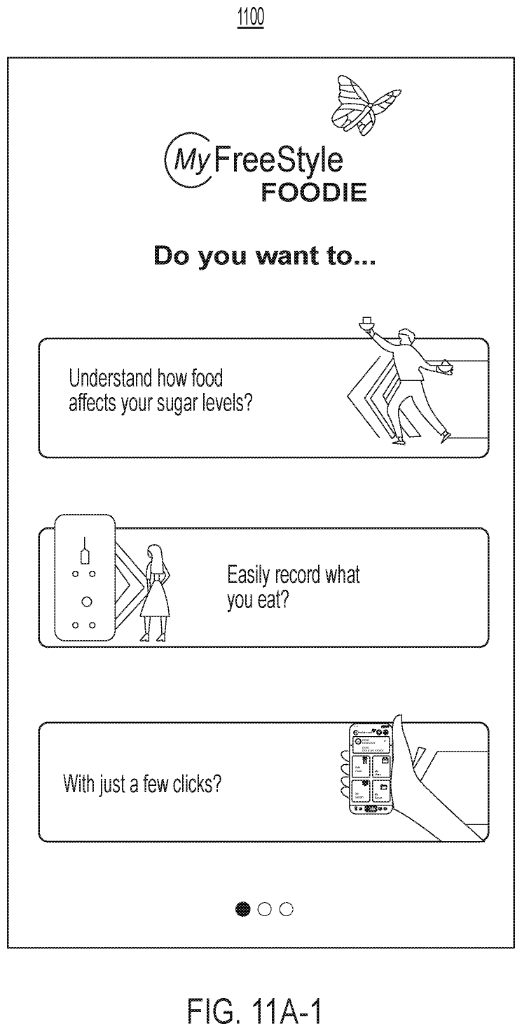

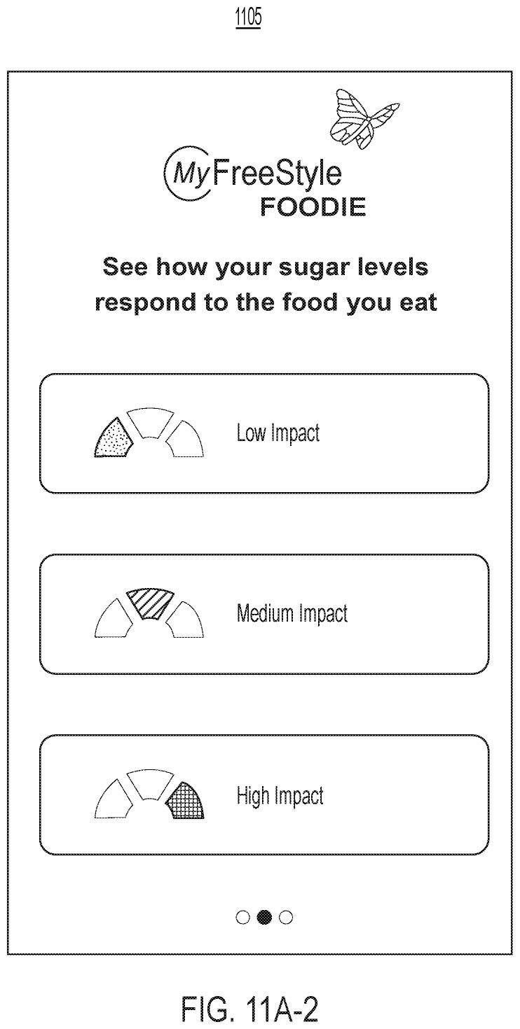

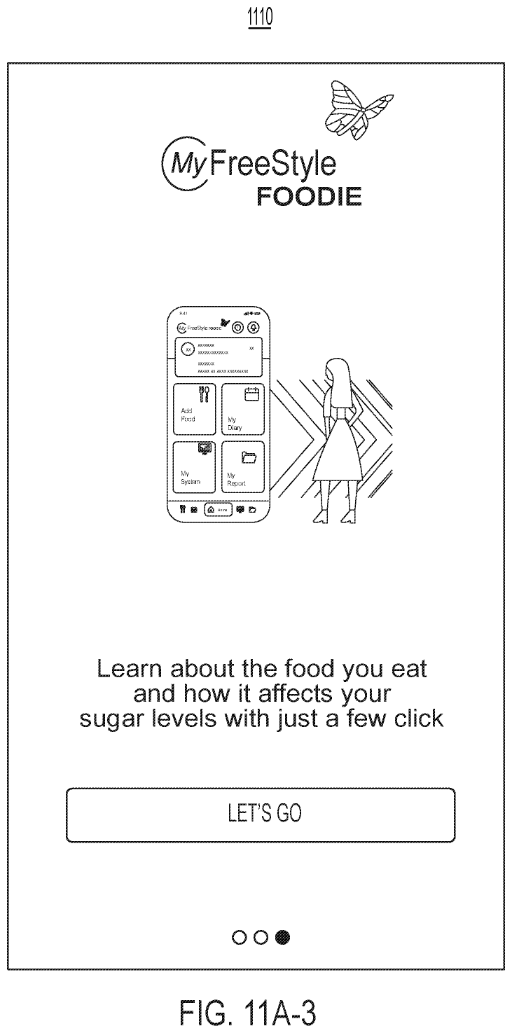

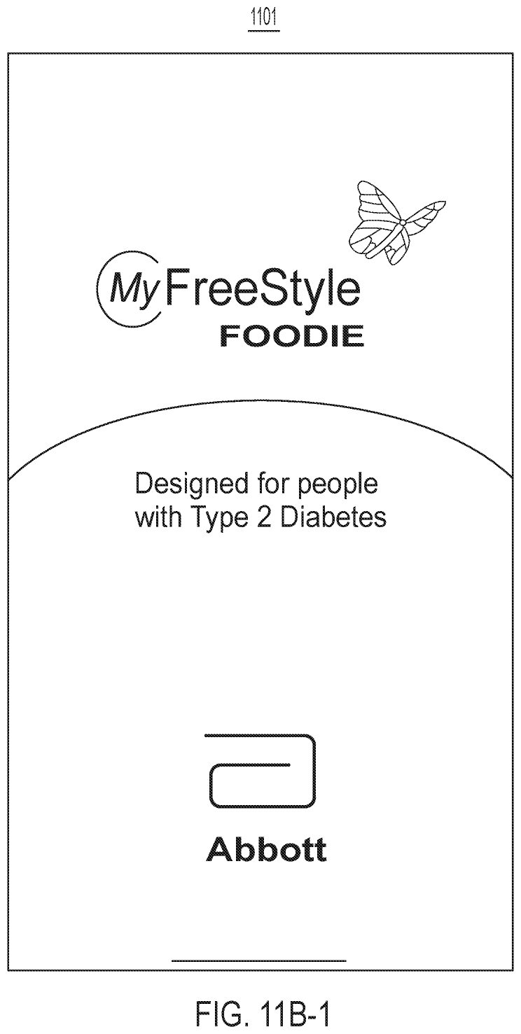

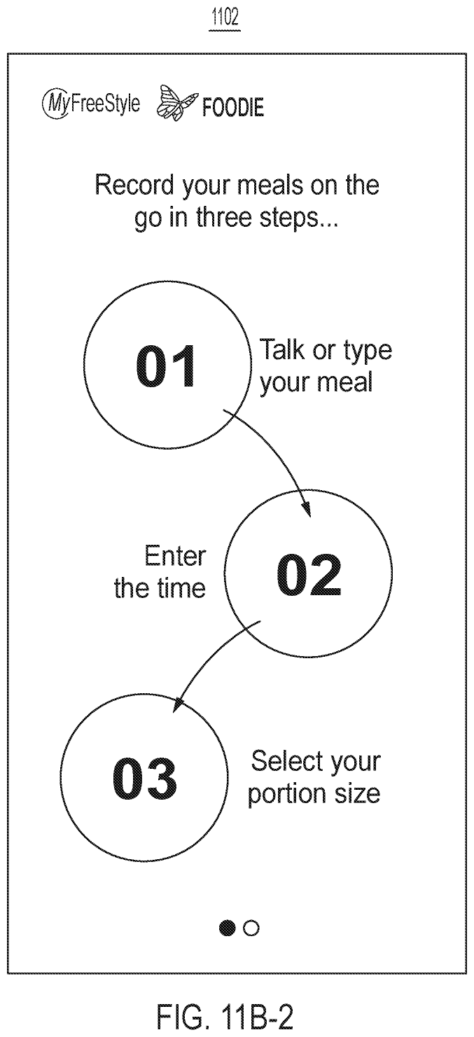

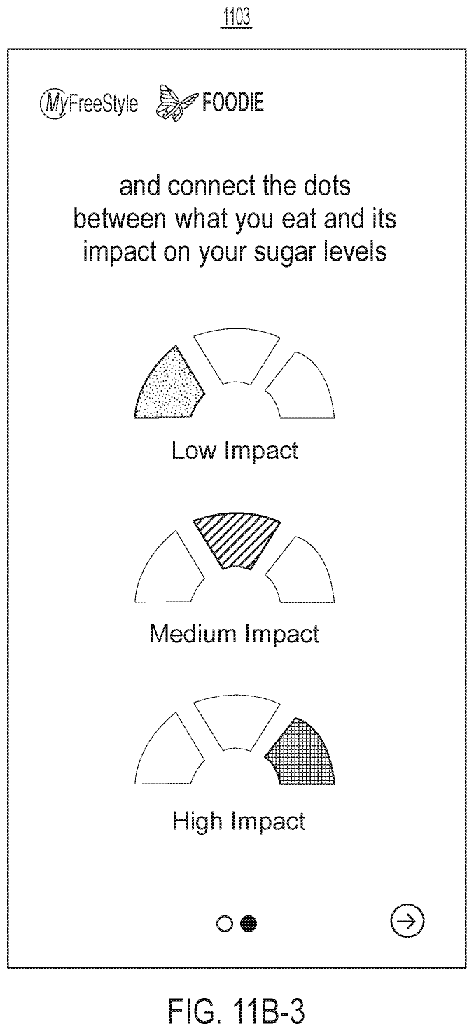

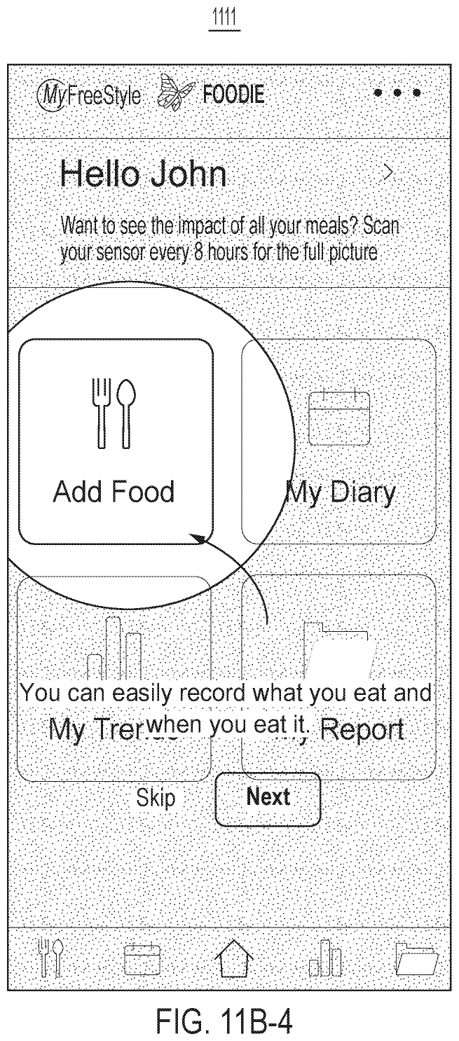

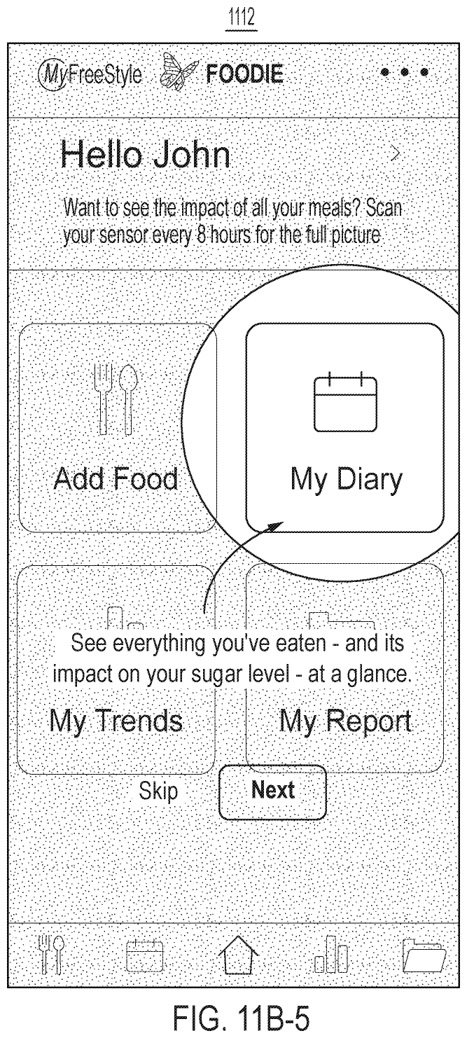

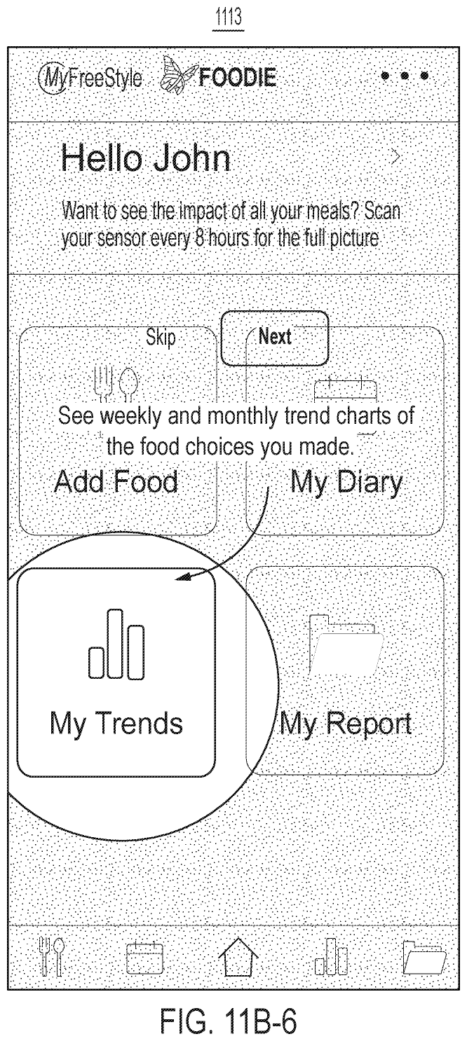

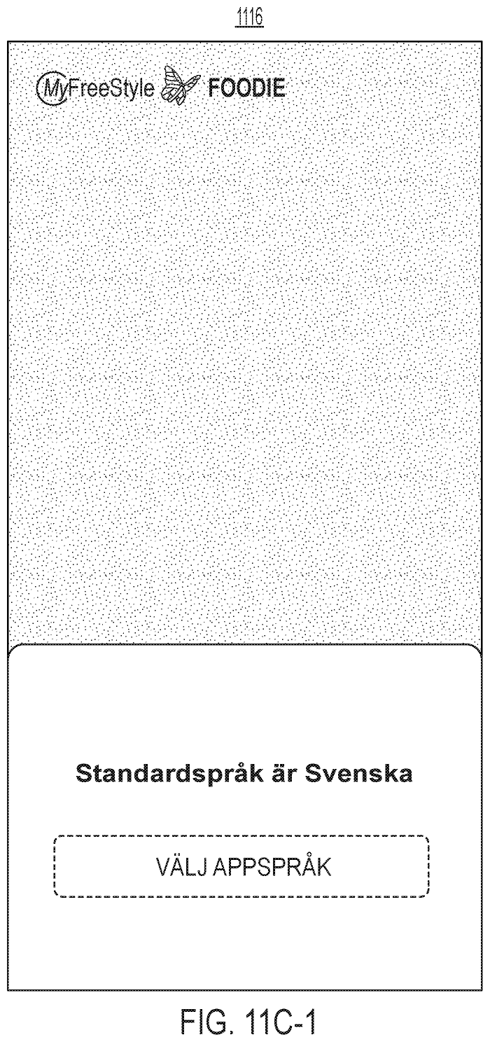

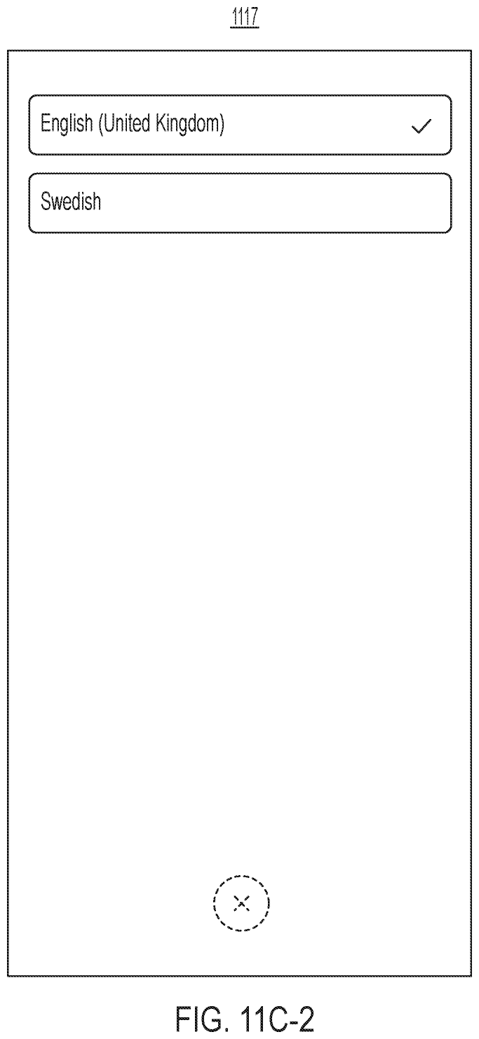

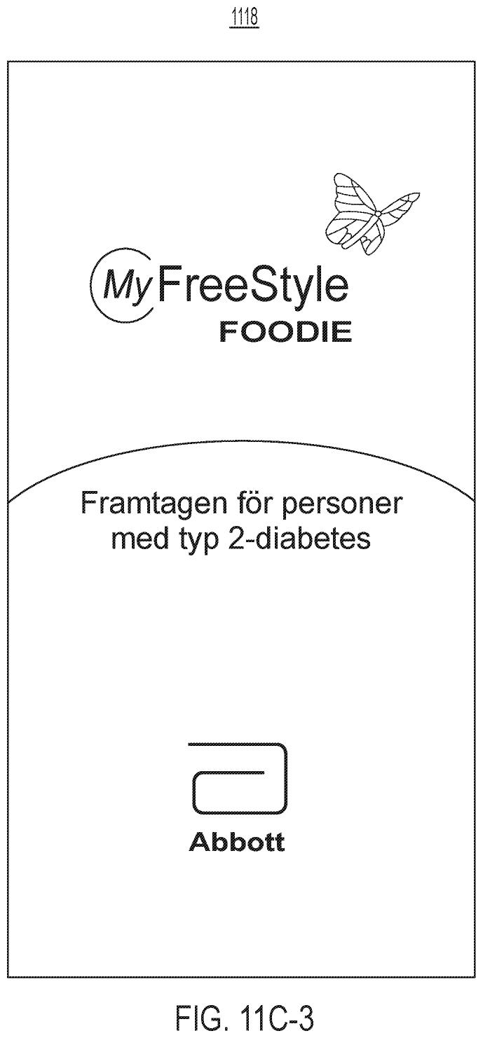

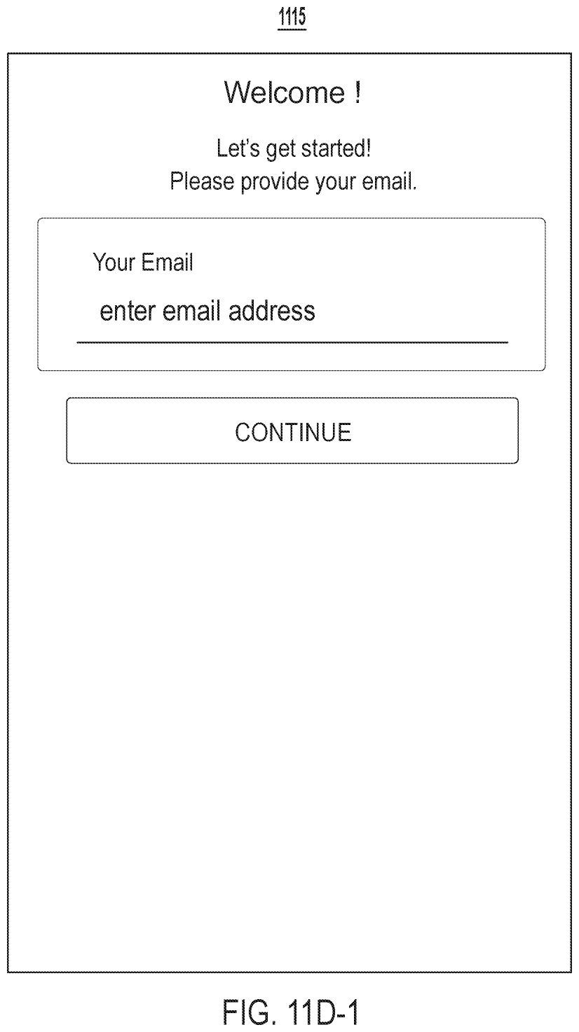

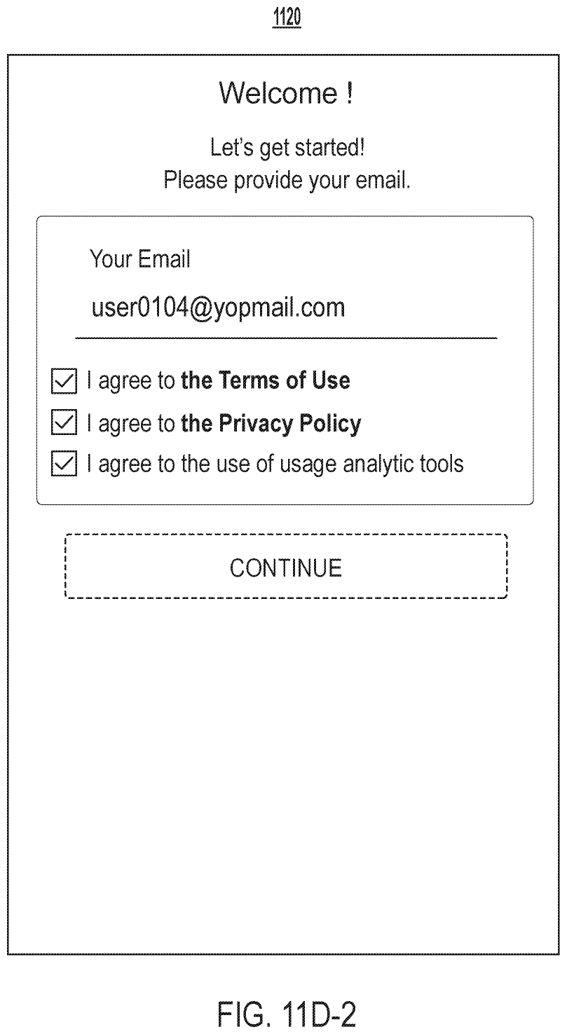

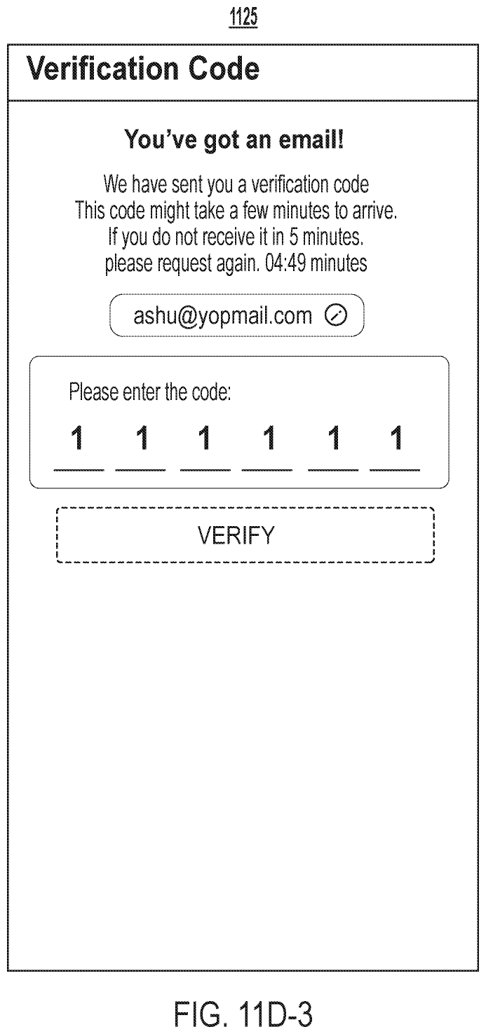

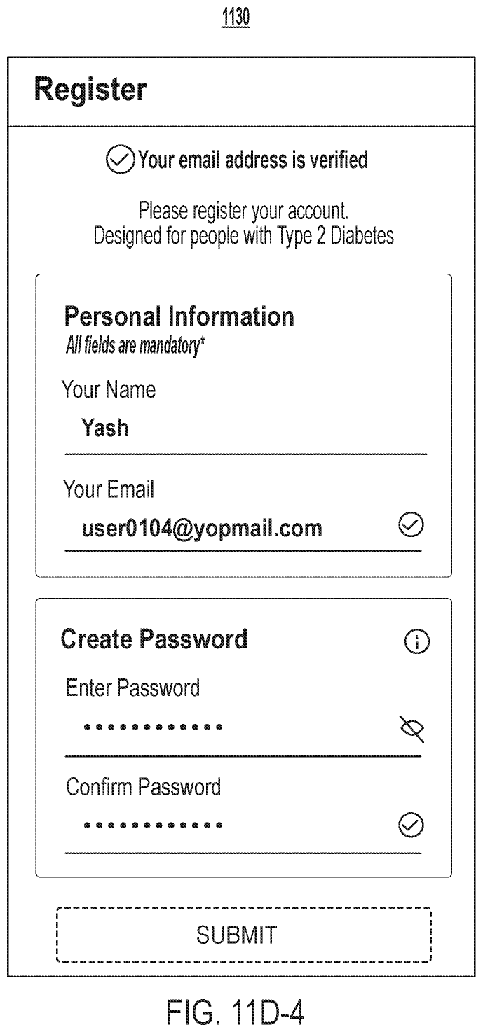

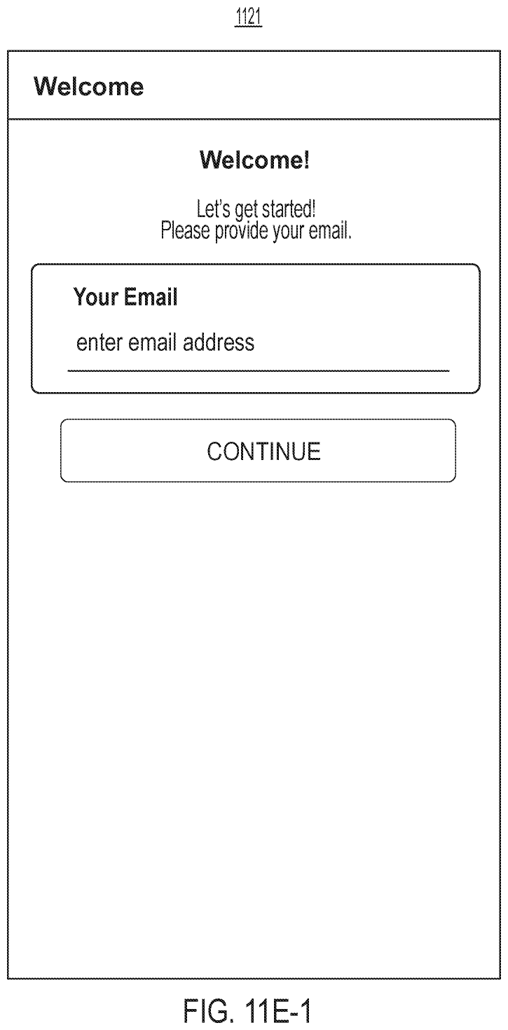

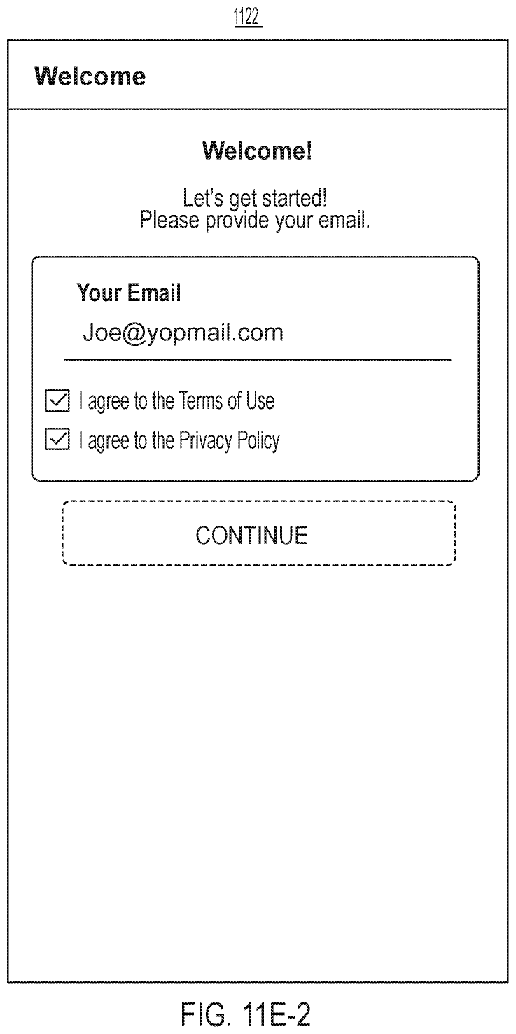

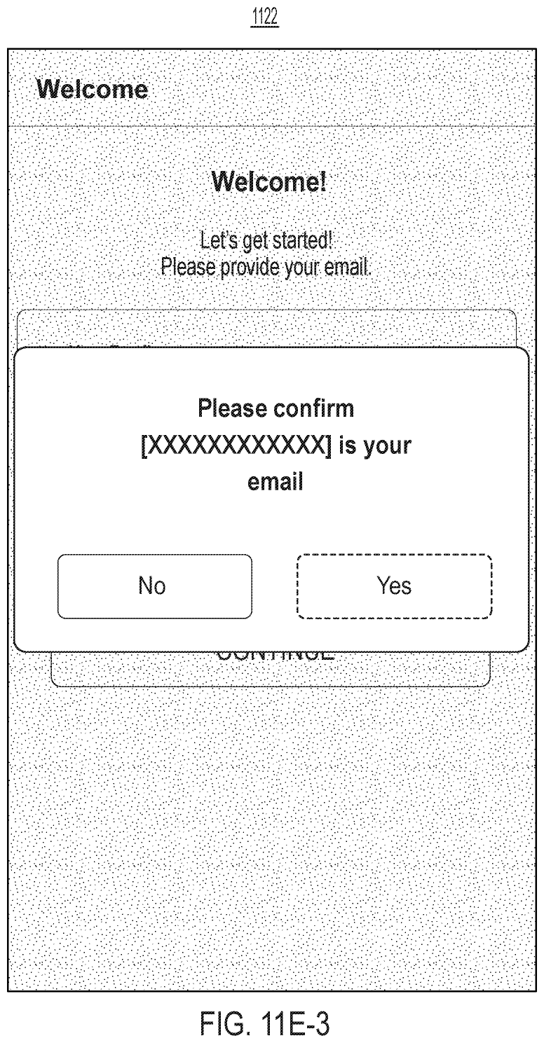

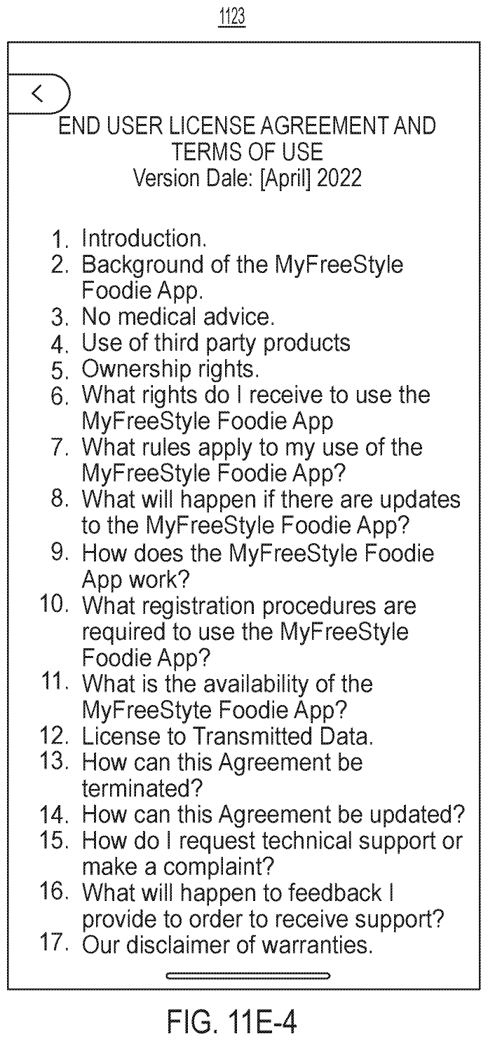

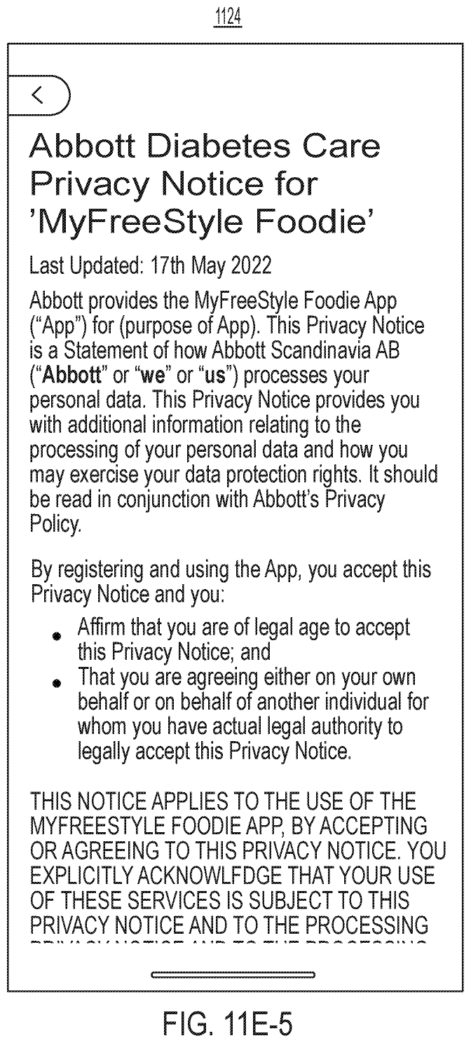

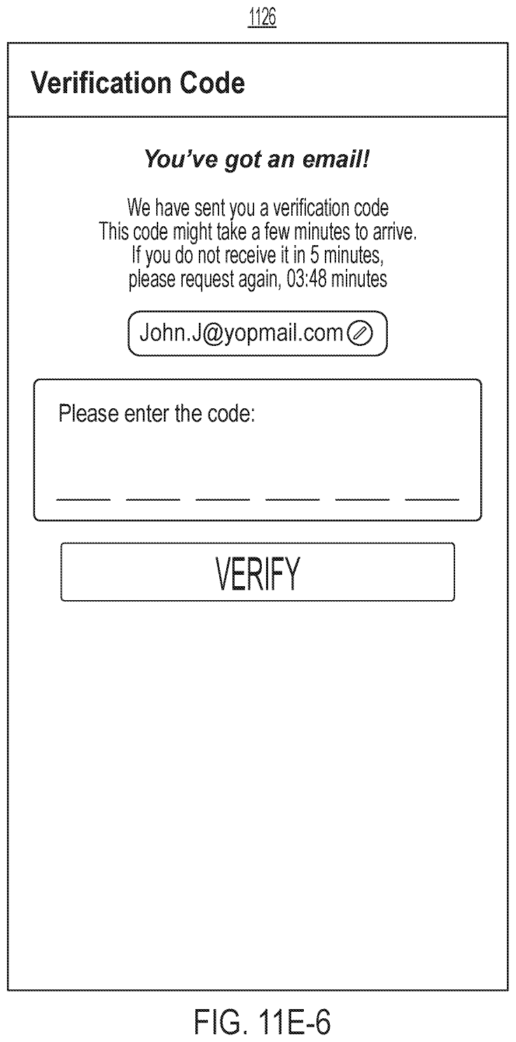

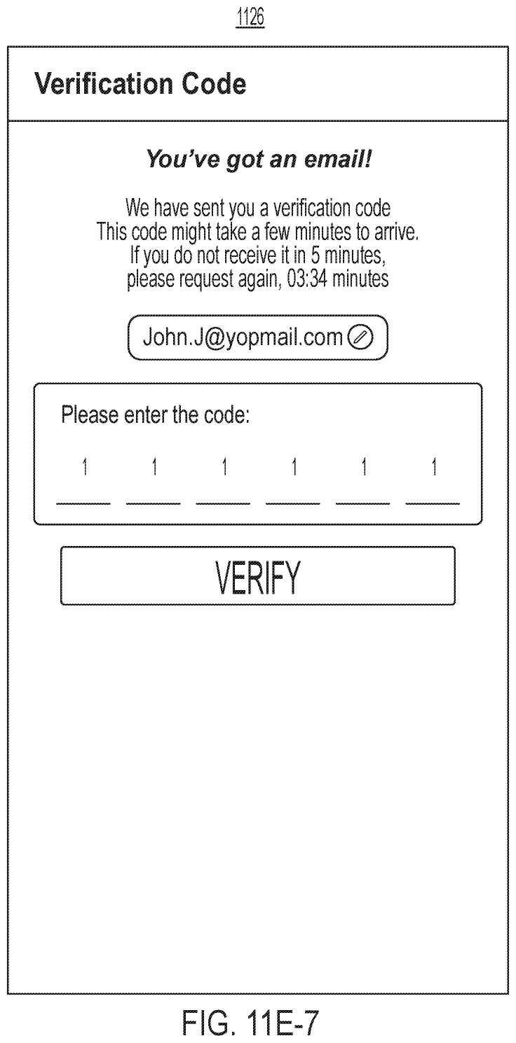

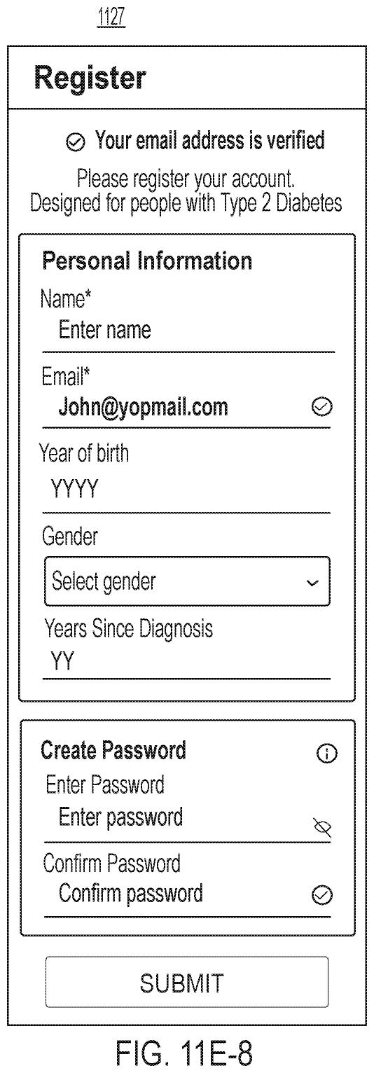

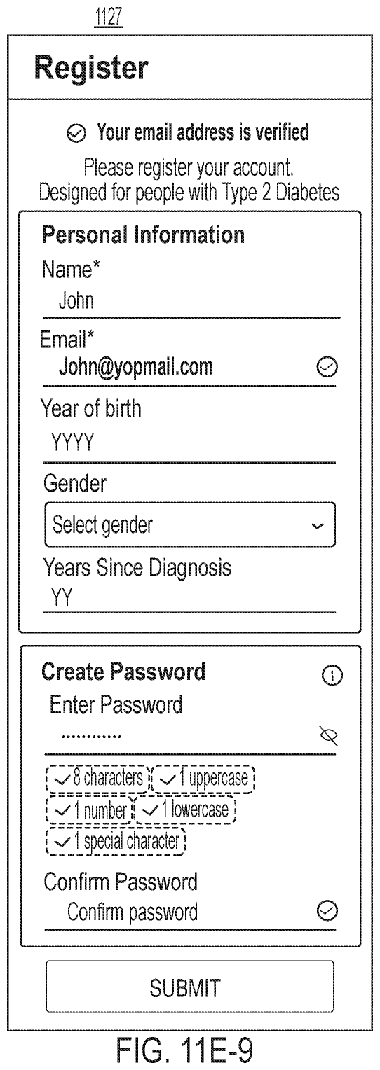

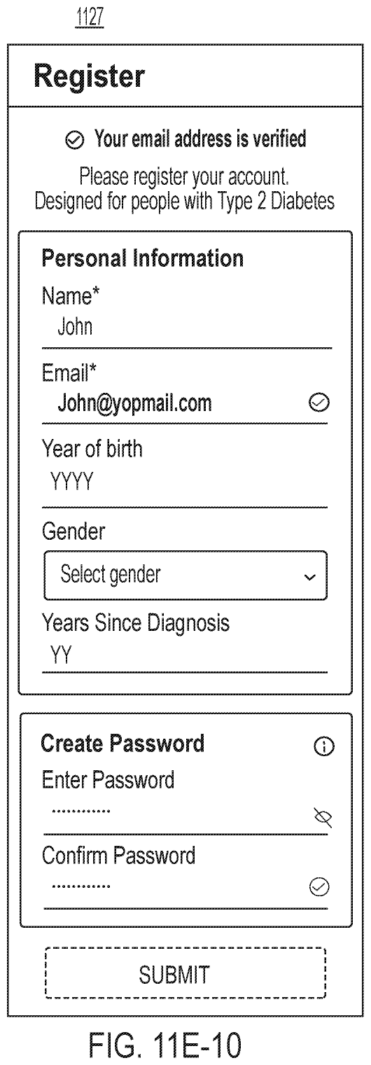

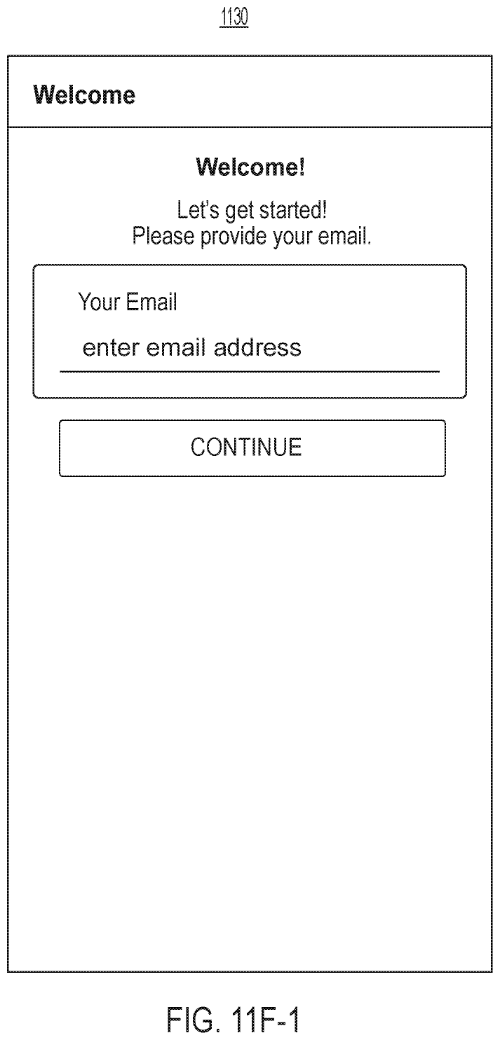

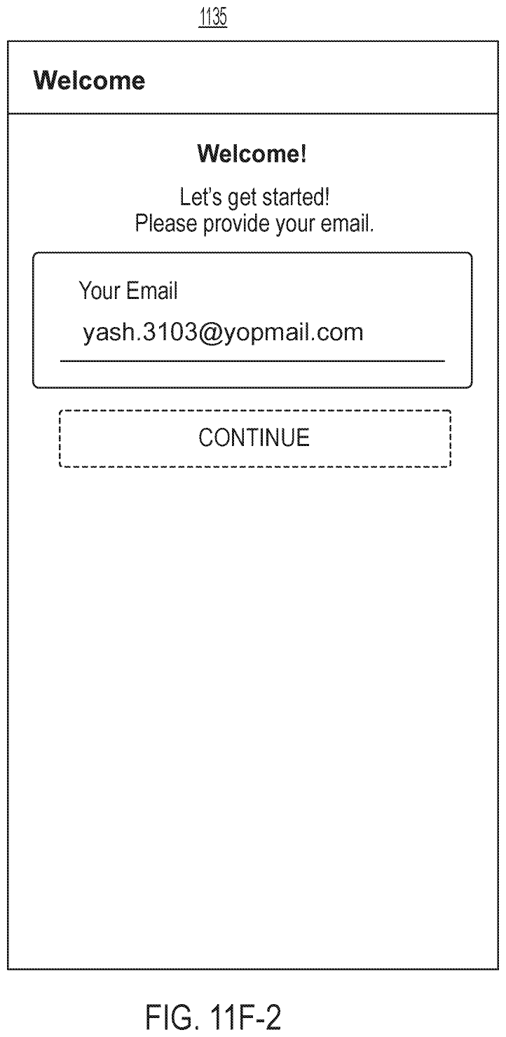

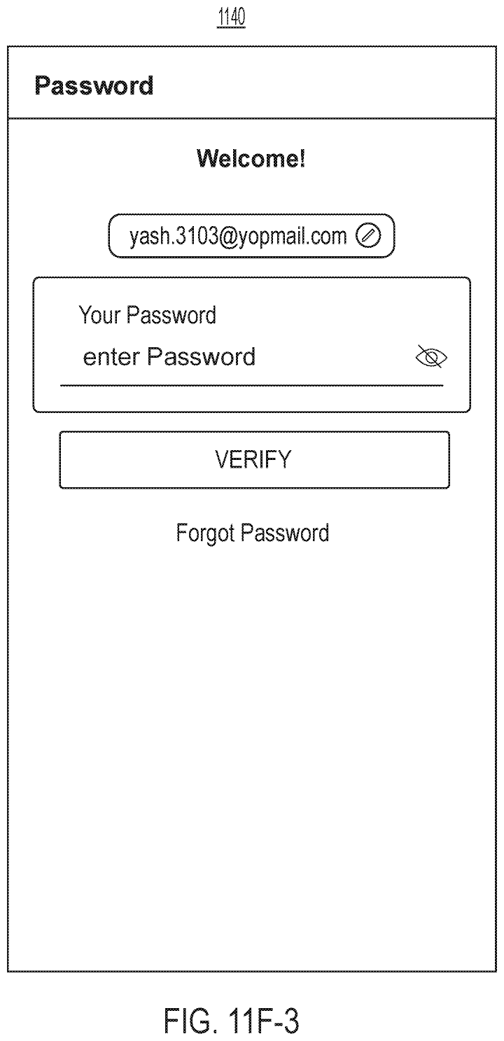

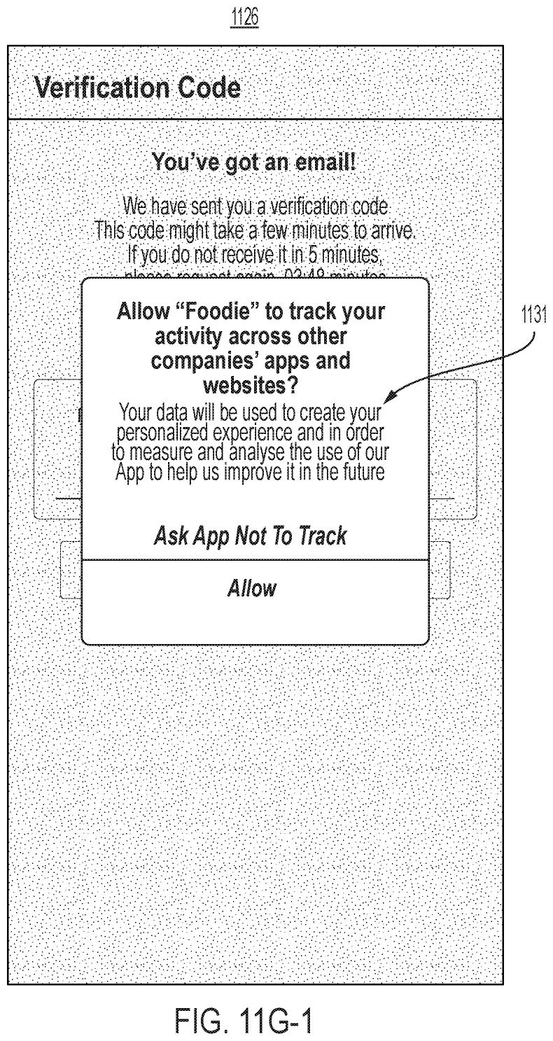

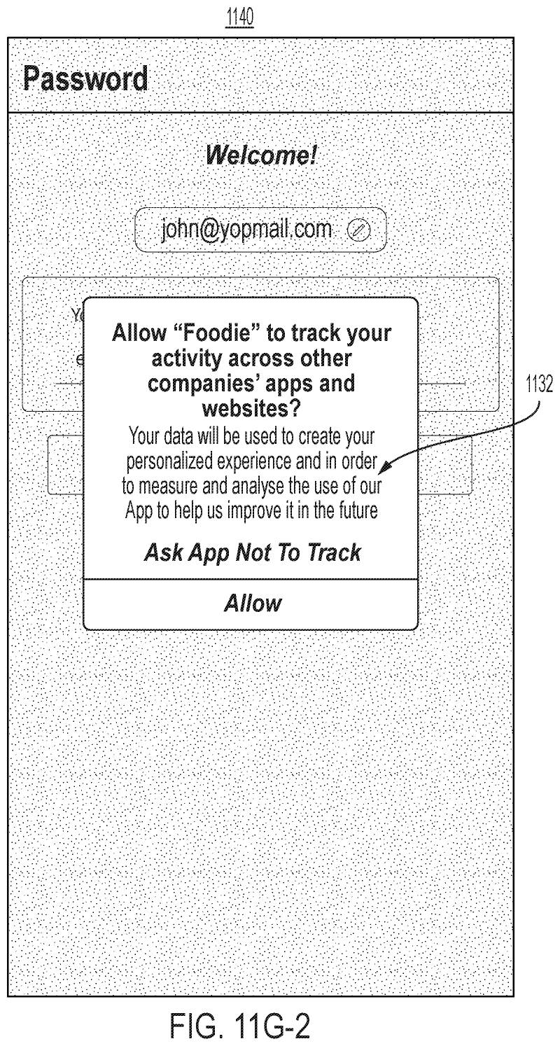

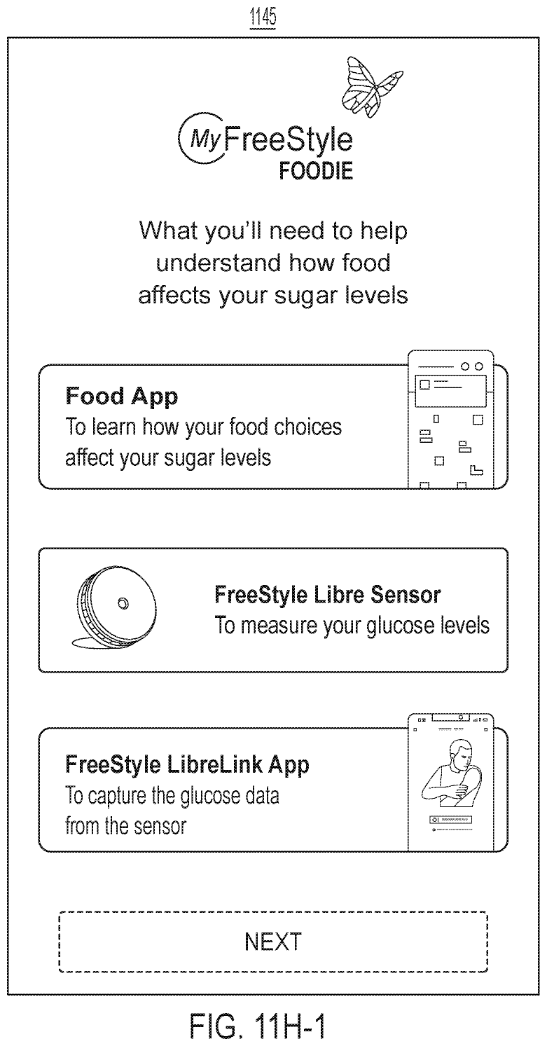

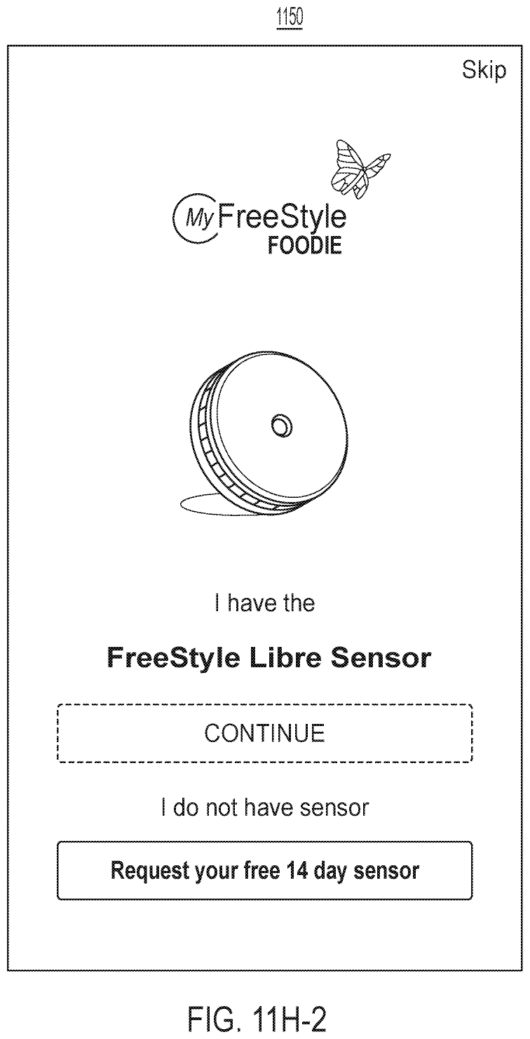

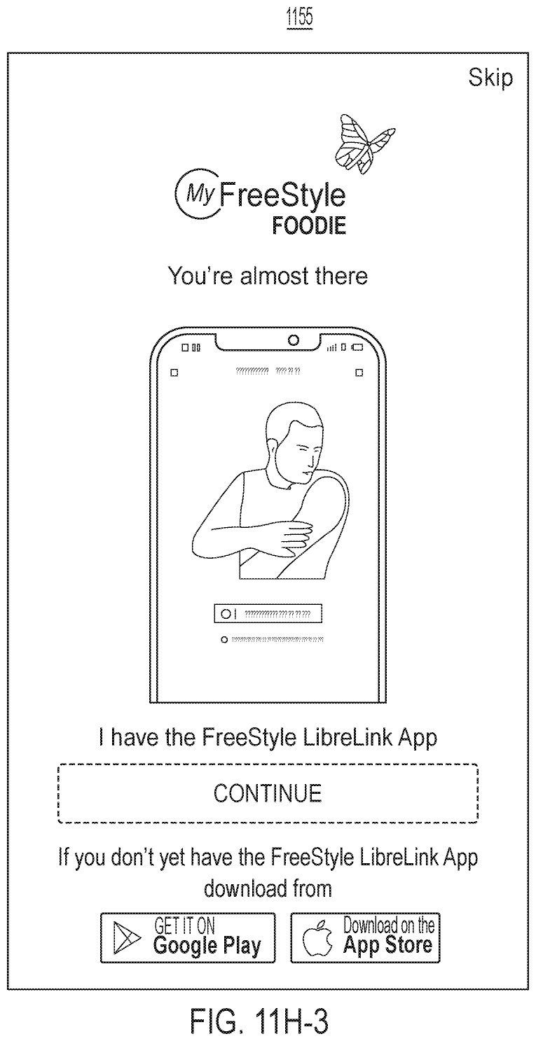

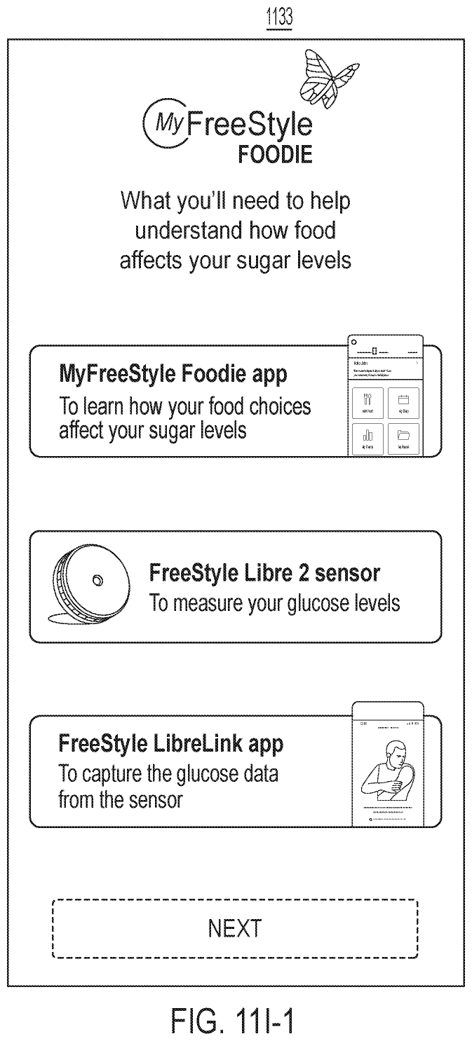

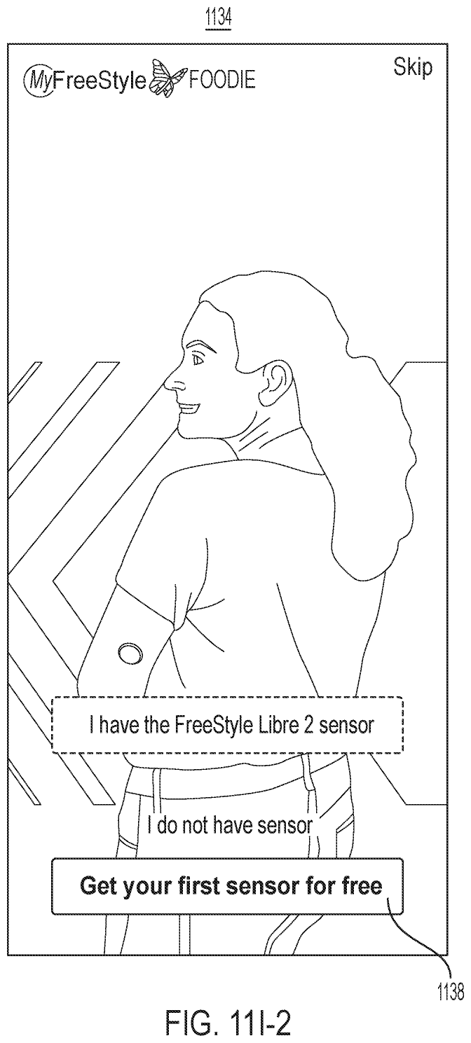

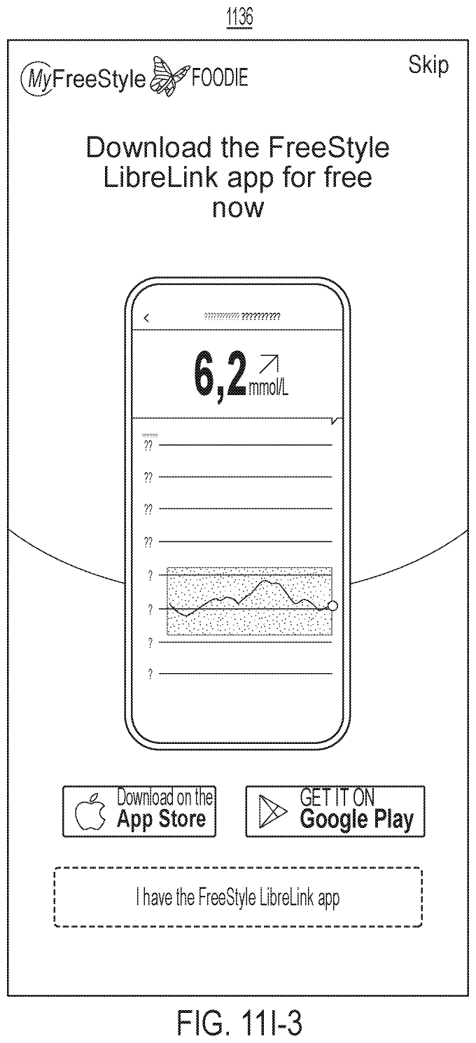

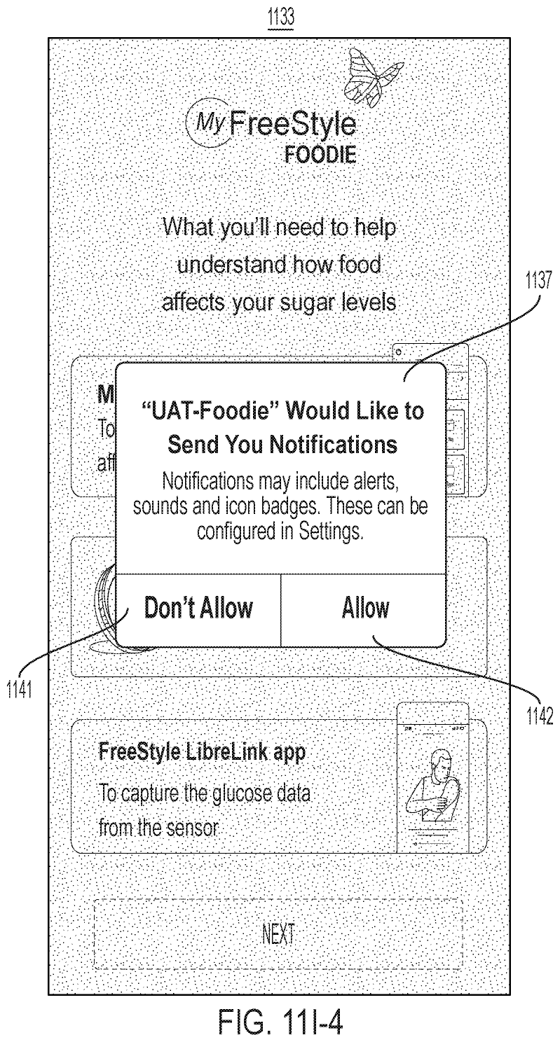

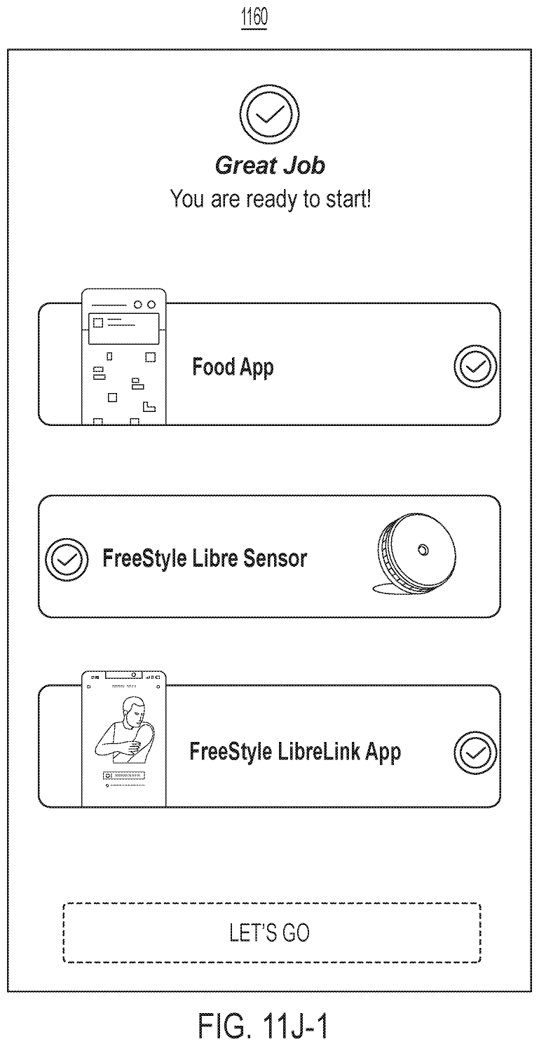

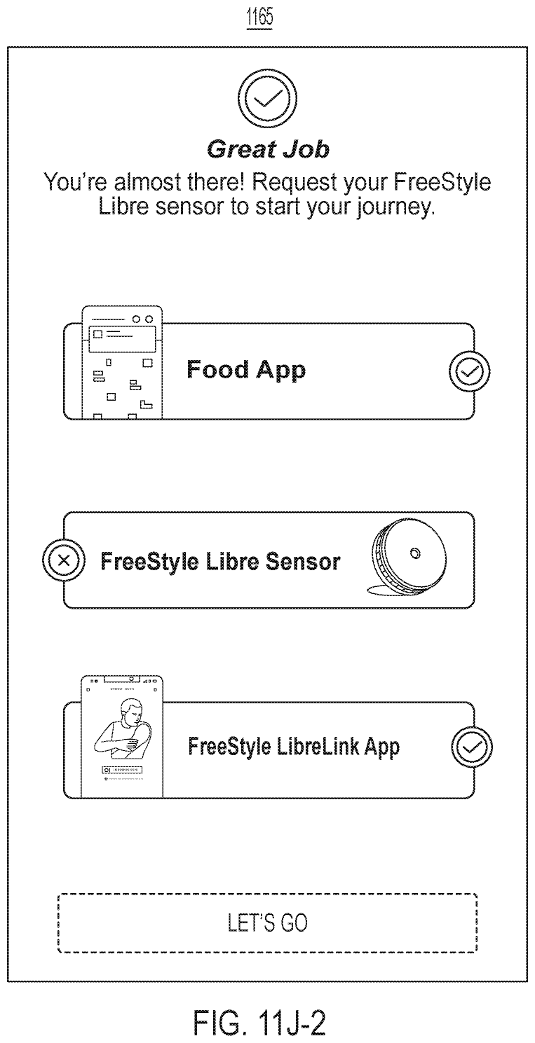

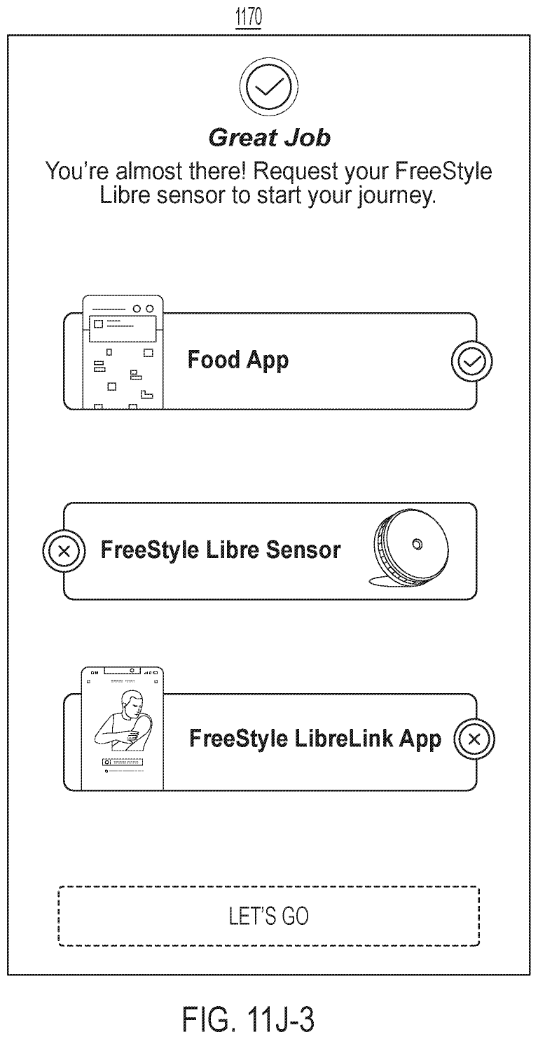

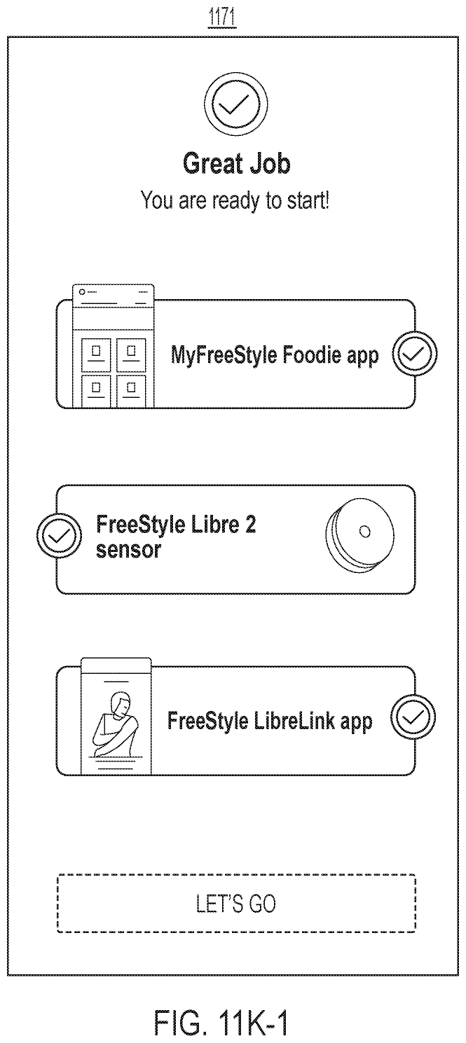

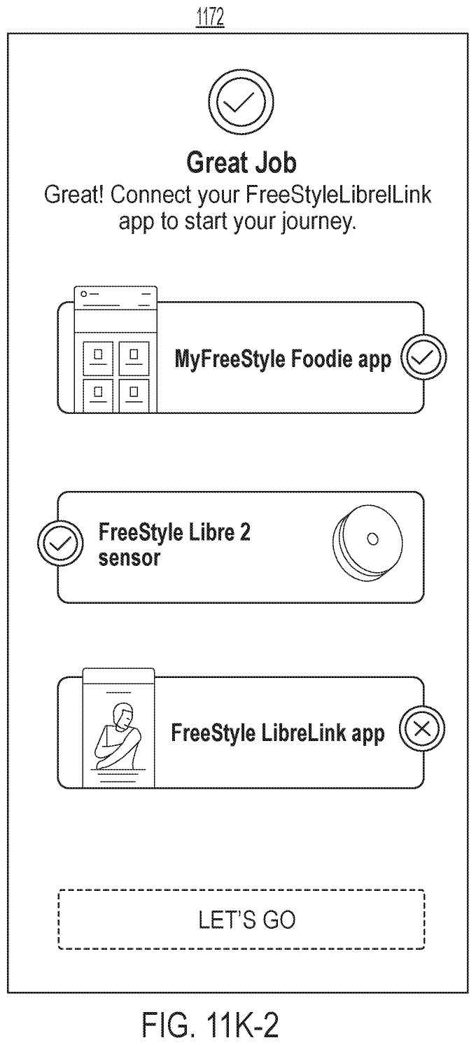

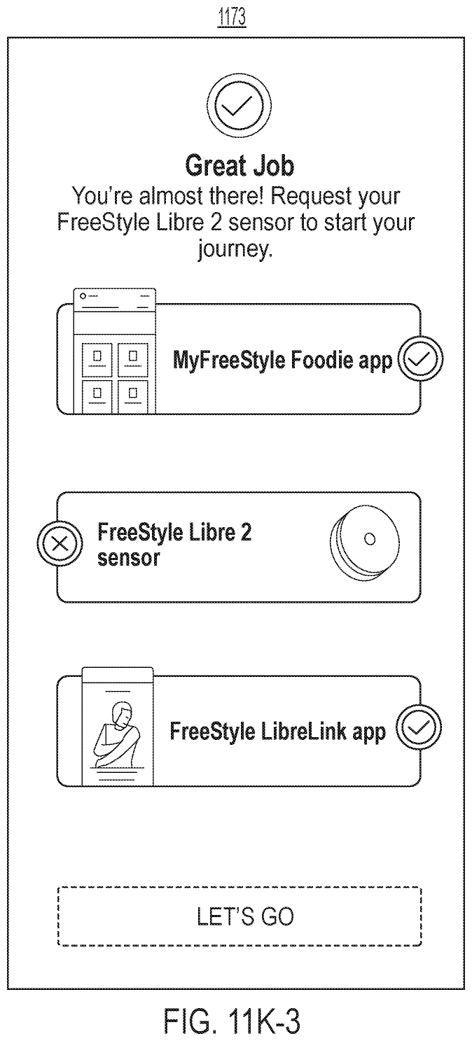

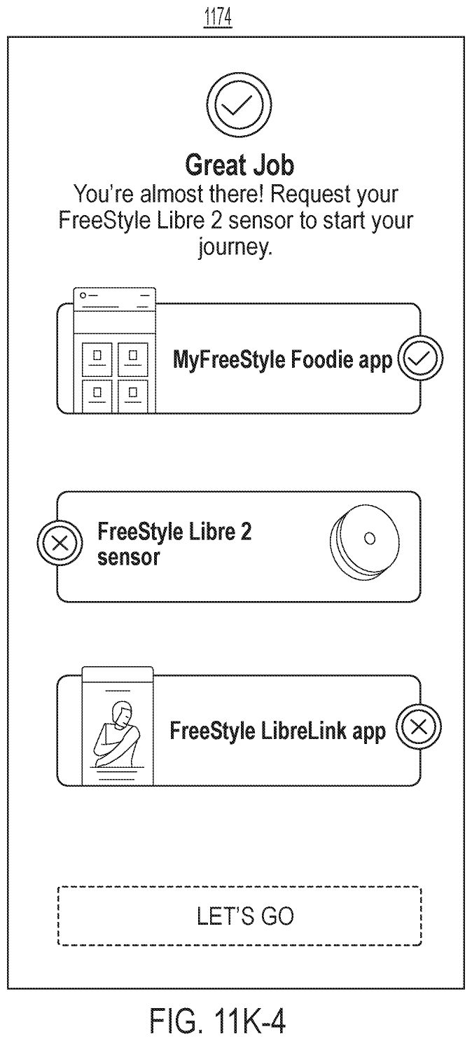

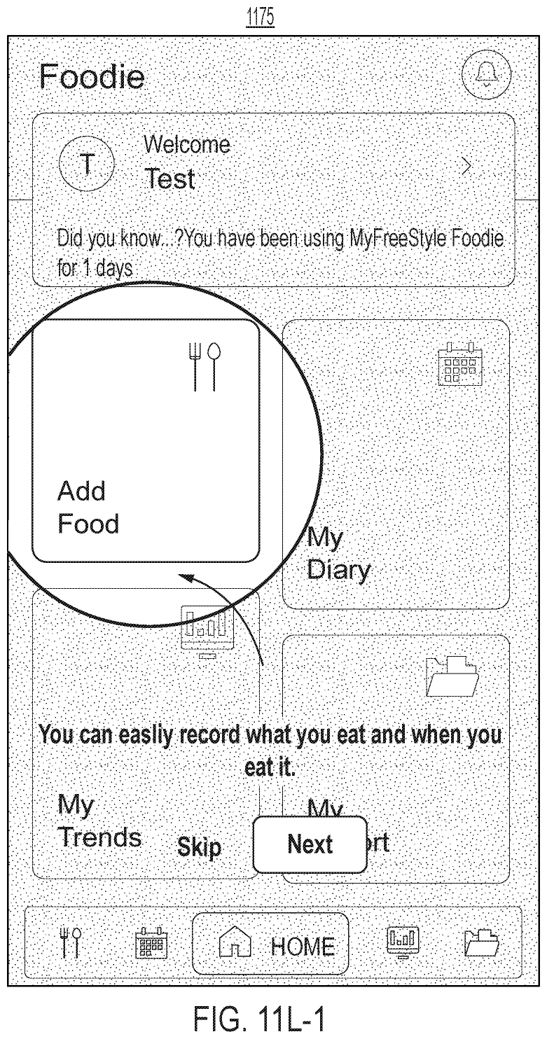

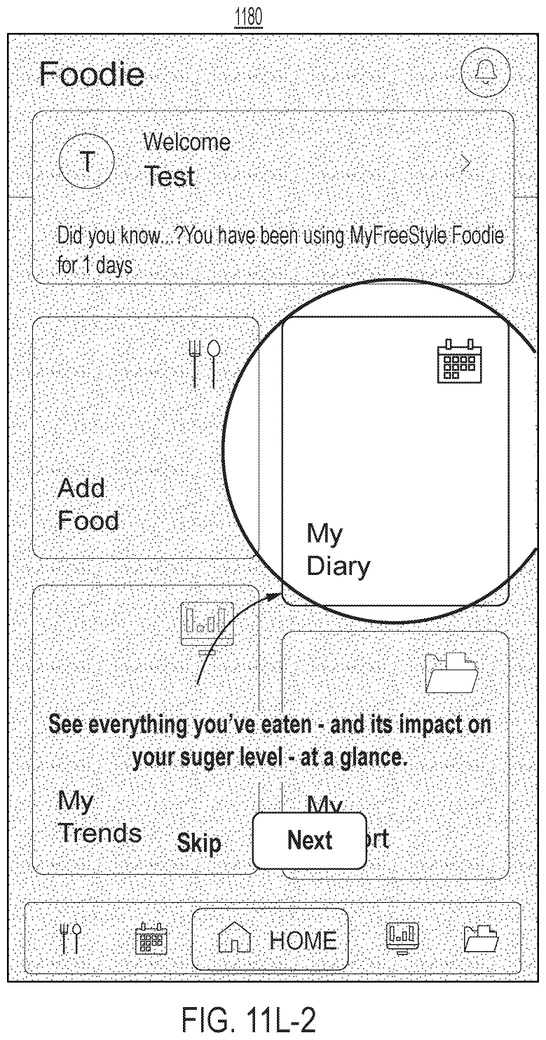

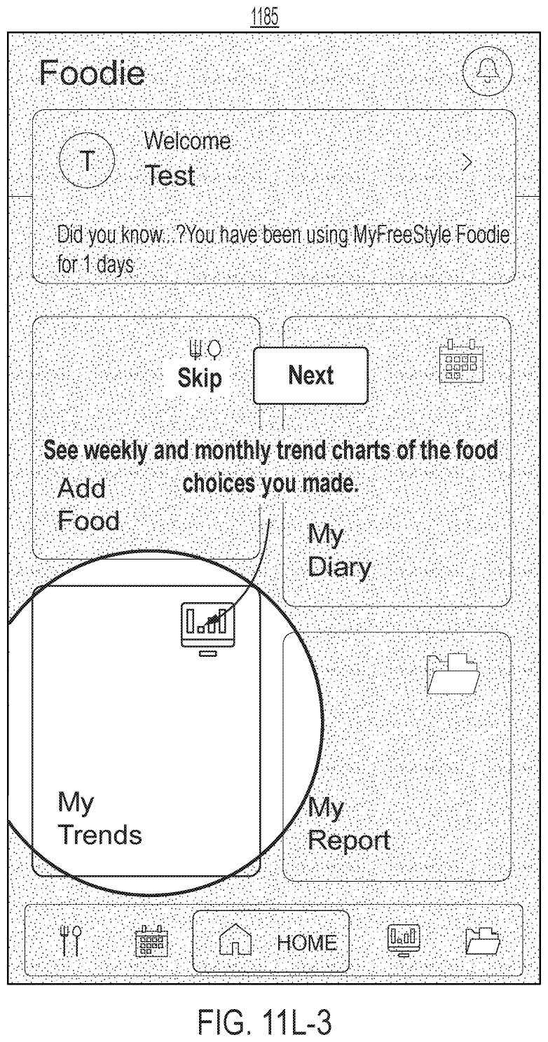

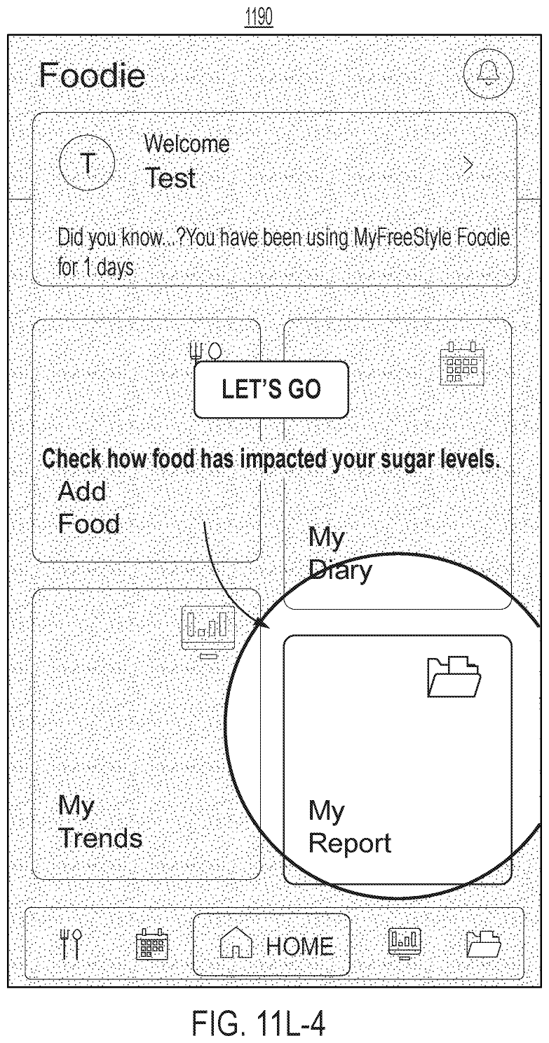

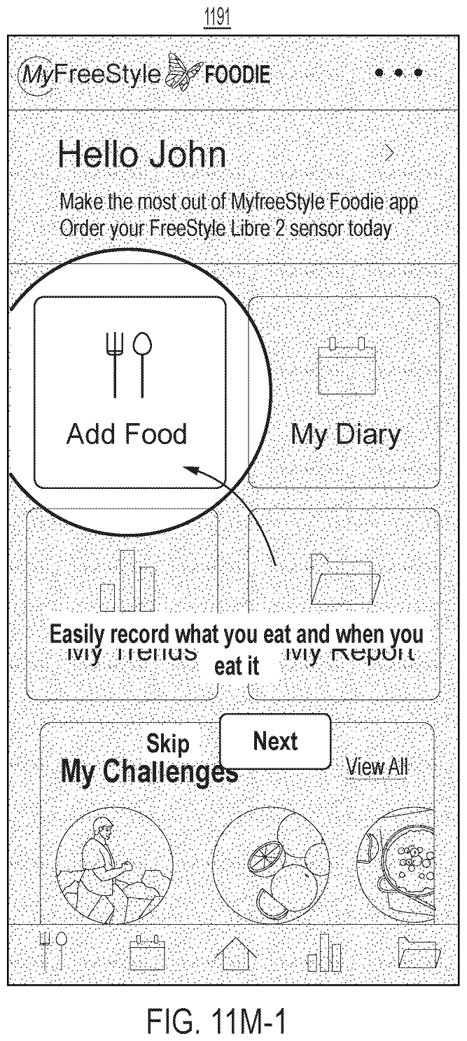

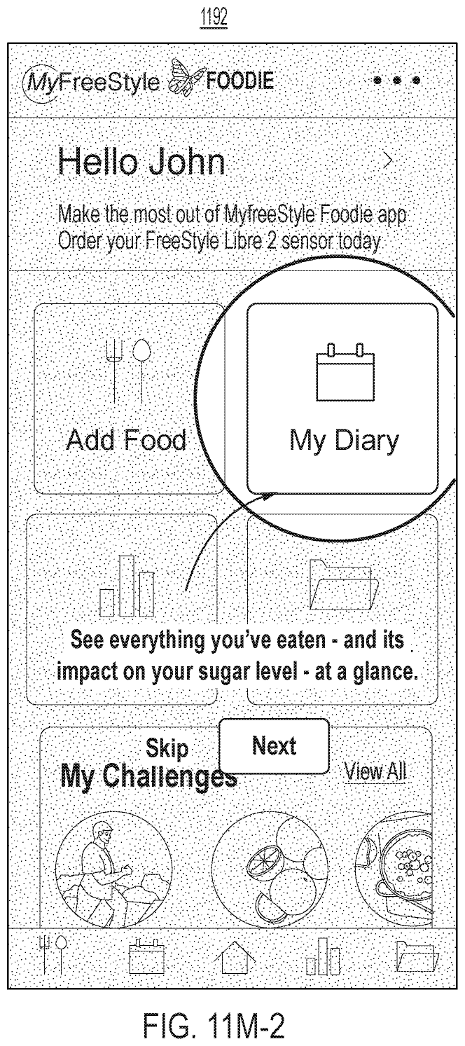

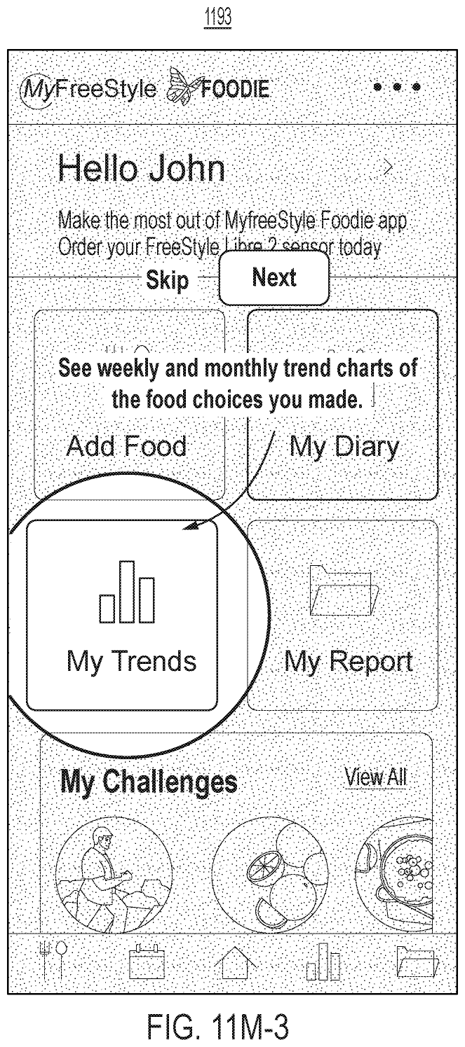

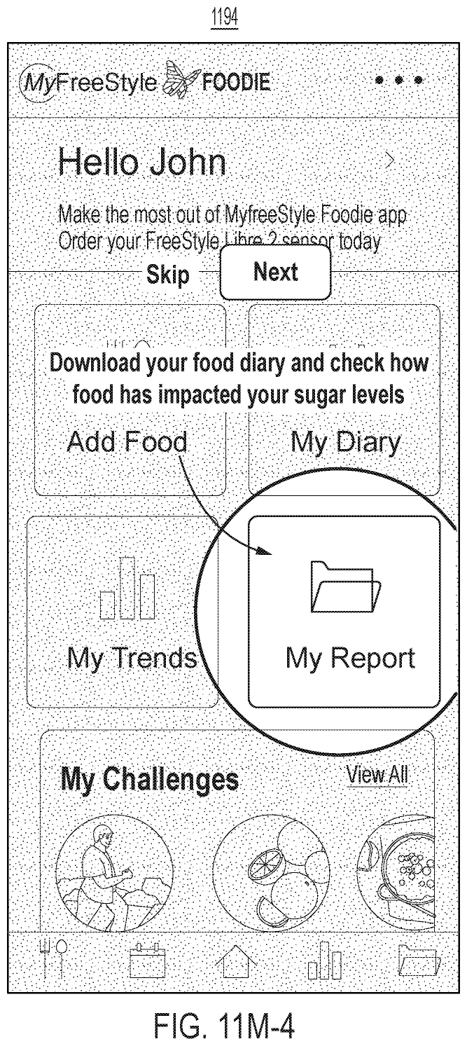

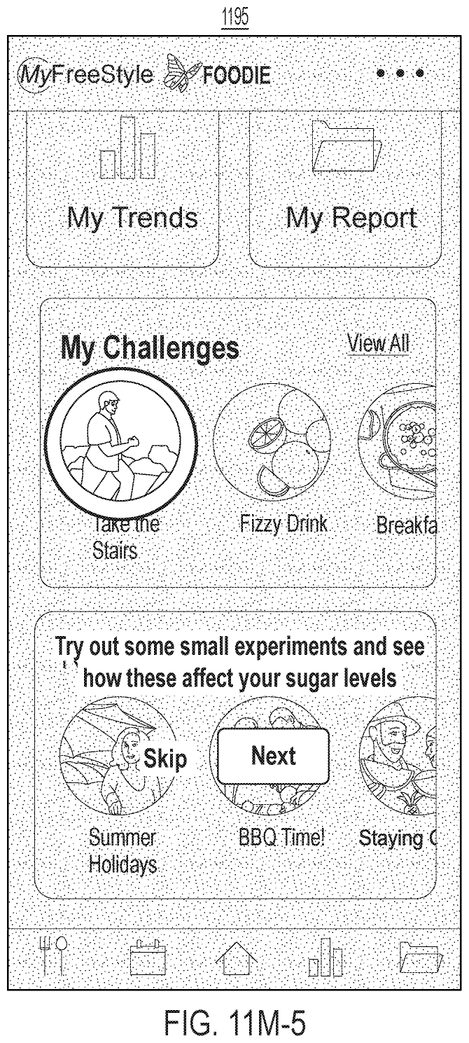

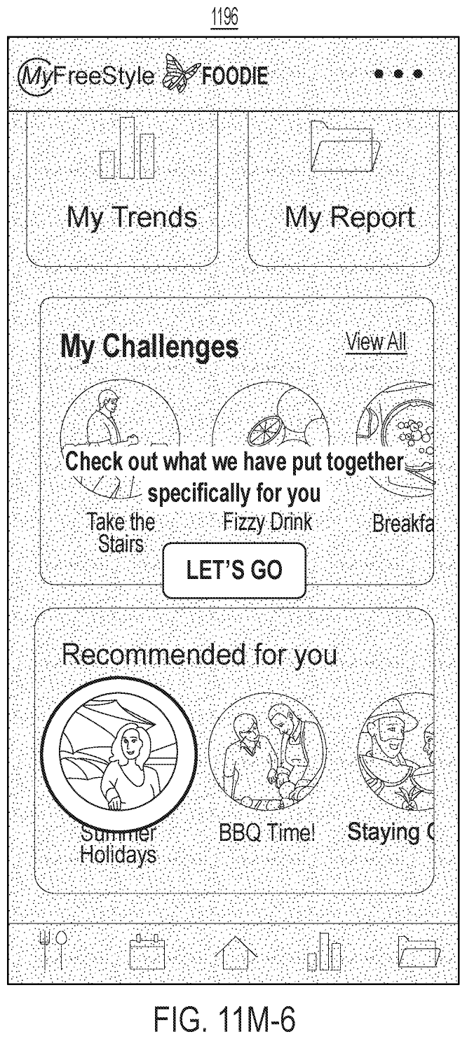

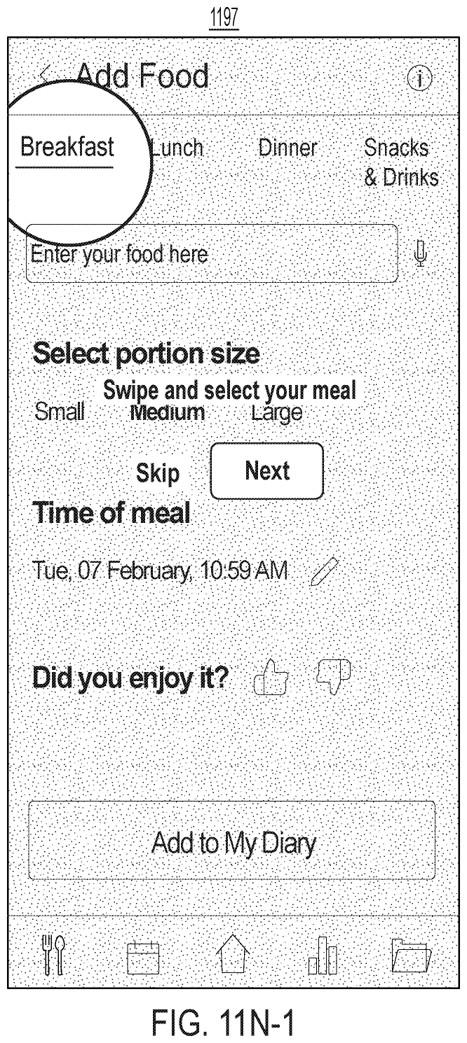

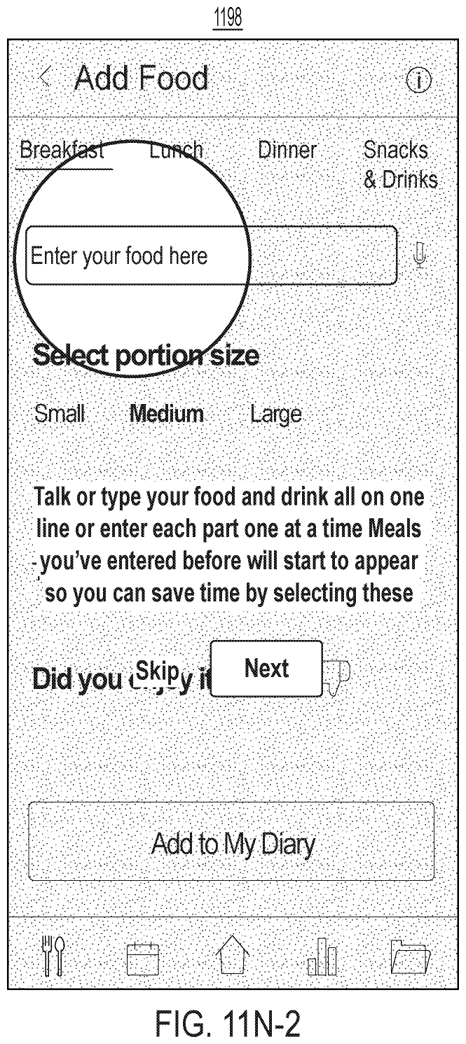

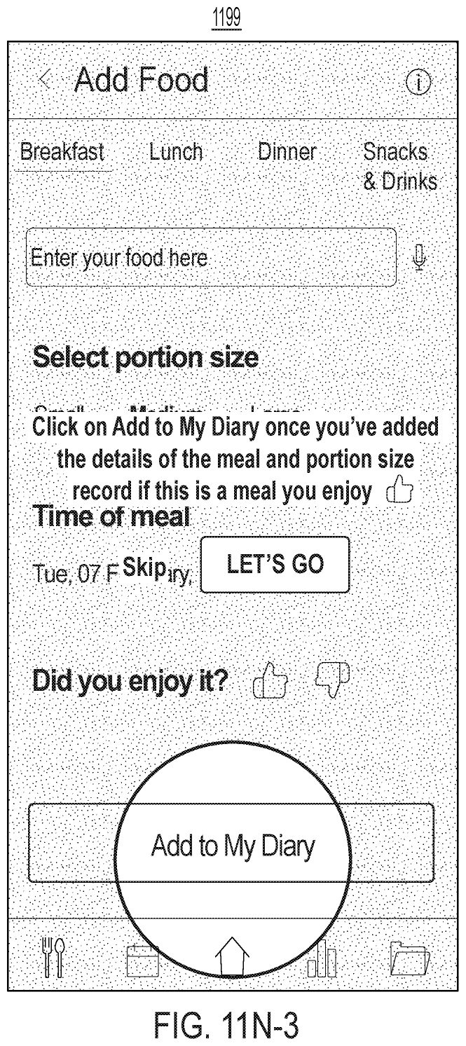

Further, FIGS. 11C-1 and 11C-2 depict language selection screens 1116 and 1117 respectively, which can be utilized by the user to change the desired language of the meal monitor application. FIG. 11C-3 depicts onboarding GUI 1118, which is similar to onboarding GUI 1101 as shown in 111B-1, except that it is configured to display content in Swedish.

Next, onboarding GUIs 1115, 1120, 1125, 1130 (FIGS. 11D-1 to 11D-4) can be utilized to register the user and create a new account. In other embodiments, onboarding GUIs 1121, 1122, 1123, 1124, 1126, 1127 (FIGS. 11E-1 to 11E-10) can be utilized to register the user and create a new account.

If an account has already been created, then GUIs 1130, 1135, 1140 (FIGS. 11F-1 to 11F-3) can be utilized to login the user. In some embodiments, and a shown in FIGS. 11G-1 and 11G-2, a pop-up modal 1131 or 1132 will be displayed on onboarding GUI 1126 or onboarding GUI 1140 to request permission for the meal monitor application to track the user's activities across other applications and websites.

Next, onboarding GUI 1145 (FIG. 11H-1) can be provided to explain the requirements of the meal monitor application to the user. Onboarding GUI 1150 (FIG. 11H-2-), for example, asks the user if they have a sensor control device. In some cases, GUI 1150 can also include a link or button to order a sensor if the user does not already have one. Onboarding GUI 1155 (FIG. 11H-3), asks the user if they have already installed the sensor app.

In other embodiments, onboarding GUI 1133 (FIG. 11I-1) can be provided to explain the requirements of the meal monitor application to the user. Onboarding GUI 1134 (FIG. 11I-2), for example, asks the user if they have a sensor control device. In some cases, onboarding GUI 1134 can also include a link or button 1138 to order a sensor if the user does not already have one. Onboarding GUI 1136 (FIG. 11I-3), asks the user if they have already installed the sensor app. In some embodiments, and as shown in FIG. 11I-4, a pop-up modal 1137 can be displayed on onboarding GUI 1133. Specifically, the pop-up modal 1137 can provide information regarding notifications. For example, the pop-up modal 1137 can inform the user that the meal monitoring application would like to send the user notification. Further, according to an aspect of the embodiments, the pop-up modal 1137 can include a “Don't Allow” button 1141 and an “Allow” button 1142 which the user can select in order to configure notification settings. This example meant to be illustrative only, and those of skill in the art will recognize that other combinations and permutations of modals can be implemented and are fully within the scope of the present disclosure.

According to another aspect of the embodiments, onboarding GUIs 1160, 1165, and 1170 (FIGS. 11J-1 to 11J-3) can be displayed to inform the user if all requirements are met (e.g., onboarding GUI 1171), or if one or more requirements are not met (e.g., onboarding GUIs 1165 and 1170). In some embodiments, onboarding GUIs 1171, 1172, 1173, 1174 (FIGS. 11K-1 to 11K-4) can be displayed to inform the user if all requirements are met (e.g., onboarding GUI 1171), or if one or more requirements are not met (e.g., onboarding GUIs 1172, 1173, and 1174).

In addition to the aforementioned onboarding GUIs, according to another aspect of the embodiments, tutorial GUIs 1175, 1180, 1185, and 1190 (FIGS. 11L-1 to 11L-4) can be optionally displayed to the user after the onboarding process is complete. In some embodiments, after the onboarding process is complete, tutorial GUIs 1191, 1192, 1193, 1194, 1195, and 1196 (FIGS. 11M-1 to 11M-6) can be optionally displayed to the user on the meal monitor application. Additionally, in some embodiments, food tutorial GUI 1197, 1198, 1199 (FIGS. 11N1- to 11N-3) can be optionally displayed to the user.

### Example Embodiments of Challenge GUIs and Features Related Thereto

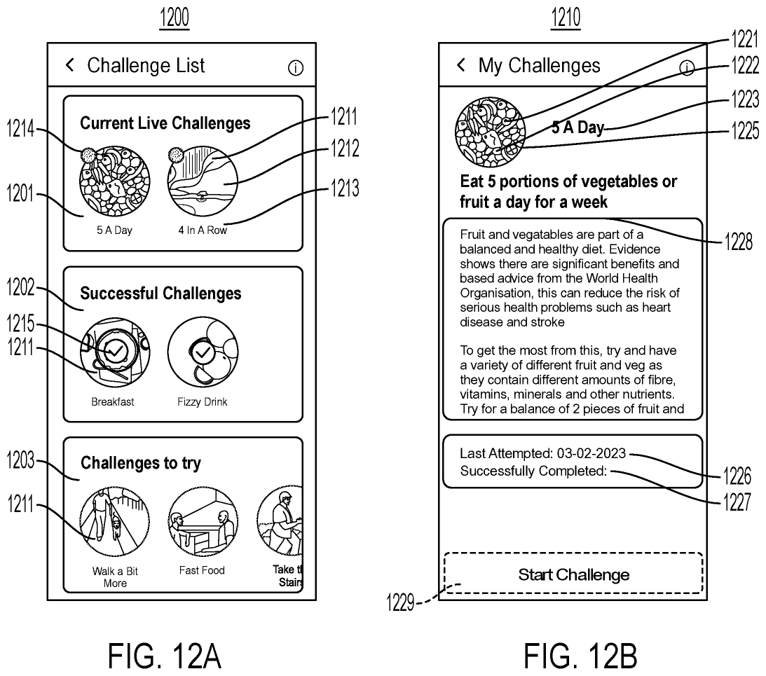

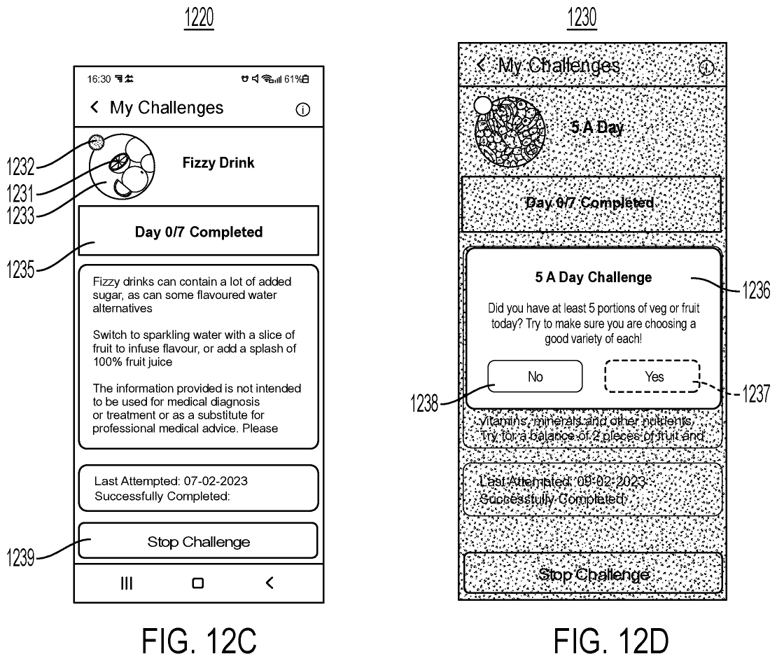

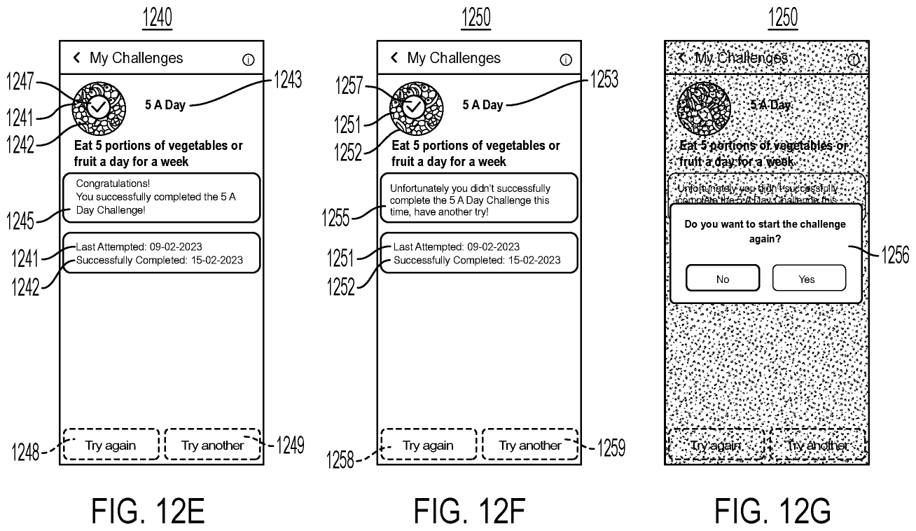

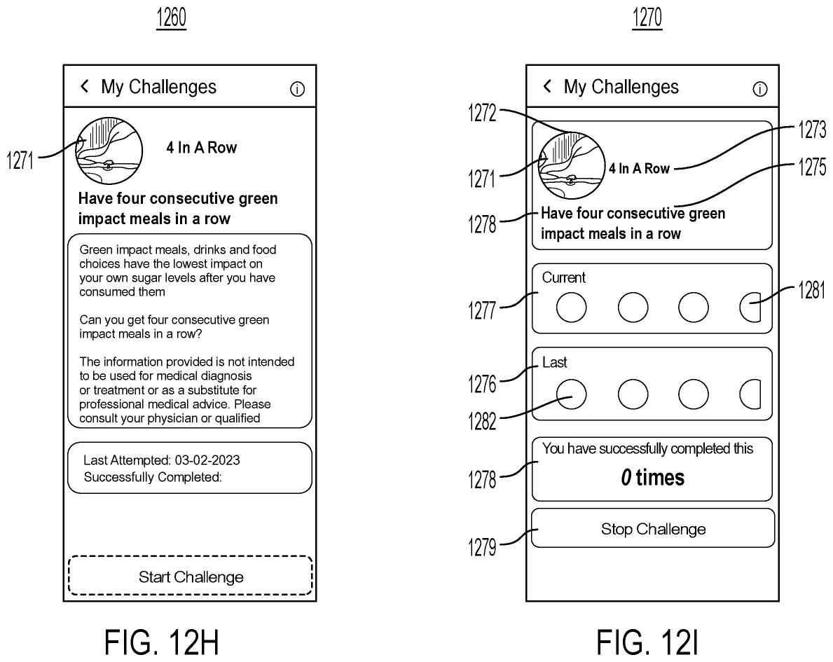

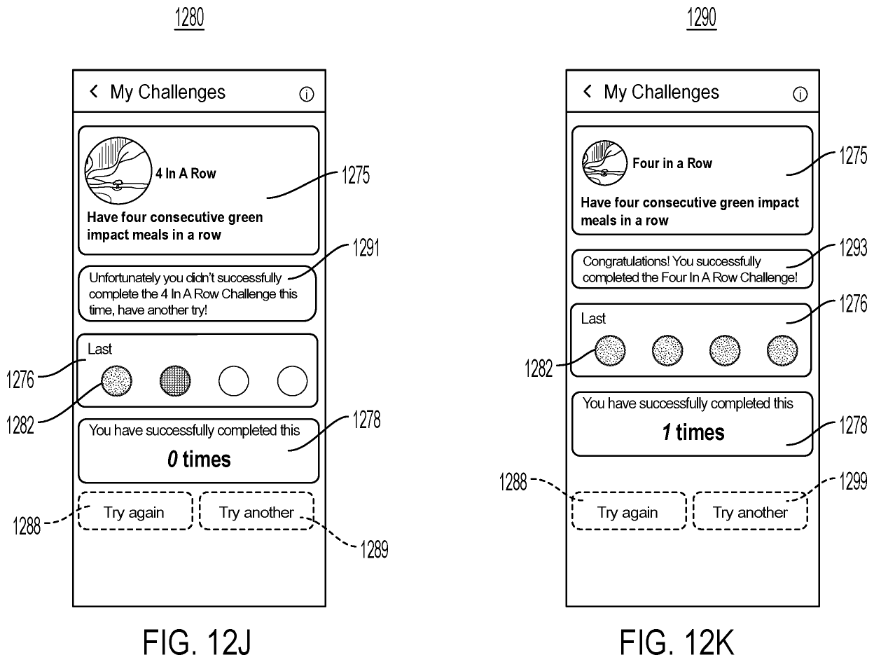

Referring next to FIGS. 12A-12K, example embodiments of challenge GUIs for a meal monitoring application are depicted. With reference to FIG. 12A, a challenge list GUI 1200 (also herein referred to as a “first challenge GUI”) is displayed. Challenge list GUI 1200 can be displayed when the user selects the “View All” link 4304 on the challenges card of home GUI 460 (as shown in FIGS. 4A-3 and 4A-4). According to some embodiments, and as depicted in FIG. 12A, the challenge list GUI 120 reflects a list of one or more challenges relating to the user's analyte response, and comprises: a live challenges card 1201 (also herein referred to as “a first challenge card”); a completed challenges card 1202 (also herein referred to as “a second challenge card”); and an unattempted challenges card 1203 (also herein referred to as “a third challenge card”).

Specifically, the live challenges card 1201 can comprise one or more challenge icons 1211 reflecting challenges which are active or currently in progress by the user. In this regard, the live challenges card 1201 lists the challenges which are live or the user is currently participating in on the meal monitoring application. The challenge icons 1211 can comprise a picture 1212 and a challenge title 1213 providing a textual description relating to the subject matter of the corresponding challenge reflected by the challenge icon 1211. In some embodiments, and as shown in FIG. 12A, a live indicator 1214 (e.g., a green dot) can be displayed on the picture 1212 of a challenge icon 1211 to indicate that the challenge represented by the challenge icon 1211 is active or currently in progress by the user. Those of skill in the art will recognize that various other live indicators 1214 can be utilized with the challenge GUIs described herein, and are fully within the scope of the present disclosure.

Further, the live challenges card 1201 can include a plurality of challenge icons 1211. In some embodiments, two or three challenge icons 1211 can be displayed on the live challenges card 1201. In some embodiments, the live challenges card 1201 can comprise a plurality of challenge icons 1211, wherein the number of challenge icons 1211 in the live challenges card 1201 exceeds the number which can be displayed on the challenge list GUI 1200 at any given time. In some embodiments, the live challenges card 1201 is responsive to a received input by the user (e.g., by a swipe gesture, a scroll gesture, a drag gesture, or some other predetermined gesture) so as to display a new set of challenge icons 1211 not previously displayed on the challenge list GUI 1200 in response to said input. For example, in response to received input, the live challenges card 1201 can display a new set of challenge icons 1211. In some embodiments, at least one of the challenge icons 1211 in the new set displayed on the live challenges card 1201 is different than at least one of the challenge icons 1211 in the original set, prior to the received input by the user.

According to another aspect of the embodiments, the challenge list GUI 1200 further comprises the completed challenges card 1202 which includes one or more challenge icons 1211 reflecting challenges which have been completed by the user or in which the user has previously participated in. In this regard, the completed challenges card 1202 lists the completed challenges in the meal monitoring application. The challenge icons 1211 can comprise a picture 1212 and a challenge title 1213 providing a textual description relating to the subject matter of the corresponding challenge reflected by the particular challenge icon 1211. In some embodiments, a participation indicator 1215 (e.g., a colored check mark, such as the green check mark shown in FIG. 12A) is overlayed on the picture displayed on a challenge icon 1211 so as to indicate that the challenge represented by the challenge icon 1211 is one in which the user has previously participated in or completed. Those of skill in the art will recognize that various other participation indicators can be utilized with the challenge GUIs described herein, and are fully within the scope of the present disclosure.

In some embodiments, two or three challenge icons 1211 can be displayed on the completed challenges card 1202. In some embodiments, the completed challenges card 1202 can comprise a plurality of challenge icons 1211, wherein the number of challenge icons 1211 in the completed challenges card 1202 exceeds the number which can be displayed on the challenge list GUI 1200 at any given time. In some embodiments, the completed challenges card 1202 is responsive to a received input by the user (e.g., by a swipe gesture, a scroll gesture, a drag gesture, or some other predetermined gesture) so as to display a new set of challenge icons 1211 not previously presented on the challenge list GUI 1200 in response to said input. In exemplar embodiments, in response to received input, the completed challenges card 1202 can display a new set of challenge icons 1211. In some embodiments, at least one of the challenge icons 1211 in the new set on the completed challenges card 1202 is different than at least one of the challenge icons 1211 in the original set, prior to the received input by the user.

Further, in some embodiments, the challenge list GUI 1200 comprises the unattempted challenges card 1203 which includes one or more challenge icons 1211 reflecting challenges which have not yet been tried by the user or in which the user has not yet participated in. The challenge icons 1211 can comprise a picture 1212 and a challenge title 1213 providing a textual description relating to the subject matter of the corresponding challenge reflected by the particular challenge icon 1211.

In some embodiments, two or three challenge icons 1211 can be displayed on the unattempted challenges card 1203. In some embodiments, the unattempted challenges card 1203 can comprise a plurality of challenge icons 1211, wherein the number of challenge icons 1211 in the unattempted challenges card 1203 exceeds the number which can be displayed on the challenge list GUI 1200 at any given time. In some embodiments, the unattempted challenges card 1203 is responsive to a received input by the user (e.g., by a swipe gesture, a scroll gesture, a drag gesture, or some other predetermined gesture) so as to display or populate additional or alternative challenge icons 1211 not previously displayed on the challenge list GUI 1200 in response to said input. In response to received input, the unattempted challenges card 1203 can display a new set of challenge icons 1211. In some embodiments, at least one of the challenge icons 1211 in the new set is different than at least one of the challenge icons 1211 in the original set, prior to the received input by the user.

FIG. 12B depicts a challenge information GUI 1210 (also herein referred to as “a second challenge GUI”) for an unattempted challenge (e.g., the “5 A Day” challenge). Specifically, the challenge information GUI 1210 for an unattempted challenge is displayed when the user selects the corresponding challenge icon 1211 from the unattempted challenges card 1203 on challenge list GUI 1200. In some embodiments, the challenge information GUI 1210 comprises a challenge profile section 1225, wherein the challenge profile section 1225 comprises a challenge icon 1221 having a picture 1222 and a challenge title 1223 providing a textual description of the unattempted challenge. In some embodiments the challenge information GUI 1210 also provides contextual information 1228 related to its respective challenge. For example, in FIG. 12B, the unattempted challenge represented by challenge information GUI 1210 relates to eating vegetables and fruits and, as such, the contextual information 1228 provides a contextual description of the importance of consuming vegetables and fruits along with a description of the challenge.

Further, the challenge information GUI 1210 can include an attempt indicator 1226, wherein the attempt indicator 1226 can be adjacent to a datestamp indicating when the challenge was last attempted. In some embodiments, if the challenge was not previously attempted, no datestamp will appear adjacent to the attempt indicator 1226. The challenge information GUI 1210 can also include a completion indicator 1227, wherein the completion indicator 1227 can be adjacent to a datestamp indicating when the challenge was last successfully completed. In some embodiments, if the challenge has not previously been completed, no datestamp will appear adjacent to the completion indicator 1227 (see, e.g., FIG. 12B).

In some embodiments, the challenge information GUI 1210 comprises a start button 1229 which, when selected by the user, can begin the challenge. Though not illustrated, once a challenge is initiated, the challenge icon 1211 will no longer be displayed in the unattempted challenges card 1202 on challenge list GUI 1200. Rather, the challenge icon 1211 will be displayed in the live challenges card 1214 on challenge list GUI 1200 to indicate the challenge is active or live. In some embodiments, a challenge will begin immediately following the user selecting the start button 1229 (FIG. 12B). In some embodiments, the challenge will not commence until the following day. For example, if the challenge the user wishes to start relates to breakfast and the challenge is started in the afternoon, then the meal monitoring application is configured to begin the selected challenge the subsequent day. According to an aspect of the embodiments, the user can contemporaneously attempt a plurality of challenges. In some embodiments, the user can start two challenges at a same time.

FIG. 12C depicts a challenge information GUI 1220 for an active challenge (e.g., the “Fizzy Drink” challenge that encourages users to opt for low-sugar beverages instead of high-sugar sodas or similar drinks). In some examples, the Fizzy Drink challenge may be outputted/recommended to users that drank one or more high-sugar sodas or similar drinks or drank a threshold number of such drinks within a predetermined period of time. The challenge information GUI 1220 for an active challenge is similar to challenge information GUI 1210 for an unattemped challenge, except that it comprises a stop button 1339 instead of a start button. In this regard, the user can select the stop button 1339 to cease continuation of the respective challenge. Further, the challenge icon 1231 depicted on challenge information GUI 1220 for an active challenge includes a live indicator 1232 (e.g., a green dot) on a picture 1233 thereon so as to indicate the challenge's live status. In some embodiments, the challenge information GUI 1220 for an active challenge further comprises a progress card 1235, wherein the progress card 1235 indicates the progress the user has made towards the challenge. According to an aspect of the embodiments, the progress card 1235 comprises a unit of measure and a unit of time to indicate progress. For example, if the challenge requires seven days to complete, then the progress card can indicate the number of days the user has completed. Further, the progress card can utilize fractional units to indicate progress (e.g., “0/7” to indicate that zero of seven days have been completed by the user). However, those of skill in the art will recognize that other units of measure and/or units of time can be utilized, including but not limited to, hours, weeks, and percentages, and are fully within the scope of the present disclosure.

FIG. 12D depicts another example embodiment of a challenge information GUI for an active challenge. According to an aspect of the embodiments, a modal 1236 can be displayed on a challenge information GUI 1230 for an active challenge, wherein the modal is configured to prompt the user to provide progress information related to the in progress active challenge. For example, and as shown in FIG. 12D, if the active challenge requires the user to consume five portions of vegetables or fruits in a day, the modal 1236 can ask the user if this challenge has been completed. The modal 1236 can comprise a “Yes” button 1237 and a “No” button 1238 which the user can select to indicate their answer to the prompt provided by the modal 1236. In some embodiments, the user is prompted on a daily basis to respond to a modal 1236 relating to a particular challenge so as to report and track progress thereof.

FIG. 12E depicts a challenge GUI 1240 (also herein referred to as “a third challenge GUI”) that is displayed to the user upon the user successfully completing a challenge or indicating as such. For example, challenge GUI 1240 shown in FIG. 12E can be displayed in response to the user selecting the “Yes” button 1237 on modal 1236. According to an aspect of the embodiments, the challenge GUI 1240 can comprise: an attempt indicator 1246; a completion indicator 1247; a challenge icon 1241 comprising a picture 1242 and challenge title 1243 representing the completed challenge; and, a congratulatory message 1245 notifying the user that the challenge has been successfully completed. Further, a participation indicator 1247 (e.g., a green checkmark) can be overlayed on the picture 1242 of the challenge icon 1241 so as to indicate that the represented challenge has been completed by the user. Further in some embodiments, the challenge GUI 1240 can comprise a “Try Again” button 1248 and a “Try Another” button 1249. In some embodiments, when the user selects the Try Again button 1248, the user can restart the challenge. In some embodiments, when the user selects the Try Another button 1249, the challenge list GUI 1200 is displayed so as to allow the user to select a different challenge.

In some embodiments, if the user was unsuccessful in completing the challenge or indicated as such, then a challenge GUI 1250 (also herein referred to as “a fourth challenge GUI”) can be displayed (FIG. 12F). For example, challenge GUI 1250 depicted in FIG. 12 can be displayed in response to the user selecting the “No” button 1238 on modal 1236 (FIG. 12D). According to an aspect of the embodiments, the challenge GUI 1250 can comprise: an attempt indicator 1251; a completion indicator 1252; a challenge icon 1251 comprising a picture 1252 and a challenge title 1253 representing the completed challenge; and, a message 1255 notifying the user that the challenge was not successfully completed. In some embodiments, and as depicted in FIG. 12F, the message 1255 can include an encouraging note to the user (e.g., “have another try!”). Further, a participation indicator 1257 (e.g., a green checkmark) can be overlayed on the picture 1252 of the challenge icon 1251 so as to indicate that the represented challenge has been completed by the user. Further in some embodiments, the challenge GUI 1250 can comprise a “Try Again” button 1258 and a “Try Another” button 1259. In some embodiments, when the user selects the Try Again button 1258, a modal 1256 (FIG. 12G) will be displayed on the challenge GUI asking the user to confirm whether they would like to restart the challenge. In some embodiments, when the user selects the Try Another button 1258, the challenge list GUI 1200 (FIG. 12A) is displayed so as to allow the user to select a different challenge.

FIG. 12H-12K depict additional exemplar embodiments of challenge GUIs for a meal monitoring application. Specifically, FIG. 12H depicts a challenge information GUI 1260 for an unattempted challenge (e.g., the “4 In A Row” challenge), which is similar to challenge information GUI 1210 depicted in FIG. 12B, except that the challenge icon 1271 in FIG. 12A does not include an indicator. FIG. 12I illustrates a challenge information GUI 1270 that is displayed after the user has started the challenge. In this embodiment, challenge information GUI 1270 comprises: (1) a challenge profile card 1275, wherein a challenge icon 1271 having a picture 1272 and a challenge title 1273 representing the challenge is displayed, along with a textual description 1278 of the challenge; (2) a historical progress card 1276; (3) a current progress card 1277; (4) a statistics card 1274; and (5) a stop button 1279.

In some embodiments, the current progress card 1277 can comprise a first progress indicator 1281 that visually illustrates the current progress the user is making towards completion of the challenge. Specifically, the first progress indicator 1281 can include a first graphical indication, such as, a first plurality of circles, wherein the first progress indicator 1281 is each circle of the first plurality of circles that comprises a first color (e.g., a green colored circle to indicate an increment of successful progress). In some embodiments, a second color (e.g., a yellow colored circle) can be utilized on the first progress indicator 1281 to indicate the user made an unsuccessful attempt towards the challenge.

Further, in some embodiments, and as shown in FIGS. 12I-12K, the historical progress card 1276 can comprise a second progress indicator 1282 that visually illustrates the progress made by the user in a previous or past cycle of the challenge. Specifically, the second progress indicator 1282 can include a second graphical indication, such as, a second plurality of circles, wherein the second progress indicator 1282 is each circle of the second plurality of circles that comprises a first color (e.g., a green colored circle to indicate an increment of successful progress). In some embodiments, and as best shown in FIG. 12J, a second color (e.g., a yellow colored circle) can be utilized on the second progress indicator 1282 to indicate the user made an unsuccessful attempt towards the challenge in its last cycle of the challenge.

In some embodiments, if the user was unsuccessful in completing the challenge, then a challenge GUI 1280 is displayed (FIG. 12J). According to an aspect of the embodiments, the meal monitor application is configured to automatically detect whether progress was successfully made towards a challenge based on meal entries or the analyte level variance value associated with meal entries. For example, the meal monitor application can automatically detect what the user consumed four consecutive “green impact” or low glycemic response meals. As such, challenge GUI 1280 depicted in FIG. 12J can be displayed in response to the meal monitor application detecting that four consecutive green impact meals were not consumed by the user.

According to an aspect of the embodiments, the challenge GUI 1280 can comprise: a challenge profile card 1275; a historical progress card 1276; a statistics card 1278; and a message 1291 notifying the user that the challenge was not successfully completed. In some embodiments, and as depicted in FIG. 12J, the message 1291 can include an encourage note to the user (e.g., “have another try!”). Further in some embodiments, the challenge GUI 1280 can comprise a “Try Again” button 1288 and a “Try Another” button 1289.

FIG. 12K depicts a challenge GUI 1290 that is displayed to the user upon the user successfully completing a challenge. In some exemplar embodiments, challenge GUI 1290 shown in FIG. 12K can be displayed in response to the meal monitoring application automatically detecting that four consecutive green impact meals were consumed by the user. According to an aspect of the embodiments, the challenge GUI 1290 can comprise: a challenge profile card 1275; a historical progress card 1276; a statistics card 1278; and a congratulatory message 1293 notifying the user that the challenge has been successfully completed. Further in some embodiments, the challenge GUI 1290 can comprise a “Try Again” button 1298 and a “Try Another” button 1299. In some embodiments, when the user selects the Try Again button 1298, the user can restart the challenge.

According to an aspect of the embodiments, the statistics card 1278 is configured to indicate the number of times the challenge has successfully been completed by the user. In some embodiments, the statistics card 1278 comprises a numerical value and a text description indicating the number of times the challenge has successfully been completed (e.g., “0 times,” as shown in FIGS. 12I-12J, and “1 times,” as shown in FIG. 12K).

### Example Embodiments of Alerts for a Meal Monitor Application

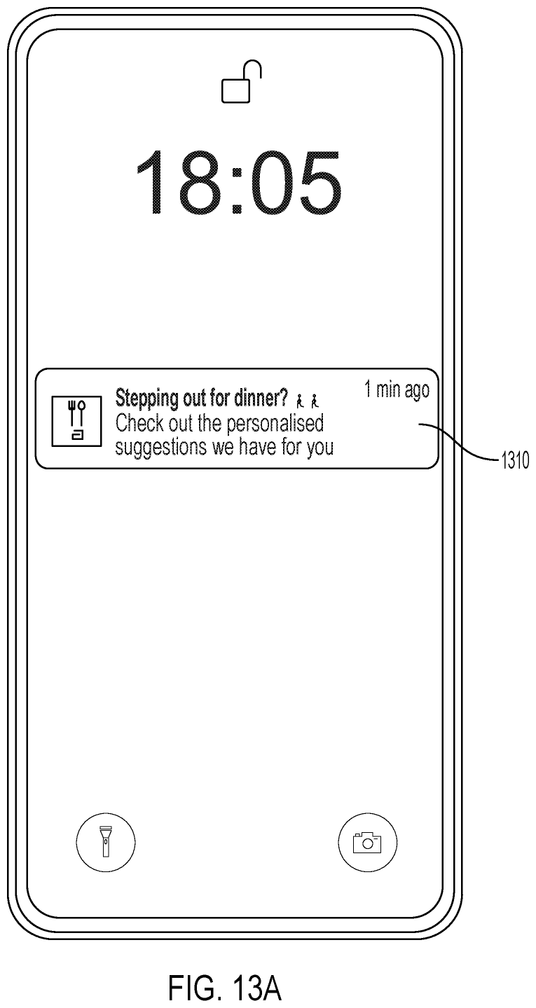

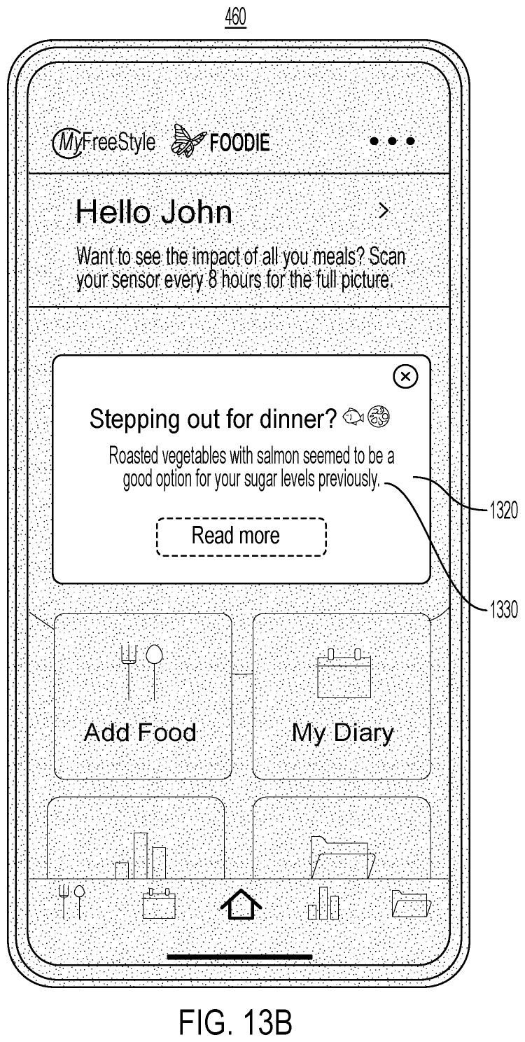

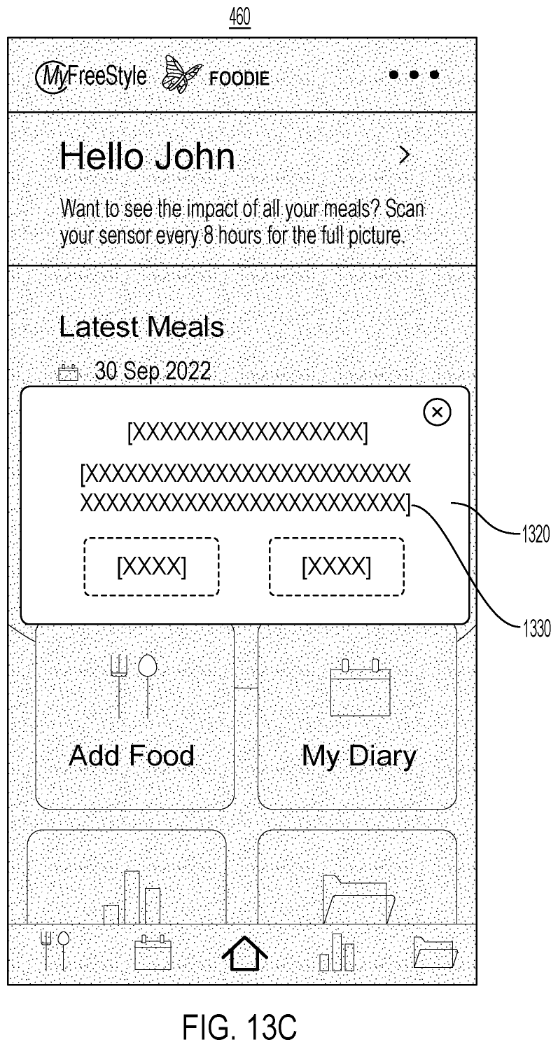

The meal monitor application may also display various alerts and notifications to remind and/or encourage the user to log meals in order to assist the user in improving or maintaining their glycemic control. In some embodiments, and as shown in FIG. 13A, a lock screen notification 1310 can appear for the meal monitoring application. For example, a lock screen notification 1310 may appear to notify the user that a high glucose meal was detected and to prompt the user to log the good based on the detected glucose spike. Alternatively, the notification 1310 may be a celebratory notification that informs the user that, e.g., they have stayed in range after a meal or that they have logged a certain number of meals.

In some embodiments, and as shown in FIG. 13B, the meal monitoring an in-app modal 1320 can be provided to the user through the meal monitoring application. In some embodiments, the in-app modal 1320 can be configured so as to partially obstruct or superimpose the underlying interface (e.g., home GUI 460) from view (FIGS. 13B and 13C). The application may also present in-app modals 1320 in response to the users tapping on a notification 1310 on the lock screen to remind and/or encourage the user. The in-app modal 1320 may be a more vibrant and visual modal within the application that provides more context for prompting action. The in-app modal 1320 may include possible answers such that the user can indicate that they did not eat anything or the user can select a link to add food, which may open a food logging screen, examples of which are described elsewhere in the application. Alternatively, the in-app modal 1320 may be a celebratory notification, which contains encouraging words and a graphic that complement the user for, e.g., staying within target. The in-app modal 1320 can present a graphic and text that nudges the user to log a meal by reminding them that many reasons may have caused the detected glucose spike and logging meals will help the user discern the cause of the glucose spike.

Further, the in-app modal 1320 can present a graphic and text that gently nudges the user to deliver a predetermined message 1330. According to an aspect of the embodiments, the messages 1330 are personalized for the user. In some embodiments, the meal monitoring application is configured to analyze past meal entries and/or the analyte level variance value associated with past meal entries so as to push a particular message 1330 to the user based on the analysis.

In exemplar embodiments, the in-app modal 1320 comprises a message 1330 suggesting food choices for the user based on their past experiences and entries. As shown in FIG. 13B, in-app modal 1320 comprises a message 1330 suggesting roasted vegetables with salmon for dinner based on the user's previous sugar levels.

According to another aspect of the embodiments, the in-app modal 1320 can comprise a message 1330 relating to the following topics areas, including but not limited to: (1) sensor scanning (e.g., prompting the user to scan their sensor before bed); (2) user profile (e.g., requesting the user to complete their profile); (3) nutrition (e.g., providing nutritional tips); and (4) meal entries (e.g., recommending food or meal changes, such as swapping certain foods out for other foods, for example, replacing pizza with chicken).

In some embodiments, in-app modal 1320 softly nudges the user. In some embodiments, in-app modal 1320 is displayed after the user selects a banner notification 1310 displayed outside of the meal monitoring application (see, e.g., FIG. 13A). In some embodiments, a banner notification 1320 that gently nudges the user can be displayed outside of the meal monitoring application (e.g., on a lock screen), wherein the banner notification 1310 comprises a message indicating that the meal monitoring application has personalized suggestions for the user (e.g., dinner recommendations, as shown in FIG. 13A).

According to an aspect of the embodiments, the in-app messaging function on the meal monitoring application requires the user to specifically opt-in to activate the in-app messages 1330. As such, the user must first grant the meal monitoring application permission to send the user in-app messages 1330 prior to the meal monitoring application populating said in-app messages 1330 for the user.

These examples are meant to be illustrative only, and those of skill in the art will recognize that other combinations and permutations of modals can be implemented and are fully within the scope of the present disclosure.

Various aspects of the present subject matter are set forth below, in review of, and/or in supplementation to, the embodiments described thus far, with the emphasis here being on the interrelation and interchangeability of the following embodiments. In other words, an emphasis is on the fact that each feature of the embodiments can be combined with each and every other feature unless explicitly stated otherwise or logically implausible. The embodiments described herein are restated and expanded upon in the following paragraphs without explicit reference to the figures.

Systems, devices, and methods for detecting, measuring and classifying meals for an individual based on analyte measurements. These results and related information can be presented to the individual to show the individual which meals are causing the most severe analyte response. These results can be organized and categorized based on preselected criteria or previous meals and results so as to organize and present the results in a format with reference to glucose as the monitored analyte. Various embodiments disclosed herein relate to methods, systems, and software applications intended to engage an individual by providing direct and timely feedback regarding the individual's meal-related glycemic response.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

To the extent the embodiments disclosed herein include or operate in association with memory, storage, and/or computer readable media, then that memory, storage, and/or computer readable media are non-transitory. Accordingly, to the extent that memory, storage, and/or computer readable media are covered by one or more claims, then that memory, storage, and/or computer readable media is only non-transitory.

In many instances, entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic) intervening entities) and the indirect coupling of two entities (with one or more non-negligible intervening entities). Where entities are shown as being directly coupled together, or described as coupled together without description of any intervening entity, it should be understood that those entities can be indirectly coupled together as well unless the context clearly dictates otherwise.

The subject matter described herein and in the accompanying figures is done so with sufficient detail and clarity to permit the inclusion of claims, at any time, in means-plus-function format pursuant to 35 U.S.C. section 112, part (f). However, a claim is to be interpreted as invoking this means-plus-function format only if the phrase “means for” is explicitly recited in that claim.

Aspects of the invention are set out in the independent claims and preferred features are set out in the dependent claims. The preferred features of the dependent claims may be provided in combination in a single embodiment and preferred features of one aspect may be provided in conjunction with other aspects.

As used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

While the embodiments are susceptible to various modifications and alternative forms, specific examples thereof have been shown in the drawings and are herein described in detail. These embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the scope of the claims by features, functions, steps, or elements that are not within that scope.

### CLAUSES

Exemplary embodiments are set out in the following numbered clauses.

Clause 1. A system for monitoring meal-related analyte responses in a user, the system comprising: a reader device, comprising: wireless communication circuitry configured to receive data indicative of an analyte level of the user, one or more processors coupled with a memory, the memory storing a meal monitoring application that, when executed by the one or more processors, causes the one or more processors to output a first challenge graphical user interface (GUI) reflecting a list of one or more challenges relating to the user's analyte response, wherein the one or more challenges comprise one or more active challenges, one or more completed challenges, and one or more unattempted challenges, the first challenge GUI comprising a first challenge card, a second challenge card, and a third challenge card, wherein the first challenge card reflects the one or more active challenges, wherein each of the one or more active challenges reflects a challenge currently in progress by the user on the meal monitoring application, wherein the second challenge card reflects the one or more completed challenges, wherein each of the one or more completed challenges reflects a challenge completed by the user, and, wherein the third challenge card reflects one or more unattempted challenges, wherein each of the one or more unattempted challenges reflects a challenge in which the user has not yet participated in.

Clause 2. The system of clause 1, wherein the first challenge card comprises one or more selectable first challenge icons, wherein each of the one or more selectable first challenge icons reflects a challenge currently in progress by the user.

Clause 3. The system of clause 2, wherein each of the one or more selectable first challenge icons comprises a first indicator, a picture and a textual description relating to the challenge currently in progress, wherein the first indicator is displayed on the picture and is configured to indicate that the challenge is currently in progress.

Clause 4. The system of clause 3, wherein the first indicator is a green dot.

Clause 5. The system of any preceding clause, wherein the second challenge card comprises one or more selectable second challenge icons, wherein each of the one or more selectable second challenge icons reflects a challenge completed by the user.

Clause 6. The system of clause 5, wherein each of the one or more selectable second challenge icons comprises a second indicator, a picture and a textual description relating to the completed challenge, wherein the second indicator is overlayed on the picture and is configured to indicate that the challenge has been completed by the user.

Clause 7. The system of clause 5, wherein each of the one or more selectable second challenge icons comprises a second indicator, a picture and a textual description relating to the completed challenge, wherein the second indicator is overlayed on the picture and is configured to indicate that the challenge has been completed by the user.

Clause 8. The system of any preceding clause, wherein the third challenge card comprises one or more selectable third challenge icons, wherein each of the one or more selectable third challenge icons reflects a challenge not yet tried by the user.

Clause 9. The system of clause 8, wherein each of the one or more selectable third challenge icons comprises a picture and a textual description relating to the completed challenge.

Clause 10. The system of any preceding clause, wherein each of the first challenge card, the second challenge card, and the third challenge card include a plurality of selectable challenge icons, wherein a first set of the plurality of selectable challenge icons is displayed on the first challenge GUI, wherein the reader device further comprises a touchscreen, and wherein the meal monitoring application, when executed by the one or more processors, further causes the one or more processors to: receive input from the touchscreen corresponding to a swipe gesture or a drag gesture, and in response to the received input, display a second set of the plurality of selectable challenge icons on the first challenge GUI, wherein at least one or more of the plurality of selectable challenge icons of the second set is different than at least one or more of the plurality of selectable challenge icons of the first set.

Clause 11. The system of any preceding clause, wherein the list of one or more challenges includes one or more selectable challenge icons, wherein each of the one or more selectable challenge icons corresponds to one of the one or more challenges relating to the user's analyte response or glucose levels, wherein the meal monitoring application, when executed by the one or more processors, further causes the one or more processors to: in response to a selection of one of the one or more selectable challenge icons, output a second challenge GUI reflecting contextual information related to the one of the one or more challenges corresponding to the selected one of the one or more selectable challenge icons.

Clause 12. The system of clause 11, wherein the second challenge GUI comprises: a challenge profile section comprising the selected one of the one or more selectable challenge icons, a picture, and a challenge title providing a textual description of the one of the one or more challenges corresponding to the selected one of the one or more selectable challenge icons; an attempt indicator configured to indicate when the one of the one or more challenges corresponding to the selected one of the one or more selectable challenge icons was last attempted by the user; and a completion indicator configured to indicate when the one of the one or more challenges corresponding to the selected one of the one or more selectable challenge icons was last successfully completed by the user.

Clause 13. The system of clause 11 or clause 12, wherein, if the one of the one or more challenges corresponding to the selected one of the one or more selectable challenge icons is an unattempted challenge, then the second challenge GUI further comprises a start button, wherein the meal monitoring application, when executed by the one or more processors, further causes the one or more processors to: in response to a selection of the start button, begin the unattempted challenge on the meal monitoring application.

Clause 14. The system of clause 13, wherein the meal monitoring application, when executed by the one or more processors, further causes the one or more processors to: in response to a selection of the start button, begin the unattempted challenge on the meal monitoring application on a following day.

Clause 15. The system of clause 11 or clause 12, wherein, if the one of the one or more challenges corresponding to the selected one of the one or more selectable challenge icons is an active challenge, then the second challenge GUI further comprises a stop button, wherein the meal monitoring application, when executed by the one or more processors, further causes the one or more processors to: in response to a selection of the stop button, cease continuation of the active challenge.

Clause 16. The system of clause 11 or clause 12, wherein, if the one of the one or more challenges corresponding to the selected one of the one or more selectable challenge icons is an active challenge, the second challenge GUI further comprises a progress card configured to indicate progress the user has made towards the active challenge, wherein the progress card comprises a unit of measure and a unit of time to indicate the progress.

Clause 17. The system of clause 16, wherein the unit of measure includes a fractional unit, and wherein the unit of time includes a number of days.

Clause 18. The system of clause 11 or clause 12, wherein a modal is displayed on the second challenge GUI if the one of the one or more challenges corresponding to the selected one of the one or more selectable challenge icons is an active challenge, wherein the modal is configured to prompt the user to provide progress information related to the active challenge in the meal monitoring application.

Clause 19. The system of clause 18, wherein the meal monitoring application is configured to detect whether the user successfully completed the active challenge based on the tracked progress, wherein the meal monitoring application, when executed by the one or more processors, further causes the one or more processors to: in response to the meal monitoring application detecting the user successfully completed the active challenge, output a third challenge GUI, wherein the third challenge GUI comprises a challenge profile section, an attempt indicator, a completion indicator, and a message congratulating the user on successfully completed the active challenge, and in response to the meal monitoring application detecting the user successfully completed the active challenge, identify the active challenge as a completed challenge, wherein the third challenge GUI further comprises a first button which, when selected by the user, is configured to restart the completed challenge, and wherein the third challenge GUI further comprises a second button which, when selected by the user, outputs the first challenge GUI, wherein the user selects a different challenge from the list of one or more challenges reflected by the first challenge GUI.

Clause 20. The system of clause 19, wherein a modal is displayed on the third challenge GUI in response to the user selecting the first button, and wherein the modal is configured to prompt the user to confirm whether the user would like to restart the completed challenge.

Clause 21. The system of clause 18, wherein the meal monitoring application is configured to detect whether the user successfully completed the active challenge based on the tracked progress, wherein the meal monitoring application, when executed by the one or more processors, further causes the one or more processors to: in response to the meal monitoring application detecting the user did not successfully complete the active challenge, output a fourth challenge GUI, wherein the fourth challenge GUI comprises a challenge profile section, an attempt indicator, a completion indicator, and a message notifying the user that the active challenge was not successfully completed, and in response to the meal monitoring application detecting the active challenge was not successfully completed, identify the active challenge as a completed challenge, wherein the fourth challenge GUI further comprises a first button which, when selected by the user, is configured to restart the completed challenge, and wherein the fourth challenge GUI further comprises a second button which, when selected by the user, outputs the first challenge GUI, wherein the user selects a different challenge from the list of one or more challenges reflected by the first challenge GUI.

Clause 22. The system of clause 21, wherein a modal is displayed on the fourth challenge GUI in response to the user selecting the first button, and wherein the modal is configured to prompt the user to confirm whether the user would like to restart the completed challenge.

Clause 23. The system of any preceding clause, wherein the meal monitoring application comprises a home GUI comprising a challenges card, wherein the challenges card comprises a selectable link, wherein the meal monitoring application, when executed by the one or more processors, further causes the one or more processors to:

in response to the user selecting the link, output the first challenge GUI.

Clause 24. The system of any preceding clause, wherein each of the one or more challenges is configured to represent a challenge directed to the user's behavior or activity that can affect the user's analyte levels.

Clause 25. A system for monitoring meal-related analyte responses in a user, the system comprising: a reader device, comprising: wireless communication circuitry configured to receive data indicative of an analyte level of the user, one or more processors coupled with a memory, the memory storing a meal monitoring application that, when executed by the one or more processors, causes the one or more processors to: receive meal information inputted by the user, wherein the meal information is configured to reflect the user's food choices; output a home graphical user interface (GUI), wherein the home GUI comprises: a plurality of selectable sections, the plurality of selection sections comprising a user profile section, a meal entry section, a trends section, a diary section, and a reports section; a meals card configured to display one or more meal listings comprising the inputted meal information related to one or more consumed meals by the user; a trends card comprising a graphical representation reflecting information related to an analyte response associated with the user's food choices; a challenges card reflecting a list of one or more challenges relating the user's analyte response or glucose levels; and a recommendations card reflecting one or more recommendations relating to the user's food choices or analyte response.

Clause 26. The system of clause 25, wherein the recommendations card comprises one or more selectable recommendation icons, wherein each of the one or more selectable recommendation icons corresponds to one of the one or more recommendations.

Clause 27. The system of clause 26, wherein each of the one or more selectable recommendation icons comprise a picture relating to the corresponding one of the one or more recommendations, and a recommendation title providing a textual description of the corresponding one of the one or more recommendations.

Clause 28. The system of any one of clauses 25 to 27, wherein the recommendations card comprises a plurality of selectable recommendation icons, wherein a first set of the plurality of selectable recommendations icons is displayed on the recommendations card, wherein the reader device further comprises a touchscreen, and wherein the meal monitoring application, when executed by the one or more processors, further causes the one or more processors to: receive input from the touchscreen corresponding to a swipe gesture or a drag gesture, and in response to the received input, display a second set of the plurality of selectable recommendations icons on the recommendations card, wherein at least one or more of the plurality of selectable recommendations icons of the second set is different than at least one or more of the plurality of selectable recommendations icons of the first set.

Clause 29. The system of any one of clauses 25 to 28, wherein the recommendations card comprises one or more selectable recommendation icons, wherein each of the one or more selectable recommendation icons corresponds to one of the one or more recommendations, wherein the meal monitoring application, when executed by the one or more processors, further causes the one or more processors to: in response to a selection of one of the one or more selectable recommendation icons, output a modal on the home GUI, wherein the modal provides contextual information related to the corresponding one of the one or more recommendations, and wherein the modal is configured to direct the user to act in accordance with the corresponding one of the one or more recommendations.

Clause 30. The system of any one of clauses 25 to 29, wherein the meal monitoring application, when executed by the one or more processors, further causes the one or more processors to: detect the user's food choices; analyze the inputted meal information; and based on the analysis, display one or more selectable recommendation icons on the recommendations card, wherein each of the one or more selectable recommendation icons reflects a recommendation related to the user's food choices or analyte response.

Clause 31. The system of any one of clauses 25 to 30, wherein the recommendations card comprises one or more selectable recommendation icons, wherein each of the one or more selectable recommendation icons corresponds to one of the one or more recommendations, wherein the meal monitoring application, when executed by the one or more processors, further causes the one or more processors to: in response to a selection of one of the one or more selectable recommendation icons, remove the selected one of the one or more selectable recommendation icons from the recommendation card, and display a new selectable recommendation icon on the recommendation card in place of the removed recommendation icon.

Clause 32. The system of any one of clauses 25 to 31, wherein the recommendations card comprises one or more selectable recommendation icons, wherein each of the one or more selectable recommendation icons corresponds to one of the one or more recommendations, wherein each of the one or more selectable recommendation icons is configured to be displayed on the recommendation card for a predetermined period of time.

Clause 33. The system of clause 32, wherein the meal monitoring application, when executed by the one or more processors, further causes the one or more processors to: detect when the predetermined period of time has been reached, in response to the predetermined period of time being reached, replace the one or more selectable recommendation icons on the recommendation card with a new set of one or more selectable recommendation icons, wherein at least one of the one or more selectable recommendation icons in the new set is different than at least one of the replaced one or more selectable recommendation icons.

Clause 34. The system of any one of clauses 25 to 33, wherein the home GUI is configured to transition between a plurality of views, wherein the plurality of views comprises at least a first view and a second view.

Clause 35. The system of clause 34, wherein the home GUI is in the first view, wherein the home GUI is configured to display the user profile section, the meal entry section, the diary section, and the meals card in the first view, wherein the reader device further comprises a touchscreen, wherein the meal monitoring application, when executed by the one or more processors, further causes the one or more processors to: receive input from the touchscreen corresponding to a scroll gesture, a swipe gesture, a pull gesture, or a drag gesture, and wherein, in response to the received input, the home GUI is configured to transition from the first view to the second view, wherein the trends card, the challenges card, and the recommendations card are displayed on the home GUI in the second view.

Clause 36. The system of any one of clauses 25 to 35, wherein the home GUI is configured to transition between a plurality of views, wherein each view of the plurality of views is different, wherein the reader device further comprises a touchscreen, wherein the meal monitoring application, when executed by the one or more processors, further causes the one or more processors to: receive input from the touchscreen corresponding to a scroll gesture, a swipe gesture, a pull gesture, or a drag gesture, and in response to the received input, display one view of the plurality of views of the home GUI.

Clause 37. The system of any one of clauses 25 to 36, wherein the meals card is configured to display one or more meal listings comprising meal information related to one or more of the most recently consumed meals.

Clause 38. The system of any one of clauses 25 to 37, wherein each of the one or more meal listings includes details of a meal consumed by the user, wherein the one or more meals listings are displayed on the meals card in chronological order, wherein a meal listing corresponding to a most recently consumed meal is displayed at a top portion of the meals card.

Clause 39. The system of any one of clauses 25 to 38, wherein each of the one or more meal listings includes details of a meal consumed by the user and the meal's corresponding meal-related analyte response.

Clause 40. The system of any one of clauses 25 to 39, wherein each of the one or more meal listings comprises: a text description of a meal consumed by the user; a portion size indicator comprising information indicating the meal was either smaller, medium, or large compared to a usual meal serving of the user; a datestamp associated with a date the meal was consumed by the user; a time stamp associated with a time the meal was consumed; and a graphical representation of an analyte response associated with the meal.

Clause 41. The system of clause 40, wherein the graphical representation comprises a plurality of segments.

Clause 42. The system of clause 41, wherein the plurality of segments includes a first segment, and wherein the first segment is indicative of the analyte response comprising a low glycemic response, wherein the plurality of segments includes a second segment, wherein the second segment is indicative of the analyte response comprising a medium glycemic response, and wherein the plurality of segments includes a third segment, wherein the third segment is indicative of the analyte response comprising a high glycemic response.

Clause 43. The system of clause 42, wherein the first segment, the second segment, and the third segment are each a different color.

Clause 44. The system of any one of clauses 25 to 43, wherein the graphical representation of the trends card is indicative of the analyte response associated with the user's food choices for a predetermined time period.

Clause 45. The system of clause 44, wherein the graphical representation of the trends card comprises a plurality of colored segments comprising a first colored segment, a second colored segment, and a third colored segment.

Clause 46. The system of clause 45, wherein the first colored segment comprises a green color indicative of a low glycemic response, wherein the second colored segment comprises a yellow color indicative of a medium glycemic response, and wherein the third colored segment comprises an orange color indicative of a high glycemic response.

Clause 47. The system of any one of clauses 25 to 40, wherein the trends card comprises a summary panel configured to provide an overall assessment of the user's food choices for a predetermined period of time.

Clause 48. The system of any one of clauses 25 to 47, wherein the trends card comprises a summary panel comprising information indicative of the analyte response associated with the user's food choices, wherein the trends card is configured to be dynamic, wherein the meal monitoring application, when executed by the one or more processors, further causes the one or more processors to: detect whether the analyte response associated with the user's food choices has provided new trend information, and in response to the new trend information being detected, populate an updated summary panel on the trends card.

Clause 49. The system of any one of clauses 25 to 48, wherein the trends card is not displayed on the home GUI when data indicative of an analyte level has not been received or associated with the inputted meal information.

Clause 50. The system of any one of clauses 25 to 49, wherein the challenges card on home GUI comprises one or more selectable challenge icons, wherein each of the one or more selectable challenge icons is configured to reflect a challenge relating to the user's analyte response or glucose levels.

Clause 51. The system of clause 50, wherein each of the one or more selectable challenge icons comprises a picture associated with the challenged reflected by the selected challenge icon, and a challenge title providing a textual description of the challenge reflected by the selected challenge icon.

Clause 52. The system of clause 51, wherein a live indicator is displayed on the picture to indicate the challenge reflected by picture is an active challenge on the meal monitoring application.

Clause 53. The system of any one of clauses 25 to 52, wherein the challenges card comprises a selectable link, wherein the meal monitoring application, when executed by the one or more processors, further causes the one or more processors to: in response to the user selecting the link, output a first challenge GUI comprising information on all challenges provided on the meal monitoring application.

Clause 54. The system of any one of clauses 25 to 53, wherein the challenges card comprises a plurality of selectable challenge icons, wherein a first set of the plurality of selectable challenge icons is displayed on the challenge card, wherein the reader device further comprises a touchscreen, and wherein the meal monitoring application, when executed by the one or more processors, further causes the one or more processors to: receive input from the touchscreen corresponding to a swipe gesture or a drag gesture, and in response to the received input, display a second set of the plurality of selectable challenge icons on the challenges, wherein at least one or more of the plurality of selectable challenge icons of the second set is different than at least one or more of the plurality of selectable challenge icons of the first set.

Clause 55. The system of any one of clauses 25 to 54, wherein the challenges card comprises a plurality of selectable challenge icons, wherein the challenges card is configured to display two or three of the plurality of selectable challenge icons on the home GUI at a same time.

Clause 56. The system of any one of clauses 25 to 55, wherein the recommendations card comprises a plurality of selectable recommendation icons, wherein the recommendations card is configured to display two or three of the plurality of selectable recommendation icons at a same time.

Clause 57. The system of any one of clauses 25 to 56, wherein the home GUI further comprises a navigation bar.

Clause 58. The system of any one of clauses 25 to 57, wherein the home GUI further comprises banner comprising a message relating to scanning a sensor and a meal impact.

Clause 59. A system for monitoring meal-related analyte responses in a subject, the system comprising: a reader device, comprising: wireless communication circuitry configured to receive data indicative of an analyte level of the subject, one or more processors coupled with a memory, the memory storing a meal monitoring application that, when executed by the one or more processors, causes the one or more processors to: identify a peak analyte level value within a predetermined time period for the received data indicative of the analyte level of the subject, determine an estimated meal start time and an initial analyte level value based on the peak analyte level value, determine an analyte level variance value, prompt the subject to enter meal information, and associate the entered meal information with the analyte level variance value.

Clause 60. The system of clause 59, wherein the reader device comprises a smart phone.

Clause 61. The system of clause 59 or clause 60, wherein the data indicative of the analyte level of the subject comprises data indicative of a glucose level.

Clause 62. The system of any one of clauses 59 to 61, further comprising a trusted computer system, wherein the trusted computer system is a cloud-computing platform comprising one or more servers.

Clause 63. The system of clause 62, wherein the trusted computer system is configured to transmit the data indicative of the analyte level of the subject to the reader device.

Clause 64. The system of any one of clauses 59 to 63, further comprising a sensor control device, wherein the sensor control device comprises an analyte sensor, and wherein at least a portion of the analyte sensor is configured to be positioned under a skin layer of the subject and in contact with a bodily fluid of the subject.

Clause 65. The system of clause 64, wherein the sensor control device further is configured to transmit the data indicative of the analyte level of the subject to the reader device.

Clause 66. The system of any one of clauses 59 to 65, wherein the wireless communication circuitry of the reader device is configured to receive the data indicative of the analyte level of the subject according to a Bluetooth or a Near Field Communication wireless protocol.

Clause 67. The system of any one of clauses 59 to 66, wherein the peak analyte level value comprises a highest glucose value over a predetermined analyte level threshold.

Clause 68. The system of clause 67, wherein the predetermined analyte level threshold is 170 mg/dL.

Clause 69. The system of clause 67, wherein the predetermined analyte level threshold is 180 mg/dL.

Clause 70. The system of clause 67, wherein the predetermined analyte level threshold is 190 mg/dL.

Clause 71. The system of any one of clauses 59 to 70, wherein the predetermined time period for the received data indicative of the analyte level of the subject comprises a last two hours of analyte data.

Clause 72. The system of any one of clauses 59 to 71, wherein the predetermined time period for the received data indicative of the analyte level of the subject comprises a last four hours of analyte data.

Clause 73. The system of any one of clauses 59 to 72, wherein the predetermined time period for the received data indicative of the analyte level of the subject comprises a last eight hours of analyte data.

Clause 74. The system of any one of clauses 59 to 73, wherein the estimated meal start time is determined by counting two hours back from a time of the peak analyte level value.

Clause 75. The system of any one of clauses 59 to 73, wherein the estimated meal start time is determined by counting three hours back from a time of the peak analyte level value.

Clause 76. The system of any one of clauses 59 to 73, wherein the estimated meal start time is determined by counting four hours back from a time of the peak analyte level value.

Clause 77. The system of any one of clauses 59 to 76, wherein the analyte level variance value is determined by subtracting the initial analyte level value from the peak analyte level value.

Clause 78. The system of any one of clauses 59 to 77, wherein the meal monitoring application, when executed by the one or more processors, further causes the one or more processors to store the meal information and associated analyte level variance value in the memory of the reader device.

Clause 79. A system for monitoring meal-related analyte responses in a subject, the system comprising: a reader device, comprising: wireless communication circuitry configured to receive data indicative of an analyte level of the subject, one or more processors coupled with a memory, the memory storing a meal monitoring application that, when executed by the one or more processors, causes the one or more processors to: receive meal information inputted by the subject, receive the data indicative of the analyte level of the subject within a predetermined amount of time after the meal information is inputted by the subject, identify a peak analyte level value for the received data indicative of the analyte level of the subject, determine an initial analyte level value, determine an analyte level variance value, and associate the entered meal information with the analyte level variance value.

Clause 80. The system of clause 79, wherein the reader device comprises a smart phone.

Clause 81. The system of clause 79 or clause 80, wherein the data indicative of the analyte level of the subject comprises data indicative of a glucose level.

Clause 82. The system of any one of clauses 79 to 81, further comprising a trusted computer system, wherein the trusted computer system is a cloud-computing platform comprising one or more servers.

Clause 83. The system of any one of clauses 79 to 82, wherein the trusted computer system is configured to transmit the data indicative of the analyte level of the subject to the reader device.

Clause 84. The system of any one of clauses 79 to 83, further comprising a sensor control device, wherein the sensor control device comprises an analyte sensor, and wherein at least a portion of the analyte sensor is configured to be positioned under a skin layer of the subject and in contact with a bodily fluid of the subject.

Clause 85. The system of clause 84, wherein the sensor control device further is configured to transmit the data indicative of the analyte level of the subject to the reader device.

Clause 86. The system of any one of clauses 79 to 85, wherein the wireless communication circuitry of the reader device is configured to receive the data indicative of the analyte level of the subject according to a Bluetooth or a Near Field Communication wireless protocol.

Clause 87. The system of any one of clauses 79 to 86, wherein the peak analyte level value comprises a highest glucose value over a predetermined analyte level threshold.

Clause 88. The system of clause 87, wherein the predetermined analyte level threshold is 170 mg/dL.

Clause 89. The system of clause 87, wherein the predetermined analyte level threshold is 180 mg/dL.

Claim 90. The system of clause 87, wherein the predetermined analyte level threshold is 190 mg/dL.

Clause 91. The system of any one of clauses 79 to 90, wherein the analyte level variance value is determined by subtracting the initial analyte level value from the peak analyte level value.

Clause 92. The system of any one of clauses 79 to 91, wherein the meal monitoring application, when executed by the one or more processors, further causes the one or more processors to store the meal information and associated analyte level variance value in the memory of the reader device.

Clause 93. The system of any one of clauses 79 to 92, wherein the meal monitoring application, when executed by the one or more processors, further causes the one or more processors to display a notification that a meal entry has not been entered after a predetermined reminder time period.

Clause 94. The system of clause 93, wherein the predetermined reminder time period is one week.

Clause 95. The system of clause 93, wherein the predetermined reminder time period is three days.

Clause 96. The system of clause 93, wherein the predetermined reminder time period is one day.

Clause 97. The system of any one of clauses 79 to 96, wherein the initial analyte level value is determined based on a time of the meal information inputted by the subject.

Clause 98. A system for monitoring meal-related analyte responses in a subject, the system comprising: a reader device, comprising: wireless communication circuitry configured to receive data indicative of an analyte level of the subject, one or more processors coupled with a memory, the memory storing a meal monitoring application that, when executed by the one or more processors, causes the one or more processors to output a diary graphical user interface (GUI), the diary GUI comprising a plurality of meal entries, wherein each meal entry of the plurality of meal entries comprises: a date of the each meal entry, a meal name, a graphical representation of an analyte level variance value associated with the each meal entry, and a numerical representation of the analyte level variance value associated with the each meal entry.

Clause 99. The system of clause 98, wherein the graphical representation of the analyte level variance value comprises a plurality of segments.

Clause 100. The system of clause 99, wherein the plurality of segments includes a first segment, and wherein the first segment is indicative of the analyte level variance value in a first analyte level variance range, and wherein the plurality of segments includes a second segment, and wherein the second segment is indicative of the analyte level variance value in a second analyte level variance range that is different from the first analyte level variance range.

Clause 101. The system of clause 100, wherein the first segment is a different color from the second segment.

Clause 102. The system of clause 100, wherein the first segment comprises a different area from the second segment.

Clause 103. The system of any one of clauses 100 to 102, wherein the first analyte level variance range is less than 70 mg/dL.

Clause 104. The system of any one of clauses 100 to 102, wherein the second analyte level variance range is between 70 mg/dL and 120 mg/dL.

Clause 105. The system of any one of clauses 100 to 104, wherein the plurality of segments further includes a third segment indicative of the analyte level variance value in a third analyte level variance range that is different from both the first analyte level variance range and the second analyte level variance range.

Clause 106. The system of clause 105, wherein the third segment is a different color from the first segment and the second segment.

Clause 107. The system of any one of clauses 98 to 106, wherein the each meal entry of the plurality of meal entries further comprises a time of the each meal entry.

Clause 108. The system of any one of clauses 98 to 107, wherein the each meal entry of the plurality of meal entries further comprises an activity field.

Clause 109. The system of any one of clauses 98 to 108, wherein the each meal entry of the plurality of meal entries further comprises a notes field.

Clause 110. The system of any one of clauses 98 to 109, wherein the diary GUI further comprises a view setting configured to display the plurality of meal entries by day or by week.

Clause 111. The system of any one of clauses 98 to 110, wherein the each meal entry of the plurality of meal entries further comprises a weighted average of the analyte level variance value.

Clause 112. The system of clause 111, wherein the weighted average of the analyte level variance value is based on a plurality of historical meal entries having a same or similar meal or food to the each meal entry.

Clause 113. The system of clause 111, wherein the weighted average of the analyte level variance value is determined by a weighted average function comprising a recency factor.

Clause 114. The system of clause 113, wherein the recency factor of the weighted average function is configured to decrement the analyte level variance value of a historical meal entry by a predetermined factor for each day between a date of the historical meal entry and a current date.

Clause 115. A system for monitoring meal-related analyte responses in a subject, the system comprising: a reader device, comprising: wireless communication circuitry configured to receive data indicative of an analyte level of the subject, one or more processors coupled with a memory, the memory storing a meal monitoring application that, when executed by the one or more processors, causes the one or more processors to output a trends graphical user interface (GUI), the trends GUI comprising a glycemic response view and a meals view, wherein the glycemic response view comprises a graphical representation reflecting a plurality of segments comprising a first segment and a second segment, wherein the first segment is indicative of a first analyte level variance range, and the second segment is indicative of a second analyte level variance range that is different from the first analyte level variance range.

Clause 116. The system of clause 115, wherein the first segment is indicative of a first set of meal entries each having an analyte level variance value within the first analyte level variance range.

Clause 117. The system of clause 115 or clause 116, wherein the second segment is indicative of a second set of meal entries each having an analyte level variance value within the second analyte level variance range.

Clause 118. The system of any one of clauses 115 to 117, wherein the meals view comprises: a plurality of meal entries, wherein each meal entry of the plurality of meal entries comprises: a date and a time of the each meal entry, a meal name, a graphical representation of an analyte level variance value associated with the each meal entry, and a numerical representation of the analyte level variance value associated with the each meal entry.

Clause 119. A method for monitoring meal-related analyte responses in a user, the method comprising: receiving, through wireless communication circuitry, data indicative of an analyte level of a user; identifying an analyte response of the user based on the data indicative of an analyte level; and outputting, by a processor coupled with a memory storing a meal monitoring application, a first challenge graphical user interface (GUI) reflecting a list of one or more challenges relating to the user's analyte response, wherein the one or more challenges comprise one or more active challenges, one or more completed challenges, and one or more unattempted challenges, the first challenge GUI comprising a first challenge card, a second challenge card, and a third challenge card; wherein the first challenge card reflects the one or more active challenges, wherein each of the one or more active challenges reflects a challenge currently in progress by the user on the meal monitoring application, wherein the second challenge card reflects the one or more completed challenges, wherein each of the one or more completed challenges reflects a challenge completed by the user, and, wherein the third challenge card reflects one or more unattempted challenges, wherein each of the one or more unattempted challenges reflects a challenge in which the user has not yet participated in.

Clause 120. A method for monitoring meal-related analyte responses in a user, the method comprising: receiving, by a processor coupled with a memory storing a meal monitoring application, inputted meal information by the user, wherein the meal information is reflects the user's food choices; and outputting, by the processor, a home graphical user interface (GUI) comprising: a plurality of selectable sections, wherein the plurality of selection sections comprises a user profile section, a meal entry section, a trends section, a diary section, and a reports section; a meals card configured to display one or more meal listings comprising the inputted meal information related to one or more consumed meals by the user; a trends card comprising a graphical representation reflecting information related to an analyte response associated with the user's food choices; a challenges card reflecting a list of one or more challenges relating the user's analyte response or glucose levels; and a recommendations card reflecting one or more recommendations relating to the user's food choices or analyte response.

Clause 121. A method for monitoring meal-related analyte responses in a user, the method comprising: receiving, through wireless communication circuitry, data indicative of an analyte level of a user; identifying, by a processor coupled with a memory storing a meal monitoring application, a peak analyte level value within a predetermined time period for the received data indicative of the analyte level of the subject; determining, by the processor, an estimated meal start time and an initial analyte level value based on the peak analyte level value; determining, by the processor, an analyte level variance value; prompting, by the processor, the subject to enter meal information; and associating, by the processor, the entered meal information with the analyte level variance value.

Clause 122. A method for monitoring meal-related analyte responses in a user, the method comprising: receiving, through wireless communication circuitry, data indicative of an analyte level of a user; receiving, by a processor coupled with a memory storing a meal monitoring application, meal information inputted by the user; receiving, by the processor, the data indicative of the analyte level of the user within a predetermined amount of time after the meal information is inputted by the user; identifying, by the processor, a peak analyte level value for the received data indicative of the analyte level of the subject; determining, by the processor, an initial analyte level value; determining, by the processor, an analyte level variance value; and associating, by the processor, the entered meal information with the analyte level variance value.

Clause 123. A method for monitoring meal-related analyte responses in a user, the method comprising: receiving, through wireless communication circuitry, data indicative of an analyte level of a user; outputting, by a processor coupled with a memory storing a meal monitoring application, a diary graphical user interface (GUI) comprising a plurality of meal entries, wherein each meal entry of the plurality of meal entries comprises: a date of the each meal entry, a meal name, a graphical representation of an analyte level variance value associated with the each meal entry, and a numerical representation of the analyte level variance value associated with the each meal entry.

Clause 124. A method for monitoring meal-related analyte responses in a user, the method comprising: receiving, through wireless communication circuitry, data indicative of an analyte level of a user; and outputting, by a processor coupled with a memory storing a meal monitoring application, a trends graphical user interface (GUI) comprising a glycemic response view and a meals view, wherein the glycemic response view comprises a graphical representation reflecting a plurality of segments comprising a first segment and a second segment, wherein the first segment is indicative of a first analyte level variance range, and the second segment is indicative of a second analyte level variance range that is different from the first analyte level variance range.

1-58. (canceled)

59. A system for monitoring meal-related glucose responses in a subject, the system comprising:
a reader device, comprising:
wireless communication circuitry configured to receive data indicative of a glucose level of the subject,
one or more processors coupled with a memory, the memory storing a meal monitoring application that, when executed by the one or more processors, causes the one or more processors to:
identify a peak glucose level value within a predetermined time period for the received data indicative of the glucose level of the subject,
determine an estimated meal start time and an initial glucose level value based on the peak glucose level value,
determine a glucose level variance value,
prompt the subject to enter meal information, and
associate the entered meal information with the glucose level variance value.

60. The system of claim 59, wherein the reader device comprises a smart phone.

61-62. (canceled)

63. The system of claim 59, further comprising a trusted computer system, wherein the trusted computer system is a cloud-computing platform comprising one or more servers, wherein the trusted computer system is configured to transmit the data indicative of the glucose level of the subject to the reader device.

64. The system of claim 59, further comprising a sensor control device, wherein the sensor control device comprises a glucose sensor, and wherein at least a portion of the glucose sensor is configured to be positioned under a skin layer of the subject and in contact with a bodily fluid of the subject.

65. (canceled)

66. The system of claim 59, wherein the wireless communication circuitry of the reader device is configured to receive the data indicative of the glucose level of the subject according to a Bluetooth or a Near Field Communication wireless protocol.

67. The system of claim 59, wherein the peak glucose level value comprises a highest glucose value over a predetermined glucose level threshold.

68. The system of claim 67, wherein the predetermined glucose level threshold is 170 mg/dL.

69. The system of claim 67, wherein the predetermined glucose level threshold is 180 mg/dL.

70. The system of claim 67, wherein the predetermined glucose level threshold is 190 mg/dL.

71. The system of claim 59, wherein the predetermined time period for the received data indicative of the glucose level of the subject comprises a last two hours of glucose data.

72. The system of claim 59, wherein the predetermined time period for the received data indicative of the glucose level of the subject comprises a last four hours of glucose data.

73. The system of claim 59, wherein the predetermined time period for the received data indicative of the glucose level of the subject comprises a last eight hours of glucose data.

74. The system of claim 59, wherein the estimated meal start time is determined by counting two hours back from a time of the peak analyte level value.

75. The system of claim 59, wherein the estimated meal start time is determined by counting three hours back from a time of the peak glucose level value.

76. The system of claim 59, wherein the estimated meal start time is determined by counting four hours back from a time of the peak glucose level value.

77. The system of claim 59, wherein the glucose level variance value is determined by subtracting the initial glucose level value from the peak glucose level value.

78. The system of claim 59, wherein the meal monitoring application, when executed by the one or more processors, further causes the one or more processors to store the meal information and associated glucose level variance value in the memory of the reader device.

79-124. (canceled)
